SEQUENCE LISTING (91 sequences)

SEQ ID NO: 1 - DNA, 10697 bp, synthetic construct
  misc_feature 1..10697: note = Synthetic polynucleotide
  source 1..10697: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 2 - DNA, 11355 bp, synthetic construct
  misc_feature 1..11355: note = Synthetic polynucleotide
  source 1..11355: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacagtccggaaagccaccatgggccgctgctgcttctacaccgccggcaccctgagcctgctgctgctggtgaccagcgtgaccctgctggtggcccgcgtgttccagaaggccgtggaccagagcatcgagaagaagatcgtgctgcgcaacggcaccgaggccttcgacagctgggagaagccccccctgcccgtgtacacccagttctacttcttcaacgtgaccaaccccgaggagatcctgcgcggcgagaccccccgcgtggaggaggtgggcccctacacctaccgcgagctgcgcaacaaggccaacatccagttcggcgacaacggcaccaccatcagcgccgtgagcaacaaggcctacgtgttcgagcgcgaccagagcgtgggcgaccccaagatcgacctgatccgcaccctgaacatccccgtgctgaccgtgatcgagtggagccaggtgcacttcctgcgcgagatcatcgaggccatgctgaaggcctaccagcagaagctgttcgtgacccacaccgtggacgagctgctgtggggctacaaggacgagatcctgagcctgatccacgtgttccgccccgacatcagcccctacttcggcctgttctacgagaagaacggcaccaacgacggcgactacgtgttcctgaccggcgaggacagctacctgaacttcaccaagatcgtggagtggaacggcaagaccagcctggactggtggatcaccgacaagtgcaacatgatcaacggcaccgacggcgacagcttccaccccctgatcaccaaggacgaggtgctgtacgtgttccccagcgacttctgccgcagcgtgtacatcaccttcagcgactacgagagcgtgcagggcctgcccgccttccgctacaaggtgcccgccgagatcctggccaacaccagcgacaacgccggcttctgcatccccgagggcaactgcctgggcagcggcgtgctgaacgtgagcatctgcaagaacggcgcccccatcatcatgagcttcccccacttctaccaggccgacgagcgcttcgtgagcgccatcgagggcatgcaccccaaccaggaggaccacgagaccttcgtggacatcaaccccctgaccggcatcatcctgaaggccgccaagcgcttccagatcaacatctacgtgaagaagctggacgacttcgtggagaccggcgacatccgcaccatggtgttccccgtgatgtacctgaacgagagcgtgcacatcgacaaggagaccgccagccgcctgaagagcatgatcaacaccaccctgatcatcaccaacatcccctacatcatcatggccctgggcgtgttcttcggcctggtgttcacctggctggcctgcaagggccagggcagcatggacgagggcaccgccgacgagcgcgcccccctgatccgcacctgattgtggccgaaccgccgaactcagaggccggccccagaaaacccgagcgagtagggggcggcgcgcaggagggaggagaactgggggcgcgggaggctggtgggtgtggggggtggagatgtagaagatgtgacgccgcggcccggcgggtgccagattagcggacgcggtgcccgcggttgcaacgggatcccgggcgctgcagcttgggaggcggctctccccaggcggcgtccgcggagacacccatccgtgaaccccaggtcccgggccgccggctcgccgcgcaccaggggccggcggacagaagagcggccgagcggctcgaggctgggggaccgcgggcgcggccgcgcgctgccgggcgggaggctggggggccggggccggggccgtgccccggagcgggtcggaggccggggccggggccgggggacggcggctccccgcgcggctccagcggctcggggatcccggccgggccccgcagggaccatgatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 3 - DNA, 11420 bp, synthetic construct
  misc_feature 1..11420: note = Synthetic polynucleotide
  source 1..11420: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacagtccggaaagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttgggggggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacagtccggaaagccaccatgggccgctgctgcttctacaccgccggcaccctgagcctgctgctgctggtgaccagcgtgaccctgctggtggcccgcgtgttccagaaggccgtggaccagagcatcgagaagaagatcgtgctgcgcaacggcaccgaggccttcgacagctgggagaagccccccctgcccgtgtacacccagttctacttcttcaacgtgaccaaccccgaggagatcctgcgcggcgagaccccccgcgtggaggaggtgggcccctacacctaccgcgagctgcgcaacaaggccaacatccagttcggcgacaacggcaccaccatcagcgccgtgagcaacaaggcctacgtgttcgagcgcgaccagagcgtgggcgaccccaagatcgacctgatccgcaccctgaacatccccgtgctgaccgtgatcgagtggagccaggtgcacttcctgcgcgagatcatcgaggccatgctgaaggcctaccagcagaagctgttcgtgacccacaccgtggacgagctgctgtggggctacaaggacgagatcctgagcctgatccacgtgttccgccccgacatcagcccctacttcggcctgttctacgagaagaacggcaccaacgacggcgactacgtgttcctgaccggcgaggacagctacctgaacttcaccaagatcgtggagtggaacggcaagaccagcctggactggtggatcaccgacaagtgcaacatgatcaacggcaccgacggcgacagcttccaccccctgatcaccaaggacgaggtgctgtacgtgttccccagcgacttctgccgcagcgtgtacatcaccttcagcgactacgagagcgtgcagggcctgcccgccttccgctacaaggtgcccgccgagatcctggccaacaccagcgacaacgccggcttctgcatccccgagggcaactgcctgggcagcggcgtgctgaacgtgagcatctgcaagaacggcgcccccatcatcatgagcttcccccacttctaccaggccgacgagcgcttcgtgagcgccatcgagggcatgcaccccaaccaggaggaccacgagaccttcgtggacatcaaccccctgaccggcatcatcctgaaggccgccaagcgcttccagatcaacatctacgtgaagaagctggacgacttcgtggagaccggcgacatccgcaccatggtgttccccgtgatgtacctgaacgagagcgtgcacatcgacaaggagaccgccagccgcctgaagagcatgatcaacaccaccctgatcatcaccaacatcccctacatcatcatggccctgggcgtgttcttcggcctggtgttcacctggctggcctgcaagggccagggcagcatggacgagggcaccgccgacgagcgcgcccccctgatccgcacctgacccaggggactcaatcagcctcgaagacatgataagatacattgatgagtttggacaaaccacaacaagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtatgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 4 - DNA, 11171 bp, synthetic construct
  misc_feature 1..11171: note = Synthetic polynucleotide
  source 1..11171: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctcctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccaccgcagccctaggaatgcatctagacaattgtactaaccttcttctctttcctctcctgacagtccggaaagccaccatgggccgctgctgcttctacaccgccggcaccctgagcctgctgctgctggtgaccagcgtgaccctgctggtggcccgcgtgttccagaaggccgtggaccagagcatcgagaagaagatcgtgctgcgcaacggcaccgaggccttcgacagctgggagaagccccccctgcccgtgtacacccagttctacttcttcaacgtgaccaaccccgaggagatcctgcgcggcgagaccccccgcgtggaggaggtgggcccctacacctaccgcgagctgcgcaacaaggccaacatccagttcggcgacaacggcaccaccatcagcgccgtgagcaacaaggcctacgtgttcgagcgcgaccagagcgtgggcgaccccaagatcgacctgatccgcaccctgaacatccccgtgctgaccgtgatcgagtggagccaggtgcacttcctgcgcgagatcatcgaggccatgctgaaggcctaccagcagaagctgttcgtgacccacaccgtggacgagctgctgtggggctacaaggacgagatcctgagcctgatccacgtgttccgccccgacatcagcccctacttcggcctgttctacgagaagaacggcaccaacgacggcgactacgtgttcctgaccggcgaggacagctacctgaacttcaccaagatcgtggagtggaacggcaagaccagcctggactggtggatcaccgacaagtgcaacatgatcaacggcaccgacggcgacagcttccaccccctgatcaccaaggacgaggtgctgtacgtgttccccagcgacttctgccgcagcgtgtacatcaccttcagcgactacgagagcgtgcagggcctgcccgccttccgctacaaggtgcccgccgagatcctggccaacaccagcgacaacgccggcttctgcatccccgagggcaactgcctgggcagcggcgtgctgaacgtgagcatctgcaagaacggcgcccccatcatcatgagcttcccccacttctaccaggccgacgagcgcttcgtgagcgccatcgagggcatgcaccccaaccaggaggaccacgagaccttcgtggacatcaaccccctgaccggcatcatcctgaaggccgccaagcgcttccagatcaacatctacgtgaagaagctggacgacttcgtggagaccggcgacatccgcaccatggtgttccccgtgatgtacctgaacgagagcgtgcacatcgacaaggagaccgccagccgcctgaagagcatgatcaacaccaccctgatcatcaccaacatcccctacatcatcatggccctgggcgtgttcttcggcctggtgttcacctggctggcctgcaagggccagggcagcatggacgagggcaccgccgacgagcgcgcccccctgatccgcaccgagggcagaggaagtcttctgacatgcggagacgtggaagagaatcccggccctatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 5 - DNA, 11309 bp, synthetic construct
  misc_feature 1..11309: note = Synthetic polynucleotide
  source 1..11309: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctcctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccaccgcagccctaggaatgcatctagacaattgtactaaccttcttctctttcctctcctgacagtccggaaagccaccatgtacgccctgttcctgctggccagcctgctgggcgccgccctggccggccccgtgctgggcctgaaggagtgcacccgcggcagcgccgtgtggtgccagaacgtgaagaccgccagcgactgcggcgccgtgaagcactgcctgcagaccgtgtggaacaagcccaccgtgaagagcctgccctgcgacatctgcaaggacgtggtgaccgccgccggcgacatgctgaaggacaacgccaccgaggaggagatcctggtgtacctggagaagacctgcgactggctgcccaagcccaacatgagcgccagctgcaaggagatcgtggacagctacctgcccgtgatcctggacatcatcaagggcgagatgagccgccccggcgaggtgtgcagcgccctgaacctgtgcgagagcctgcagaagcacctggccgagctgaaccaccagaagcagctggagagcaacaagatccccgagctggacatgaccgaggtggtggcccccttcatggccaacatccccctgctgctgtacccccaggacggcccccgcagcaagccccagcccaaggacaacggcgacgtgtgccaggactgcatccagatggtgaccgacatccagaccgccgtgcgcaccaacagcaccttcgtgcaggccctggtggagcacgtgaaggaggagtgcgaccgcctgggccccggcatggccgacatctgcaagaactacatcagccagtacagcgagatcgccatccagatgatgatgcacatgcagcccaaggagatctgcgccctggtgggcttctgcgacgaggtgaaggagatgcccatgcagaccctggtgcccgccaaggtggccagcaagaacgtgatccccgccctggagctggtggagcccatcaagaagcacgaggtgcccgccaagagcgacgtgtactgcgaggtgtgcgagttcctggtgaaggaggtgaccaagctgatcgacaacaacaagaccgagaaggagatcctggacgccttcgacaagatgtgcagcaagctgcccaagagcctgagcgaggagtgccaggaggtggtggacacctacggcagcagcatcctgagcatcctgctggaggaggtgagccccgagctggtgtgcagcatgctgcacctgtgcagcggcacccgcctgcccgccctgaccgtgcacgtgacccagcccaaggacggcggcttctgcgaggtgtgcaagaagctggtgggctacctggaccgcaacctggagaagaacagcaccaagcaggagatcctggccgccctggagaagggctgcagcttcctgcccgacccctaccagaagcagtgcgaccagttcgtggccgagtacgagcccgtgctgatcgagatcctggtggaggtgatggaccccagcttcgtgtgcctgaagatcggcgcctgccccagcgcccacaagcccctgctgggcaccgagaagtgcatctggggccccagctactggtgccagaacaccgagaccgccgcccagtgcaacgccgtggagcactgcaagcgccacgtgtggaacgagggcagaggaagtcttctgacatgcggagacgtggaagagaatcccggccctatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 6 - DNA, 11293 bp, synthetic construct
  misc_feature 1..11293: note = Synthetic polynucleotide
  source 1..11293: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacagtccggaaagccaccatgtacgccctgttcctgctggccagcctgctgggcgccgccctggccggccccgtgctgggcctgaaggagtgcacccgcggcagcgccgtgtggtgccagaacgtgaagaccgccagcgactgcggcgccgtgaagcactgcctgcagaccgtgtggaacaagcccaccgtgaagagcctgccctgcgacatctgcaaggacgtggtgaccgccgccggcgacatgctgaaggacaacgccaccgaggaggagatcctggtgtacctggagaagacctgcgactggctgcccaagcccaacatgagcgccagctgcaaggagatcgtggacagctacctgcccgtgatcctggacatcatcaagggcgagatgagccgccccggcgaggtgtgcagcgccctgaacctgtgcgagagcctgcagaagcacctggccgagctgaaccaccagaagcagctggagagcaacaagatccccgagctggacatgaccgaggtggtggcccccttcatggccaacatccccctgctgctgtacccccaggacggcccccgcagcaagccccagcccaaggacaacggcgacgtgtgccaggactgcatccagatggtgaccgacatccagaccgccgtgcgcaccaacagcaccttcgtgcaggccctggtggagcacgtgaaggaggagtgcgaccgcctgggccccggcatggccgacatctgcaagaactacatcagccagtacagcgagatcgccatccagatgatgatgcacatgcagcccaaggagatctgcgccctggtgggcttctgcgacgaggtgaaggagatgcccatgcagaccctggtgcccgccaaggtggccagcaagaacgtgatccccgccctggagctggtggagcccatcaagaagcacgaggtgcccgccaagagcgacgtgtactgcgaggtgtgcgagttcctggtgaaggaggtgaccaagctgatcgacaacaacaagaccgagaaggagatcctggacgccttcgacaagatgtgcagcaagctgcccaagagcctgagcgaggagtgccaggaggtggtggacacctacggcagcagcatcctgagcatcctgctggaggaggtgagccccgagctggtgtgcagcatgctgcacctgtgcagcggcacccgcctgcccgccctgaccgtgcacgtgacccagcccaaggacggcggcttctgcgaggtgtgcaagaagctggtgggctacctggaccgcaacctggagaagaacagcaccaagcaggagatcctggccgccctggagaagggctgcagcttcctgcccgacccctaccagaagcagtgcgaccagttcgtggccgagtacgagcccgtgctgatcgagatcctggtggaggtgatggaccccagcttcgtgtgcctgaagatcggcgcctgccccagcgcccacaagcccctgctgggcaccgagaagtgcatctggggccccagctactggtgccagaacaccgagaccgccgcccagtgcaacgccgtggagcactgcaagcgccacgtgtggaactgattgtggccgaaccgccgaactcagaggccggccccagaaaacccgagcgagtagggggcggcgcgcaggagggaggagaactgggggcgcgggaggctggtgggtgtggggggtggagatgtagaagatgtgacgccgcggcccggcgggtgccagattagcggacgcggtgcccgcggttgcaacgggatcccgggcgctgcagcttgggaggcggctctccccaggcggcgtccgcggagacacccatccgtgaaccccaggtcccgggccgccggctcgccgcgcaccaggggccggcggacagaagagcggccgagcggctcgaggctgggggaccgcgggcgcggccgcgcgctgccgggcgggaggctggggggccggggccggggccgtgccccggagcgggtcggaggccggggccggggccgggggacggcggctccccgcgcggctccagcggctcggggatcccggccgggccccgcagggaccatgatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcaataaaatatctttattttcattacatctgtgtgttggttttttgtgtggagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 7 - DNA, 10700 bp, synthetic construct
  misc_feature 1..10700: note = Synthetic polynucleotide
  source 1..10700: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 8 - DNA, 10700 bp, synthetic construct
  misc_feature 1..10700: note = Synthetic polynucleotide
  source 1..10700: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactattagatctgatggccgcgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 9 - DNA, 10700 bp, synthetic construct
  misc_feature 1..10700: note = Synthetic polynucleotide
  source 1..10700: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 10 - DNA, 10700 bp, synthetic construct
  misc_feature 1..10700: note = Synthetic polynucleotide
  source 1..10700: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaagcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 11 - DNA, 11188 bp, synthetic construct
  misc_feature 1..11188: note = Synthetic polynucleotide
  source 1..11188: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactattagatctgatggccgcgctagctctgggtatttaagcccgagtgagcacgcagggtctccattttgaagcgggaggttacgcgttcgtcgactactagtgggtaccagagcgtggtgactgagatgttttctaggaaacacaaaagatacaaaaaagaacacgtggaaggatagccaaaaaggggggctgcccccatttcctgcaccccgctgcgatggctggcaccatttggaagacttcgagatacactgttgagcgcagtaagacaacagtgtatctcgaagtcttccagatggggccagccggtccactctgtatccaggccagttctgcaaggcgttcgaggaccacccccctcccctcgccaccagggtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgtttcccatggtgaatccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 12 - DNA, 11187 bp, synthetic construct
  misc_feature 1..11187: note = Synthetic polynucleotide
  source 1..11187: mol_type = other DNA
ctagaaccggtgacgtctcccatggtgaagcttggatctgaattcggtacctagttataatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtcttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagcgtggtgactgagatgttttctaggaaacacaaaagatacaaaaaagaacacgtggaaggatagccaaaaaggggggctgcccccatttcctgcaccccgctgcgatggctggcaccatttggaagacttcgagatacactgttgagcgcagtaagacaacagtgtatctcgaagtcttccagatggggccagccggtccactctgtatccaggccagttctgcaaggcgttcgaggaccacccccctcccctcgccaccagggtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgtttcccatggtgaatccctaggtt

SEQ ID NO: 13 - DNA, 10960 bp, synthetic construct
  misc_feature 1..10960: note = Synthetic polynucleotide
  source 1..10960: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtcctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccacctttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 14 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 15 - DNA, 1608 bp, Homo sapiens
  source 1..1608: mol_type = other DNA
atggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacag

SEQ ID NO: 16 - protein, 524 aa, Homo sapiens
MYALFLLASLLGAALAGPVLGLKECTRGSAVWCQNVKTASDCGAVKHCLQTVWNKPTVKSLPCDICKDVVTAAGDMLKDNATEEEILVYLEKTCDWLPKPNMSASCKEIVDSYLPVILDIIKGEMSRPGEVCSALNLCESLQKHLAELNHQKQLESNKIPELDMTEVVAPFMANIPLLLYPQDGPRSKPQPKDNGDVCQDCIQMVTDIQTAVRTNSTFVQALVEHVKEECDRLGPGMADICKNYISQYSEIAIQMMMHMQPKEICALVGFCDEVKEMPMQTLVPAKVASKNVIPALELVEPIKKHEVPAKSDVYCEVCEFLVKEVTKLIDNNKTEKEILDAFDKMCSKLPKSLSEECQEVVDTYGSSILSILLEEVSPELVCSMLHLCSGTRLPALTVHVTQPKDGGFCEVCKKLVGYLDRNLEKNSTKQEILAALEKGCSFLPDPYQKQCDQFVAEYEPVLIEILVEVMDPSFVCLKIGACPSAHKPLLGTEKCIWGPSYWCQNTETAAQCNAVEHCKRHVWN

SEQ ID NO: 17 - DNA, 1572 bp, Homo sapiens
  source 1..1572: mol_type = other DNA
atgtacgccctgttcctgctggccagcctgctgggcgccgccctggccggccccgtgctgggcctgaaggagtgcacccgcggcagcgccgtgtggtgccagaacgtgaagaccgccagcgactgcggcgccgtgaagcactgcctgcagaccgtgtggaacaagcccaccgtgaagagcctgccctgcgacatctgcaaggacgtggtgaccgccgccggcgacatgctgaaggacaacgccaccgaggaggagatcctggtgtacctggagaagacctgcgactggctgcccaagcccaacatgagcgccagctgcaaggagatcgtggacagctacctgcccgtgatcctggacatcatcaagggcgagatgagccgccccggcgaggtgtgcagcgccctgaacctgtgcgagagcctgcagaagcacctggccgagctgaaccaccagaagcagctggagagcaacaagatccccgagctggacatgaccgaggtggtggcccccttcatggccaacatccccctgctgctgtacccccaggacggcccccgcagcaagccccagcccaaggacaacggcgacgtgtgccaggactgcatccagatggtgaccgacatccagaccgccgtgcgcaccaacagcaccttcgtgcaggccctggtggagcacgtgaaggaggagtgcgaccgcctgggccccggcatggccgacatctgcaagaactacatcagccagtacagcgagatcgccatccagatgatgatgcacatgcagcccaaggagatctgcgccctggtgggcttctgcgacgaggtgaaggagatgcccatgcagaccctggtgcccgccaaggtggccagcaagaacgtgatccccgccctggagctggtggagcccatcaagaagcacgaggtgcccgccaagagcgacgtgtactgcgaggtgtgcgagttcctggtgaaggaggtgaccaagctgatcgacaacaacaagaccgagaaggagatcctggacgccttcgacaagatgtgcagcaagctgcccaagagcctgagcgaggagtgccaggaggtggtggacacctacggcagcagcatcctgagcatcctgctggaggaggtgagccccgagctggtgtgcagcatgctgcacctgtgcagcggcacccgcctgcccgccctgaccgtgcacgtgacccagcccaaggacggcggcttctgcgaggtgtgcaagaagctggtgggctacctggaccgcaacctggagaagaacagcaccaagcaggagatcctggccgccctggagaagggctgcagcttcctgcccgacccctaccagaagcagtgcgaccagttcgtggccgagtacgagcccgtgctgatcgagatcctggtggaggtgatggaccccagcttcgtgtgcctgaagatcggcgcctgccccagcgcccacaagcccctgctgggcaccgagaagtgcatctggggccccagctactggtgccagaacaccgagaccgccgcccagtgcaacgccgtggagcactgcaagcgccacgtgtggaac

SEQ ID NO: 18 - protein, 478 aa, Homo sapiens
MGRCCFYTAGTLSLLLLVTSVTLLVARVFQKAVDQSIEKKIVLRNGTEAFDSWEKPPLPVYTQFYFFNVTNPEEILRGETPRVEEVGPYTYRELRNKANIQFGDNGTTISAVSNKAYVFERDQSVGDPKIDLIRTLNIPVLTVIEWSQVHFLREIIEAMLKAYQQKLFVTHTVDELLWGYKDEILSLIHVFRPDISPYFGLFYEKNGTNDGDYVFLTGEDSYLNFTKIVEWNGKTSLDWWITDKCNMINGTDGDSFHPLITKDEVLYVFPSDFCRSVYITFSDYESVQGLPAFRYKVPAEILANTSDNAGFCIPEGNCLGSGVLNVSICKNGAPIIMSFPHFYQADERFVSAIEGMHPNQEDHETFVDINPLTGIILKAAKRFQINIYVKKLDDFVETGDIRTMVFPVMYLNESVHIDKETASRLKSMINTTLIITNIPYIIMALGVFFGLVFTWLACKGQGSMDEGTADERAPLIRT

SEQ ID NO: 19 - DNA, 1434 bp, Homo sapiens
  source 1..1434: mol_type = other DNA
atgggccgctgctgcttctacaccgccggcaccctgagcctgctgctgctggtgaccagcgtgaccctgctggtggcccgcgtgttccagaaggccgtggaccagagcatcgagaagaagatcgtgctgcgcaacggcaccgaggccttcgacagctgggagaagccccccctgcccgtgtacacccagttctacttcttcaacgtgaccaaccccgaggagatcctgcgcggcgagaccccccgcgtggaggaggtgggcccctacacctaccgcgagctgcgcaacaaggccaacatccagttcggcgacaacggcaccaccatcagcgccgtgagcaacaaggcctacgtgttcgagcgcgaccagagcgtgggcgaccccaagatcgacctgatccgcaccctgaacatccccgtgctgaccgtgatcgagtggagccaggtgcacttcctgcgcgagatcatcgaggccatgctgaaggcctaccagcagaagctgttcgtgacccacaccgtggacgagctgctgtggggctacaaggacgagatcctgagcctgatccacgtgttccgccccgacatcagcccctacttcggcctgttctacgagaagaacggcaccaacgacggcgactacgtgttcctgaccggcgaggacagctacctgaacttcaccaagatcgtggagtggaacggcaagaccagcctggactggtggatcaccgacaagtgcaacatgatcaacggcaccgacggcgacagcttccaccccctgatcaccaaggacgaggtgctgtacgtgttccccagcgacttctgccgcagcgtgtacatcaccttcagcgactacgagagcgtgcagggcctgcccgccttccgctacaaggtgcccgccgagatcctggccaacaccagcgacaacgccggcttctgcatccccgagggcaactgcctgggcagcggcgtgctgaacgtgagcatctgcaagaacggcgcccccatcatcatgagcttcccccacttctaccaggccgacgagcgcttcgtgagcgccatcgagggcatgcaccccaaccaggaggaccacgagaccttcgtggacatcaaccccctgaccggcatcatcctgaaggccgccaagcgcttccagatcaacatctacgtgaagaagctggacgacttcgtggagaccggcgacatccgcaccatggtgttccccgtgatgtacctgaacgagagcgtgcacatcgacaaggagaccgccagccgcctgaagagcatgatcaacaccaccctgatcatcaccaacatcccctacatcatcatggccctgggcgtgttcttcggcctggtgttcacctggctggcctgcaagggccagggcagcatggacgagggcaccgccgacgagcgcgcccccctgatccgcacc

SEQ ID NO: 20 - DNA, 23 bp, synthetic construct
  misc_feature 1..23: note = Synthetic polynucleotide
  source 1..23: mol_type = other DNA
tggaagacttcgagatacactgt

SEQ ID NO: 21 - DNA, 23 bp, synthetic construct
  misc_feature 1..23: note = Synthetic polynucleotide
  source 1..23: mol_type = other DNA
acagtgtatctcgaagtcttcca

SEQ ID NO: 22 - DNA, 21 bp, synthetic construct
  misc_feature 1..21: note = Synthetic polynucleotide
  source 1..21: mol_type = other DNA
tttagaaataagtggtagtca

SEQ ID NO: 23 - DNA, 21 bp, synthetic construct
  misc_feature 1..21: note = Synthetic polynucleotide
  source 1..21: mol_type = other DNA
tgactaccacttatttctaaa

SEQ ID NO: 24 - DNA, 19 bp, synthetic construct
  misc_feature 1..19: note = Synthetic polynucleotide
  source 1..19: mol_type = other DNA
agggtatcaagactacgaa

SEQ ID NO: 25 - DNA, 19 bp, synthetic construct
  misc_feature 1..19: note = Synthetic polynucleotide
  source 1..19: mol_type = other DNA
ttcgtagtcttgataccct

SEQ ID NO: 26 - DNA, 19 bp, synthetic construct
  misc_feature 1..19: note = Synthetic polynucleotide
  source 1..19: mol_type = other DNA
tattagatctgatggccgc

SEQ ID NO: 27 - DNA, 20 bp, synthetic construct
  misc_feature 1..20: note = Synthetic polynucleotide
  source 1..20: mol_type = other DNA
ctccatcactaggggttcct

SEQ ID NO: 28 - DNA, 60 bp, synthetic construct
  misc_feature 1..60: note = Synthetic polynucleotide
  source 1..60: mol_type = other DNA
agctctgggtatttaagcccgagtgagcacgcagggtctccattttgaagcgggaggtta

SEQ ID NO: 29 - DNA, 145 bp, unidentified
  misc_feature 1..145: note = AAV2 ITR
  source 1..145: mol_type = genomic DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 30 - protein, 927 aa, Homo sapiens
MGTQDPGNMGTGVPASEQISCAKEDPQVYCPEETGGTKDVQVTDCKSPEDSRPPKETDCCNPEDSGQLMVSYEGKAMGYQVPPFGWRICLAHEFTEKRKPFQANNVSLSNMIKHIGMGLRYLQWWYRKTHVEKKTPFIDMINSVPLRQIYGCPLGGIGGGTITRGWRGQFCRWQLNPGMYQHRTVIADQFTVCLRREGQTVYQQVLSLERPSVLRSWNWGLCGYFAFYHALYPRAWTVYQLPGQNVTLTCRQITPILPHDYQDSSLPVGVFVWDVENEGDEALDVSIMFSMRNGLGGGDDAPGGLWNEPFCLERSGETVRGLLLHHPTLPNPYTMAVAARVTAATTVTHITAFDPDSTGQQVWQDLLQDGQLDSPTGQSTPTQKGVGIAGAVCVSSKLRPRGQCRLEFSLAWDMPRIMFGAKGQVHYRRYTRFFGQDGDAAPALSHYALCRYAEWEERISAWQSPVLDDRSLPAWYKSALFNELYFLADGGTVWLEVLEDSLPEELGRNMCHLRPTLRDYGRFGYLEGQEYRMYNTYDVHFYASFALIMLWPKLELSLQYDMALATLREDLTRRRYLMSGVMAPVKRRNVIPHDIGDPDDEPWLRVNAYLIHDTADWKDLNLKFVLQVYRDYYLTGDQNFLKDMWPVCLAVMESEMKFDKDHDGLIENGGYADQTYDGWVTTGPSAYCGGLWLAAVAVMVQMAALCGAQDIQDKFSSILSRGQEAYERLLWNGRYYNYDSSSRPQSRSVMSDQCAGQWFLKACGLGEGDTEVFPTQHVVRALQTIFELNVQAFAGGAMGAVNGMQPHGVPDKSSVQSDEVWVGVVYGLAATMIQEGLTWEGFQTAEGCYRTVWERLGLAFQTPEAYCQQRVFRSLAYMRPLSIWAMQLALQQQQHKKASWPKVKQGTGLRTGPMFGPKEAMANLSPE

SEQ ID NO: 31 - DNA, 2781 bp, Homo sapiens
  source 1..2781: mol_type = other DNA
atgggcacccaggaccccggcaacatgggcaccggcgtgcccgccagcgagcagatcagctgcgccaaggaggacccccaggtgtactgccccgaggagaccggcggcaccaaggacgtgcaggtgaccgactgcaagagccccgaggacagccgcccccccaaggagaccgactgctgcaaccccgaggacagcggccagctgatggtgagctacgagggcaaggccatgggctaccaggtgccccccttcggctggcgcatctgcctggcccacgagttcaccgagaagcgcaagcccttccaggccaacaacgtgagcctgagcaacatgatcaagcacatcggcatgggcctgcgctacctgcagtggtggtaccgcaagacccacgtggagaagaagacccccttcatcgacatgatcaacagcgtgcccctgcgccagatctacggctgccccctgggcggcatcggcggcggcaccatcacccgcggctggcgcggccagttctgccgctggcagctgaaccccggcatgtaccagcaccgcaccgtgatcgccgaccagttcaccgtgtgcctgcgccgcgagggccagaccgtgtaccagcaggtgctgagcctggagcgccccagcgtgctgcgcagctggaactggggcctgtgcggctacttcgccttctaccacgccctgtacccccgcgcctggaccgtgtaccagctgcccggccagaacgtgaccctgacctgccgccagatcacccccatcctgccccacgactaccaggacagcagcctgcccgtgggcgtgttcgtgtgggacgtggagaacgagggcgacgaggccctggacgtgagcatcatgttcagcatgcgcaacggcctgggcggcggcgacgacgcccccggcggcctgtggaacgagcccttctgcctggagcgcagcggcgagaccgtgcgcggcctgctgctgcaccaccccaccctgcccaacccctacaccatggccgtggccgcccgcgtgaccgccgccaccaccgtgacccacatcaccgccttcgaccccgacagcaccggccagcaggtgtggcaggacctgctgcaggacggccagctggacagccccaccggccagagcacccccacccagaagggcgtgggcatcgccggcgccgtgtgcgtgagcagcaagctgcgcccccgcggccagtgccgcctggagttcagcctggcctgggacatgccccgcatcatgttcggcgccaagggccaggtgcactaccgccgctacacccgcttcttcggccaggacggcgacgccgcccccgccctgagccactacgccctgtgccgctacgccgagtgggaggagcgcatcagcgcctggcagagccccgtgctggacgaccgcagcctgcccgcctggtacaagagcgccctgttcaacgagctgtacttcctggccgacggcggcaccgtgtggctggaggtgctggaggacagcctgcccgaggagctgggccgcaacatgtgccacctgcgccccaccctgcgcgactacggccgcttcggctacctggagggccaggagtaccgcatgtacaacacctacgacgtgcacttctacgccagcttcgccctgatcatgctgtggcccaagctggagctgagcctgcagtacgacatggccctggccaccctgcgcgaggacctgacccgccgccgctacctgatgagcggcgtgatggcccccgtgaagcgccgcaacgtgatcccccacgacatcggcgaccccgacgacgagccctggctgcgcgtgaacgcctacctgatccacgacaccgccgactggaaggacctgaacctgaagttcgtgctgcaggtgtaccgcgactactacctgaccggcgaccagaacttcctgaaggacatgtggcccgtgtgcctggccgtgatggagagcgagatgaagttcgacaaggaccacgacggcctgatcgagaacggcggctacgccgaccagacctacgacggctgggtgaccaccggccccagcgcctactgcggcggcctgtggctggccgccgtggccgtgatggtgcagatggccgccctgtgcggcgcccaggacatccaggacaagttcagcagcatcctgagccgcggccaggaggcctacgagcgcctgctgtggaacggccgctactacaactacgacagcagcagccgcccccagagccgcagcgtgatgagcgaccagtgcgccggccagtggttcctgaaggcctgcggcctgggcgagggcgacaccgaggtgttccccacccagcacgtggtgcgcgccctgcagaccatcttcgagctgaacgtgcaggccttcgccggcggcgccatgggcgccgtgaacggcatgcagccccacggcgtgcccgacaagagcagcgtgcagagcgacgaggtgtgggtgggcgtggtgtacggcctggccgccaccatgatccaggagggcctgacctgggagggcttccagaccgccgagggctgctaccgcaccgtgtgggagcgcctgggcctggccttccagacccccgaggcctactgccagcagcgcgtgttccgcagcctggcctacatgcgccccctgagcatctgggccatgcagctggccctgcagcagcagcagcacaagaaggccagctggcccaaggtgaagcagggcaccggcctgcgcaccggccccatgttcggccccaaggaggccatggccaacctgagccccgag

SEQ ID NO: 32 - DNA, 11264 bp, synthetic construct
  misc_feature 1..11264: note = Synthetic polynucleotide
  source 1..11264: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagtaagtcactgactgtctatgcctgggaaagggtgggcaggagatggggcagtgcaggaaaagtggcactatgaaccctcctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccaccgcagccctaggaatgcatctagacaattgtactaaccttcttctctttcctctcctgacagtccggaaagccaccatgggcacccaggaccccggcaacatgggcaccggcgtgcccgccagcgagcagatcagctgcgccaaggaggacccccaggtgtactgccccgaggagaccggcggcaccaaggacgtgcaggtgaccgactgcaagagccccgaggacagccgcccccccaaggagaccgactgctgcaaccccgaggacagcggccagctgatggtgagctacgagggcaaggccatgggctaccaggtgccccccttcggctggcgcatctgcctggcccacgagttcaccgagaagcgcaagcccttccaggccaacaacgtgagcctgagcaacatgatcaagcacatcggcatgggcctgcgctacctgcagtggtggtaccgcaagacccacgtggagaagaagacccccttcatcgacatgatcaacagcgtgcccctgcgccagatctacggctgccccctgggcggcatcggcggcggcaccatcacccgcggctggcgcggccagttctgccgctggcagctgaaccccggcatgtaccagcaccgcaccgtgatcgccgaccagttcaccgtgtgcctgcgccgcgagggccagaccgtgtaccagcaggtgctgagcctggagcgccccagcgtgctgcgcagctggaactggggcctgtgcggctacttcgccttctaccacgccctgtacccccgcgcctggaccgtgtaccagctgcccggccagaacgtgaccctgacctgccgccagatcacccccatcctgccccacgactaccaggacagcagcctgcccgtgggcgtgttcgtgtgggacgtggagaacgagggcgacgaggccctggacgtgagcatcatgttcagcatgcgcaacggcctgggcggcggcgacgacgcccccggcggcctgtggaacgagcccttctgcctggagcgcagcggcgagaccgtgcgcggcctgctgctgcaccaccccaccctgcccaacccctacaccatggccgtggccgcccgcgtgaccgccgccaccaccgtgacccacatcaccgccttcgaccccgacagcaccggccagcaggtgtggcaggacctgctgcaggacggccagctggacagccccaccggccagagcacccccacccagaagggcgtgggcatcgccggcgccgtgtgcgtgagcagcaagctgcgcccccgcggccagtgccgcctggagttcagcctggcctgggacatgccccgcatcatgttcggcgccaagggccaggtgcactaccgccgctacacccgcttcttcggccaggacggcgacgccgcccccgccctgagccactacgccctgtgccgctacgccgagtgggaggagcgcatcagcgcctggcagagccccgtgctggacgaccgcagcctgcccgcctggtacaagagcgccctgttcaacgagctgtacttcctggccgacggcggcaccgtgtggctggaggtgctggaggacagcctgcccgaggagctgggccgcaacatgtgccacctgcgccccaccctgcgcgactacggccgcttcggctacctggagggccaggagtaccgcatgtacaacacctacgacgtgcacttctacgccagcttcgccctgatcatgctgtggcccaagctggagctgagcctgcagtacgacatggccctggccaccctgcgcgaggacctgacccgccgccgctacctgatgagcggcgtgatggcccccgtgaagcgccgcaacgtgatcccccacgacatcggcgaccccgacgacgagccctggctgcgcgtgaacgcctacctgatccacgacaccgccgactggaaggacctgaacctgaagttcgtgctgcaggtgtaccgcgactactacctgaccggcgaccagaacttcctgaaggacatgtggcccgtgtgcctggccgtgatggagagcgagatgaagttcgacaaggaccacgacggcctgatcgagaacggcggctacgccgaccagacctacgacggctgggtgaccaccggccccagcgcctactgcggcggcctgtggctggccgccgtggccgtgatggtgcagatggccgccctgtgcggcgcccaggacatccaggacaagttcagcagcatcctgagccgcggccaggaggcctacgagcgcctgctgtggaacggccgctactacaactacgacagcagcagccgcccccagagccgcagcgtgatgagcgaccagtgcgccggccagtggttcctgaaggcctgcggcctgggcgagggcgacaccgaggtgttccccacccagcacgtggtgcgcgccctgcagaccatcttcgagctgaacgtgcaggccttcgccggcggcgccatgggcgccgtgaacggcatgcagccccacggcgtgcccgacaagagcagcgtgcagagcgacgaggtgtgggtgggcgtggtgtacggcctggccgccaccatgatccaggagggcctgacctgggagggcttccagaccgccgagggctgctaccgcaccgtgtgggagcgcctgggcctggccttccagacccccgaggcctactgccagcagcgcgtgttccgcagcctggcctacatgcgccccctgagcatctgggccatgcagctggccctgcagcagcagcagcacaagaaggccagctggcccaaggtgaagcagggcaccggcctgcgcaccggccccatgttcggccccaaggaggccatggccaacctgagccccgagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 33 - protein, 685 aa, synthetic construct
  REGION 1..685: note = Synthetic polypeptide
MAEWLLSASWQRRAKAMTAAAGSAGRAAVPLLLCALLAPGGAYVLDDSDGLGREFDGIGAVSGGGATSRLLVNYPEPYRSQILDYLFKPNFGASLHILKVEIGGDGQTTDGTEPSHMHYALDENYFRGYEWWLMKEAKKRNPNITLIGLPWSFPGWLGKGFDWPYVNLQLTAYYVVTWIVGAKRYHDLDIDYIGIWNERSYNANYIKILRKMLNYQGLQRVKIIASDNLWESISASMLLDAELFKVVDVIGAHYPGTHSAKDAKLTGKKLWSSEDFSTLNSDMGAGCWGRILNQNYINGYMTSTIAWNLVASYYEQLPYGRCGLMTAQEPWSGHYVVESPVWVSAHTTQFTQPGWYYLKTVGHLEKGGSYVALTDGLGNLTIIIETMSHKHSKCIRPFLPYFNVSQQFATFVLKGSFSEIPELQVWYTKLGKTSERFLFKQLDSLWLLDSDGSFTLSLHEDELFTLTTLTTGRKGSYPLPPKSQPFPSTYKDDFNVDYPFFSEAPNFADQTGVFEYFTNIEDPGEHHFTLRQVLNQRPITWAADASNTISIIGDYNWTNLTIKCDVYIETPDTGGVFIAGRVNKGGILIRSARGIFFWIFANGSYRVTGDLAGWIIYALGRVEVTAKKWYTLTLTIKGHFTSGMLNDKSLWTDIPVNFPKNGWAAIGTHSFEFAQFDNFLVEATR

SEQ ID NO: 34 - DNA, 2055 bp, synthetic construct
  misc_feature 1..2055: note = Synthetic polynucleotide
  source 1..2055: mol_type = other DNA
atggccgagtggctgctgagcgccagctggcagcgccgcgccaaggccatgaccgccgccgccggcagcgccggccgcgccgccgtgcccctgctgctgtgcgccctgctggcccccggcggcgcctacgtgctggacgacagcgacggcctgggccgcgagttcgacggcatcggcgccgtgagcggcggcggcgccaccagccgcctgctggtgaactaccccgagccctaccgcagccagatcctggactacctgttcaagcccaacttcggcgccagcctgcacatcctgaaggtggagatcggcggcgacggccagaccaccgacggcaccgagcccagccacatgcactacgccctggacgagaactacttccgcggctacgagtggtggctgatgaaggaggccaagaagcgcaaccccaacatcaccctgatcggcctgccctggagcttccccggctggctgggcaagggcttcgactggccctacgtgaacctgcagctgaccgcctactacgtggtgacctggatcgtgggcgccaagcgctaccacgacctggacatcgactacatcggcatctggaacgagcgcagctacaacgccaactacatcaagatcctgcgcaagatgctgaactaccagggcctgcagcgcgtgaagatcatcgccagcgacaacctgtgggagagcatcagcgccagcatgctgctggacgccgagctgttcaaggtggtggacgtgatcggcgcccactaccccggcacccacagcgccaaggacgccaagctgaccggcaagaagctgtggagcagcgaggacttcagcaccctgaacagcgacatgggcgccggctgctggggccgcatcctgaaccagaactacatcaacggctacatgaccagcaccatcgcctggaacctggtggccagctactacgagcagctgccctacggccgctgcggcctgatgaccgcccaggagccctggagcggccactacgtggtggagagccccgtgtgggtgagcgcccacaccacccagttcacccagcccggctggtactacctgaagaccgtgggccacctggagaagggcggcagctacgtggccctgaccgacggcctgggcaacctgaccatcatcatcgagaccatgagccacaagcacagcaagtgcatccgccccttcctgccctacttcaacgtgagccagcagttcgccaccttcgtgctgaagggcagcttcagcgagatccccgagctgcaggtgtggtacaccaagctgggcaagaccagcgagcgcttcctgttcaagcagctggacagcctgtggctgctggacagcgacggcagcttcaccctgagcctgcacgaggacgagctgttcaccctgaccaccctgaccaccggccgcaagggcagctaccccctgccccccaagagccagcccttccccagcacctacaaggacgacttcaacgtggactaccccttcttcagcgaggcccccaacttcgccgaccagaccggcgtgttcgagtacttcaccaacatcgaggaccccggcgagcaccacttcaccctgcgccaggtgctgaaccagcgccccatcacctgggccgccgacgccagcaacaccatcagcatcatcggcgactacaactggaccaacctgaccatcaagtgcgacgtgtacatcgagacccccgacaccggcggcgtgttcatcgccggccgcgtgaacaagggcggcatcctgatccgcagcgcccgcggcatcttcttctggatcttcgccaacggcagctaccgcgtgaccggcgacctggccggctggatcatctacgccctgggccgcgtggaggtgaccgccaagaagtggtacaccctgaccctgaccatcaagggccacttcaccagcggcatgctgaacgacaagagcctgtggaccgacatccccgtgaacttccccaagaacggctgggccgccatcggcacccacagcttcgagttcgcccagttcgacaacttcctggtggaggccacccgc

SEQ ID NO: 35 - protein, 339 aa, synthetic construct
  REGION 1..339: note = Synthetic polypeptide
MWQLWASLCCLLVLANARSRPSFHPLSDELVNYVNKRNTTWQAGHNFYNVDMSYLKRLCGTFLGGPKPPQRVMFTEDLKLPASFDAREQWPQCPTIKEIRDQGSCGSCWAFGAVEAISDRICIHTNAHVSVEVSAEDLLTCCGSMCGDGCNGGYPAEAWNFWTRKGLVSGGLYESHVGCRPYSIPPCEHHVNGSRPPCTGEGDTPKCSKICEPGYSPTYKQDKHYGYNSYSVSNSEKDIMAEIYKNGPVEGAFSVYSDFLLYKSGVYQHVTGEMMGGHAIRILGWGVENGTPYWLVANSWNTDWGDNGFFKILRGQDHCGIESEVVAGIPRTDQYWEKI

SEQ ID NO: 36 - DNA, 1017 bp, synthetic construct
  misc_feature 1..1017: note = Synthetic polynucleotide
  source 1..1017: mol_type = other DNA
atgtggcagctgtgggccagcctgtgctgcctgctggtgctggccaacgcccgcagccgccccagcttccaccccctgagcgacgagctggtgaactacgtgaacaagcgcaacaccacctggcaggccggccacaacttctacaacgtggacatgagctacctgaagcgcctgtgcggcaccttcctgggcggccccaagcccccccagcgcgtgatgttcaccgaggacctgaagctgcccgccagcttcgacgcccgcgagcagtggccccagtgccccaccatcaaggagatccgcgaccagggcagctgcggcagctgctgggccttcggcgccgtggaggccatcagcgaccgcatctgcatccacaccaacgcccacgtgagcgtggaggtgagcgccgaggacctgctgacctgctgcggcagcatgtgcggcgacggctgcaacggcggctaccccgccgaggcctggaacttctggacccgcaagggcctggtgagcggcggcctgtacgagagccacgtgggctgccgcccctacagcatccccccctgcgagcaccacgtgaacggcagccgccccccctgcaccggcgagggcgacacccccaagtgcagcaagatctgcgagcccggctacagccccacctacaagcaggacaagcactacggctacaacagctacagcgtgagcaacagcgagaaggacatcatggccgagatctacaagaacggccccgtggagggcgccttcagcgtgtacagcgacttcctgctgtacaagagcggcgtgtaccagcacgtgaccggcgagatgatgggcggccacgccatccgcatcctgggctggggcgtggagaacggcaccccctactggctggtggccaacagctggaacaccgactggggcgacaacggcttcttcaagatcctgcgcggccaggaccactgcggcatcgagagcgaggtggtggccggcatcccccgcaccgaccagtactgggagaagatc

SEQ ID NO: 37 - protein, 631 aa, synthetic construct
  REGION 1..631: note = Synthetic polypeptide
MPRYGASLRQSCPRSGREQGQDGTAGAPGLLWMGLVLALALALALALALSDSRVLWAPAEAHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 38 - DNA, 1896 bp, synthetic construct
  misc_feature 1..1896: note = Synthetic polynucleotide
  source 1..1896: mol_type = other DNA
atgccccgctacggcgccagcctgcgccagagctgcccccgcagcggccgcgagcagggccaggacggcaccgccggcgcccccggcctgctgtggatgggcctggtgctggccctggccctggccctggccctggccctggccctgagcgacagccgcgtgctgtgggcccccgccgaggcccaccccctgagcccccagggccaccccgcccgcctgcaccgcatcgtgccccgcctgcgcgacgtgttcggctggggcaacctgacctgccccatctgcaagggcctgttcaccgccatcaacctgggcctgaagaaggagcccaacgtggcccgcgtgggcagcgtggccatcaagctgtgcaacctgctgaagatcgccccccccgccgtgtgccagagcatcgtgcacctgttcgaggacgacatggtggaggtgtggcgccgcagcgtgctgagccccagcgaggcctgcggcctgctgctgggcagcacctgcggccactgggacatcttcagcagctggaacatcagcctgcccaccgtgcccaagcccccccccaagccccccagcccccccgcccccggcgcccccgtgagccgcatcctgttcctgaccgacctgcactgggaccacgactacctggagggcaccgaccccgactgcgccgaccccctgtgctgccgccgcggcagcggcctgccccccgccagccgccccggcgccggctactggggcgagtacagcaagtgcgacctgcccctgcgcaccctggagagcctgctgagcggcctgggccccgccggccccttcgacatggtgtactggaccggcgacatccccgcccacgacgtgtggcaccagacccgccaggaccagctgcgcgccctgaccaccgtgaccgccctggtgcgcaagttcctgggccccgtgcccgtgtaccccgccgtgggcaaccacgagagcacccccgtgaacagcttccccccccccttcatcgagggcaaccacagcagccgctggctgtacgaggccatggccaaggcctgggagccctggctgcccgccgaggccctgcgcaccctgcgcatcggcggcttctacgccctgagcccctaccccggcctgcgcctgatcagcctgaacatgaacttctgcagccgcgagaacttctggctgctgatcaacagcaccgaccccgccggccagctgcagtggctggtgggcgagctgcaggccgccgaggaccgcggcgacaaggtgcacatcatcggccacatcccccccggccactgcctgaagagctggagctggaactactaccgcatcgtggcccgctacgagaacaccctggccgcccagttcttcggccacacccacgtggacgagttcgaggtgttctacgacgaggagaccctgagccgccccctggccgtggccttcctggcccccagcgccaccacctacatcggcctgaaccccggctaccgcgtgtaccagatcgacggcaactacagcggcagcagccacgtggtgctggaccacgagacctacatcctgaacctgacccaggccaacatccccggcgccatcccccactggcagctgctgtaccgcgcccgcgagacctacggcctgcccaacaccctgcccaccgcctggcacaacctggtgtaccgcatgcgcggcgacatgcagctgttccagaccttctggttcctgtaccacaagggccacccccccagcgagccctgcggcaccccctgccgcctggccaccctgtgcgcccagctgagcgcccgcgccgacagccccgccctgtgccgccacctgatgcccgacggcagcctgcccgaggcccagagcctgtggccccgccccctgttctgctaa

SEQ ID NO: 39 - DNA, 11329 bp, synthetic construct
  misc_feature 1..11329: note = Synthetic polynucleotide
  source 1..11329: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacagtccggaaagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacaggagggcagaggaagtcttctgacatgcggagacgtggaagagaatcccggccctatggccgagtggctgctgagcgccagctggcagcgccgcgccaaggccatgaccgccgccgccggcagcgccggccgcgccgccgtgcccctgctgctgtgcgccctgctggcccccggcggcgcctacgtgctggacgacagcgacggcctgggccgcgagttcgacggcatcggcgccgtgagcggcggcggcgccaccagccgcctgctggtgaactaccccgagccctaccgcagccagatcctggactacctgttcaagcccaacttcggcgccagcctgcacatcctgaaggtggagatcggcggcgacggccagaccaccgacggcaccgagcccagccacatgcactacgccctggacgagaactacttccgcggctacgagtggtggctgatgaaggaggccaagaagcgcaaccccaacatcaccctgatcggcctgccctggagcttccccggctggctgggcaagggcttcgactggccctacgtgaacctgcagctgaccgcctactacgtggtgacctggatcgtgggcgccaagcgctaccacgacctggacatcgactacatcggcatctggaacgagcgcagctacaacgccaactacatcaagatcctgcgcaagatgctgaactaccagggcctgcagcgcgtgaagatcatcgccagcgacaacctgtgggagagcatcagcgccagcatgctgctggacgccgagctgttcaaggtggtggacgtgatcggcgcccactaccccggcacccacagcgccaaggacgccaagctgaccggcaagaagctgtggagcagcgaggacttcagcaccctgaacagcgacatgggcgccggctgctggggccgcatcctgaaccagaactacatcaacggctacatgaccagcaccatcgcctggaacctggtggccagctactacgagcagctgccctacggccgctgcggcctgatgaccgcccaggagccctggagcggccactacgtggtggagagccccgtgtgggtgagcgcccacaccacccagttcacccagcccggctggtactacctgaagaccgtgggccacctggagaagggcggcagctacgtggccctgaccgacggcctgggcaacctgaccatcatcatcgagaccatgagccacaagcacagcaagtgcatccgccccttcctgccctacttcaacgtgagccagcagttcgccaccttcgtgctgaagggcagcttcagcgagatccccgagctgcaggtgtggtacaccaagctgggcaagaccagcgagcgcttcctgttcaagcagctggacagcctgtggctgctggacagcgacggcagcttcaccctgagcctgcacgaggacgagctgttcaccctgaccaccctgaccaccggccgcaagggcagctaccccctgccccccaagagccagcccttccccagcacctacaaggacgacttcaacgtggactaccccttcttcagcgaggcccccaacttcgccgaccagaccggcgtgttcgagtacttcaccaacatcgaggaccccggcgagcaccacttcaccctgcgccaggtgctgaaccagcgccccatcacctgggccgccgacgccagcaacaccatcagcatcatcggcgactacaactggaccaacctgaccatcaagtgcgacgtgtacatcgagacccccgacaccggcggcgtgttcatcgccggccgcgtgaacaagggcggcatcctgatccgcagcgcccgcggcatcttcttctggatcttcgccaacggcagctaccgcgtgaccggcgacctggccggctggatcatctacgccctgggccgcgtggaggtgaccgccaagaagtggtacaccctgaccctgaccatcaagggccacttcaccagcggcatgctgaacgacaagagcctgtggaccgacatccccgtgaacttccccaagaacggctgggccgccatcggcacccacagcttcgagttcgcccagttcgacaacttcctggtggaggccacccgctgacaattgttaattaagtttaaaccctcgaggccgcaagcaataaaatatctttattttcattacatctgtgtgttggttttttgtgtggagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 40 - DNA, 11776 bp, synthetic construct
  misc_feature 1..11776: note = Synthetic polynucleotide
  source 1..11776: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacagtccggaaagccaccatggccgagtggctgctgagcgccagctggcagcgccgcgccaaggccatgaccgccgccgccggcagcgccggccgcgccgccgtgcccctgctgctgtgcgccctgctggcccccggcggcgcctacgtgctggacgacagcgacggcctgggccgcgagttcgacggcatcggcgccgtgagcggcggcggcgccaccagccgcctgctggtgaactaccccgagccctaccgcagccagatcctggactacctgttcaagcccaacttcggcgccagcctgcacatcctgaaggtggagatcggcggcgacggccagaccaccgacggcaccgagcccagccacatgcactacgccctggacgagaactacttccgcggctacgagtggtggctgatgaaggaggccaagaagcgcaaccccaacatcaccctgatcggcctgccctggagcttccccggctggctgggcaagggcttcgactggccctacgtgaacctgcagctgaccgcctactacgtggtgacctggatcgtgggcgccaagcgctaccacgacctggacatcgactacatcggcatctggaacgagcgcagctacaacgccaactacatcaagatcctgcgcaagatgctgaactaccagggcctgcagcgcgtgaagatcatcgccagcgacaacctgtgggagagcatcagcgccagcatgctgctggacgccgagctgttcaaggtggtggacgtgatcggcgcccactaccccggcacccacagcgccaaggacgccaagctgaccggcaagaagctgtggagcagcgaggacttcagcaccctgaacagcgacatgggcgccggctgctggggccgcatcctgaaccagaactacatcaacggctacatgaccagcaccatcgcctggaacctggtggccagctactacgagcagctgccctacggccgctgcggcctgatgaccgcccaggagccctggagcggccactacgtggtggagagccccgtgtgggtgagcgcccacaccacccagttcacccagcccggctggtactacctgaagaccgtgggccacctggagaagggcggcagctacgtggccctgaccgacggcctgggcaacctgaccatcatcatcgagaccatgagccacaagcacagcaagtgcatccgccccttcctgccctacttcaacgtgagccagcagttcgccaccttcgtgctgaagggcagcttcagcgagatccccgagctgcaggtgtggtacaccaagctgggcaagaccagcgagcgcttcctgttcaagcagctggacagcctgtggctgctggacagcgacggcagcttcaccctgagcctgcacgaggacgagctgttcaccctgaccaccctgaccaccggccgcaagggcagctaccccctgccccccaagagccagcccttccccagcacctacaaggacgacttcaacgtggactaccccttcttcagcgaggcccccaacttcgccgaccagaccggcgtgttcgagtacttcaccaacatcgaggaccccggcgagcaccacttcaccctgcgccaggtgctgaaccagcgccccatcacctgggccgccgacgccagcaacaccatcagcatcatcggcgactacaactggaccaacctgaccatcaagtgcgacgtgtacatcgagacccccgacaccggcggcgtgttcatcgccggccgcgtgaacaagggcggcatcctgatccgcagcgcccgcggcatcttcttctggatcttcgccaacggcagctaccgcgtgaccggcgacctggccggctggatcatctacgccctgggccgcgtggaggtgaccgccaagaagtggtacaccctgaccctgaccatcaagggccacttcaccagcggcatgctgaacgacaagagcctgtggaccgacatccccgtgaacttccccaagaacggctgggccgccatcggcacccacagcttcgagttcgcccagttcgacaacttcctggtggaggccacccgctgattgtggccgaaccgccgaactcagaggccggccccagaaaacccgagcgagtagggggcggcgcgcaggagggaggagaactgggggcgcgggaggctggtgggtgtggggggtggagatgtagaagatgtgacgccgcggcccggcgggtgccagattagcggacgcggtgcccgcggttgcaacgggatcccgggcgctgcagcttgggaggcggctctccccaggcggcgtccgcggagacacccatccgtgaaccccaggtcccgggccgccggctcgccgcgcaccaggggccggcggacagaagagcggccgagcggctcgaggctgggggaccgcgggcgcggccgcgcgctgccgggcgggaggctggggggccggggccggggccgtgccccggagcgggtcggaggccggggccggggccgggggacggcggctccccgcgcggctccagcggctcggggatcccggccgggccccgcagggaccatgatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcaataaaatatctttattttcattacatctgtgtgttggttttttgtgtggagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 41 - DNA, 11348 bp, synthetic construct
  misc_feature 1..11348: note = Synthetic polynucleotide
  source 1..11348: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctcctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccaccgcagccctaggaatgcatctagacaattgtactaaccttcttctctttcctctcctgacagtccggaaagccaccatgtggcagctgtgggccagcctgtgctgcctgctggtgctggccaacgcccgcagccgccccagcttccaccccctgagcgacgagctggtgaactacgtgaacaagcgcaacaccacctggcaggccggccacaacttctacaacgtggacatgagctacctgaagcgcctgtgcggcaccttcctgggcggccccaagcccccccagcgcgtgatgttcaccgaggacctgaagctgcccgccagcttcgacgcccgcgagcagtggccccagtgccccaccatcaaggagatccgcgaccagggcagctgcggcagctgctgggccttcggcgccgtggaggccatcagcgaccgcatctgcatccacaccaacgcccacgtgagcgtggaggtgagcgccgaggacctgctgacctgctgcggcagcatgtgcggcgacggctgcaacggcggctaccccgccgaggcctggaacttctggacccgcaagggcctggtgagcggcggcctgtacgagagccacgtgggctgccgcccctacagcatccccccctgcgagcaccacgtgaacggcagccgccccccctgcaccggcgagggcgacacccccaagtgcagcaagatctgcgagcccggctacagccccacctacaagcaggacaagcactacggctacaacagctacagcgtgagcaacagcgagaaggacatcatggccgagatctacaagaacggccccgtggagggcgccttcagcgtgtacagcgacttcctgctgtacaagagcggcgtgtaccagcacgtgaccggcgagatgatgggcggccacgccatccgcatcctgggctggggcgtggagaacggcaccccctactggctggtggccaacagctggaacaccgactggggcgacaacggcttcttcaagatcctgcgcggccaggaccactgcggcatcgagagcgaggtggtggccggcatcccccgcaccgaccagtactgggagaagatcgagggcagaggaagtcttctgacatgcggagacgtggaagagaatcccggccctatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 42 - DNA, 11433 bp, synthetic construct
  misc_feature 1..11433: note = Synthetic polynucleotide
  source 1..11433: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctcctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccaccgcagccctaggaatgcatctagacaattgtactaaccttcttctctttcctctcctgacagtccggaaagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacaggagggcagaggaagtcttctgacatgcggagacgtggaagagaatcccggccctatgccccgctacggcgccagcctgcgccagagctgcccccgcagcggccgcgagcagggccaggacggcaccgccggcgcccccggcctgctgtggatgggcctggtgctggccctggccctggccctggccctggccctggccctgagcgacagccgcgtgctgtgggcccccgccgaggcccaccccctgagcccccagggccaccccgcccgcctgcaccgcatcgtgccccgcctgcgcgacgtgttcggctggggcaacctgacctgccccatctgcaagggcctgttcaccgccatcaacctgggcctgaagaaggagcccaacgtggcccgcgtgggcagcgtggccatcaagctgtgcaacctgctgaagatcgccccccccgccgtgtgccagagcatcgtgcacctgttcgaggacgacatggtggaggtgtggcgccgcagcgtgctgagccccagcgaggcctgcggcctgctgctgggcagcacctgcggccactgggacatcttcagcagctggaacatcagcctgcccaccgtgcccaagcccccccccaagccccccagcccccccgcccccggcgcccccgtgagccgcatcctgttcctgaccgacctgcactgggaccacgactacctggagggcaccgaccccgactgcgccgaccccctgtgctgccgccgcggcagcggcctgccccccgccagccgccccggcgccggctactggggcgagtacagcaagtgcgacctgcccctgcgcaccctggagagcctgctgagcggcctgggccccgccggccccttcgacatggtgtactggaccggcgacatccccgcccacgacgtgtggcaccagacccgccaggaccagctgcgcgccctgaccaccgtgaccgccctggtgcgcaagttcctgggccccgtgcccgtgtaccccgccgtgggcaaccacgagagcacccccgtgaacagcttccccccccccttcatcgagggcaaccacagcagccgctggctgtacgaggccatggccaaggcctgggagccctggctgcccgccgaggccctgcgcaccctgcgcatcggcggcttctacgccctgagcccctaccccggcctgcgcctgatcagcctgaacatgaacttctgcagccgcgagaacttctggctgctgatcaacagcaccgaccccgccggccagctgcagtggctggtgggcgagctgcaggccgccgaggaccgcggcgacaaggtgcacatcatcggccacatcccccccggccactgcctgaagagctggagctggaactactaccgcatcgtggcccgctacgagaacaccctggccgcccagttcttcggccacacccacgtggacgagttcgaggtgttctacgacgaggagaccctgagccgccccctggccgtggccttcctggcccccagcgccaccacctacatcggcctgaaccccggctaccgcgtgtaccagatcgacggcaactacagcggcagcagccacgtggtgctggaccacgagacctacatcctgaacctgacccaggccaacatccccggcgccatcccccactggcagctgctgtaccgcgcccgcgagacctacggcctgcccaacaccctgcccaccgcctggcacaacctggtgtaccgcatgcgcggcgacatgcagctgttccagaccttctggttcctgtaccacaagggccacccccccagcgagccctgcggcaccccctgccgcctggccaccctgtgcgcccagctgagcgcccgcgccgacagccccgccctgtgccgccacctgatgcccgacggcagcctgcccgaggcccagagcctgtggccccgccccctgttctgctaatgacaattgttaattaagtttaaaccctcgaggccgcaagcaataaaatatctttattttcattacatctgtgtgttggttttttgtgtggagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 43 - DNA, 11776 bp, synthetic construct; identical to SEQ ID NO: 40
  misc_feature 1..11776: note = Synthetic polynucleotide
  source 1..11776: mol_type = other DNA

SEQ ID NO: 44 - DNA, 11064 bp, synthetic construct
  misc_feature 1..11064: note = Synthetic polynucleotide
  source 1..11064: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacagtccggaaagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttgggggggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacagtccggaaagccaccatgtggcagctgtgggccagcctgtgctgcctgctggtgctggccaacgcccgcagccgccccagcttccaccccctgagcgacgagctggtgaactacgtgaacaagcgcaacaccacctggcaggccggccacaacttctacaacgtggacatgagctacctgaagcgcctgtgcggcaccttcctgggcggccccaagcccccccagcgcgtgatgttcaccgaggacctgaagctgcccgccagcttcgacgcccgcgagcagtggccccagtgccccaccatcaaggagatccgcgaccagggcagctgcggcagctgctgggccttcggcgccgtggaggccatcagcgaccgcatctgcatccacaccaacgcccacgtgagcgtggaggtgagcgccgaggacctgctgacctgctgcggcagcatgtgcggcgacggctgcaacggcggctaccccgccgaggcctggaacttctggacccgcaagggcctggtgagcggcggcctgtacgagagccacgtgggctgccgcccctacagcatccccccctgcgagcaccacgtgaacggcagccgccccccctgcaccggcgagggcgacacccccaagtgcagcaagatctgcgagcccggctacagccccacctacaagcaggacaagcactacggctacaacagctacagcgtgagcaacagcgagaaggacatcatggccgagatctacaagaacggccccgtggagggcgccttcagcgtgtacagcgacttcctgctgtacaagagcggcgtgtaccagcacgtgaccggcgagatgatgggcggccacgccatccgcatcctgggctggggcgtggagaacggcaccccctactggctggtggccaacagctggaacaccgactggggcgacaacggcttcttcaagatcctgcgcggccaggaccactgcggcatcgagagcgaggtggtggccggcatcccccgcaccgaccagtactgggagaagatctgacccaggggactcagcggccgctcgagtctagagggcccgtttaaacccgctgatcagcctcgaagacatgataagatacattgatgagtttggacaaaccacaacaagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtatgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 45 - protein, 250 aa, synthetic construct
  REGION 1..250: note = Synthetic polypeptide
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 46 - DNA, 750 bp, synthetic construct
  misc_feature 1..750: note = Synthetic polynucleotide
  source 1..750: mol_type = other DNA
atggagaagggccccgtgcgcgcccccgccgagaagccccgcggcgcccgctgcagcaacggcttccccgagcgcgaccccccccgccccggccccagccgccccgccgagaagcccccccgccccgaggccaagagcgcccagcccgccgacggctggaagggcgagcgcccccgcagcgaggaggacaacgagctgaacctgcccaacctggccgccgcctacagcagcatcctgagcagcctgggcgagaacccccagcgccagggcctgctgaagaccccctggcgcgccgccagcgccatgcagttcttcaccaagggctaccaggagaccatcagcgacgtgctgaacgacgccatcttcgacgaggaccacgacgagatggtgatcgtgaaggacatcgacatgttcagcatgtgcgagcaccacctggtgcccttcgtgggcaaggtgcacatcggctacctgcccaacaagcaggtgctgggcctgagcaagctggcccgcatcgtggagatctacagccgccgcctgcaggtgcaggagcgcctgaccaagcagatcgccgtggccatcaccgaggccctgcgccccgccggcgtgggcgtggtggtggaggccacccacatgtgcatggtgatgcgcggcgtgcagaagatgaacagcaagaccgtgaccagcaccatgctgggcgtgttccgcgaggaccccaagacccgcgaggagttcctgaccctgatccgcagc

SEQ ID NO: 47 - protein, 203 aa, synthetic construct
  REGION 1..203: note = Synthetic polypeptide
MGSRDHLFKVLVVGDAAVGKTSLVQRYSQDSFSKHYKSTVGVDFALKVLQWSDYEIVRLQLWDIAGQERFTSMTRLYYRDASACVIMFDVTNATTFSNSQRWKQDLDSKLTLPNGEPVPCLLLANKCDLSPWAVSRDQIDRFSKENGFTGWTETSVKENKNINEAMRVLIEKMMRNSTEDIMSLSTQGDYINLQTKSSSWSCC

SEQ ID NO: 48 - DNA, 609 bp, synthetic construct
  misc_feature 1..609: note = Synthetic polynucleotide
  source 1..609: mol_type = other DNA
atgggcagccgcgaccacctgttcaaggtgctggtggtgggcgacgccgccgtgggcaagaccagcctggtgcagcgctacagccaggacagcttcagcaagcactacaagagcaccgtgggcgtggacttcgccctgaaggtgctgcagtggagcgactacgagatcgtgcgcctgcagctgtgggacatcgccggccaggagcgcttcaccagcatgacccgcctgtactaccgcgacgccagcgcctgcgtgatcatgttcgacgtgaccaacgccaccaccttcagcaacagccagcgctggaagcaggacctggacagcaagctgaccctgcccaacggcgagcccgtgccctgcctgctgctggccaacaagtgcgacctgagcccctgggccgtgagccgcgaccagatcgaccgcttcagcaaggagaacggcttcaccggctggaccgagaccagcgtgaaggagaacaagaacatcaacgaggccatgcgcgtgctgatcgagaagatgatgcgcaacagcaccgaggacatcatgagcctgagcacccagggcgactacatcaacctgcagaccaagagcagcagctggagctgctgc

SEQ ID NO: 49 - protein, 796 aa, synthetic construct
  REGION 1..796: note = Synthetic polypeptide
MPTTQQSPQDEQEKLLDEAIQAVKVQSFQMKRCLDKNKLMDALKHASNMLGELRTSMLSPKSYYELYMAISDELHYLEVYLTDEFAKGRKVADLYELVQYAGNIIPRLYLLITVGVVYVKSFPQSRKDILKDLVEMCRGVQHPLRGLFLRNYLLQCTRNILPDEGEPTDEETTGDISDSMDFVLLNFAEMNKLWVRMQHQGHSRDREKRERERQELRILVGTNLVRLSQLEGVNVERYKQIVLTGILEQVVNCRDALAQEYLMECIIQVFPDEFHLQTLNPFLRACAELHQNVNVKNIIIALIDRLALFAHREDGPGIPADIKLFDIFSQQVATVIQSRQDMPSEDVVSLQVSLINLAMKCYPDRVDYVDKVLETTVEIFNKLNLEHIATSSAVSKELTRLLKIPVDTYNNILTVLKLKHFHPLFEYFDYESRKSMSCYVLSNVLDYNTEIVSQDQVDSIMNLVSTLIQDQPDQPVEDPDPEDFADEQSLVGRFIHLLRSEDPDQQYLILNTARKHFGAGGNQRIRFTLPPLVFAAYQLAFRYKENSKVDDKWEKKCQKIFSFAHQTISALIKAELAELPLRLFLQGALAAGEIGFENHETVAYEFMSQAFSLYEDEISDSKAQLAAITLIIGTFERMKCFSEENHEPLRTQCALAASKLLKKPDQGRAVSTCAHLFWSGRNTDKNGEELHGGKRVMECLKKALKIANQCMDPSLQVQLFIEILNRYIYFYEKENDAVTIQVLNQLIQKIREDLPNLESSEETEQINKHFHNTLEHLRLRRESPESEGPIYEGLIL

SEQ ID NO: 50 - DNA, 2388 bp, synthetic construct
  misc_feature 1..2388: note = Synthetic polynucleotide
  source 1..2388: mol_type = other DNA
atgcccaccacccagcagagcccccaggacgagcaggagaagctgctggacgaggccatccaggccgtgaaggtgcagagcttccagatgaagcgctgcctggacaagaacaagctgatggacgccctgaagcacgccagcaacatgctgggcgagctgcgcaccagcatgctgagccccaagagctactacgagctgtacatggccatcagcgacgagctgcactacctggaggtgtacctgaccgacgagttcgccaagggccgcaaggtggccgacctgtacgagctggtgcagtacgccggcaacatcatcccccgcctgtacctgctgatcaccgtgggcgtggtgtacgtgaagagcttcccccagagccgcaaggacatcctgaaggacctggtggagatgtgccgcggcgtgcagcaccccctgcgcggcctgttcctgcgcaactacctgctgcagtgcacccgcaacatcctgcccgacgagggcgagcccaccgacgaggagaccaccggcgacatcagcgacagcatggacttcgtgctgctgaacttcgccgagatgaacaagctgtgggtgcgcatgcagcaccagggccacagccgcgaccgcgagaagcgcgagcgcgagcgccaggagctgcgcatcctggtgggcaccaacctggtgcgcctgagccagctggagggcgtgaacgtggagcgctacaagcagatcgtgctgaccggcatcctggagcaggtggtgaactgccgcgacgccctggcccaggagtacctgatggagtgcatcatccaggtgttccccgacgagttccacctgcagaccctgaaccccttcctgcgcgcctgcgccgagctgcaccagaacgtgaacgtgaagaacatcatcatcgccctgatcgaccgcctggccctgttcgcccaccgcgaggacggccccggcatccccgccgacatcaagctgttcgacatcttcagccagcaggtggccaccgtgatccagagccgccaggacatgcccagcgaggacgtggtgagcctgcaggtgagcctgatcaacctggccatgaagtgctaccccgaccgcgtggactacgtggacaaggtgctggagaccaccgtggagatcttcaacaagctgaacctggagcacatcgccaccagcagcgccgtgagcaaggagctgacccgcctgctgaagatccccgtggacacctacaacaacatcctgaccgtgctgaagctgaagcacttccaccccctgttcgagtacttcgactacgagagccgcaagagcatgagctgctacgtgctgagcaacgtgctggactacaacaccgagatcgtgagccaggaccaggtggacagcatcatgaacctggtgagcaccctgatccaggaccagcccgaccagcccgtggaggaccccgaccccgaggacttcgccgacgagcagagcctggtgggccgcttcatccacctgctgcgcagcgaggaccccgaccagcagtacctgatcctgaacaccgcccgcaagcacttcggcgccggcggcaaccagcgcatccgcttcaccctgccccccctggtgttcgccgcctaccagctggccttccgctacaaggagaacagcaaggtggacgacaagtgggagaagaagtgccagaagatcttcagcttcgcccaccagaccatcagcgccctgatcaaggccgagctggccgagctgcccctgcgcctgttcctgcagggcgccctggccgccggcgagatcggcttcgagaaccacgagaccgtggcctacgagttcatgagccaggccttcagcctgtacgaggacgagatcagcgacagcaaggcccagctggccgccatcaccctgatcatcggcaccttcgagcgcatgaagtgcttcagcgaggagaaccacgagcccctgcgcacccagtgcgccctggccgccagcaagctgctgaagaagcccgaccagggccgcgccgtgagcacctgcgcccacctgttctggagcggccgcaacaccgacaagaacggcgaggagctgcacggcggcaagcgcgtgatggagtgcctgaagaaggccctgaagatcgccaaccagtgcatggaccccagcctgcaggtgcagctgttcatcgagatcctgaaccgctacatctacttctacgagaaggagaacgacgccgtgaccatccaggtgctgaaccagctgatccagaagatccgcgaggacctgcccaacctggagagcagcgaggagaccgagcagatcaacaagcacttccacaacaccctggagcacctgcgcctgcgccgcgagagccccgagagcgagggccccatctacgagggcctgatcctg

SEQ ID NO: 51 - DNA, 11081 bp, synthetic construct
  misc_feature 1..11081: note = Synthetic polynucleotide
  source 1..11081: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctcctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccaccgcagccctaggaatgcatctagacaattgtactaaccttcttctctttcctctcctgacagtccggaaagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacaggagggcagaggaagtcttctgacatgcggagacgtggaagagaatcccggccctatggagaagggccccgtgcgcgcccccgccgagaagccccgcggcgcccgctgcagcaacggcttccccgagcgcgaccccccccgccccggccccagccgccccgccgagaagcccccccgccccgaggccaagagcgcccagcccgccgacggctggaagggcgagcgcccccgcagcgaggaggacaacgagctgaacctgcccaacctggccgccgcctacagcagcatcctgagcagcctgggcgagaacccccagcgccagggcctgctgaagaccccctggcgcgccgccagcgccatgcagttcttcaccaagggctaccaggagaccatcagcgacgtgctgaacgacgccatcttcgacgaggaccacgacgagatggtgatcgtgaaggacatcgacatgttcagcatgtgcgagcaccacctggtgcccttcgtgggcaaggtgcacatcggctacctgcccaacaagcaggtgctgggcctgagcaagctggcccgcatcgtggagatctacagccgccgcctgcaggtgcaggagcgcctgaccaagcagatcgccgtggccatcaccgaggccctgcgccccgccggcgtgggcgtggtggtggaggccacccacatgtgcatggtgatgcgcggcgtgcagaagatgaacagcaagaccgtgaccagcaccatgctgggcgtgttccgcgaggaccccaagacccgcgaggagttcctgaccctgatccgcagctgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 52 - DNA, 10940 bp, synthetic construct
  misc_feature 1..10940: note = Synthetic polynucleotide
  source 1..10940: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctcctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccaccgcagccctaggaatgcatctagacaattgtactaaccttcttctctttcctctcctgacagtccggaaagccaccatgggcagccgcgaccacctgttcaaggtgctggtggtgggcgacgccgccgtgggcaagaccagcctggtgcagcgctacagccaggacagcttcagcaagcactacaagagcaccgtgggcgtggacttcgccctgaaggtgctgcagtggagcgactacgagatcgtgcgcctgcagctgtgggacatcgccggccaggagcgcttcaccagcatgacccgcctgtactaccgcgacgccagcgcctgcgtgatcatgttcgacgtgaccaacgccaccaccttcagcaacagccagcgctggaagcaggacctggacagcaagctgaccctgcccaacggcgagcccgtgccctgcctgctgctggccaacaagtgcgacctgagcccctgggccgtgagccgcgaccagatcgaccgcttcagcaaggagaacggcttcaccggctggaccgagaccagcgtgaaggagaacaagaacatcaacgaggccatgcgcgtgctgatcgagaagatgatgcgcaacagcaccgaggacatcatgagcctgagcacccagggcgactacatcaacctgcagaccaagagcagcagctggagctgctgcgagggcagaggaagtcttctgacatgcggagacgtggaagagaatcccggccctatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 53 - DNA, 10934 bp, synthetic construct
  misc_feature 1..10934: note = Synthetic polynucleotide
  source 1..10934: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctcctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccaccgcagccctaggaatgcatctagacaattgtactaaccttcttctctttcctctcctgacagtccggaaagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgattgtggccgaaccgccgaactcagaggccggccccagaaaacccgagcgagtagggggcggcgcgcaggagggaggagaactgggggcgcgggaggctggtgggtgtggggggtggagatgtagaagatgtgacgccgcggcccggcgggtgccagattagcggacgcggtgcccgcggttgcaacgggatcccgggcgctgcagcttgggaggcggctctccccaggcggcgtccgcggagacacccatccgtgaaccccaggtcccgggccgccggctcgccgcgcaccaggggccggcggacagaagagcggccgagcggctcgaggctgggggaccgcgggcgcggccgcgcgctgccgggcgggaggctggggggccggggccggggccgtgccccggagcgggtcggaggccggggccggggccgggggacggcggctccccgcgcggctccagcggctcggggatcccggccgggccccgcagggaccatgatggagaagggccccgtgcgcgcccccgccgagaagccccgcggcgcccgctgcagcaacggcttccccgagcgcgaccccccccgccccggccccagccgccccgccgagaagcccccccgccccgaggccaagagcgcccagcccgccgacggctggaagggcgagcgcccccgcagcgaggaggacaacgagctgaacctgcccaacctggccgccgcctacagcagcatcctgagcagcctgggcgagaacccccagcgccagggcctgctgaagaccccctggcgcgccgccagcgccatgcagttcttcaccaagggctaccaggagaccatcagcgacgtgctgaacgacgccatcttcgacgaggaccacgacgagatggtgatcgtgaaggacatcgacatgttcagcatgtgcgagcaccacctggtgcccttcgtgggcaaggtgcacatcggctacctgcccaacaagcaggtgctgggcctgagcaagctggcccgcatcgtggagatctacagccgccgcctgcaggtgcaggagcgcctgaccaagcagatcgccgtggccatcaccgaggccctgcgccccgccggcgtgggcgtggtggtggaggccacccacatgtgcatggtgatgcgcggcgtgcagaagatgaacagcaagaccgtgaccagcaccatgctgggcgtgttccgcgaggaccccaagacccgcgaggagttcctgaccctgatccgcagctgacaattgttaattaagtttaaaccctcgaggccgcaagccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 54 - DNA, 11138 bp, synthetic construct
  misc_feature 1..11138: note = Synthetic polynucleotide
  source 1..11138: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagtaagtcactgactgtctatgcctgggaaagggtgggcaggagatggggcagtgcaggaaaagtggcactatgaaccctcctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccaccgtgatatcacaaggtcccagggctggggtcagaaattctctcccgagggaatgaagccacaggagccaagagcaggaggaccaaggccctggcgaaggccgtggcctcgttcaagtaaaagatcctagtacagtgcaggtcccaatgtgtactaggatcttttacttgaacggggacgccggcatccgggctcaggacccccctctctgccagaggcaccaacaccagagttcacaaatcagtctcctgccctttgcatgtagcaaagcagccctaggaatgcatctagacaattgtactaaccttcttctctttcctctcctgacagtccggaaagccaccatgcccaccacccagcagagcccccaggacgagcaggagaagctgctggacgaggccatccaggccgtgaaggtgcagagcttccagatgaagcgctgcctggacaagaacaagctgatggacgccctgaagcacgccagcaacatgctgggcgagctgcgcaccagcatgctgagccccaagagctactacgagctgtacatggccatcagcgacgagctgcactacctggaggtgtacctgaccgacgagttcgccaagggccgcaaggtggccgacctgtacgagctggtgcagtacgccggcaacatcatcccccgcctgtacctgctgatcaccgtgggcgtggtgtacgtgaagagcttcccccagagccgcaaggacatcctgaaggacctggtggagatgtgccgcggcgtgcagcaccccctgcgcggcctgttcctgcgcaactacctgctgcagtgcacccgcaacatcctgcccgacgagggcgagcccaccgacgaggagaccaccggcgacatcagcgacagcatggacttcgtgctgctgaacttcgccgagatgaacaagctgtgggtgcgcatgcagcaccagggccacagccgcgaccgcgagaagcgcgagcgcgagcgccaggagctgcgcatcctggtgggcaccaacctggtgcgcctgagccagctggagggcgtgaacgtggagcgctacaagcagatcgtgctgaccggcatcctggagcaggtggtgaactgccgcgacgccctggcccaggagtacctgatggagtgcatcatccaggtgttccccgacgagttccacctgcagaccctgaaccccttcctgcgcgcctgcgccgagctgcaccagaacgtgaacgtgaagaacatcatcatcgccctgatcgaccgcctggccctgttcgcccaccgcgaggacggccccggcatccccgccgacatcaagctgttcgacatcttcagccagcaggtggccaccgtgatccagagccgccaggacatgcccagcgaggacgtggtgagcctgcaggtgagcctgatcaacctggccatgaagtgctaccccgaccgcgtggactacgtggacaaggtgctggagaccaccgtggagatcttcaacaagctgaacctggagcacatcgccaccagcagcgccgtgagcaaggagctgacccgcctgctgaagatccccgtggacacctacaacaacatcctgaccgtgctgaagctgaagcacttccaccccctgttcgagtacttcgactacgagagccgcaagagcatgagctgctacgtgctgagcaacgtgctggactacaacaccgagatcgtgagccaggaccaggtggacagcatcatgaacctggtgagcaccctgatccaggaccagcccgaccagcccgtggaggaccccgaccccgaggacttcgccgacgagcagagcctggtgggccgcttcatccacctgctgcgcagcgaggaccccgaccagcagtacctgatcctgaacaccgcccgcaagcacttcggcgccggcggcaaccagcgcatccgcttcaccctgccccccctggtgttcgccgcctaccagctggccttccgctacaaggagaacagcaaggtggacgacaagtgggagaagaagtgccagaagatcttcagcttcgcccaccagaccatcagcgccctgatcaaggccgagctggccgagctgcccctgcgcctgttcctgcagggcgccctggccgccggcgagatcggcttcgagaaccacgagaccgtggcctacgagttcatgagccaggccttcagcctgtacgaggacgagatcagcgacagcaaggcccagctggccgccatcaccctgatcatcggcaccttcgagcgcatgaagtgcttcagcgaggagaaccacgagcccctgcgcacccagtgcgccctggccgccagcaagctgctgaagaagcccgaccagggccgcgccgtgagcacctgcgcccacctgttctggagcggccgcaacaccgacaagaacggcgaggagctgcacggcggcaagcgcgtgatggagtgcctgaagaaggccctgaagatcgccaaccagtgcatggaccccagcctgcaggtgcagctgttcatcgagatcctgaaccgctacatctacttctacgagaaggagaacgacgccgtgaccatccaggtgctgaaccagctgatccagaagatccgcgaggacctgcccaacctggagagcagcgaggagaccgagcagatcaacaagcacttccacaacaccctggagcacctgcgcctgcgccgcgagagccccgagagcgagggccccatctacgagggcctgatcctgtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 55 - protein, 242 aa, synthetic construct
  REGION 1..242: note = Synthetic polypeptide
MPRGFTWLRYLGIFLGVALGNEPLEMWPLTQNEECTVTGFLRDKLQYRSRLQYMKHYFPINYKISVPYEGVFRIANVTRLQRAQVSERELRYLWVLVSLSATESVQDVLLEGHPSWKYLQEVETLLLNVQQGLTDVEVSPKVESVLSLLNAPGPNLKLVRPKALLDNCFRVMELLYCSCCKQSSVLNWQDCEVPSPQSCSPEPSLQYAATQLYPPPPWSPSSPPHSTGSVRPVRAQGEGLLP

SEQ ID NO: 56 - DNA, 729 bp, synthetic construct
  misc_feature 1..729: note = Synthetic polynucleotide
  source 1..729: mol_type = other DNA
atgccccgcggcttcacctggctgcgctacctgggcatcttcctgggcgtggccctgggcaacgagcccctggagatgtggcccctgacccagaacgaggagtgcaccgtgaccggcttcctgcgcgacaagctgcagtaccgcagccgcctgcagtacatgaagcactacttccccatcaactacaagatcagcgtgccctacgagggcgtgttccgcatcgccaacgtgacccgcctgcagcgcgcccaggtgagcgagcgcgagctgcgctacctgtgggtgctggtgagcctgagcgccaccgagagcgtgcaggacgtgctgctggagggccaccccagctggaagtacctgcaggaggtggagaccctgctgctgaacgtgcagcagggcctgaccgacgtggaggtgagccccaaggtggagagcgtgctgagcctgctgaacgcccccggccccaacctgaagctggtgcgccccaaggccctgctggacaactgcttccgcgtgatggagctgctgtactgcagctgctgcaagcagagcagcgtgctgaactggcaggactgcgaggtgcccagcccccagagctgcagccccgagcccagcctgcagtacgccgccacccagctgtaccccccccccccctggagccccagcagccccccccacagcaccggcagcgtgcgccccgtgcgcgcccagggcgagggcctgctgccctaa

SEQ ID NO: 57 - protein, 230 aa, synthetic construct
  REGION 1..230: note = Synthetic polypeptide
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 58 - DNA, 690 bp, synthetic construct
  misc_feature 1..690: note = Synthetic polynucleotide
  source 1..690: mol_type = other DNA
atggagcccctgcgcctgctgatcctgctgttcgtgaccgagctgagcggcgcccacaacaccaccgtgttccagggcgtggccggccagagcctgcaggtgagctgcccctacgacagcatgaagcactggggccgccgcaaggcctggtgccgccagctgggcgagaagggcccctgccagcgcgtggtgagcacccacaacctgtggctgctgagcttcctgcgccgctggaacggcagcaccgccatcaccgacgacaccctgggcggcaccctgaccatcaccctgcgcaacctgcagccccacgacgccggcctgtaccagtgccagagcctgcacggcagcgaggccgacaccctgcgcaaggtgctggtggaggtgctggccgaccccctggaccaccgcgacgccggcgacctgtggttccccggcgagagcgagagcttcgaggacgcccacgtggagcacagcatcagccgcagcctgctggagggcgagatccccttcccccccaccagcatcctgctgctgctggcctgcatcttcctgatcaagatcctggccgccagcgccctgtgggccgccgcctggcacggccagaagcccggcacccacccccccagcgagctggactgcggccacgaccccggctaccagctgcagaccctgcccggcctgcgcgacacc

SEQ ID NO: 59 - DNA, 11060 bp, synthetic construct
  misc_feature 1..11060: note = Synthetic polynucleotide
  source 1..11060: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctcctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccaccgcagccctaggaatgcatctagacaattgtactaaccttcttctctttcctctcctgacagtccggaaagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacaggagggcagaggaagtcttctgacatgcggagacgtggaagagaatcccggccctatgccccgcggcttcacctggctgcgctacctgggcatcttcctgggcgtggccctgggcaacgagcccctggagatgtggcccctgacccagaacgaggagtgcaccgtgaccggcttcctgcgcgacaagctgcagtaccgcagccgcctgcagtacatgaagcactacttccccatcaactacaagatcagcgtgccctacgagggcgtgttccgcatcgccaacgtgacccgcctgcagcgcgcccaggtgagcgagcgcgagctgcgctacctgtgggtgctggtgagcctgagcgccaccgagagcgtgcaggacgtgctgctggagggccaccccagctggaagtacctgcaggaggtggagaccctgctgctgaacgtgcagcagggcctgaccgacgtggaggtgagccccaaggtggagagcgtgctgagcctgctgaacgcccccggccccaacctgaagctggtgcgccccaaggccctgctggacaactgcttccgcgtgatggagctgctgtactgcagctgctgcaagcagagcagcgtgctgaactggcaggactgcgaggtgcccagcccccagagctgcagccccgagcccagcctgcagtacgccgccacccagctgtaccccccccccccctggagccccagcagccccccccacagcaccggcagcgtgcgccccgtgcgcgcccagggcgagggcctgctgccctaatgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 60 - DNA, 10913 bp, synthetic construct
  misc_feature 1..10913: note = Synthetic polynucleotide
  source 1..10913: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctcctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccaccgcagccctaggaatgcatctagacaattgtactaaccttcttctctttcctctcctgacagtccggaaagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgattgtggccgaaccgccgaactcagaggccggccccagaaaacccgagcgagtagggggcggcgcgcaggagggaggagaactgggggcgcgggaggctggtgggtgtggggggtggagatgtagaagatgtgacgccgcggcccggcgggtgccagattagcggacgcggtgcccgcggttgcaacgggatcccgggcgctgcagcttgggaggcggctctccccaggcggcgtccgcggagacacccatccgtgaaccccaggtcccgggccgccggctcgccgcgcaccaggggccggcggacagaagagcggccgagcggctcgaggctgggggaccgcgggcgcggccgcgcgctgccgggcgggaggctggggggccggggccggggccgtgccccggagcgggtcggaggccggggccggggccgggggacggcggctccccgcgcggctccagcggctcggggatcccggccgggccccgcagggaccatgatgccccgcggcttcacctggctgcgctacctgggcatcttcctgggcgtggccctgggcaacgagcccctggagatgtggcccctgacccagaacgaggagtgcaccgtgaccggcttcctgcgcgacaagctgcagtaccgcagccgcctgcagtacatgaagcactacttccccatcaactacaagatcagcgtgccctacgagggcgtgttccgcatcgccaacgtgacccgcctgcagcgcgcccaggtgagcgagcgcgagctgcgctacctgtgggtgctggtgagcctgagcgccaccgagagcgtgcaggacgtgctgctggagggccaccccagctggaagtacctgcaggaggtggagaccctgctgctgaacgtgcagcagggcctgaccgacgtggaggtgagccccaaggtggagagcgtgctgagcctgctgaacgcccccggccccaacctgaagctggtgcgccccaaggccctgctggacaactgcttccgcgtgatggagctgctgtactgcagctgctgcaagcagagcagcgtgctgaactggcaggactgcgaggtgcccagcccccagagctgcagccccgagcccagcctgcagtacgccgccacccagctgtaccccccccccccctggagccccagcagccccccccacagcaccggcagcgtgcgccccgtgcgcgcccagggcgagggcctgctgccctaatgacaattgttaattaagtttaaaccctcgaggccgcaagccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 61 - DNA, 11209 bp, synthetic construct
  misc_feature 1..11209: note = Synthetic polynucleotide
  source 1..11209: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacagtccggaaagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttgggggatatcaaactgcctgtttgggcttctcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagcttacttgccacaactgccaagcaagtttcgctgagtttgacacatggatccctgtggatcaactgccctaggactccgtttgcacccatgtgacactgttgactttgccctgacgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagccatatcgaattctgctggggctactggcaggtaaggaggaaggaggctgaggggagggggcccctgggagggagcctgccctgggttgctaaccatctcctctctgccaaaagtccggaaagccaccatggagcccctgcgcctgctgatcctgctgttcgtgaccgagctgagcggcgcccacaacaccaccgtgttccagggcgtggccggccagagcctgcaggtgagctgcccctacgacagcatgaagcactggggccgccgcaaggcctggtgccgccagctgggcgagaagggcccctgccagcgcgtggtgagcacccacaacctgtggctgctgagcttcctgcgccgctggaacggcagcaccgccatcaccgacgacaccctgggcggcaccctgaccatcaccctgcgcaacctgcagccccacgacgccggcctgtaccagtgccagagcctgcacggcagcgaggccgacaccctgcgcaaggtgctggtggaggtgctggccgaccccctggaccaccgcgacgccggcgacctgtggttccccggcgagagcgagagcttcgaggacgcccacgtggagcacagcatcagccgcagcctgctggagggcgagatccccttcccccccaccagcatcctgctgctgctggcctgcatcttcctgatcaagatcctggccgccagcgccctgtgggccgccgcctggcacggccagaagcccggcacccacccccccagcgagctggactgcggccacgaccccggctaccagctgcagaccctgcccggcctgcgcgacacctgacccaggggactcagcggccgctcgagtctagagggcccgtttaaacccgctgatcagcctcgaagacatgataagatacattgatgagtttggacaaaccacaacaagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtatgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 62 - DNA, 11459 bp, synthetic construct
  misc_feature 1..11459: note = Synthetic polynucleotide
  source 1..11459: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttgggggggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacagtccggaaagccaccatgccccgcggcttcacctggctgcgctacctgggcatcttcctgggcgtggccctgggcaacgagcccctggagatgtggcccctgacccagaacgaggagtgcaccgtgaccggcttcctgcgcgacaagctgcagtaccgcagccgcctgcagtacatgaagcactacttccccatcaactacaagatcagcgtgccctacgagggcgtgttccgcatcgccaacgtgacccgcctgcagcgcgcccaggtgagcgagcgcgagctgcgctacctgtgggtgctggtgagcctgagcgccaccgagagcgtgcaggacgtgctgctggagggccaccccagctggaagtacctgcaggaggtggagaccctgctgctgaacgtgcagcagggcctgaccgacgtggaggtgagccccaaggtggagagcgtgctgagcctgctgaacgcccccggccccaacctgaagctggtgcgccccaaggccctgctggacaactgcttccgcgtgatggagctgctgtactgcagctgctgcaagcagagcagcgtgctgaactggcaggactgcgaggtgcccagcccccagagctgcagccccgagcccagcctgcagtacgccgccacccagctgtaccccccccccccctggagccccagcagccccccccacagcaccggcagcgtgcgccccgtgcgcgcccagggcgagggcctgctgccctaatgacccaggggactcagcggccgctcgagtctagagggcccgtttaaacccgctgatcagcctcgaagacatgataagatacattgatgagtttggacaaaccacaacaagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtatgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 63 - protein, 274 aa, synthetic construct
  REGION 1..274: note = Synthetic polypeptide
MGKSLSHLPLHSSKEDAYDGVTSENMRNGLVNSEVHNEDGRNGDVSQFPYVEFTGRDSVTCPTCQGTGRIPRGQENQLVALIPYSDQRLRPRRTKLYVMASVFVCLLLSGLAVFFLFPRSIDVKYIGVKSAYVSYDVQKRTIYLNITNTLNITNNNYYSVEVENITAQVQFSKTVIGKARLNNITIIGPLDMKQIDYTVPTVIAEEMSYMYDFCTLISIKVHNIVLMMQVTVTTTYFGHSEQISQERYQYVDCGRNTTYQLGQSEYLNVLQPQQ

SEQ ID NO: 64 - DNA, 825 bp, synthetic construct
  misc_feature 1..825: note = Synthetic polynucleotide
  source 1..825: mol_type = other DNA
atgggcaagagcctgagccacctgcccctgcacagcagcaaggaggacgcctacgacggcgtgaccagcgagaacatgcgcaacggcctggtgaacagcgaggtgcacaacgaggacggccgcaacggcgacgtgagccagttcccctacgtggagttcaccggccgcgacagcgtgacctgccccacctgccagggcaccggccgcatcccccgcggccaggagaaccagctggtggccctgatcccctacagcgaccagcgcctgcgcccccgccgcaccaagctgtacgtgatggccagcgtgttcgtgtgcctgctgctgagcggcctggccgtgttcttcctgttcccccgcagcatcgacgtgaagtacatcggcgtgaagagcgcctacgtgagctacgacgtgcagaagcgcaccatctacctgaacatcaccaacaccctgaacatcaccaacaacaactactacagcgtggaggtggagaacatcaccgcccaggtgcagttcagcaagaccgtgatcggcaaggcccgcctgaacaacatcaccatcatcggccccctggacatgaagcagatcgactacaccgtgcccaccgtgatcgccgaggagatgagctacatgtacgacttctgcaccctgatcagcatcaaggtgcacaacatcgtgctgatgatgcaggtgaccgtgaccaccacctacttcggccacagcgagcagatcagccaggagcgctaccagtacgtggactgcggccgcaacaccacctaccagctgggccagagcgagtacctgaacgtgctgcagccccagcagtaa

SEQ ID NO: 65 - DNA, 267 bp, synthetic construct
  misc_feature 1..267: note = Synthetic polynucleotide
  source 1..267: mol_type = other DNA
gtgatatcacaaggtcccagggctggggtcagaaattctctcccgagggaatgaagccacaggagccaagagcaggaggaccaaggccctggcgaaggccgtggcctcgttcaagtaaaagatcctagtacagtgcaggtcccaatgtgtactaggatcttttacttgaacggggacgccggcatccgggctcaggacccccctctctgccagaggcaccaacaccagagttcacaaatcagtctcctgccctttgcatgtagcaaa

SEQ ID NO: 66 - DNA, 267 bp, synthetic construct
  misc_feature 1..267: note = Synthetic polynucleotide
  source 1..267: mol_type = other DNA
tttgctacatgcaaagggcaggagactgatttgtgaactctggtgttggtgcctctggcagagaggggggtcctgagcccggatgccggcgtccccgttcaagtaaaagatcctagtacacattgggacctgcactgtactaggatcttttacttgaacgaggccacggccttcgccagggccttggtcctcctgctcttggctcctgtggcttcattccctcgggagagaatttctgaccccagccctgggaccttgtgatatcac

SEQ ID NO: 67 - protein, 593 aa, synthetic construct
  REGION 1..593: note = Synthetic polypeptide
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 68 - DNA, 1779 bp, synthetic construct
  misc_feature 1..1779: note = Synthetic polynucleotide
  source 1..1779: mol_type = other DNA
atgtggaccctggtgagctgggtggccctgaccgccggcctggtggccggcacccgctgccccgacggccagttctgccccgtggcctgctgcctggaccccggcggcgccagctacagctgctgccgccccctgctggacaagtggcccaccaccctgagccgccacctgggcggcccctgccaggtggacgcccactgcagcgccggccacagctgcatcttcaccgtgagcggcaccagcagctgctgccccttccccgaggccgtggcctgcggcgacggccaccactgctgcccccgcggcttccactgcagcgccgacggccgcagctgcttccagcgcagcggcaacaacagcgtgggcgccatccagtgccccgacagccagttcgagtgccccgacttcagcacctgctgcgtgatggtggacggcagctggggctgctgccccatgccccaggccagctgctgcgaggaccgcgtgcactgctgcccccacggcgccttctgcgacctggtgcacacccgctgcatcacccccaccggcacccaccccctggccaagaagctgcccgcccagcgcaccaaccgcgccgtggccctgagcagcagcgtgatgtgccccgacgcccgcagccgctgccccgacggcagcacctgctgcgagctgcccagcggcaagtacggctgctgccccatgcccaacgccacctgctgcagcgaccacctgcactgctgcccccaggacaccgtgtgcgacctgatccagagcaagtgcctgagcaaggagaacgccaccaccgacctgctgaccaagctgcccgcccacaccgtgggcgacgtgaagtgcgacatggaggtgagctgccccgacggctacacctgctgccgcctgcagagcggcgcctggggctgctgccccttcacccaggccgtgtgctgcgaggaccacatccactgctgccccgccggcttcacctgcgacacccagaagggcacctgcgagcagggcccccaccaggtgccctggatggagaaggcccccgcccacctgagcctgcccgacccccaggccctgaagcgcgacgtgccctgcgacaacgtgagcagctgccccagcagcgacacctgctgccagctgaccagcggcgagtggggctgctgccccatccccgaggccgtgtgctgcagcgaccaccagcactgctgcccccagggctacacctgcgtggccgagggccagtgccagcgcggcagcgagatcgtggccggcctggagaagatgcccgcccgccgcgccagcctgagccacccccgcgacatcggctgcgaccagcacaccagctgccccgtgggccagacctgctgccccagcctgggcggcagctgggcctgctgccagctgccccacgccgtgtgctgcgaggaccgccagcactgctgccccgccggctacacctgcaacgtgaaggcccgcagctgcgagaaggaggtggtgagcgcccagcccgccaccttcctggcccgcagcccccacgtgggcgtgaaggacgtggagtgcggcgagggccacttctgccacgacaaccagacctgctgccgcgacaaccgccagggctgggcctgctgcccctaccgccagggcgtgtgctgcgccgaccgccgccactgctgccccgccggcttccgctgcgccgcccgcggcaccaagtgcctgcgccgcgaggccccccgctgggacgcccccctgcgcgaccccgccctgcgccagctgctg

SEQ ID NO: 69 - DNA, 10871 bp, synthetic construct
  misc_feature 1..10871: note = Synthetic polynucleotide
  source 1..10871: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgtggaccctggtgagctgggtggccctgaccgccggcctggtggccggcacccgctgccccgacggccagttctgccccgtggcctgctgcctggaccccggcggcgccagctacagctgctgccgccccctgctggacaagtggcccaccaccctgagccgccacctgggcggcccctgccaggtggacgcccactgcagcgccggccacagctgcatcttcaccgtgagcggcaccagcagctgctgccccttccccgaggccgtggcctgcggcgacggccaccactgctgcccccgcggcttccactgcagcgccgacggccgcagctgcttccagcgcagcggcaacaacagcgtgggcgccatccagtgccccgacagccagttcgagtgccccgacttcagcacctgctgcgtgatggtggacggcagctggggctgctgccccatgccccaggccagctgctgcgaggaccgcgtgcactgctgcccccacggcgccttctgcgacctggtgcacacccgctgcatcacccccaccggcacccaccccctggccaagaagctgcccgcccagcgcaccaaccgcgccgtggccctgagcagcagcgtgatgtgccccgacgcccgcagccgctgccccgacggcagcacctgctgcgagctgcccagcggcaagtacggctgctgccccatgcccaacgccacctgctgcagcgaccacctgcactgctgcccccaggacaccgtgtgcgacctgatccagagcaagtgcctgagcaaggagaacgccaccaccgacctgctgaccaagctgcccgcccacaccgtgggcgacgtgaagtgcgacatggaggtgagctgccccgacggctacacctgctgccgcctgcagagcggcgcctggggctgctgccccttcacccaggccgtgtgctgcgaggaccacatccactgctgccccgccggcttcacctgcgacacccagaagggcacctgcgagcagggcccccaccaggtgccctggatggagaaggcccccgcccacctgagcctgcccgacccccaggccctgaagcgcgacgtgccctgcgacaacgtgagcagctgccccagcagcgacacctgctgccagctgaccagcggcgagtggggctgctgccccatccccgaggccgtgtgctgcagcgaccaccagcactgctgcccccagggctacacctgcgtggccgagggccagtgccagcgcggcagcgagatcgtggccggcctggagaagatgcccgcccgccgcgccagcctgagccacccccgcgacatcggctgcgaccagcacaccagctgccccgtgggccagacctgctgccccagcctgggcggcagctgggcctgctgccagctgccccacgccgtgtgctgcgaggaccgccagcactgctgccccgccggctacacctgcaacgtgaaggcccgcagctgcgagaaggaggtggtgagcgcccagcccgccaccttcctggcccgcagcccccacgtgggcgtgaaggacgtggagtgcggcgagggccacttctgccacgacaaccagacctgctgccgcgacaaccgccagggctgggcctgctgcccctaccgccagggcgtgtgctgcgccgaccgccgccactgctgccccgccggcttccgctgcgccgcccgcggcaccaagtgcctgcgccgcgaggccccccgctgggacgcccccctgcgcgaccccgccctgcgccagctgctgtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 70 - DNA, 4151 bp, synthetic construct
  misc_feature 1..4151: note = Synthetic polynucleotide
  source 1..4151: mol_type = other DNA
gggaggttacgcgttcgtcgactactagtgggtaccagagcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacagtccggaaagccaccatgtacgccctgttcctgctggccagcctgctgggcgccgccctggccggccccgtgctgggcctgaaggagtgcacccgcggcagcgccgtgtggtgccagaacgtgaagaccgccagcgactgcggcgccgtgaagcactgcctgcagaccgtgtggaacaagcccaccgtgaagagcctgccctgcgacatctgcaaggacgtggtgaccgccgccggcgacatgctgaaggacaacgccaccgaggaggagatcctggtgtacctggagaagacctgcgactggctgcccaagcccaacatgagcgccagctgcaaggagatcgtggacagctacctgcccgtgatcctggacatcatcaagggcgagatgagccgccccggcgaggtgtgcagcgccctgaacctgtgcgagagcctgcagaagcacctggccgagctgaaccaccagaagcagctggagagcaacaagatccccgagctggacatgaccgaggtggtggcccccttcatggccaacatccccctgctgctgtacccccaggacggcccccgcagcaagccccagcccaaggacaacggcgacgtgtgccaggactgcatccagatggtgaccgacatccagaccgccgtgcgcaccaacagcaccttcgtgcaggccctggtggagcacgtgaaggaggagtgcgaccgcctgggccccggcatggccgacatctgcaagaactacatcagccagtacagcgagatcgccatccagatgatgatgcacatgcagcccaaggagatctgcgccctggtgggcttctgcgacgaggtgaaggagatgcccatgcagaccctggtgcccgccaaggtggccagcaagaacgtgatccccgccctggagctggtggagcccatcaagaagcacgaggtgcccgccaagagcgacgtgtactgcgaggtgtgcgagttcctggtgaaggaggtgaccaagctgatcgacaacaacaagaccgagaaggagatcctggacgccttcgacaagatgtgcagcaagctgcccaagagcctgagcgaggagtgccaggaggtggtggacacctacggcagcagcatcctgagcatcctgctggaggaggtgagccccgagctggtgtgcagcatgctgcacctgtgcagcggcacccgcctgcccgccctgaccgtgcacgtgacccagcccaaggacggcggcttctgcgaggtgtgcaagaagctggtgggctacctggaccgcaacctggagaagaacagcaccaagcaggagatcctggccgccctggagaagggctgcagcttcctgcccgacccctaccagaagcagtgcgaccagttcgtggccgagtacgagcccgtgctgatcgagatcctggtggaggtgatggaccccagcttcgtgtgcctgaagatcggcgcctgccccagcgcccacaagcccctgctgggcaccgagaagtgcatctggggccccagctactggtgccagaacaccgagaccgccgcccagtgcaacgccgtggagcactgcaagcgccacgtgtggaacagaagaaagagaggaagtggagagggcagaggaagtcttctgacatgcggagacgtggaagagaatcccggccctatgtggaccctggtgagctgggtggccctgaccgccggcctggtggccggcacccgctgccccgacggccagttctgccccgtggcctgctgcctggaccccggcggcgccagctacagctgctgccgccccctgctggacaagtggcccaccaccctgagccgccacctgggcggcccctgccaggtggacgcccactgcagcgccggccacagctgcatcttcaccgtgagcggcaccagcagctgctgccccttccccgaggccgtggcctgcggcgacggccaccactgctgcccccgcggcttccactgcagcgccgacggccgcagctgcttccagcgcagcggcaacaacagcgtgggcgccatccagtgccccgacagccagttcgagtgccccgacttcagcacctgctgcgtgatggtggacggcagctggggctgctgccccatgccccaggccagctgctgcgaggaccgcgtgcactgctgcccccacggcgccttctgcgacctggtgcacacccgctgcatcacccccaccggcacccaccccctggccaagaagctgcccgcccagcgcaccaaccgcgccgtggccctgagcagcagcgtgatgtgccccgacgcccgcagccgctgccccgacggcagcacctgctgcgagctgcccagcggcaagtacggctgctgccccatgcccaacgccacctgctgcagcgaccacctgcactgctgcccccaggacaccgtgtgcgacctgatccagagcaagtgcctgagcaaggagaacgccaccaccgacctgctgaccaagctgcccgcccacaccgtgggcgacgtgaagtgcgacatggaggtgagctgccccgacggctacacctgctgccgcctgcagagcggcgcctggggctgctgccccttcacccaggccgtgtgctgcgaggaccacatccactgctgccccgccggcttcacctgcgacacccagaagggcacctgcgagcagggcccccaccaggtgccctggatggagaaggcccccgcccacctgagcctgcccgacccccaggccctgaagcgcgacgtgccctgcgacaacgtgagcagctgccccagcagcgacacctgctgccagctgaccagcggcgagtggggctgctgccccatccccgaggccgtgtgctgcagcgaccaccagcactgctgcccccagggctacacctgcgtggccgagggccagtgccagcgcggcagcgagatcgtggccggcctggagaagatgcccgcccgccgcgccagcctgagccacccccgcgacatcggctgcgaccagcacaccagctgccccgtgggccagacctgctgccccagcctgggcggcagctgggcctgctgccagctgccccacgccgtgtgctgcgaggaccgccagcactgctgccccgccggctacacctgcaacgtgaaggcccgcagctgcgagaaggaggtggtgagcgcccagcccgccaccttcctggcccgcagcccccacgtgggcgtgaaggacgtggagtgcggcgagggccacttctgccacgacaaccagacctgctgccgcgacaaccgccagggctgggcctgctgcccctaccgccagggcgtgtgctgcgccgaccgccgccactgctgccccgccggcttccgctgcgccgcccgcggcaccaagtgcctgcgccgcgaggccccccgctgggacgcccccctgcgcgaccccgccctgcgccagctgctgtgacaattgttaattaagtttaaaccctcgaggccgcaagcaataaaatatctttattttcattacatctgtgtgttggttttttgtgtgacaattgttaattaagtttaaacgttcgaggccgcaagcgagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctc

SEQ ID NO: 71 - DNA, 23 bp, synthetic construct
  misc_feature 1..23: note = Synthetic polynucleotide
  source 1..23: mol_type = other DNA
aagagggtgttctctatgtaggc

SEQ ID NO: 72 - DNA, 22 bp, synthetic construct
  misc_feature 1..22: note = Synthetic polynucleotide
  source 1..22: mol_type = other DNA
gctcctccaacatttgtcactt

SEQ ID NO: 73 - DNA, 23 bp, synthetic construct
  misc_feature 1..23: note = Synthetic polynucleotide
  source 1..23: mol_type = other DNA
acacagtacctaccgttatagca

SEQ ID NO: 74 - DNA, 23 bp, synthetic construct
  misc_feature 1..23: note = Synthetic polynucleotide
  source 1..23: mol_type = other DNA
tgttgtcacagtaacttgcatca

SEQ ID NO: 75 - DNA, 19 bp, synthetic construct
  misc_feature 1..19: note = Synthetic polynucleotide
  source 1..19: mol_type = other DNA
ctgggctacactgagcacc

SEQ ID NO: 76 - DNA, 21 bp, synthetic construct
  misc_feature 1..21: note = Synthetic polynucleotide
  source 1..21: mol_type = other DNA
aagtggtcgttgagggcaatg

SEQ ID NO: 77 - DNA, 20 bp, synthetic construct
  misc_feature 1..20: note = Synthetic polynucleotide
  source 1..20: mol_type = other DNA
tattagatctgatggccgcg

SEQ ID NO: 78 - DNA, 20 bp, synthetic construct
  misc_feature 1..20: note = Synthetic polynucleotide
  source 1..20: mol_type = other DNA
tccatcactaggggttcctg

SEQ ID NO: 79 - DNA, 4013 bp, synthetic construct
  misc_feature 1..4013: note = Synthetic polynucleotide
  source 1..4013: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 80 - DNA, 4013 bp, synthetic construct; identical to SEQ ID NO: 79
  misc_feature 1..4013: note = Synthetic polynucleotide
  source 1..4013: mol_type = other DNA

SEQ ID NO: 81 - DNA, 4162 bp, synthetic construct
  misc_feature 1..4162: note = Synthetic polynucleotide
  source 1..4162: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctcagcgctgtaattagcgcttggtttaatgacggcttgttggaggcttgctgaaggctgtatgctgttgtctttagaaataagtggtagtcaagtgaagccacagatgtgactaccacttatttctaaaaggacacaaggcctgttactagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggcccacatgtacactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 82 - DNA, 4184 bp, synthetic construct
  misc_feature 1..4184: note = Synthetic polynucleotide
  source 1..4184: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgtggaccctggtgagctgggtggccctgaccgccggcctggtggccggcacccgctgccccgacggccagttctgccccgtggcctgctgcctggaccccggcggcgccagctacagctgctgccgccccctgctggacaagtggcccaccaccctgagccgccacctgggcggcccctgccaggtggacgcccactgcagcgccggccacagctgcatcttcaccgtgagcggcaccagcagctgctgccccttccccgaggccgtggcctgcggcgacggccaccactgctgcccccgcggcttccactgcagcgccgacggccgcagctgcttccagcgcagcggcaacaacagcgtgggcgccatccagtgccccgacagccagttcgagtgccccgacttcagcacctgctgcgtgatggtggacggcagctggggctgctgccccatgccccaggccagctgctgcgaggaccgcgtgcactgctgcccccacggcgccttctgcgacctggtgcacacccgctgcatcacccccaccggcacccaccccctggccaagaagctgcccgcccagcgcaccaaccgcgccgtggccctgagcagcagcgtgatgtgccccgacgcccgcagccgctgccccgacggcagcacctgctgcgagctgcccagcggcaagtacggctgctgccccatgcccaacgccacctgctgcagcgaccacctgcactgctgcccccaggacaccgtgtgcgacctgatccagagcaagtgcctgagcaaggagaacgccaccaccgacctgctgaccaagctgcccgcccacaccgtgggcgacgtgaagtgcgacatggaggtgagctgccccgacggctacacctgctgccgcctgcagagcggcgcctggggctgctgccccttcacccaggccgtgtgctgcgaggaccacatccactgctgccccgccggcttcacctgcgacacccagaagggcacctgcgagcagggcccccaccaggtgccctggatggagaaggcccccgcccacctgagcctgcccgacccccaggccctgaagcgcgacgtgccctgcgacaacgtgagcagctgccccagcagcgacacctgctgccagctgaccagcggcgagtggggctgctgccccatccccgaggccgtgtgctgcagcgaccaccagcactgctgcccccagggctacacctgcgtggccgagggccagtgccagcgcggcagcgagatcgtggccggcctggagaagatgcccgcccgccgcgccagcctgagccacccccgcgacatcggctgcgaccagcacaccagctgccccgtgggccagacctgctgccccagcctgggcggcagctgggcctgctgccagctgccccacgccgtgtgctgcgaggaccgccagcactgctgccccgccggctacacctgcaacgtgaaggcccgcagctgcgagaaggaggtggtgagcgcccagcccgccaccttcctggcccgcagcccccacgtgggcgtgaaggacgtggagtgcggcgagggccacttctgccacgacaaccagacctgctgccgcgacaaccgccagggctgggcctgctgcccctaccgccagggcgtgtgctgcgccgaccgccgccactgctgccccgccggcttccgctgcgccgcccgcggcaccaagtgcctgcgccgcgaggccccccgctgggacgcccccctgcgcgaccccgccctgcgccagctgctgtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggcccacatgtacactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 83 - DNA, 4184 bp, synthetic construct; identical to SEQ ID NO: 82
  misc_feature 1..4184: note = Synthetic polynucleotide
  source 1..4184: mol_type = other DNA

SEQ ID NO: 84 - DNA, 4578 bp, synthetic construct
  misc_feature 1..4578: note = Synthetic polynucleotide
  source 1..4578: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagcaaaaaaattgtcatcctcccacggtggccatttgttccatgtgagtgctagtaacaggccttgtgtcctttgtagactatttgcacactgcatctgtggcttcactcagtgtgcaaatagtctacaagacaacagcatacagccttcagcaagcctccagtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgtttcccatggtgaagcttggatctgatccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgtggaccctggtgagctgggtggccctgaccgccggcctggtggccggcacccgctgccccgacggccagttctgccccgtggcctgctgcctggaccccggcggcgccagctacagctgctgccgccccctgctggacaagtggcccaccaccctgagccgccacctgggcggcccctgccaggtggacgcccactgcagcgccggccacagctgcatcttcaccgtgagcggcaccagcagctgctgccccttccccgaggccgtggcctgcggcgacggccaccactgctgcccccgcggcttccactgcagcgccgacggccgcagctgcttccagcgcagcggcaacaacagcgtgggcgccatccagtgccccgacagccagttcgagtgccccgacttcagcacctgctgcgtgatggtggacggcagctggggctgctgccccatgccccaggccagctgctgcgaggaccgcgtgcactgctgcccccacggcgccttctgcgacctggtgcacacccgctgcatcacccccaccggcacccaccccctggccaagaagctgcccgcccagcgcaccaaccgcgccgtggccctgagcagcagcgtgatgtgccccgacgcccgcagccgctgccccgacggcagcacctgctgcgagctgcccagcggcaagtacggctgctgccccatgcccaacgccacctgctgcagcgaccacctgcactgctgcccccaggacaccgtgtgcgacctgatccagagcaagtgcctgagcaaggagaacgccaccaccgacctgctgaccaagctgcccgcccacaccgtgggcgacgtgaagtgcgacatggaggtgagctgccccgacggctacacctgctgccgcctgcagagcggcgcctggggctgctgccccttcacccaggccgtgtgctgcgaggaccacatccactgctgccccgccggcttcacctgcgacacccagaagggcacctgcgagcagggcccccaccaggtgccctggatggagaaggcccccgcccacctgagcctgcccgacccccaggccctgaagcgcgacgtgccctgcgacaacgtgagcagctgccccagcagcgacacctgctgccagctgaccagcggcgagtggggctgctgccccatccccgaggccgtgtgctgcagcgaccaccagcactgctgcccccagggctacacctgcgtggccgagggccagtgccagcgcggcagcgagatcgtggccggcctggagaagatgcccgcccgccgcgccagcctgagccacccccgcgacatcggctgcgaccagcacaccagctgccccgtgggccagacctgctgccccagcctgggcggcagctgggcctgctgccagctgccccacgccgtgtgctgcgaggaccgccagcactgctgccccgccggctacacctgcaacgtgaaggcccgcagctgcgagaaggaggtggtgagcgcccagcccgccaccttcctggcccgcagcccccacgtgggcgtgaaggacgtggagtgcggcgagggccacttctgccacgacaaccagacctgctgccgcgacaaccgccagggctgggcctgctgcccctaccgccagggcgtgtgctgcgccgaccgccgccactgctgccccgccggcttccgctgcgccgcccgcggcaccaagtgcctgcgccgcgaggccccccgctgggacgcccccctgcgcgaccccgccctgcgccagctgctgtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 85 - DNA, 4162 bp, synthetic construct; identical to SEQ ID NO: 81
  misc_feature 1..4162: note = Synthetic polynucleotide
  source 1..4162: mol_type = other DNA

SEQ ID NO: 86 - DNA, 3977 bp, synthetic construct
  misc_feature 1..3977: note = Synthetic polynucleotide
  source 1..3977: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgtacgccctgttcctgctggccagcctgctgggcgccgccctggccggccccgtgctgggcctgaaggagtgcacccgcggcagcgccgtgtggtgccagaacgtgaagaccgccagcgactgcggcgccgtgaagcactgcctgcagaccgtgtggaacaagcccaccgtgaagagcctgccctgcgacatctgcaaggacgtggtgaccgccgccggcgacatgctgaaggacaacgccaccgaggaggagatcctggtgtacctggagaagacctgcgactggctgcccaagcccaacatgagcgccagctgcaaggagatcgtggacagctacctgcccgtgatcctggacatcatcaagggcgagatgagccgccccggcgaggtgtgcagcgccctgaacctgtgcgagagcctgcagaagcacctggccgagctgaaccaccagaagcagctggagagcaacaagatccccgagctggacatgaccgaggtggtggcccccttcatggccaacatccccctgctgctgtacccccaggacggcccccgcagcaagccccagcccaaggacaacggcgacgtgtgccaggactgcatccagatggtgaccgacatccagaccgccgtgcgcaccaacagcaccttcgtgcaggccctggtggagcacgtgaaggaggagtgcgaccgcctgggccccggcatggccgacatctgcaagaactacatcagccagtacagcgagatcgccatccagatgatgatgcacatgcagcccaaggagatctgcgccctggtgggcttctgcgacgaggtgaaggagatgcccatgcagaccctggtgcccgccaaggtggccagcaagaacgtgatccccgccctggagctggtggagcccatcaagaagcacgaggtgcccgccaagagcgacgtgtactgcgaggtgtgcgagttcctggtgaaggaggtgaccaagctgatcgacaacaacaagaccgagaaggagatcctggacgccttcgacaagatgtgcagcaagctgcccaagagcctgagcgaggagtgccaggaggtggtggacacctacggcagcagcatcctgagcatcctgctggaggaggtgagccccgagctggtgtgcagcatgctgcacctgtgcagcggcacccgcctgcccgccctgaccgtgcacgtgacccagcccaaggacggcggcttctgcgaggtgtgcaagaagctggtgggctacctggaccgcaacctggagaagaacagcaccaagcaggagatcctggccgccctggagaagggctgcagcttcctgcccgacccctaccagaagcagtgcgaccagttcgtggccgagtacgagcccgtgctgatcgagatcctggtggaggtgatggaccccagcttcgtgtgcctgaagatcggcgcctgccccagcgcccacaagcccctgctgggcaccgagaagtgcatctggggccccagctactggtgccagaacaccgagaccgccgcccagtgcaacgccgtggagcactgcaagcgccacgtgtggaactgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggcccacatgtacactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 87 - DNA, 4013 bp, synthetic construct; identical to SEQ ID NO: 79
  misc_feature 1..4013: note = Synthetic polynucleotide
  source 1..4013: mol_type = other DNA

SEQ ID NO: 88 - DNA, 4625 bp, synthetic construct
  misc_feature 1..4625: note = Synthetic polynucleotide
  source 1..4625: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacagtccggaaagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagagaagaaagagaggaagtggagagggcagaggaagtcttctgacatgcggagacgtggaagagaatcccggccctatggccgagtggctgctgagcgccagctggcagcgccgcgccaaggccatgaccgccgccgccggcagcgccggccgcgccgccgtgcccctgctgctgtgcgccctgctggcccccggcggcgcctacgtgctggacgacagcgacggcctgggccgcgagttcgacggcatcggcgccgtgagcggcggcggcgccaccagccgcctgctggtgaactaccccgagccctaccgcagccagatcctggactacctgttcaagcccaacttcggcgccagcctgcacatcctgaaggtggagatcggcggcgacggccagaccaccgacggcaccgagcccagccacatgcactacgccctggacgagaactacttccgcggctacgagtggtggctgatgaaggaggccaagaagcgcaaccccaacatcaccctgatcggcctgccctggagcttccccggctggctgggcaagggcttcgactggccctacgtgaacctgcagctgaccgcctactacgtggtgacctggatcgtgggcgccaagcgctaccacgacctggacatcgactacatcggcatctggaacgagcgcagctacaacgccaactacatcaagatcctgcgcaagatgctgaactaccagggcctgcagcgcgtgaagatcatcgccagcgacaacctgtgggagagcatcagcgccagcatgctgctggacgccgagctgttcaaggtggtggacgtgatcggcgcccactaccccggcacccacagcgccaaggacgccaagctgaccggcaagaagctgtggagcagcgaggacttcagcaccctgaacagcgacatgggcgccggctgctggggccgcatcctgaaccagaactacatcaacggctacatgaccagcaccatcgcctggaacctggtggccagctactacgagcagctgccctacggccgctgcggcctgatgaccgcccaggagccctggagcggccactacgtggtggagagccccgtgtgggtgagcgcccacaccacccagttcacccagcccggctggtactacctgaagaccgtgggccacctggagaagggcggcagctacgtggccctgaccgacggcctgggcaacctgaccatcatcatcgagaccatgagccacaagcacagcaagtgcatccgccccttcctgccctacttcaacgtgagccagcagttcgccaccttcgtgctgaagggcagcttcagcgagatccccgagctgcaggtgtggtacaccaagctgggcaagaccagcgagcgcttcctgttcaagcagctggacagcctgtggctgctggacagcgacggcagcttcaccctgagcctgcacgaggacgagctgttcaccctgaccaccctgaccaccggccgcaagggcagctaccccctgccccccaagagccagcccttccccagcacctacaaggacgacttcaacgtggactaccccttcttcagcgaggcccccaacttcgccgaccagaccggcgtgttcgagtacttcaccaacatcgaggaccccggcgagcaccacttcaccctgcgccaggtgctgaaccagcgccccatcacctgggccgccgacgccagcaacaccatcagcatcatcggcgactacaactggaccaacctgaccatcaagtgcgacgtgtacatcgagacccccgacaccggcggcgtgttcatcgccggccgcgtgaacaagggcggcatcctgatccgcagcgcccgcggcatcttcttctggatcttcgccaacggcagctaccgcgtgaccggcgacctggccggctggatcatctacgccctgggccgcgtggaggtgaccgccaagaagtggtacaccctgaccctgaccatcaagggccacttcaccagcggcatgctgaacgacaagagcctgtggaccgacatccccgtgaacttccccaagaacggctgggccgccatcggcacccacagcttcgagttcgcccagttcgacaacttcctggtggaggccacccgctgacaattgttaattaagtttaaaccctcgaggccgcaagcaataaaatatctttattttcattacatctgtgtgttggttttttgtgttgtacactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 89 - DNA, 4606 bp, synthetic construct
  misc_feature 1..4606: note = Synthetic polynucleotide
  source 1..4606: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctcctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccaccgcagccctaggaatgcatctagacaattgtactaaccttcttctctttcctctcctgacagtccggaaagccaccatgtacgccctgttcctgctggccagcctgctgggcgccgccctggccggccccgtgctgggcctgaaggagtgcacccgcggcagcgccgtgtggtgccagaacgtgaagaccgccagcgactgcggcgccgtgaagcactgcctgcagaccgtgtggaacaagcccaccgtgaagagcctgccctgcgacatctgcaaggacgtggtgaccgccgccggcgacatgctgaaggacaacgccaccgaggaggagatcctggtgtacctggagaagacctgcgactggctgcccaagcccaacatgagcgccagctgcaaggagatcgtggacagctacctgcccgtgatcctggacatcatcaagggcgagatgagccgccccggcgaggtgtgcagcgccctgaacctgtgcgagagcctgcagaagcacctggccgagctgaaccaccagaagcagctggagagcaacaagatccccgagctggacatgaccgaggtggtggcccccttcatggccaacatccccctgctgctgtacccccaggacggcccccgcagcaagccccagcccaaggacaacggcgacgtgtgccaggactgcatccagatggtgaccgacatccagaccgccgtgcgcaccaacagcaccttcgtgcaggccctggtggagcacgtgaaggaggagtgcgaccgcctgggccccggcatggccgacatctgcaagaactacatcagccagtacagcgagatcgccatccagatgatgatgcacatgcagcccaaggagatctgcgccctggtgggcttctgcgacgaggtgaaggagatgcccatgcagaccctggtgcccgccaaggtggccagcaagaacgtgatccccgccctggagctggtggagcccatcaagaagcacgaggtgcccgccaagagcgacgtgtactgcgaggtgtgcgagttcctggtgaaggaggtgaccaagctgatcgacaacaacaagaccgagaaggagatcctggacgccttcgacaagatgtgcagcaagctgcccaagagcctgagcgaggagtgccaggaggtggtggacacctacggcagcagcatcctgagcatcctgctggaggaggtgagccccgagctggtgtgcagcatgctgcacctgtgcagcggcacccgcctgcccgccctgaccgtgcacgtgacccagcccaaggacggcggcttctgcgaggtgtgcaagaagctggtgggctacctggaccgcaacctggagaagaacagcaccaagcaggagatcctggccgccctggagaagggctgcagcttcctgcccgacccctaccagaagcagtgcgaccagttcgtggccgagtacgagcccgtgctgatcgagatcctggtggaggtgatggaccccagcttcgtgtgcctgaagatcggcgcctgccccagcgcccacaagcccctgctgggcaccgagaagtgcatctggggccccagctactggtgccagaacaccgagaccgccgcccagtgcaacgccgtggagcactgcaagcgccacgtgtggaacagaagaaagagaggaagtggagagggcagaggaagtcttctgacatgcggagacgtggaagagaatcccggccctatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgtacactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 90 - DNA, 10870 bp, synthetic construct
  misc_feature 1..10870: note = Nucleotide sequence of first strand of PR006A vector
  source 1..10870: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgtggaccctggtgagctgggtggccctgaccgccggcctggtggccggcacccgctgccccgacggccagttctgccccgtggcctgctgcctggaccccggcggcgccagctacagctgctgccgccccctgctggacaagtggcccaccaccctgagccgccacctgggcggcccctgccaggtggacgcccactgcagcgccggccacagctgcatcttcaccgtgagcggcaccagcagctgctgccccttccccgaggccgtggcctgcggcgacggccaccactgctgcccccgcggcttccactgcagcgccgacggccgcagctgcttccagcgcagcggcaacaacagcgtgggcgccatccagtgccccgacagccagttcgagtgccccgacttcagcacctgctgcgtgatggtggacggcagctggggctgctgccccatgccccaggccagctgctgcgaggaccgcgtgcactgctgcccccacggcgccttctgcgacctggtgcacacccgctgcatcacccccaccggcacccaccccctggccaagaagctgcccgcccagcgcaccaaccgcgccgtggccctgagcagcagcgtgatgtgccccgacgcccgcagccgctgccccgacggcagcacctgctgcgagctgcccagcggcaagtacggctgctgccccatgcccaacgccacctgctgcagcgaccacctgcactgctgcccccaggacaccgtgtgcgacctgatccagagcaagtgcctgagcaaggagaacgccaccaccgacctgctgaccaagctgcccgcccacaccgtgggcgacgtgaagtgcgacatggaggtgagctgccccgacggctacacctgctgccgcctgcagagcggcgcctggggctgctgccccttcacccaggccgtgtgctgcgaggaccacatccactgctgccccgccggcttcacctgcgacacccagaagggcacctgcgagcagggcccccaccaggtgccctggatggagaaggcccccgcccacctgagcctgcccgacccccaggccctgaagcgcgacgtgccctgcgacaacgtgagcagctgccccagcagcgacacctgctgccagctgaccagcggcgagtggggctgctgccccatccccgaggccgtgtgctgcagcgaccaccagcactgctgcccccagggctacacctgcgtggccgagggccagtgccagcgcggcagcgagatcgtggccggcctggagaagatgcccgcccgccgcgccagcctgagccacccccgcgacatcggctgcgaccagcacaccagctgccccgtgggccagacctgctgccccagcctgggcggcagctgggcctgctgccagctgccccacgccgtgtgctgcgaggaccgccagcactgctgccccgccggctacacctgcaacgtgaaggcccgcagctgcgagaaggaggtggtgagcgcccagcccgccaccttcctggcccgcagcccccacgtgggcgtgaaggacgtggagtgcggcgagggccacttctgccacgacaaccagacctgctgccgcgacaaccgccagggctgggcctgctgcccctaccgccagggcgtgtgctgcgccgaccgccgccactgctgccccgccggcttccgctgcgccgcccgcggcaccaagtgcctgcgccgcgaggccccccgctgggacgcccccctgcgcgaccccgccctgcgccagctgctgtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggcccacatgtacactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 91 - DNA, 10870 bp, synthetic construct
  misc_feature 1..10870: note = Nucleotide sequence of second strand of PR006A vector
  source 1..10870: mol_type = other DNA
gactgccggacgtcgacttggcgcgccctcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggttttctctacactgaagtcttgatggcatgcgctttagtctcctcactcatgaggactgcatatctaagagctctgtgacctcacttgtgcctggtgtccagggtaaacaaatcttcttgcctttgaaaattagaggatgcagactggtctcctctactgcactggactgactaacctgactggagctcttgttcacactgagaagaagctggtggactcttctttggctctgaaaggcagatttattttatctcatggagttaggctagagcatcttgtcagagggaatgatgtttgaagtccatgaggcttctaatgattagggctggtatgcagcaaacaaccatgactgcatttgtctctgatgcagattcctaacttctgatatgttcaaaactctgcttcctgattcagttgctctctttaacaaatgcagtgattcttttgataatttgaggggaaaaaatgtctctgtgttgtgtttcctccttgctggtgatccacagtaaggggttgaaaataatctctgacattttttagttaaagccttcctttcattcttcatggctactgcagcacaaagctctttcttgttttgcagaaggagtgagagcaaggtgttgggtgagttctcagtgccccagccccagcctgaagaggagactgaatgcctgccctctggctgtggctcagagtgaaaggaaaacacatacagcaggagtctgtgtagggattgggctccagccatcagagcggggggagcatcatgaaatgtttgtaagctctccaccatccagaatgactttgcagctctgagcagctgcacccatgactagtccttcacagtatgctgatgtgagatattctgttctttgaagatgggtagtgaccagaatcaggaggatgtgagaaatgtggagcatttgagagtggaactcctgcagctgttgaagctagaacacaacagacactgcagcttactactcctgagcctggccaggctggtagttttgtttttcttggccaagctctctgggaatagttctgcctctggggtcagctttccatgcaggggctgagatgaacacataaagcaggattcagcttggaggcttctgagagggatggagataaaagatggtgtggtagatggtaagaggtggtatggtagatggcagagtatggcagaagatgtagacaggatgtcagaaaatgagatggtagttgcaagagtaggctgtagacagtgggagatggtgagatgagagatgcagaagatggtcagagagcaggatgttgggagaggctaggaggtgagacaccagggttggagtgtggactgcaggctttgttaggctggagaggtgagtagctgtggagagaggatgacctgcacttgtctgaatctaacagaacctaaattagaaactttctcttgcagttctctctctggctcaggctggcttgtcttggggactgtctgtcactctggaacagggtggttagagctgtggctttcatggtacttctcttcactgggctctgatagcctggggctgctggtactatctgcttgcacttgctttgaggaggccaagaacatcctgtgtatttccagactctttcttgttctgtttgtcctctcaggctattccttgtgctcctgatgtctctgtctttacttccaaaactatttattaaaaaatctaagattcattcatgtttaaggggaattatgctaattctttcatacctaatgggataaaactaactgtgaaattacagaccatcttgtgtaaaatcttaatctcagcagtcacatttgctgttgaagtgagtatgaacaaacactgatatgtggagggctctggtgcaaaggtttgtaggctctgcctgtgttgcatagagcagctcatctgaagtggattagcactgcttctgagcatggaaggtcaggctgaagtcatgatgcctaattagggctggagaggacatcacagaaacagtggctcttgggtttgtcctgggtagggcagaggtaaggttgaggagagctagaaaactcaggaaaagaaaaggctcaggagaaagaagacagagagttccagtgagcagagctgcctctgctgctaggcttgtggtacacaggttaaagtttggccttgtggcattcaacagcctttgcatcctactgaccacactagccaggcagttgaggctcagggctggctttcttgcacttggagaacctttcctaccttcaaatgcttgaaccagaataccagagaaaaaatgcaactctgaaagcacaggcattctgtgaatagccatccagatttctgcaggctaaggaaaacagagatggaggtctatgagggaaagaagagaagtcctcatgagggctgataccagacagttgagtaggacactgagcacactcaccaccagatgctctgtctaagctgtagtttactaggaggttgtttattctctgagtcagatttgcttctgttttaagaaccctcacagcctctctttctatctattctgatgaaggttgagcctgaagaagctggagcagaaagggtcttctgcctcatgttctgctgataaactttgagaatcctgcctaagtctgacctagattttggctccaaaagccagggcttcttatctctgtcaagaccaaacctggatgcagtcagaggttgggcacagcctttgctccaaggctcctgggcacagtgcccacctcccatttgagcttgcagcaaggctattatgagaaactttcctcctcttccttcaagtctccacctattagctctgcaagagtgcagaaggctgcacagcactgcagatggagaaggtggcaggggcagcaggtggcaggggctccagggaactgtggtcagagttgattcagagcaggctaagagtggagtgactgaagacagaagctggaggcacatcaaccattacaagacagcttgacttttgctagaacccttggcttcaggtgatgagaataatggtagacactggttgtttggcttgatagcttaaaaaaggacaaccattataatggaagcagtttctctgagaaacaggatgttggcaagggcttagtgctcctgactgctgatgctggagtctggcttccctgaccccagtagcagtgggacttagaagcatctcaggctccaggttctcacagctgactcctccttcctgaactgtaagaaacagaaatccacttgctgctctaaagtggggtcactttaatggaaggaacagccaacatatttggagaggggctggtgtggggcttcttactgagagtgggcagccctgagcactggacaaactcaactgtgggcaacctgggtgggaagcagctgtggagatggggaagccatgctaatcagtgacatcatctattctaagtttcctacctctgaatgaatagttctgctggcaaaaatctgcttttttaagttgatacaaatgtgtcctgtcaaggaagtagagctccttctcaatccagcacatcagtaccataacttgttcctgtgcatttggttaaagatggtgataacatcatttatcagtaagtgcagccaggcatgatgcactatctccttgcatttagagcctaattttggccaaagaagtcttcatagaaaaggacttcccactcaaagtagtcctgagtagttctactgattttattattagtagagagggtttcatcatgttggccaggctggtctgcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagaccatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtcttttgggcttctatatacattttagaatgaggttggcaagttatcctgcaggaattcctcgagaccgtacgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacctgcagggaattcagtgtacatgtgggccccaaaaaagctgtttgttatcgtggatctctccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgacggtatcgatgcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatccaggtggcaacacaggcgagcagccaaggaaaggacgatgatttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattatcgataagcttgcggcctcgagggtttaaacttaattaacaattgtcacagcagctggcgcagggcggggtcgcgcaggggggcgtcccagcggggggcctcgcggcgcaggcacttggtgccgcgggcggcgcagcggaagccggcggggcagcagtggcggcggtcggcgcagcacacgccctggcggtaggggcagcaggcccagccctggcggttgtcgcggcagcaggtctggttgtcgtggcagaagtggccctcgccgcactccacgtccttcacgcccacgtgggggctgcgggccaggaaggtggcgggctgggcgctcaccacctccttctcgcagctgcgggccttcacgttgcaggtgtagccggcggggcagcagtgctggcggtcctcgcagcacacggcgtggggcagctggcagcaggcccagctgccgcccaggctggggcagcaggtctggcccacggggcagctggtgtgctggtcgcagccgatgtcgcgggggtggctcaggctggcgcggcgggcgggcatcttctccaggccggccacgatctcgctgccgcgctggcactggccctcggccacgcaggtgtagccctgggggcagcagtgctggtggtcgctgcagcacacggcctcggggatggggcagcagccccactcgccgctggtcagctggcagcaggtgtcgctgctggggcagctgctcacgttgtcgcagggcacgtcgcgcttcagggcctgggggtcgggcaggctcaggtgggcgggggccttctccatccagggcacctggtgggggccctgctcgcaggtgcccttctgggtgtcgcaggtgaagccggcggggcagcagtggatgtggtcctcgcagcacacggcctgggtgaaggggcagcagccccaggcgccgctctgcaggcggcagcaggtgtagccgtcggggcagctcacctccatgtcgcacttcacgtcgcccacggtgtgggcgggcagcttggtcagcaggtcggtggtggcgttctccttgctcaggcacttgctctggatcaggtcgcacacggtgtcctgggggcagcagtgcaggtggtcgctgcagcaggtggcgttgggcatggggcagcagccgtacttgccgctgggcagctcgcagcaggtgctgccgtcggggcagcggctgcgggcgtcggggcacatcacgctgctgctcagggccacggcgcggttggtgcgctgggcgggcagcttcttggccagggggtgggtgccggtgggggtgatgcagcgggtgtgcaccaggtcgcagaaggcgccgtgggggcagcagtgcacgcggtcctcgcagcagctggcctggggcatggggcagcagccccagctgccgtccaccatcacgcagcaggtgctgaagtcggggcactcgaactggctgtcggggcactggatggcgcccacgctgttgttgccgctgcgctggaagcagctgcggccgtcggcgctgcagtggaagccgcgggggcagcagtggtggccgtcgccgcaggccacggcctcggggaaggggcagcagctgctggtgccgctcacggtgaagatgcagctgtggccggcgctgcagtgggcgtccacctggcaggggccgcccaggtggcggctcagggtggtgggccacttgtccagcagggggcggcagcagctgtagctggcgccgccggggtccaggcagcaggccacggggcagaactggccgtcggggcagcgggtgccggccaccaggccggcggtcagggccacccagctcaccagggtccacatggtggctcaaccggtgatatcttcgaacggatcccacagtcgtggaagactttcccttcggatcttcgaggaattctttgccaaaatgatgagacagcacaataaccagcacgttgcccaggagctgtaggaaaaagaagaaggcatgaacatggttagcagaggctctagctcccggagcccctcaaggctttcacgcagccacagaaaagaaacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactaggtaccgaattcagatccaagcttcaccatgggagacgtcaccggttctagaacctagggagctctggtacccactagtagtcgacgaacgcgtaacctcccgcttcaaaatggagaccctgcgtgctcactcgggcttaaatacccagagctagcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

(19) United States
(12) Patent Application Publication
(10) Pub. No.: US 2024/0287471 A1
(43) Pub. Date: Aug. 29, 2024

(54) GENE THERAPIES FOR LYSOSOMAL DISORDERS

(71) Applicant: Prevail Therapeutics, Inc., New York, NY (US)

(72) Inventors: Asa ABELIOVICH, New York, NY (US); Laura HECKMAN, New York, NY (US); Herve RHINN, New York, NY (US); Li Chin WONG, New York, NY (US); Hsuan-Ni LIN, New York, NY (US); Franz HEFTI, New York, NY (US)

(21) Appl. No.: 18/646,957

(22) Filed: Apr. 26, 2024

Related U.S. Application Data
(62) Division of application No. 16/846,065, filed on Apr. 10, 2020, now Pat. No. 11,999,974.
(60) Provisional application No. 62/988,665, filed on Mar. 12, 2020.
(60) Provisional application No. 62/960,471, filed on Jan. 13, 2020.
(60) Provisional application No. 62/954,089, filed on Dec. 27, 2019.
(60) Provisional application No. 62/934,450, filed on Nov. 12, 2019.
(60) Provisional application No. 62/831,846, filed on Apr. 10, 2019.

(51) Int. Cl. C12N 7/00; A61K 48/00; A61P 25/28; C07K 14/435; C12N 9/02; G01N 33/68

(52) U.S. Cl. CPC C12N 7/00; A61K 48/0075; A61K 48/0083; A61P 25/28; C07K 14/435; C12N 9/0071; C12Y 114/18001; G01N 33/6803; C12N 2750/14132; C12N 2750/14133; C12N 2750/14143; G01N 2333/435

(57) ABSTRACT

The disclosure relates to compositions and methods for treatment of diseases associated with aberrant lysosomal function, such as fronto-temporal dementia (FTD). The disclosure also provides expression constructs comprising a transgene encoding progranulin or a portion thereof. The disclosure provides methods of treating FTD by administering such expression constructs to a subject in need thereof.

### CROSS-REFERENCE TO RELATED APPLICATIONS

This application is a division of U.S. patent application Ser. No. 16/846,065, filed Apr. 10, 2020, which claims priority to U.S. Provisional Patent Application No. 62/988,665, filed on Mar. 12, 2020, U.S. Provisional Patent Application No. 62/960,471, filed on Jan. 13, 2020, U.S. Provisional Patent Application No. 62/954,089, filed on Dec. 27, 2019, U.S. Provisional Patent Application No. 62/934,450, filed on Nov. 12, 2019 and U.S. Provisional Patent Application No. 62/831,846, filed on Apr. 10, 2019. The disclosure of each of these applications is incorporated herein by reference in its entirety.

### REFERENCE TO AN ELECTRONIC SEQUENCE LISTING

The contents of the electronic sequence listing (PRVL_010_06US_SeqList_ST26.xml; Size: 535,200 bytes; and Date of Creation: Apr. 25, 2024) are herein incorporated by reference in their entirety.

### FIELD

The disclosure relates to the field of gene therapy and methods of using same.

### BACKGROUND

Gaucher disease is a rare inborn error of glycosphingolipid metabolism due to deficiency of lysosomal acid β-glucocerebrosidase (Gcase, “GBA”). Patients suffer from non-CNS symptoms and findings including hepatosplenomegaly, bone marrow insufficiency leading to pancytopenia, lung disorders and fibrosis, and bone defects. In addition, a significant number of patients suffer from neurological manifestations, including defective saccadic eye movements and gaze, seizures, cognitive deficits, developmental delay, and movement disorders including Parkinson's disease. Several therapeutics exist that address the peripheral disease and the principal clinical manifestations in hematopoietic bone marrow and viscera, including enzyme replacement therapies as described below, chaperone-like small molecule drugs that bind to defective Gcase and improve stability, and substrate reduction therapy that block the production of substrate that accumulate in Gaucher disease leading to symptoms and findings. However, other aspects of Gaucher disease (particularly those affecting the skeleton and brain) appear refractory to treatment.

Progranulin (PGRN) is an additional protein linked to lysosomal function. PGRN is encoded by the GRN gene. GRN haploinsufficiency in humans leads to an approximately 90% risk of developing FTD-GRN (fronto-temporal dementia with GRN mutation), a neurodegenerative disease characterized by impairment of executive function, changes in behavior, and language difficulties, accompanied by atrophy of the frontal and temporal lobes. No disease-modifying therapies are available for patients with FTD.

### SUMMARY

Provided herein is a method for treating a subject having or suspected of having fronto-temporal dementia with a GRN mutation, the method comprising administering to the subject a recombinant adeno-associated virus (rAAV) comprising: (i) a rAAV vector comprising a nucleic acid comprising an expression construct comprising a promoter operably linked to a transgene insert encoding a PGRN protein, wherein the transgene insert comprises the nucleotide sequence of SEQ ID NO: 68; and (ii) an AAV9 capsid protein. In some embodiments, the rAAV is administered to a subject at a dose ranging from about 1×10¹³ vector genomes (vg) to about 7×10¹⁴ vg. In some embodiments, the rAAV is administered via an injection into the cisterna magna.

In some embodiments, the promoter operably linked to a transgene insert encoding a PGRN protein is a chicken beta actin (CBA) promoter. In some embodiments, the rAAV vector further comprises a cytomegalovirus (CMV) enhancer. In some embodiments, the rAAV vector further comprises a Woodchuck Hepatitis Virus Posttranscriptional Regulatory Element (WPRE). In some embodiments, the rAAV vector further comprises a Bovine Growth Hormone polyA signal tail. In some embodiments, the nucleic acid comprises two adeno-associated virus inverted terminal repeats (ITR) sequences flanking the expression construct. In some embodiments, each ITR sequence is a wild-type AAV2 ITR sequence. In some embodiments, the rAAV vector further comprises a TRY region between the 5′ ITR and the expression construct, wherein the TRY region comprises SEQ ID NO: 28.

Provided herein is a method for treating a subject having or suspected of having fronto-temporal dementia with a GRN mutation, the method comprising administering to the subject a rAAV comprising: (i) a rAAV vector comprising a nucleic acid comprising, in 5′ to 3′ order: (a) an AAV2 ITR; (b) a CMV enhancer; (c) a CBA promoter; (d) a transgene insert encoding a PGRN protein, wherein the transgene insert comprises the nucleotide sequence of SEQ ID NO: 68; (e) a WPRE; (f) a Bovine Growth Hormone polyA signal tail; and (g) an AAV2 ITR; and (ii) an AAV9 capsid protein. In some embodiments, the rAAV is administered to a subject at a dose ranging from about 1×10¹³ vg to about 7×10¹⁴ vg. In some embodiments, the rAAV is administered via an injection into the cisterna magna.

In some embodiments, the rAAV is administered in a formulation comprising about 20 mM Tris, pH 8.0, about 1 mM MgCl₂, about 200 mM NaCl, and about 0.001% w/v poloxamer 188.

Provided herein is a pharmaceutical composition comprising (i) a rAAV comprising: (a) a rAAV vector comprising a nucleic acid comprising an expression construct comprising a promoter operably linked to a transgene insert encoding a PGRN protein, wherein the transgene insert comprises the nucleotide sequence of SEQ ID NO: 68; and (b) an AAV9 capsid protein; and (ii) about 20 mM Tris, pH 8.0, (iii) about 1 mM MgCl₂, (iv) about 200 mM NaCl, and (v) about 0.001% w/v poloxamer 188.

Provided herein is a rAAV comprising: (a) a rAAV vector comprising a nucleic acid comprising an expression construct comprising a promoter operably linked to a transgene insert encoding a PGRN protein, wherein the transgene insert comprises the nucleotide sequence of SEQ ID NO: 68; and (b) an AAV9 capsid protein, for use in a method of treating fronto-temporal dementia with a GRN mutation in a subject.

Provided herein is a method of quantifying a PGRN protein level in a cerebrospinal fluid (CSF) sample, the method comprising: (1) diluting the CSF sample in a master mix containing dithiothreitol (DTT) and sample buffer; (2) loading the diluted CSF sample, an anti-progranulin antibody, a secondary antibody that detects the anti-progranulin antibody, luminol and peroxide into wells of a capillary cartridge; (3) loading the capillary cartridge into an automated Western blot immunoassay instrument; (4) using the automated Western blot immunoassay instrument to calculate signal intensity, peak area, and signal-to-noise ratio; and (5) quantifying a progranulin protein level in the CSF sample as the peak area of immunoreactivity to the anti-progranulin antibody.

### BRIEF DESCRIPTION OF THE DRAWINGS

FIG. 1 is a schematic depicting one embodiment of a vector comprising an expression construct encoding Gcase (e.g., GBA1 or a portion thereof).

FIG. 2 is a schematic depicting one embodiment of a vector comprising an expression construct encoding Gcase (e.g., GBA1 or a portion thereof) and LIMP2 (SCARB2) or a portion thereof. The coding sequences of Gcase and LIMP2 are separated by an internal ribosomal entry site (IRES).

FIG. 3 is a schematic depicting one embodiment of a vector comprising an expression construct encoding Gcase (e.g., GBA1 or a portion thereof) and LIMP2 (SCARB2) or a portion thereof. Expression of the coding sequences of Gcase and LIMP2 are each driven by a separate promoter.

FIG. 4 is a schematic depicting one embodiment of a vector comprising an expression construct encoding Gcase (e.g., GBA1 or a portion thereof), LIMP2 (SCARB2) or a portion thereof, and an interfering RNA for α-Syn.

FIG. 5 is a schematic depicting one embodiment of a vector comprising an expression construct encoding Gcase (e.g., GBA1 or a portion thereof), Prosaposin (e.g., PSAP or a portion thereof), and an interfering RNA for α-Syn.

FIG. 6 is a schematic depicting one embodiment of a vector comprising an expression construct encoding Gcase (e.g., GBA1 or a portion thereof) and Prosaposin (e.g., PSAP or a portion thereof). The coding sequences of Gcase and Prosaposin are separated by an internal ribosomal entry site (IRES).

FIG. 7 is a schematic depicting one embodiment of a vector comprising an expression construct encoding a Gcase (e.g., GBA1 or a portion thereof). In this embodiment, the vector comprises a CBA promoter element (CBA), consisting of four parts: the CMV enhancer (CMVe), CBA promoter (CBAp), Exon 1, and intron (int) to constitutively express the codon optimized coding sequence of human GBA1. The 3′ region also contains a WPRE regulatory element followed by a bGH polyA tail. Three transcriptional regulatory activation sites are included at the 5′ end of the promoter region: TATA, RBS, and YY1. The flanking ITRs allow for the correct packaging of the intervening sequences. Two variants of the 5′ ITR sequence (inset box) were evaluated; these have several nucleotide differences within the 20-nucleotide “D” region of wild-type AAV2 ITR. In some embodiments, an rAAV vector contains the “D” domain nucleotide sequence shown on the top line. In some embodiments, a rAAV vector comprises a mutant “D” domain (e.g., an “S” domain, with the nucleotide changes shown on the bottom line).

FIG. 8 is a schematic depicting one embodiment of the vector described in FIG. 6

FIG. 9 shows representative data for delivery of an rAAV comprising a transgene encoding a Gcase (e.g., GBA1 or a portion thereof) in a CBE mouse model of Parkinson's disease. Daily IP delivery of PBS vehicle, 25 mg/kg CBE, 37.5 mg/kg CBE, or 50 mg/kg CBE (left to right) initiated at P8. Survival (top left) was checked two times a day and weight (top right) was checked daily. All groups started with n=8. Behavior was assessed by total distance traveled in Open Field (bottom left) at P23 and latency to fall on Rotarod (bottom middle) at P24. Levels of the GCase substrates were analyzed in the cortex of mice in the PBS and 25 mg/kg CBE treatment groups both with (Day 3) and without (Day 1) CBE withdrawal. Aggregate GluSph and GalSph levels (bottom right) are shown as pmol per mg wet weight of the tissue. Means are presented. Error bars are SEM. *p<0.05; **p<0.01; ***p<0.001, nominal p-values for treatment groups by linear regression.

FIG. 10 is a schematic depicting one embodiment of a study design for maximal rAAV dose in a CBE mouse model. Briefly, rAAV was delivered by ICV injection at P3, and daily CBE treatment was initiated at P8. Behavior was assessed in the Open Field and Rotarod assays at P24-25 and substrate levels were measured at P36 and P38.

FIG. 11 shows representative data for in-life assessment of maximal rAAV dose in a CBE mouse model. At P3, mice were treated with either excipient or 8.8e9 vg rAAV-GBA1 via ICV delivery. Daily IP delivery of either PBS or 25 mg/kg CBE was initiated at P8. At the end of the study, half the mice were sacrificed one day after their last CBE dose at P36 (Day 1) while the remaining half went through 3 days of CBE withdrawal before sacrifice at P38 (Day3). All treatment groups (excipient+PBS n=8, rAAV-GBA1+PBS n=7, excipient+CBE n=8, and variant+CBE n=9) were weighed daily (top left), and the weight at P36 was analyzed (top right). Behavior was assessed by total distance traveled in Open Field at P23 (bottom left) and latency to fall on Rotarod at P24 (bottom right), evaluated for each animal as the median across 3 trials. Due to lethality, n=7 for the excipient+CBE group for the behavioral assays, while n=8 for all other groups. Means across animals are presented. Error bars are SEM. *p<0.05; ***p<0.001, nominal p-values for treatment groups by linear regression in the CBE-treated animals.

FIG. 12 shows representative data for biochemical assessment of maximal rAAV dose in a CBE mouse model. The cortex of all treatment groups (excipient+PBS n=8, variant+PBS n=7, excipient+CBE n=7, and variant+CBE n=9) was used to measure GCase activity (top left), GluSph levels (top right), GluCer levels (bottom left), and vector genomes (bottom right) in the groups before (Day 1) or after (Day 3) CBE withdrawal. Biodistribution is shown as vector genomes per 1 μg of genomic DNA. Means are presented. Error bars are SEM. (*)p<0.1; **p<0.01; ***p<0.001, nominal p-values for treatment groups by linear regression in the CBE-treated animals, with collection days and gender corrected for as covariates.

FIG. 13 shows representative data for behavioral and biochemical correlations in a CBE mouse model after administration of excipient+PBS, excipient+CBE, and variant+CBE treatment groups. Across treatment groups, performance on Rotarod was negatively correlated with GluCer accumulation (A, p=0.0012 by linear regression), and GluSph accumulation was negatively correlated with increased GCase activity (B, p=0.0086 by linear regression).

FIG. 14 shows representative data for biodistribution of variant in a CBE mouse model. Presence of vector genomes was assessed in the liver, spleen, kidney, and gonads for all treatment groups (excipient+PBS n=8, variant+PBS n=7, excipient+CBE n=7, and variant+CBE n=9). Biodistribution is shown as vector genomes per 1 μg of genomic DNA. Vector genome presence was quantified by quantitative PCR using a vector reference standard curve; genomic DNA concentration was evaluated by A260 optical density measurement. Means are presented. Error bars are SEM. *p<0.05; **p<0.01; ***p<0.001, nominal p-values for treatment groups by linear regression in the CBE-treated animals, with collection days and gender corrected for as covariates.

FIG. 15 shows representative data for in-life assessment of rAAV dose ranging in a CBE mouse model. Mice received excipient or one of three different doses of rAAV-GBA1 by ICV delivery at P3: 3.2e9 vg, 1.0e10 vg, or 3.2e10 vg. At P8, daily IP treatment of 25 mg/kg CBE was initiated. Mice that received excipient and CBE or excipient and PBS served as controls. All treatment groups started with n=10 (5M/5F) per group. All mice were sacrificed one day after their final CBE dose (P38-P40). All treatment groups were weighed daily, and their weight was analyzed at P36. Motor performance was assessed by latency to fall on Rotarod at P24 and latency to traverse the Tapered Beam at P30. Due to early lethality, the number of mice participating in the behavioral assays was: excipient+PBS n=10, excipient+CBE n=9, and 3.2e9 vg rAAV-GBA1+CBE n=6, 1.0e10 vg rAAV-GBA1+CBE n=10, 3.2e10 vg rAAV-GBA1+CBE n=7. Means are presented. Error bars are SEM; * p<0.05; **p<0.01 for nominal p-values by linear regression in the CBE-treated groups, with gender corrected for as a covariate.

FIG. 16 shows representative data for biochemical assessment of rAAV dose ranging in a CBE mouse model. The cortex of all treatment groups (excipient+PBS n=10, excipient+CBE n=9, and 3.2e9 vg rAAV-GBA1+CBE n=6, 1.0e10 vg rAAV-GBA1+CBE n=10, 3.2e10 vg rAAV-GBA1+CBE n=7) was used to measure GCase activity, GluSph levels, GluCer levels, and vector genomes. GCase activity is shown as ng of GCase per mg of total protein. GluSph and GluCer levels are shown as pmol per mg wet weight of the tissue. Biodistribution is shown as vector genomes per 1 μg of genomic DNA. Vector genome presence was quantified by quantitative PCR using a vector reference standard curve; genomic DNA concentration was evaluated by A260 optical density measurement. Vector genome presence was also measured in the liver (E). Means are presented. Error bars are SEM. **p<0.01; ***p<0.001 for nominal p-values by linear regression in the CBE-treated groups, with gender corrected for as a covariate.

FIG. 17 shows representative data for tapered beam analysis in maximal dose rAAV-GBA1 in a genetic mouse model. Motor performance of the treatment groups (WT+excipient, n=5), 4L/PS-NA+excipient (n=6), and 4L/PS-NA+rAAV-GBA1 (n=5)) was assayed by Beam Walk 4 weeks post rAAV-GBA1 administration. The total slips and active time are shown as total over 5 trials on different beams. Speed and slips per speed are shown as the average over 5 trials on different beams. Means are presented. Error bars are SEM.

FIG. 18 shows representative data for in vitro expression of rAAV constructs encoding progranulin (PGRN) protein. The left panel shows a standard curve of progranulin (PGRN) ELISA assay. The bottom panel shows a dose-response of PGRN expression measured by ELISA assay in cell lysates of HEK293T cells transduced with rAAV. MOI=multiplicity of infection (vector genomes per cell).

FIG. 19 shows representative data for in vitro expression of rAAV constructs encoding GBA1 in combination with Prosaposin (PSAP), SCARB2, and/or one or more inhibitory nucleic acids. Data indicate transfection of HEK293 cells with each construct resulted in overexpression of the transgenes of interest relative to mock transfected cells.

FIG. 20 is a schematic depicting an rAAV vectors comprising a “D” region located on the “outside” of the ITR (e.g., proximal to the terminus of the ITR relative to the transgene insert or expression construct) (top) and a wild-type rAAV vectors having ITRs on the “inside” of the vector (e.g., proximal to the transgene insert of the vector).

FIG. 21 a schematic depicting one embodiment of a vector comprising an expression construct encoding GBA2 or a portion thereof, and an interfering RNA for α-Syn.

FIG. 22 is a schematic depicting one embodiment of a vector comprising an expression construct encoding Gcase (e.g., GBA1 or a portion thereof) and Galactosylceramidase (e.g., GALC or a portion thereof). Expression of the coding sequences of Gcase and Galactosylceramidase are separated by a T2A self-cleaving peptide sequence.

FIG. 23 is a schematic depicting one embodiment of a vector comprising an expression construct encoding Gcase (e.g., GBA1 or a portion thereof) and Galactosylceramidase (e.g., GALC or a portion thereof). Expression of the coding sequences of Gcase and Galactosylceramidase are separated by a T2A self-cleaving peptide sequence.

FIG. 24 is a schematic depicting one embodiment of a vector comprising an expression construct encoding Gcase (e.g., GBA1 or a portion thereof), Cathepsin B (e.g., CTSB or a portion thereof), and an interfering RNA for α-Syn. Expression of the coding sequences of Gcase and Cathepsin B are separated by a T2A self-cleaving peptide sequence.

FIG. 25 is a schematic depicting one embodiment of a vector comprising an expression construct encoding Gcase (e.g., GBA1 or a portion thereof), Sphingomyelin phosphodiesterase 1 (e.g., SMPD1 a portion thereof, and an interfering RNA for α-Syn.

FIG. 26 is a schematic depicting one embodiment of a vector comprising an expression construct encoding Gcase (e.g., GBA1 or a portion thereof) and Galactosylceramidase (e.g., GALC or a portion thereof). The coding sequences of Gcase and Galactosylceramidase are separated by an internal ribosomal entry site (IRES).

FIG. 27 is a schematic depicting one embodiment of a vector comprising an expression construct encoding Gcase (e.g., GBA1 or a portion thereof) and Cathepsin B (e.g., CTSB or a portion thereof). Expression of the coding sequences of Gcase and Cathepsin B are each driven by a separate promoter.

FIG. 28 is a schematic depicting one embodiment of a vector comprising an expression construct encoding Gcase (e.g., GBA1 or a portion thereof), GCH1 (e.g., GCH1 or a portion thereof), and an interfering RNA for α-Syn. The coding sequences of Gcase and GCH1 are separated by an T2A self-cleaving peptide sequence

FIG. 29 is a schematic depicting one embodiment of a vector comprising an expression construct encoding Gcase (e.g., GBA1 or a portion thereof), RAB7L1 (e.g., RAB7L1 or a portion thereof), and an interfering RNA for α-Syn. The coding sequences of Gcase and RAB7L1 are separated by an T2A self-cleaving peptide sequence.

FIG. 30 is a schematic depicting one embodiment of a vector comprising an expression construct encoding Gcase (e.g., GBA1 or a portion thereof), GCH1 (e.g., GCH1 or a portion thereof), and an interfering RNA for α-Syn. Expression of the coding sequences of Gcase and GCH1 are an internal ribosomal entry site (IRES).

FIG. 31 is a schematic depicting one embodiment of a vector comprising an expression construct encoding VPS35 (e.g., VPS35 or a portion thereof) and interfering RNAs for α-Syn and TMEM106B.

FIG. 32 is a schematic depicting one embodiment of a vector comprising an expression construct encoding Gcase (e.g., GBA1 or a portion thereof), IL-34 (e.g., IL34 or a portion thereof), and an interfering RNA for α-Syn. The coding sequences of Gcase and IL-34 are separated by T2A self-cleaving peptide sequence.

FIG. 33 is a schematic depicting one embodiment of a vector comprising an expression construct encoding Gcase (e.g., GBA1 or a portion thereof) and IL-34 (e.g., IL34 or a portion thereof). The coding sequences of Gcase and IL-34 are separated by an internal ribosomal entry site (IRES).

FIG. 34 is a schematic depicting one embodiment of a vector comprising an expression construct encoding Gcase (e.g., GBA1 or a portion thereof) and TREM2 (e.g., TREM2 or a portion thereof). Expression of the coding sequences of Gcase and TREM2 are each driven by a separate promoter.

FIG. 35 is a schematic depicting one embodiment of a vector comprising an expression construct encoding Gcase (e.g., GBA1 or a portion thereof) and IL-34 (e.g., IL34 or a portion thereof). Expression of the coding sequences of Gcase and IL-34 are each driven by a separate promoter.

FIG. 36A-FIG. 36B show representative data for overexpression of TREM2 and GBA1 in HEK293 cells relative to control transduced cells, as measured by qPCR and ELISA. FIG. 36A shows data for overexpression of TREM2. FIG. 36B shows data for overexpression of GBA1 from the same construct.

FIG. 37 shows representative data indicating successful silencing of SNCA in vitro by GFP reporter assay (top) and α-Syn assay (bottom).

FIG. 38 shows representative data indicating successful silencing of TMEM106B in vitro by GFP reporter assay (top) and α-Syn assay (bottom).

FIG. 39 is a schematic depicting one embodiment of a vector comprising an expression construct encoding PGRN.

FIG. 40 shows data for transduction of HEK293 cells using rAAVs having ITRs with wild-type (circles) or alternative (e.g., “outside”; squares) placement of the “D” sequence. The rAAVs having ITRs placed on the “outside” were able to transduce cells as efficiently as rAAVs having wild-type ITRs.

FIG. 41 is a schematic depicting one embodiment of a vector comprising an expression construct encoding Gcase (e.g., GBA1 or a portion thereof).

FIG. 42 is a schematic depicting one embodiment of a vector comprising an expression construct encoding Gcase (e.g., GBA1 or a portion thereof).

FIG. 43 is a schematic depicting one embodiment of a vector comprising an expression construct encoding Gcase (e.g., GBA1 or a portion thereof) and an interfering RNA for α-Syn.

FIG. 44 is a schematic depicting one embodiment of a vector comprising an expression construct encoding PGRN.

FIG. 45 is a schematic depicting one embodiment of a vector comprising an expression construct encoding PGRN.

FIG. 46 is a schematic depicting one embodiment of a vector comprising an expression construct encoding PGRN and an interfering RNA for microtubule-associated protein tau (MAPT).

FIG. 47 is a schematic depicting one embodiment of a vector comprising an expression construct encoding Gcase (e.g., GBA1 or a portion thereof) and an interfering RNA for α-Syn.

FIG. 48 is a schematic depicting one embodiment of a vector comprising an expression construct encoding PSAP.

FIG. 49 is a schematic depicting one embodiment of a vector comprising an expression construct encoding Gcase (e.g., GBA1 or a portion thereof).

FIG. 50 is a schematic depicting one embodiment of a vector comprising an expression construct encoding Gcase (e.g., GBA1 or a portion thereof) and Galactosylceramidase (e.g., GALC or a portion thereof).

FIG. 51 is a schematic depicting one embodiment of a plasmid comprising an rAAV vector that includes an expression construct encoding Gcase (e.g., GBA1 or a portion thereof), Prosaposin (e.g., PSAP or a portion thereof), and an interfering RNA for α-Syn.

FIG. 52A shows that iPSC-derived neuronal stem cell (NSC) lines from patients with FTD-GRN mutations secreted less progranulin than NSC lines derived from healthy control subjects. Statistics were determined using an unpaired t-test; *=p<0.05, **=p<0.01, ***=p<0.001. Data is presented as mean±SEM.

FIG. 52B shows results from dose-ranging PR006A transduction in FTD-GRN mutation carrier neuronal cultures. NSCs were seeded at an equal density and differentiated into neurons. On day 7, neurons were transduced with excipient or the indicated amounts of PR006A for 72 hours. Secreted progranulin expression was measured from the cell media by ELISA and normalized to volume (n=3-4; mean±SEM). Black dashed line represents endogenous levels of secreted progranulin from Control neurons (excipient-treated). Secreted progranulin was not detectable in excipient-treated FTD-GRN neurons. Statistics were determined using ANOVA followed by Tukey HSD and statistical comparison of each condition to excipient-treated Control neurons is indicated on the graph, *=p<0.05, ***=p<0.001. LLOQ=lower limit of quantitation; MOI=multiplicity of infection.

FIG. 52C shows that PR006 treatment of neuronal cultures rescued the defective maturation of a key lysosomal protease, cathepsin D, in FTD-GRN neuronal cultures. NSCs were seeded at equal concentrations and differentiated into neurons. On day 7, neurons were transduced with excipient or PR006A at an MOI of 5.3×10⁵ for 72 hours. Neurons were lysed, and lysates were analyzed on the Protein Simple Western Jess system with an anti-cathepsin D (CTSD) primary antibody. Bands corresponding to both the mature cathepsin D (matCTSD) and pro-cathepsin D (proCTSD) were detected, and the area under the curve was quantified for each band and normalized to an internal total protein normalization signal. The matCTSD/proCTSD ratio in excipient or PR006A treated FTD-GRN neurons was determined; the y-axis depicts the matCTSD/proCTSD ratio as a percent of the ratio of excipient-treated Control neurons (n=3; mean±SEM). Statistics were determined using a paired t-test, *=p<0.05.

FIG. 52D and FIG. 52F show that PR006A reduces TDP-43 pathology in FTD-GRN neuronal cultures. NSCs were seeded at equal concentrations and differentiated into neurons. On day 7, neurons were transduced with excipient or PR006A at an MOI of 5.3×10⁵ and collected 21 days after transduction. FIG. 52D: Neurons were lysed, and the Triton-X insoluble protein fraction was isolated and analyzed on the Protein Simple Western Jess system with an anti-TDP-43 antibody (#12892-AP-1). A band corresponding to TDP-43 was detected, and the area under the curve was quantified for each band and normalized to the total protein concentration of the insoluble fraction. The y-axis depicts the amount of insoluble TDP-43 as a percent of excipient treated levels normalized separately for each FTD-GRN cell line (n=3; mean±SEM). FIG. 52D shows that PR006 treatment decreased insoluble TDP-43, a hallmark of FTD-GRN pathology, in FTD-GRN neuronal cultures. FIG. 52F: Quantification of nuclear TDP-43 signal from immunofluorescence images of iPSC-derived neurons treated with PR006A. The TDP-43 signal intensity per nucleus in excipient or PR006A treated FTD-GRN neurons was determined; the y-axis depicts the TDP-43 signal intensity per nucleus as a percent of the TDP-43 signal intensity per nucleus of excipient treated Control neurons (n=145-306 cells; mean±SEM). TDP-43 was measured using an anti-TDP-43 antibody (#12892-AP-1) and nuclear area was determined by DAPI stain. FIG. 52F shows that PR006 treatment increased nuclear TDP-43 expression levels in FTD-GRN neuronal cultures to near wild-type control levels. Statistics were determined using an unpaired t-test, **=p<0.01, ***=p<0.001.

FIG. 52E shows that iPSC-derived NSC lines from patients with FTD-GRN mutations expressed less progranulin than NSC lines derived from healthy control subjects. Statistics were determined using an unpaired t-test; *=p<0.05, **=p<0.01, ***=p<0.001. Data is presented as mean±SEM.

FIG. 52G is a series of images showing that neuronal stem cell (NSC) lines from human FTD-GRN and human control cell lines were successfully differentiated into neuronal cultures. Control and FTD-GRN NSC lines (FTD-GRN #1 and FTD-GRN #2) were differentiated into neurons after a period of 7 days, as indicated by cell morphology and immunofluorescence staining for neuronal markers (NeuN [red]; MAP2 or Tau as labeled at left [green]). DAPI (blue) was used to stain the nucleus.

FIG. 53A-FIG. 53C are a series of bar graphs depicting the results of experiments analyzing biodistribution and progranulin expression in the CNS in adult dose-ranging PR006A FTD-GRN mouse model study. 4-month-old Grn KO mice were given PR006A or excipient by ICV administration. They were sacrificed 3 months after the treatment with excipient (red) or PR006A at dose of 1.1×10⁹ vg (2.7×10⁹ vg/g brain), 1.1×10¹⁰ vg (2.7×10¹⁰ vg/g brain), or 1.1×10¹¹ vg (2.7×10¹¹ vg/g brain) (blue) for biochemical endpoints in the CNS. FIG. 53A: Presence of vector genomes was assessed in the cerebral cortex and spinal cord, and biodistribution is shown as vector genomes per μg of gDNA on a log scale (n=8-10/group; mean±SEM). Vector genome presence was quantified by qPCR using a vector reference standard curve. Dashed line (at 50 vector genomes/μg gDNA) represents the threshold for positive vector presence. FIG. 53B: PR006A-encoded GRN RNA expression was assessed by quantitative RT-PCR (qRT-PCR) in the cerebral cortex (n=8-10/group; mean±SEM). The number of GRN copies (specific to our codon optimized PR006A sequence) was normalized to 1 μg of total RNA and is shown on a log scale. FIG. 53C: Progranulin protein levels were measured using a human-specific progranulin ELISA in the brain and spinal cord (n=8-10/group; mean±SEM). Tissue progranulin levels were normalized to total protein concentration. The lower limit of quantitation (LLOQ) is indicated by a dashed gray line. For tissue ELISA assays, LLOQ (ng/mg) values are determined by dividing the assay LLOQ (ng/mL) by the total protein concentration average from all samples. A simple line corresponding to the treatment group legend color on the x-axis without error bars indicates that all animals in that group were 0. Statistical analysis was conducted using ANOVA followed by Dunnett's test to compare to the excipient treated Grn KO mouse group; *=p<0.05, **=p<0.01, ***=p<0.001. vg=vector genomes; LLOQ=lower limit of quantitation; SC=spinal cord.

FIG. 53D-FIG. 53E are a series of bar graphs depicting the results of experiments analyzing peripheral tissue biodistribution and progranulin expression in adult dose-ranging PR006A FTD-GRN mouse model study. 4-month-old Grn KO mice were given PR006A or excipient by ICV administration. They were sacrificed 3 months after the treatment with excipient (red) or PR006A at dose of 1.1×10⁹ vg (2.7×10⁹ vg/g brain), 1.1×10¹⁰ vg (2.7×10¹⁰ vg/g brain), or 1.1×10¹¹ vg (2.7×10¹¹ vg/g brain) (blue) for biochemical endpoints in the liver, heart, lung, kidney, spleen, and gonads. FIG. 53D: Presence of vector genomes was assessed, and biodistribution is shown as vector genomes per μg of gDNA on a log scale (n=8-10/group; mean±SEM). Vector genome presence was quantified by qPCR using a vector reference standard curve. Dashed line (at 50 vector genomes/μg gDNA) represents the threshold for positive vector presence. FIG. 53E: Progranulin protein levels were measured using an ELISA (n=8-10/group; mean±SEM). Tissue progranulin levels were normalized to total protein concentration. A simple line corresponding to the treatment group legend color on the x-axis without error bars indicates that all animals in that group were 0. Statistical analysis was conducted using ANOVA followed by Dunnett's test to compare to the excipient treated Grn KO mouse group; *=p<0.05, ***=p<0.001. vg=vector genomes.

FIG. 53F is a bar graph depicting the results of experiments analyzing progranulin levels in the plasma in the adult dose-ranging PR006A FTD-GRN mouse model study. 4-month-old Grn KO mice were given PR006A or excipient by ICV administration. They were sacrificed 3 months after the treatment with excipient (red) or PR006A at dose of 1.1×10⁹ vg (2.7×10⁹ vg/g brain), 1.1×10¹⁰ vg (2.7×10¹⁰ vg/g brain), or 1.1×10¹¹ vg (2.7×10¹¹ vg/g brain) (blue) for biochemical endpoints in the plasma. Progranulin protein levels were measured using a human-specific progranulin ELISA in plasma (n=8-10/group; mean±SEM). Plasma levels are shown on a log scale. The lower limit of quantitation (LLOQ) is indicated by a dashed gray line. Statistical analysis was conducted using ANOVA followed by Dunnett's test to compare to the excipient treated Grn KO mouse group; *=p<0.05, **=p<0.01, ***=p<0.001. LLOQ=lower limit of quantitation. vg=vector genomes.

FIG. 53G-FIG. 53H are a series of bar graphs depicting the results of experiments showing reduced lysosomal and neuropathology defects in adult dose-ranging PR006A FTD-GRN adult mouse model study. 4-month-old Grn KO mice were given PR006A or excipient by ICV administration. They were sacrificed for analysis 3 months after the treatment with excipient (red) or PR006A at dose of 1.1×10⁹ vg (2.7×10⁹ vg/g brain), 1.1×10¹⁰ vg (2.7×10¹⁰ vg/g brain), or 1.1×10¹¹ vg (2.7×10¹¹ vg/g brain) (blue). Lipofuscinosis was analyzed by two independent methods: (1) scoring of H&E-stained brain sections by a pathologist, and (2) quantification of lipofuscin autofluorescence from IHC sections. FIG. 53G: Lipofuscin accumulation (autofluorescent lipofuscin granules) was semi-quantitatively scored in H&E-stained sections in different brain regions by a blinded board-certified pathologist according to the following grading scheme: 0=no lipofuscin observed; 1=very small granules of lipofuscin (<2 μm) scattered throughout region; 2=increased density of small granule accumulation, and/or development of larger granules (>2-3 μm); 3=multifocal regions with a high density of lipofuscin granules visible from a low objective power; 4=widespread lipofuscin accumulation. Lipofuscin severity scores in the cerebral cortex, hippocampus, and thalamus/hypothalamus brain regions is shown (n=8-10/group). FIG. 53H: IHC analysis of ubiquitin was performed and quantified in the cerebral cortex, hippocampus, and thalamus. The size of above-threshold immunoreactive objects (immunoreactive object size [μm2] is shown for ubiquitin (n=8-10/group; mean±SEM). Statistics were determined by ANOVA followed by Dunnett's test to compare to the excipient treated Grn KO mouse group, *=p<0.05, **=p<0.01, ***=p<0.001. vg=vector genomes; WT=wildtype.

FIG. 53I-FIG. 53K are a series of bar graphs depicting the results of experiments showing decreased neuroinflammatory markers in adult dose-ranging PR006A FTD-GRN mouse model study. 4-month-old Grn KO mice were given PR006A or excipient by ICV administration. They were sacrificed for analysis 3 months after the treatment with excipient (red) or PR006A at dose of 1.1×10⁹ vg (2.7×10⁹ vg/g brain), 1.1×10¹⁰ vg (2.7×10¹⁰ vg/g brain), or 1.1×10¹¹ vg (2.7×10¹¹ vg/g brain) (blue). FIG. 53I: Gene expression (mRNA levels) of Tnf and Cd68 was measured by qRT-PCR in the somatosensory cortex (mean±SEM; n=8-10/group). Gene expression was normalized to the housekeeping gene Ppib. FIG. 53J-FIG. 53K: IHC analysis of Iba1 (FIG. 53J) and GFAP (FIG. 53K) was performed and quantified in fixed brain sections in the cerebral cortex, hippocampus, and thalamus. The percent of the area of interest that is covered by above-threshold objects (immunoreactive area [%]) is shown (mean±SEM; n=8-10/group). Statistics were determined using ANOVA with Dunnett's adjustment comparing each group to the excipient treated Grn KO mouse group, *=p<0.05, ***=p<0.001. vg=vector genomes; WT=wildtype.

FIG. 53L-FIG. 53N are a series of bar graphs depicting the results of experiments showing decreased gene expression of lysosomal and immune pathways in adult dose-ranging PR006A FTD-GRN mouse model study. 4-month-old Grn KO mice were given PR006A or excipient by ICV administration. They were sacrificed for analysis 3 months after the treatment with excipient (red) or PR006A at dose of 1.1×10⁹ vg (2.7×10⁹ vg/g brain), 1.1×10¹⁰ vg (2.7×10¹⁰ vg/g brain), or 1.1×10¹¹ vg (2.7×10¹¹ vg/g brain) (blue). RNA sequencing was performed in cerebral cortex samples from in ICV-treated Grn KO mice and from age-matched WT C57BL/6J mice (gray). Gene Set Variation Analysis (GSVA) methodology was used to compare mRNA expression levels of previously published gene signatures that are dysregulated in excipient treated Grn KO mice compared to WT mice. Data shown are the GSVA activity scores for curated gene sets from two published studies and one HALLMARK pathway. FIG. 53L: Cellular Component: Vacuole (GO: 0005773), FIG. 53M: Lysosome, and FIG. 53N: Complement System (HALLMARK pathway) (median±range; n=8-10/group). Statistical analysis was conducted using ANOVA followed by Dunnett's test to compare to the excipient-treated Grn KO mouse group while controlling for the family-wise Type I error rate, ***=p<0.001. GSVA=gene set variation analysis; vg=vector genomes; WT=wildtype.

FIG. 54A is a series of bar graphs depicting the results of experiments analyzing biodistribution of PR006A transgene quantified by qPCR. Transgene levels were analyzed using qPCR methodologies in NHPs 182 days after ICM injection of either excipient, low dose of PR006A (6.5×10⁹ vg/g brain), or high dose of PR006A (6.5×10¹⁰ vg/g brain). Each bar represents the average±SEM of 3 animals per group; the yellow line indicates the lower limit of quantitation at 50 vg/μg DNA.

FIG. 54B is a series of bar graphs depicting the results of experiments analyzing levels of anti-drug antibody to human progranulin. Antibodies to progranulin in NHP serum and CSF samples at Day 29 and Day 182 post-treatment with either excipient, a low dose of PR006A (6.5×10⁹ vg/g brain), or a high dose of PR006A (6.5×10¹⁰ vg/g brain). Data represents the mean SEM.

FIG. 54C is a series of bar graphs depicting the results of experiments analyzing expression of PR006A transgene (GRN). GRN expression levels were determined in NHP cortex, hippocampus and ventral mesencephalon collected on Day 183 using RT-qPCR. Data is presented as mean±SEM.

FIG. 54D is a bar graph depicting the results of experiments analyzing progranulin levels in the CSF quantified by Simple Western™ (Jess) platform. Progranulin levels were determined in NHP CSF samples that were collected at Day 183, determined by a Simple Western™ (Jess) analysis. CSF samples from NHPs treated with excipient, low dose of PR006A (6.5×10⁹ vg/g brain weight) or high dose of PR006A (6.5×10¹⁰ vg/g brain weight). Data presented is mean±SEM; P-value: *p<0.05, by one-way dose dependence response analysis using William's trend test.

FIG. 55 is a graph showing selectivity and specificity results for the automated Western Jess assay. Progranulin protein levels in FTD patient CSF samples were detected at 58 kDa by Jess. Group (A): heterozygous FTD patients and groups (B) and (C): familial non-carrier or normal individuals. Data are presented as mean±standard error of the mean (SEM). SEM values are shown as vertical error bars.

FIG. 56 is a graph showing Progranulin levels in FTD patient CSF samples detected by ELISA. Group (A): heterozygous FTD patients and groups (B) and (C): familial non-carrier or normal individuals. Data are presented as mean±standard error of the mean (SEM). SEM values are shown as vertical error bars.

FIG. 57 is a gel image of each CSF sample run in duplicate on the Jess automated Western platform. Samples were analyzed at a 4-fold dilution using the primary antibody Adipogen PG-359-7. The first lane is the molecular weight standards, and on the right is the band identification used to calculate the immunoreactivities reported in Example 14.

FIG. 58A-FIG. 58B are a series of plots showing the measurement of human PGRN expression levels. Human PGRN expression levels were determined in non-human primate (NHP) CSF samples that were collected at Day 180, using a Simple Western™ (Jess) analysis. CSF from NHPs treated with excipient (“Excipient”), low dose of PR006A (6.5×10⁹ vg/g brain weight; “low”) or high dose of PR006 (6.5×10¹⁰ vg/g brain weight; “high”) were analyzed. The data is expressed as average immunoreactivity peak area (FIG. 58A), or fold change over excipient-treated animals (FIG. 58B). Each dot represents a single CSF sample from one NHP (mean of the technical duplicate) and the box represents the mean value +/− standard error of the three individual NHPs.

FIG. 59A-FIG. 59C are a series of bar graphs depicting the results of experiments analyzing biodistribution and progranulin expression in the CNS in an aged FTD-GRN mouse model following PR006A treatment. Tissue samples were collected from 18-month old Grn KO mice 2 months after receiving ICV excipient (red) or 9.7×10¹⁰ vg (2.4×10¹¹ vg/g brain) PR006A (blue). FIG. 59A: Presence of vector genomes was assessed in the cerebral cortex and spinal cord (mean±SEM; n=4/group). Biodistribution is shown as vector genomes per 1 μg of gDNA on a log scale. Vector genome presence was quantified by qPCR using a vector reference standard curve. Dashed line (at 50 vector genomes/μg gDNA) represents the threshold for positive vector presence. FIG. 59B-FIG. 59C: Progranulin protein levels were measured using an ELISA in CNS tissues (brain and spinal cord (FIG. 59B)), and CSF (FIG. 59C) (mean±SEM; n=4/group). Tissue progranulin levels were normalized to total protein concentration, and CSF levels of progranulin were normalized to fluid volume. The lower limit of quantitation (LLOQ) is indicated by a dashed gray line. For tissue ELISA assays, LLOQ (ng/mg) values were determined by dividing the assay LLOQ (ng/mL) by the total protein concentration average from all samples. A simple red line on the x-axis without error bars indicates that all animals in that group were 0. Statistical analyses were performed using Kruskal-Wallis; *=p<0.05, **=p<0.01, ***=p<0.001. vg=vector genomes; LLOQ=lower limit of quantitation; SC=spinal cord.

FIG. 59D-FIG. 59E are a series of bar graphs and images depicting the results of experiments showing reduced lysosomal and neuropathology defects in an aged FTD-GRN mouse model following PR006A treatment. Tissue samples were collected from 18-month old Grn KO mice 2 months after receiving ICV excipient (red) or 9.7×10¹⁰ vg (2.4×10¹¹ vg/g brain) PR006A (blue). Lipofuscinosis was analyzed by scoring of H&E-stained brain sections by a pathologist. FIG. 59D: Representative lipofuscin images from the thalamus/hypothalamus region of brain sections. White arrowheads indicate examples of lipofuscin accumulation. A summary of lipofuscin severity scores in the cerebral cortex, hippocampus, and thalamus/hypothalamus of H&E-stained slides from brain sections that were evaluated for autofluorescent lipofuscin granules is provided. Lipofuscin accumulation was semi-quantitatively scored by a blinded board-certified pathologist according to the following grading scheme: 0=no lipofuscin observed; 1=very small granules of lipofuscin (<2 μm) scattered throughout region; 2=increased density of small granule accumulation, and/or development of larger granules (>2-3 μm); 3=multifocal regions with a high density of lipofuscin granules visible from a low objective power; 4=widespread lipofuscin accumulation. FIG. 59E: IHC analysis of ubiquitin (n=4/group) was performed and quantified in the cerebral cortex, hippocampus, and thalamus. The positive cell density (cells/mm²) for each region is shown (mean±SEM). Statistics were determined using a t-test, *=p<0.05, **=p<0.01. vg=vector genomes.

FIG. 59F-FIG. 59I are a series of bar graphs depicting the results of experiments showing decreased neuroinflammation markers in an aged FTD-GRN mouse model following PR006A treatment. Tissue samples were collected from 18-month old Grn KO mice 2 months after receiving ICV excipient (red) or 9.7×10¹⁰ vg (2.4×10¹¹ vg/g brain) PR006A (blue). FIG. 59F: Gene expression of Tnf and Cd68 was measured by qRT-PCR in the somatosensory cortex (mean±SEM; n=4/group). Gene expression was normalized to the housekeeping gene Ppib. (FIG. 59G) Protein expression of the proinflammatory cytokine TNFα was measured in the cerebral cortex using a Mesoscale Discovery mouse pro-inflammatory cytokine assay (mean±SEM; n=4/group). Cerebral cortices were homogenized, and protein expression levels were normalized to total protein concentration of tissue lysates. FIG. 59H-FIG. 59I: IHC analysis of Iba1 (FIG. 59H) and GFAP (FIG. 59I) was performed and quantified in fixed brain sections. A compilation of the positive cell density (cells/mm²) from the three brain regions analyzed (cerebral cortex, hippocampus, and thalamus) is shown (mean±SEM; n=3-4/group). Statistical analyses were performed using a t-test, *=p<0.05. vg=vector genomes.

FIG. 60 is a graph depicting a dose-response curve of HEK293T cells transduced with PR006A (n=2; mean±SEM). An equal number of cells were transduced with varying amounts of PR006A. After 72 hours, progranulin protein levels in the cell media were measured using an ELISA assay.

FIG. 61 is a diagram of a study design for maximal dose PR006A in an aged FTD-GRN mouse model. 10 μl excipient (control) or PR006A at a dose of 9.7×10¹⁰ vg (2.4×10¹¹ vg/g brain) was delivered by ICV injection to two cohorts of Grn KO mice: (1) 16 months old at time of injection (n=4-5/group; PRV-2018-027) and (2) 14 months old at time of injection (n=1/excipient-treated group; n=3/PR006A-treated group; PRV-2019-002). The animals were sacrificed two months post-injection. CNS and peripheral tissues were collected to analyze PR006A biodistribution (qPCR), progranulin protein expression (ELISA), and histopathology (H&E). Expression of proinflammatory markers, lipofuscin accumulation, and ubiquitin accumulation were assessed in the brain.

FIG. 62A-FIG. 62B are bar graphs showing results for peripheral tissue biodistribution and progranulin expression in an aged FTD-GRN mouse model following PR006A treatment. Tissue samples were collected from 18-month old Grn KO mice 2 months after receiving ICV excipient (red) or 9.7×10¹⁰ vg (2.4×10¹¹ vg/g brain) PR006A (blue). FIG. 62A: Presence of vector genomes was assessed in the liver, heart, lung, kidney, spleen, and gonads (mean±SEM; n=4/group). Biodistribution is shown as vector genomes per μg of gDNA on a log scale. Vector genome presence was quantified by qPCR using a vector reference standard. FIG. 62B: Progranulin protein levels were measured using an ELISA (mean±SEM; n=4/group). Tissue progranulin levels were normalized to total protein concentration. A simple red line on the x-axis without error bars indicates that all animals in that group were 0. Statistical analyses were performed using Kruskal-Wallis; *=p<0.05, **=p<0.01, ***=p<0.001. vg=vector genomes.

FIG. 63 is a diagram of a study design for dose-ranging PR006A in an adult FTD-GRN mouse model 10 μl excipient (control) or PR006A at dose of 1.1×10⁹ vg (2.7×10⁹ vg/g brain), 1.1×10¹⁰ vg (2.7×10¹⁰ vg/g brain), or 1.1×10¹¹ vg (2.7×10¹¹ vg/g brain) PR006A was delivered by ICV injection into 4-month-old Grn KO mice (n=10/group). The animals were sacrificed three months post-injection, when the mice were 7 months old. CNS and peripheral tissues were collected to analyze PR006A biodistribution (qPCR), progranulin protein expression (ELISA), and histopathology (H&E). Expression of proinflammatory markers, lipofuscin accumulation, ubiquitin accumulation, and global gene expression changes were assessed in the brain.

FIG. 64 is a schematic depicting one embodiment of a recombinant adeno-associated virus vector (PR006A) comprising an expression construct encoding human progranulin. “bp” refers to “base pairs”. “kan” refers to a gene that confers resistance to kanamycin. “GRN” refers to “progranulin”. “ITR” refers to an adeno-associated virus inverted terminal repeat sequence. “TRY” refers to a sequence comprising three transcriptional regulatory activation sites: TATA, RBS, and YY1. “CBAp” refers to a chicken 3-actin promoter. “CMVe” refers to a cytomegalovirus enhancer. “WPRE” refers to a woodchuck hepatitis virus post-transcriptional regulatory element. “bGH” refers to a bovine Growth Hormone polyA signal tail. “int” refers to an intron. The nucleotide sequences of the two strands of PR006A are provided in SEQ ID NOs: 90 and 91.

### DETAILED DESCRIPTION

The disclosure is based, in part, on compositions and methods for expression of combinations of certain gene products (e.g., gene products associated with CNS disease) in a subject. A gene product can be a protein, a fragment (e.g., portion) of a protein, an interfering nucleic acid that inhibits a CNS disease-associated gene, etc. In some embodiments, a gene product is a protein or a protein fragment encoded by a CNS disease-associated gene. In some embodiments, a gene product is an interfering nucleic acid (e.g., shRNA, siRNA, miRNA, amiRNA, etc.) that inhibits a CNS disease-associated gene.

A CNS disease-associated gene refers to a gene encoding a gene product that is genetically, biochemically or functionally associated with a CNS disease, such as FTD (fronto-temporal dementia) or PD (Parkinson's disease). For example, individuals having a pathogenic mutation in the GRN gene (which encodes the protein PGRN) have an increased risk of developing FTD compared to individuals that do not have a mutation in GRN. Similarly, individuals having mutations in the GBA1 gene (which encodes the protein Gcase), have been observed to be have an increased risk of developing PD compared to individuals that do not have a mutation in GBA1. In another example, PD is associated with accumulation of protein aggregates comprising α-Synuclein (α-Syn) protein; accordingly, SNCA (which encodes α-Syn) is a PD-associated gene. In some embodiments, an expression cassette described herein encodes a wild-type or non-mutant form of a CNS disease-associated gene (or coding sequence thereof). Examples of CNS disease-associated genes are listed in Table 1.

TABLE 1
Examples of CNS disease-associated genes

| Name | Gene | Function | NCBI Accession No. |
|---|---|---|---|
| Lysosome membrane | SCARB2/LIMP2 | lysosomal receptor | NP_005497.1 |
| protein 2 | | for | (Isoform 1), |
| | | glucosylceramidase | NP_001191184.1 |
| | | (GBA targeting) | (Isoform 2) |
| Prosaposin | PSAP | precursor for | AAH01503.1, |
| | | saposins A, B, C, | AAH07612.1, |
| | | and D, which | AAH04275.1, |
| | | localize to the | AAA60303.1 |
| | | lysosomal | |
| | | compartment and | |
| | | facilitate the | |
| | | catabolism of | |
| | | glycosphingolipids | |
| | | with short | |
| | | oligosaccharide | |
| | | groups | |
| beta-Glucocerebrosidase | GBA1 | cleaves the beta- | NP_001005742.1 |
| | | glucosidic linkage | (Isoform 1), |
| | | of glucocerebroside | NP_001165282.1 |
| | | | (Isoform 2), |
| | | | NP_001165283.1 |
| | | | (Isoform 3) |
| Non-lysosomal | GBA2 | catalyzes the | NP_065995.1 |
| Glucosylceramidase | | conversion of | (Isoform 1), |
| | | glucosylceramide to | NP_001317589.1 |
| | | free glucose and | (Isoform 2) |
| | | ceramide | |
| Galactosylceramidase | GALC | removes galactose | EAW81359.1 |
| | | from ceramide | (Isoform |
| | | derivatives | CRA_a), |
| | | | EAW81360.1 |
| | | | (Isoform |
| | | | CRA_b), |
| | | | EAW81362.1 |
| | | | (Isoform |
| | | | CRA_c) |
| Sphingomyelin | SMPDI | converts | EAW68726.1 |
| phosphodiesterase 1 | | sphingomyelin to | (Isoform |
| | | ceramide | CRA_a), |
| | | | EAW68727.1 |
| | | | (Isoform |
| | | | CRA_b), |
| | | | EAW68728.1 |
| | | | (Isoform |
| | | | CRA_c), |
| | | | EAW68729.1 |
| | | | (Isoform |
| | | | CRA_d) |
| Cathepsin B | CTSB | thiol protease | AAC37547.1, |
| | | believed to | AAH95408.1, |
| | | participate in | AAH10240.1 |
| | | intracellular | |
| | | degradation and | |
| | | turnover of | |
| | | proteins; also | |
| | | implicated in tumor | |
| | | invasion and | |
| | | metastasis | |
| RAB7, member RAS | RAB7L1 | regulates vesicular | AAH02585.1 |
| oncogene family-like 1 | | transport | |
| Vacuolar protein sorting- | VPS35 | component of | NP_060676.2 |
| associated protein 35 | | retromer cargo- | |
| | | selective complex | |
| GTP cyclohydrolase 1 | GCH1 | responsible for | AAH25415.1 |
| | | hydrolysis of | |
| | | guanosine | |
| | | triphosphate to form | |
| | | 7.8- | |
| | | dihydroneopterin | |
| | | triphosphate | |
| Interleukin 34 | IL34 | increases growth or | AAH29804.1 |
| | | survival of | |
| | | monocytes; elicits | |
| | | activity by binding | |
| | | the Colony | |
| | | stimulating factor 1 | |
| | | receptor | |
| Triggering receptor | TREM2 | forms a receptor | AAF69824.1 |
| expressed on myeloid | | signaling complex | |
| cells 2 | | with the TYRO | |
| | | protein tyrosine | |
| | | kinase binding | |
| | | protein; functions in | |
| | | immune response | |
| | | and may be | |
| | | involved in chronic | |
| | | inflammation | |
| Progranulin | PGRN | plays a role in | NP_002087.1 |
| | | development, | |
| | | inflammation, cell | |
| | | proliferation and | |
| | | protein homeostasis | |

In addition to Gaucher disease patients (who possess mutations in both chromosomal alleles of GBA1 gene), patients with mutations in only one allele of GBA1 are at highly increased risk of Parkinson's disease (PD). The severity of PD symptoms—which include gait difficulty, a tremor at rest, rigidity, and often depression, sleep difficulties, and cognitive decline—correlate with the degree of enzyme activity reduction. Thus, Gaucher disease patients have the most severe course, whereas patient with a single mild mutation in GBA1 typically have a more benign course. Mutation carriers are also at high risk of other PD-related disorders, including Lewy Body Dementia, characterized by executive dysfunction, psychosis, and a PD-like movement disorder, and multi-system atrophy, with characteristic motor and cognitive impairments. No therapies exist that alter the inexorable course of these disorders.

Deficits in enzymes such as Gcase (e.g., the gene product of GBA1 gene), as well as common variants in many genes implicated in lysosome function or trafficking of macromolecules to the lysosome (e.g., Lysosomal Membrane Protein 1 (LIMP), also referred to as SCARB2), have been associated with increased PD risk and/or risk of Gaucher disease (e.g., neuronopathic Gaucher disease, such as Type 2 Gaucher disease or Type 3 Gaucher disease). The disclosure is based, in part, on expression constructs (e.g., vectors) encoding one or more genes, for example Gcase, GBA2, prosaposin, progranulin (PGRN), LIMP2, GALC, CTSB, SMPD1, GCH1, RAB7, VPS35, IL-34, TREM2, TMEM106B, or a combination of any of the foregoing (or portions thereof), associated with central nervous system (CNS) diseases, for example Gaucher disease, PD, etc. In some embodiments, combinations of gene products described herein act together (e.g., synergistically) to reduce one or more signs and symptoms of a CNS disease when expressed in a subject.

Accordingly, in some aspects, the disclosure provides an isolated nucleic acid comprising an expression construct encoding a Gcase (e.g., the gene product of GBA1 gene). In some embodiments, the isolated nucleic acid comprises a Gcase-encoding sequence that has been codon optimized (e.g., codon optimized for expression in mammalian cells, for example human cells). In some embodiments, the nucleic acid sequence encoding the Gcase encodes a protein comprising an amino acid sequence as set forth in SEQ ID NO: 14 (e.g., as set forth in NCBI Reference Sequence NP_000148.2). In some embodiments, the isolated nucleic acid comprises the sequence set forth in SEQ ID NO: 15. In some embodiments the expression construct comprises adeno-associated virus (AAV) inverted terminal repeats (ITRs), for example AAV ITRs flanking the nucleic acid sequence encoding the Gcase protein.

In some aspects, the disclosure provides an isolated nucleic acid comprising an expression construct encoding Prosaposin (e.g., the gene product of PSAP gene). In some embodiments, the isolated nucleic acid comprises a prosaposin-encoding sequence that has been codon optimized (e.g., codon optimized for expression in mammalian cells, for example human cells). In some embodiments, the nucleic acid sequence encoding the prosaposin encodes a protein comprising an amino acid sequence as set forth in SEQ ID NO: 16 (e.g., as set forth in NCBI Reference Sequence NP_002769.1). In some embodiments, the isolated nucleic acid comprises the sequence set forth in SEQ ID NO: 17. In some embodiments the expression construct comprises adeno-associated virus (AAV) inverted terminal repeats (ITRs), for example AAV ITRs flanking the nucleic acid sequence encoding the prosaposin protein.

In some aspects, the disclosure provides an isolated nucleic acid comprising an expression construct encoding LIMP2/SCARB2 (e.g., the gene product of SCARB2 gene). In some embodiments, the isolated nucleic acid comprises a SCARB2-encoding sequence that has been codon optimized (e.g., codon optimized for expression in mammalian cells, for example human cells). In some embodiments, the nucleic acid sequence encoding the LIMP2/SCARB2 encodes a protein comprising an amino acid sequence as set forth in SEQ ID NO: 18 (e.g., as set forth in NCBI Reference Sequence NP_005497.1). In some embodiments, the isolated nucleic acid comprises the sequence set forth in SEQ ID NO: 29. In some embodiments the expression construct comprises adeno-associated virus (AAV) inverted terminal repeats (ITRs), for example AAV ITRs flanking the nucleic acid sequence encoding the SCARB2 protein.

In some aspects, the disclosure provides an isolated nucleic acid comprising an expression construct encoding GBA2 protein (e.g., the gene product of GBA2 gene). In some embodiments, the isolated nucleic acid comprises a GBA2-encoding sequence that has been codon optimized (e.g., codon optimized for expression in mammalian cells, for example human cells). In some embodiments, the nucleic acid sequence encoding the GBA2 encodes a protein comprising an amino acid sequence as set forth in SEQ ID NO: 30 (e.g., as set forth in NCBI Reference Sequence NP_065995.1). In some embodiments, the isolated nucleic acid comprises the sequence set forth in SEQ ID NO: 31. In some embodiments the expression construct comprises adeno-associated virus (AAV) inverted terminal repeats (ITRs), for example AAV ITRs flanking the nucleic acid sequence encoding the GBA2 protein.

In some aspects, the disclosure provides an isolated nucleic acid comprising an expression construct encoding GALC protein (e.g., the gene product of GALC gene). In some embodiments, the isolated nucleic acid comprises a GALC-encoding sequence that has been codon optimized (e.g., codon optimized for expression in mammalian cells, for example human cells). In some embodiments, the nucleic acid sequence encoding the GALC encodes a protein comprising an amino acid sequence as set forth in SEQ ID NO: 33 (e.g., as set forth in NCBI Reference Sequence NP_000144.2). In some embodiments, the isolated nucleic acid comprises the sequence set forth in SEQ ID NO: 34. In some embodiments the expression construct comprises adeno-associated virus (AAV) inverted terminal repeats (ITRs), for example AAV ITRs flanking the nucleic acid sequence encoding the GALC protein.

In some aspects, the disclosure provides an isolated nucleic acid comprising an expression construct encoding CTSB protein (e.g., the gene product of CTSB gene). In some embodiments, the isolated nucleic acid comprises a CTSB-encoding sequence that has been codon optimized (e.g., codon optimized for expression in mammalian cells, for example human cells). In some embodiments, the nucleic acid sequence encoding the CTSB encodes a protein comprising an amino acid sequence as set forth in SEQ ID NO: 35 (e.g., as set forth in NCBI Reference Sequence NP_001899.1). In some embodiments, the isolated nucleic acid comprises the sequence set forth in SEQ ID NO: 36. In some embodiments the expression construct comprises adeno-associated virus (AAV) inverted terminal repeats (ITRs), for example AAV ITRs flanking the nucleic acid sequence encoding the CTSB protein.

In some aspects, the disclosure provides an isolated nucleic acid comprising an expression construct encoding SMPD1 protein (e.g., the gene product of SMPD1 gene). In some embodiments, the isolated nucleic acid comprises a SMPD1-encoding sequence that has been codon optimized (e.g., codon optimized for expression in mammalian cells, for example human cells). In some embodiments, the nucleic acid sequence encoding the SMPD1 encodes a protein comprising an amino acid sequence as set forth in SEQ ID NO: 37 (e.g., as set forth in NCBI Reference Sequence NP_000534.3). In some embodiments, the isolated nucleic acid comprises the sequence set forth in SEQ ID NO: 38. In some embodiments the expression construct comprises adeno-associated virus (AAV) inverted terminal repeats (ITRs), for example AAV ITRs flanking the nucleic acid sequence encoding the SMPD1 protein.

In some aspects, the disclosure provides an isolated nucleic acid comprising an expression construct encoding GCH1 protein (e.g., the gene product of GCH1 gene). In some embodiments, the isolated nucleic acid comprises a GCH1-encoding sequence that has been codon optimized (e.g., codon optimized for expression in mammalian cells, for example human cells). In some embodiments, the nucleic acid sequence encoding the GCH1 encodes a protein comprising an amino acid sequence as set forth in SEQ ID NO: 45 (e.g., as set forth in NCBI Reference Sequence NP_000534.3). In some embodiments, the isolated nucleic acid comprises the sequence set forth in SEQ ID NO: 46. In some embodiments the expression construct comprises adeno-associated virus (AAV) inverted terminal repeats (ITRs), for example AAV ITRs flanking the nucleic acid sequence encoding the GCH1 protein.

In some aspects, the disclosure provides an isolated nucleic acid comprising an expression construct encoding RAB7L protein (e.g., the gene product of RAB7L gene). In some embodiments, the isolated nucleic acid comprises a RAB7L-encoding sequence that has been codon optimized (e.g., codon optimized for expression in mammalian cells, for example human cells). In some embodiments, the nucleic acid sequence encoding the RAB7L encodes a protein comprising an amino acid sequence as set forth in SEQ ID NO: 47 (e.g., as set forth in NCBI Reference Sequence NP_003920.1). In some embodiments, the isolated nucleic acid comprises the sequence set forth in SEQ ID NO: 48. In some embodiments the expression construct comprises adeno-associated virus (AAV) inverted terminal repeats (ITRs), for example AAV ITRs flanking the nucleic acid sequence encoding the RAB7L protein.

In some aspects, the disclosure provides an isolated nucleic acid comprising an expression construct encoding VPS35 protein (e.g., the gene product of VPS35 gene). In some embodiments, the isolated nucleic acid comprises a VPS35-encoding sequence that has been codon optimized (e.g., codon optimized for expression in mammalian cells, for example human cells). In some embodiments, the nucleic acid sequence encoding the VPS35 encodes a protein comprising an amino acid sequence as set forth in SEQ ID NO: 49 (e.g., as set forth in NCBI Reference Sequence NP_060676.2). In some embodiments, the isolated nucleic acid comprises the sequence set forth in SEQ ID NO: 50. In some embodiments the expression construct comprises adeno-associated virus (AAV) inverted terminal repeats (ITRs), for example AAV ITRs flanking the nucleic acid sequence encoding the VPS35 protein.

In some aspects, the disclosure provides an isolated nucleic acid comprising an expression construct encoding IL-34 protein (e.g., the gene product of IL34 gene). In some embodiments, the isolated nucleic acid comprises a IL-34-encoding sequence that has been codon optimized (e.g., codon optimized for expression in mammalian cells, for example human cells). In some embodiments, the nucleic acid sequence encoding the IL-34 encodes a protein comprising an amino acid sequence as set forth in SEQ ID NO: 55 (e.g., as set forth in NCBI Reference Sequence NP_689669.2). In some embodiments, the isolated nucleic acid comprises the sequence set forth in SEQ ID NO: 56. In some embodiments the expression construct comprises adeno-associated virus (AAV) inverted terminal repeats (ITRs), for example AAV ITRs flanking the nucleic acid sequence encoding the IL-34 protein.

In some aspects, the disclosure provides an isolated nucleic acid comprising an expression construct encoding TREM2 protein (e.g., the gene product of TREMgene). In some embodiments, the isolated nucleic acid comprises a TREM2-encoding sequence that has been codon optimized (e.g., codon optimized for expression in mammalian cells, for example human cells). In some embodiments, the nucleic acid sequence encoding the TREM2 encodes a protein comprising an amino acid sequence as set forth in SEQ ID NO: 57 (e.g., as set forth in NCBI Reference Sequence NP_061838.1). In some embodiments, the isolated nucleic acid comprises the sequence set forth in SEQ ID NO: 58. In some embodiments the expression construct comprises adeno-associated virus (AAV) inverted terminal repeats (ITRs), for example AAV ITRs flanking the nucleic acid sequence encoding the TREM2 protein.

In some aspects, the disclosure provides an isolated nucleic acid comprising an expression construct encoding TMEM106B protein (e.g., the gene product of TMEM106B gene). In some embodiments, the isolated nucleic acid comprises a TMEM106B-encoding sequence that has been codon optimized (e.g., codon optimized for expression in mammalian cells, for example human cells). In some embodiments, the nucleic acid sequence encoding the TMEM106B encodes a protein comprising an amino acid sequence as set forth in SEQ ID NO: 63 (e.g., as set forth in NCBI Reference Sequence NP_060844.2). In some embodiments, the isolated nucleic acid comprises the sequence set forth in SEQ ID NO: 64. In some embodiments the expression construct comprises adeno-associated virus (AAV) inverted terminal repeats (ITRs), for example AAV ITRs flanking the nucleic acid sequence encoding the TMEM106B protein.

In some aspects, the disclosure provides an isolated nucleic acid comprising an expression construct encoding progranulin (e.g., the gene product of PGRN gene). In some embodiments, the isolated nucleic acid comprises a prosaposin-encoding sequence that has been codon optimized (e.g., codon optimized for expression in mammalian cells, for example human cells). In some embodiments, the nucleic acid sequence encoding the progranulin (PGRN) encodes a protein comprising an amino acid sequence as set forth in SEQ ID NO: 67 (e.g., as set forth in NCBI Reference Sequence NP_002078.1). In some embodiments, the isolated nucleic acid comprises the sequence set forth in SEQ ID NO: 68. In some embodiments the expression construct comprises adeno-associated virus (AAV) inverted terminal repeats (ITRs), for example AAV ITRs flanking the nucleic acid sequence encoding the prosaposin protein.

In some aspects, the disclosure provides an isolated nucleic acid comprising an expression construct encoding a first gene product and a second gene product, wherein each gene product independently is selected from the gene products, or portions thereof, set forth in Table 1.

In some embodiments, a first gene product or a second gene product is a Gcase protein, or a portion thereof. In some embodiments, a first gene product is a Gcase protein and a second gene product is selected from GBA2, prosaposin, progranulin, LIMP2, GALC, CTSB, SMPD1, GCH1, RAB7, VPS35, IL-34, TREM2, and TMEM106B.

In some embodiments, an expression construct encodes (e.g., alone or in addition to another gene product) an interfering nucleic acid (e.g., shRNA, miRNA, dsRNA, etc.). In some embodiments, an interfering nucleic acid inhibits expression of α-Synuclein (α-Synuclein). In some embodiments, an interfering nucleic acid that targets α-Synuclein comprises a sequence set forth in any one of SEQ ID NOs: 20-25. In some embodiments, an interfering nucleic acid that targets α-Synuclein binds to (e.g., hybridizes with) a sequence set forth in any one of SEQ ID NO: 20-25.

In some embodiments, an interfering nucleic acid inhibits expression of TMEM106B. In some embodiments, an interfering nucleic acid that targets TMEM106B comprises a sequence set forth in SEQ ID NO: 64 or 65. In some embodiments, an interfering nucleic acid that targets TMEM106B binds to (e.g., hybridizes with) a sequence set forth in SEQ ID NO: 64 or 65.

In some embodiments, an expression construct further comprises one or more promoters. In some embodiments, a promoter is a chicken-beta actin (CBA) promoter, a CAG promoter, a CD68 promoter, or a JeT promoter. In some embodiments, a promoter is a RNA pol II promoter or an RNA pol III promoter (e.g., U6, etc.).

In some embodiments, an expression construct further comprises an internal ribosomal entry site (IRES). In some embodiments, an IRES is located between a first gene product and a second gene product.

In some embodiments, an expression construct further comprises a self-cleaving peptide coding sequence. In some embodiments, a self-cleaving peptide is a T2A peptide.

In some embodiments, an expression construct comprises two adeno-associated virus (AAV) inverted terminal repeat (ITR) sequences. In some embodiments, ITR sequences flank a first gene product and a second gene product (e.g., are arranged as follows from 5′-end to 3′-end: ITR-first gene product-second gene product-ITR). In some embodiments, one of the ITR sequences of an isolated nucleic acid lacks a functional terminal resolution site (trs). For example, in some embodiments, one of the ITRs is a ΔITR.

The disclosure relates, in some aspects, to rAAV vectors comprising an ITR having a modified “D” region (e.g., a D sequence that is modified relative to wild-type AAV2 ITR, SEQ ID NO: 29). In some embodiments, the ITR having the modified D region is the 5′ ITR of the rAAV vector. In some embodiments, a modified “D” region comprises an “S” sequence, for example as set forth in SEQ ID NO: 26. In some embodiments, the ITR having the modified “D” region is the 3′ ITR of the rAAV vector. In some embodiments, a modified “D” region comprises a 3′ITR in which the “D” region is positioned at the 3′ end of the ITR (e.g., on the outside or terminal end of the ITR relative to the transgene insert of the vector). In some embodiments, a modified “D” region comprises a sequence as set forth in SEQ ID NO: 26 or 27.

In some embodiments, an isolated nucleic acid (e.g., an rAAV vector) comprises a TRY region. In some embodiments, a TRY region comprises the sequence set forth in SEQ ID NO: 28.

In some embodiments, an isolated nucleic acid described by the disclosure comprises or consists of, or encodes a peptide having, the sequence set forth in any one of SEQ ID NOs: 1-91.

In some aspects, the disclosure provides a vector comprising an isolated nucleic acid as described by the disclosure. In some embodiments, a vector is a plasmid, or a viral vector. In some embodiments, a viral vector is a recombinant AAV (rAAV) vector or a Baculovirus vector. In some embodiments, an rAAV vector is single-stranded (e.g., single-stranded DNA).

In some embodiments, the disclosure provides a host cell comprising an isolated nucleic acid as described by the disclosure or a vector as described by the disclosure.

In some embodiments, the disclosure provides a recombinant adeno-associated virus (rAAV) comprising a capsid protein and an isolated nucleic acid or a vector as described by the disclosure.

In some embodiments, a capsid protein is capable of crossing the blood-brain barrier, for example an AAV9 capsid protein or an AAVrh.10 capsid protein. In some embodiments, an rAAV transduces neuronal cells and non-neuronal cells of the central nervous system (CNS).

In some aspects, the disclosure provides a method for treating a subject having or suspected of having or suspected of having a central nervous system (CNS) disease, the method comprising administering to the subject a composition (e.g., a composition comprising an isolated nucleic acid or a vector or a rAAV) as described by the disclosure. In some embodiments, the CNS disease is a neurodegenerative disease, such as a neurodegenerative disease listed in Table 12. In some embodiments, the CNS disease is a synucleinopathy, such as a synucleinopathy listed in Table 13. In some embodiments, the CNS disease is a tauopathy, such as a tauopathy listed in Table 14. In some embodiments, the CNS disease is a lysosomal storage disease, such as a lysosomal storage disease listed in Table 15. In some embodiments, the lysosomal storage disease is neuronopathic Gaucher disease, such as Type 2 Gaucher disease or Type 3 Gaucher disease.

In some embodiments, the disclosure provides a method for treating a subject having or suspected of having Parkinson's disease, the method comprising administering to the subject a composition (e.g., a composition comprising an isolated nucleic acid or a vector or a rAAV) as described by the disclosure.

In some embodiments, the disclosure provides a method for treating a subject having or suspected of having fronto-temporal dementia (FTD), FTD with GRN mutation, FTD with tau mutation, FTD with C9orf72 mutation, ceroid lipofuscinosis, Parkinson's disease, Alzheimer's disease, corticobasal degeneration, motor neuron disease, or Gaucher disease, the method comprising administering to the subject an rAAV encoding Progranulin (PGRN), wherein the PGRN is encoded by the nucleic acid sequence in SEQ ID NO:68; and wherein the rAAV comprises a capsid protein having an AAV9 serotype.

In some embodiments, the disclosure provides a method for treating a subject having or suspected of having FTD with a GRN mutation, the method comprising administering to the subject an rAAV encoding Progranulin (PGRN), wherein the PGRN is encoded by the nucleic acid sequence in SEQ ID NO:68; and wherein the rAAV comprises a capsid protein having an AAV9 serotype. In some embodiments, the rAAV is administered to a subject at a dose of about 3.5×10¹³ vector genomes (vg), about 7.0×10¹³ vg, or about 1.4×10¹⁴ vg. In some embodiments, the rAAV is administered via an injection into the cisterna magna.

In some embodiments, a composition comprises a nucleic acid (e.g., an rAAV genome, for example encapsidated by AAV capsid proteins) that encodes two or more gene products (e.g., CNS disease-associated gene products), for example 2, 3, 4, 5, or more gene products described in this application. In some embodiments, a composition comprises two or more (e.g., 2, 3, 4, 5, or more) different nucleic acids (e.g., two or more rAAV genomes, for example separately encapsidated by AAV capsid proteins), each encoding one or more different gene products. In some embodiments, two or more different compositions are administered to a subject, each composition comprising one or more nucleic acids encoding different gene products. In some embodiments, different gene products are operably linked to the same promoter type (e.g., the same promoter). In some embodiments, different gene products are operably linked to different promoters.

### Isolated Nucleic Acids and Vectors

An isolated nucleic acid may be DNA or RNA. The disclosure provides, in some aspects, isolated nucleic acids (e.g., rAAV vectors) comprising an expression construct encoding one or more PD-associated genes, for example a Gcase (e.g., the gene product of GBA1 gene) or a portion thereof. Gcase, also referred to as β-glucocerebrosidase or GBA, refers to a lysosomal protein that cleaves the beta-glucosidic linkage of the chemical glucocerebroside, an intermediate in glycolipid metabolism. In humans, Gcase is encoded by the GBA1 gene, located on chromosome 1. In some embodiments, GBA1 encodes a peptide that is represented by NCBI Reference Sequence NP_000148.2 (SEQ ID NO: 14). In some embodiments, an isolated nucleic acid comprises a Gcase-encoding sequence that has been codon optimized (e.g., codon optimized for expression in mammalian cells, for example human cells), such as the sequence set forth in SEQ ID NO: 15.

In some aspects, the disclosure provides an isolated nucleic acid comprising an expression construct encoding Prosaposin (e.g., the gene product of PSAP gene). Prosaposin is a precursor glycoprotein for sphingolipid activator proteins (saposins) A, B, C, and D, which facilitate the catabolism of glycosphingolipids with short oligosaccharide groups. In humans, the PSAP gene is located on chromosome 10. In some embodiments, PSAP encodes a peptide that is represented by NCBI Reference Sequence NP_002769.1 (e.g., SEQ ID NO: 16). In some embodiments, an isolated nucleic acid comprises a prosaposin-encoding sequence that has been codon optimized (e.g., codon optimized for expression in mammalian cells, for example human cells), such as the sequence set forth in SEQ ID NO: 17.

Aspects of the disclosure relate to an isolated nucleic acid comprising an expression construct encoding LIMP2/SCARB2 (e.g., the gene product of SCARB2 gene). SCARB2 refers to a membrane protein that regulates lysosomal and endosomal transport within a cell. In humans, SCARB2 gene is located on chromosome 4. In some embodiments, the SCARB2 gene encodes a peptide that is represented by NCBI Reference Sequence NP_005497.1 (SEQ ID NO: 18). In some embodiments, an isolated nucleic acid comprises the sequence set forth in SEQ ID NO: 19. In some embodiments the isolated nucleic acid comprises a SCARB2-encoding sequence that has been codon optimized.

Aspects of the disclosure relate to an isolated nucleic acid comprising an expression construct encoding GBA2 protein (e.g., the gene product of GBA2 gene). GBA2 protein refers to non-lysosomal glucosylceramidase. In humans, GBA2 gene is located on chromosome 9. In some embodiments, the GBA2 gene encodes a peptide that is represented by NCBI Reference Sequence NP_065995.1 (SEQ ID NO: 30). In some embodiments, an isolated nucleic acid comprises the sequence set forth in SEQ ID NO: 31. In some embodiments the isolated nucleic acid comprises a GBA2-encoding sequence that has been codon optimized.

Aspects of the disclosure relate to an isolated nucleic acid comprising an expression construct encoding GALC protein (e.g., the gene product of GALC gene). GALC protein refers to galactosylceramidase (or galactocerebrosidase), which is an enzyme that hydrolyzes galactose ester bonds of galactocerebroside, galactosylsphingosine, lactosylceramide, and monogalactosyldiglyceride. In humans, GALC gene is located on chromosome 14. In some embodiments, the GALC gene encodes a peptide that is represented by NCBI Reference Sequence NP_000144.2 (SEQ ID NO: 33). In some embodiments, an isolated nucleic acid comprises the sequence set forth in SEQ ID NO: 34. In some embodiments the isolated nucleic acid comprises a GALC-encoding sequence that has been codon optimized.

Aspects of the disclosure relate to an isolated nucleic acid comprising an expression construct encoding CTSB protein (e.g., the gene product of CTSB gene). CTSB protein refers to cathepsin B, which is a lysosomal cysteine protease that plays an important role in intracellular proteolysis. In humans, CTSB gene is located on chromosome 8. In some embodiments, the CTSB gene encodes a peptide that is represented by NCBI Reference Sequence NP_001899.1 (SEQ ID NO: 35). In some embodiments, an isolated nucleic acid comprises the sequence set forth in SEQ ID NO: 36. In some embodiments the isolated nucleic acid comprises a CTSB-encoding sequence that has been codon optimized.

Aspects of the disclosure relate to an isolated nucleic acid comprising an expression construct encoding SMPD1 protein (e.g., the gene product of SMPD1 gene). SMPD1 protein refers to sphingomyelin phosphodiesterase 1, which is a hydrolase enzyme that is involved in sphingolipid metabolism. In humans, SMPD1 gene is located on chromosome 11. In some embodiments, the SMPD1 gene encodes a peptide that is represented by NCBI Reference Sequence NP_000534.3 (SEQ ID NO: 37). In some embodiments, an isolated nucleic acid comprises the sequence set forth in SEQ ID NO: 38. In some embodiments the isolated nucleic acid comprises a SMPD1-encoding sequence that has been codon optimized.

Aspects of the disclosure relate to an isolated nucleic acid comprising an expression construct encoding GCH1 protein (e.g., the gene product of GCH1 gene). GCH1 protein refers to GTP cyclohydrolase 1, which is a hydrolase enzyme that is part of the folate and biopterin biosynthesis pathways. In humans, GCH1 gene is located on chromosome 14. In some embodiments, the GCH1 gene encodes a peptide that is represented by NCBI Reference Sequence NP_000152.1 (SEQ ID NO: 45). In some embodiments, an isolated nucleic acid comprises the sequence set forth in SEQ ID NO: 46. In some embodiments the isolated nucleic acid comprises a GCH1-encoding sequence that has been codon optimized.

Aspects of the disclosure relate to an isolated nucleic acid comprising an expression construct encoding RAB7L protein (e.g., the gene product of RAB7L gene). RAB7L protein refers to RAB7, member RAS oncogene family-like 1, which is a GTP binding protein. In humans, RAB7L gene is located on chromosome 1. In some embodiments, the RAB7L gene encodes a peptide that is represented by NCBI Reference Sequence NP_003920.1 (SEQ ID NO: 47). In some embodiments, an isolated nucleic acid comprises the sequence set forth in SEQ ID NO: 48. In some embodiments the isolated nucleic acid comprises a RAB7L-encoding sequence that has been codon optimized.

Aspects of the disclosure relate to an isolated nucleic acid comprising an expression construct encoding VPS35 protein (e.g., the gene product of VPS35 gene). VPS35 protein refers to vacuolar protein sorting-associated protein 35, which is part of a protein complex involved in retrograde transport of proteins from endosomes to the trans-Golgi network. In humans, VPS35 gene is located on chromosome 16. In some embodiments, the VPS35 gene encodes a peptide that is represented by NCBI Reference Sequence NP_060676.2 (SEQ ID NO: 49). In some embodiments, an isolated nucleic acid comprises the sequence set forth in SEQ ID NO: 50. In some embodiments the isolated nucleic acid comprises a VPS35-encoding sequence that has been codon optimized.

Aspects of the disclosure relate to an isolated nucleic acid comprising an expression construct encoding IL-34 protein (e.g., the gene product of IL34 gene). IL-34 protein refers to interleukin 34, which is a cytokine that increases growth and survival of monocytes. In humans, IL34 gene is located on chromosome 16. In some embodiments, the IL34 gene encodes a peptide that is represented by NCBI Reference Sequence NP_689669.2 (SEQ ID NO: 55). In some embodiments, an isolated nucleic acid comprises the sequence set forth in SEQ ID NO: 56. In some embodiments the isolated nucleic acid comprises a IL-34-encoding sequence that has been codon optimized.

Aspects of the disclosure relate to an isolated nucleic acid comprising an expression construct encoding TREM2 protein (e.g., the gene product of TREM2 gene). TREM2 protein refers to triggering receptor expressed on myeloid cells 2, which is an immunoglobulin superfamily receptor found on myeloid cells. In humans, TREM2 gene is located on chromosome 6. In some embodiments, the TREM2 gene encodes a peptide that is represented by NCBI Reference Sequence NP_061838.1 (SEQ ID NO: 57). In some embodiments, the isolated nucleic acid comprises the sequence set forth in SEQ ID NO: 58. In some embodiments an isolated nucleic acid comprises a TREM2-encoding sequence that has been codon optimized.

Aspects of the disclosure relate to an isolated nucleic acid comprising an expression construct encoding TMEM106B protein (e.g., the gene product of TMEM106B gene). TMEM106B protein refers to transmembrane protein 106B, which is a protein involved in dendrite morphogenesis and regulation of lysosomal trafficking. In humans, TMEM106B gene is located on chromosome 7. In some embodiments, the TMEM106B gene encodes a peptide that is represented by NCBI Reference Sequence NP_060844.2 (SEQ ID NO: 62). In some embodiments, an isolated nucleic acid comprises the sequence set forth in SEQ ID NO: 63. In some embodiments the isolated nucleic acid comprises a TMEM106B-encoding sequence that has been codon optimized.

Aspects of the disclosure relate to an isolated nucleic acid comprising an expression construct encoding progranulin protein (e.g., the gene product of PGRN gene). PGRN protein refers to progranulin, which is a protein involved in development, inflammation, cell proliferation and protein homeostasis. In humans, the PGRN gene is located on chromosome 17. In some embodiments, the PGRN gene encodes a peptide that is represented by NCBI Reference Sequence NP_002078.1 (SEQ ID NO: 67). In some embodiments, an isolated nucleic acid comprises the sequence set forth in SEQ ID NO: 68. In some embodiments the isolated nucleic acid comprises a PGRN-encoding sequence that has been codon optimized. In some embodiments, the nucleic acid further comprises a chicken 3-actin (CBA) promoter and a cytomegalovirus enhancer (CMVe).

In some aspects, the disclosure provides an automated Western blot immunoassay to quantify a PGRN protein level in a cerebrospinal fluid (CSF) sample. In some embodiments, the immunoassay is a capillary-based automated Western blot immunoassay platform, where all steps, such as protein separation, immunoprobing, washing, and detection by chemiluminescence, occur in a capillary cartridge. In some embodiments, a CSF sample is from a human or a non-human primate. In some aspects, the immunoassay allows detection of differences in PGRN protein levels in the presence of circulating antibody. In some aspects, the disclosure provides a method of quantifying a progranulin protein level in a CSF sample, the method comprising: (1) diluting the CSF sample (e.g., a 4-fold dilution); (2) loading the CSF sample; an anti-progranulin antibody; a secondary antibody that detects the anti-progranulin antibody, luminol, and peroxide into wells of a capillary cartridge; (3) loading the capillary cartridge into an automated Western blot immunoassay instrument; (4) using the automated Western blot immunoassay instrument to calculate one or more of: signal intensity, peak area, signal-to-noise ratio and total protein normalization parameters; and (5) quantifying a progranulin protein level in the CSF sample as the peak area of immunoreactivity to the anti-progranulin antibody. In some embodiments, the CSF sample is diluted in a master mix comprising dithiothreitol (DTT) and sample buffer. The master mix may further comprise other proprietary components. In some aspects, the anti-progranulin antibody detects human progranulin. In some embodiments, a progranulin protein level is quantified from the calculated parameters using software that controls the automated Western blot immunoassay instrument. In some embodiments, the software is Compass software for Simple Western™ (ProteinSimple, San Jose, CA).

In some embodiments, the disclosure provides a method of quantifying a progranulin protein level in a cerebrospinal fluid (CSF) sample, the method comprising: (1) diluting the CSF sample (e.g., a 4-fold dilution) in a master mix containing dithiothreitol (DTT) and sample buffer; (2) loading the diluted CSF sample, an anti-progranulin antibody; a secondary antibody that detects the anti-progranulin antibody, luminol, and peroxide into wells of a capillary cartridge; (3) loading the capillary cartridge into an automated Western blot immunoassay instrument; (4) using the automated Western blot immunoassay instrument to calculate signal intensity, peak area, and signal-to-noise ratio; and (5) quantifying a progranulin protein level in the CSF sample as the peak area of immunoreactivity to the anti-progranulin antibody.

In some aspects, the disclosure provides an isolated nucleic acid comprising an expression construct encoding a first gene product and a second gene product, wherein each gene product independently is selected from the gene products, or portions thereof, set forth in Table 1.

In some embodiments, an isolated nucleic acid or vector (e.g., rAAV vector) described by the disclosure comprises or consists of a sequence set forth in any one of SEQ ID NOs: 1-91. In some embodiments, an isolated nucleic acid or vector (e.g., rAAV vector) described by the disclosure comprises or consists of a sequence that is complementary (e.g., the complement of) a sequence set forth in any one of SEQ ID NOs: 1-91. In some embodiments, an isolated nucleic acid or vector (e.g., rAAV vector) described by the disclosure comprises or consists of a sequence that is a reverse complement of a sequence set forth in any one of SEQ ID NOs: 1-91. In some embodiments, an isolated nucleic acid or vector (e.g., rAAV vector) described by the disclosure comprises or consists of a portion of a sequence set forth in any one of SEQ ID NOs: 1-91. A portion may comprise at least 25%, 50%, 60%, 70%, 80%, 90%, 95%, or 99% of a sequence set forth in any one of SEQ ID NOs: 1-91. In some embodiments, a nucleic acid sequence described by the disclosure is a nucleic acid sense strand (e.g., 5′ to 3′ strand), or in the context of a viral sequences a plus (+) strand. In some embodiments, a nucleic acid sequence described by the disclosure is a nucleic acid antisense strand (e.g., 3′ to 5′ strand), or in the context of viral sequences a minus (−) strand.

In some embodiments, a gene product is encoded by a coding portion (e.g., a cDNA) of a naturally occurring gene. In some embodiments, a first gene product is a protein (or a fragment thereof) encoded by the GBA1 gene. In some embodiments, a gene product is a protein (or a fragment thereof) encoded by another gene listed in Table 1, for example the SCARB2/LIMP2 gene or the PSAP gene. However, the skilled artisan recognizes that the order of expression of a first gene product (e.g., Gcase) and a second gene product (e.g., LIMP2, etc.) can generally be reversed (e.g., LIMP2 is the first gene product and Gcase is the second gene product). In some embodiments, a gene product is a fragment (e.g., portion) of a gene listed in Table 1. A protein fragment may comprise about 50%, about 60%, about 70%, about 80% about 90% or about 99% of a protein encoded by the genes listed in Table 1. In some embodiments, a protein fragment comprises between 50% and 99.9% (e.g., any value between 50% and 99.9%) of a protein encoded by a gene listed in Table 1.

In some embodiments, an expression construct is monocistronic (e.g., the expression construct encodes a single fusion protein comprising a first gene product and a second gene product). In some embodiments, an expression construct is polycistronic (e.g., the expression construct encodes two distinct gene products, for example two different proteins or protein fragments).

A polycistronic expression vector may comprise a one or more (e.g., 1, 2, 3, 4, 5, or more) promoters. Any suitable promoter can be used, for example, a constitutive promoter, an inducible promoter, an endogenous promoter, a tissue-specific promoter (e.g., a CNS-specific promoter), etc. In some embodiments, a promoter is a chicken beta-actin promoter (CBA promoter), a CAG promoter (for example as described by Alexopoulou et al. (2008) BMC Cell Biol. 9:2; doi: 10.1186/1471-2121-9-2), a CD68 promoter, or a JeT promoter (for example as described by Tornoe et al. (2002) Gene 297(1-2):21-32). In some embodiments, a promoter is operably-linked to a nucleic acid sequence encoding a first gene product, a second gene product, or a first gene product and a second gene product. In some embodiments, an expression cassette comprises one or more additional regulatory sequences, including but not limited to transcription factor binding sequences, intron splice sites, poly(A) addition sites, enhancer sequences, repressor binding sites, or any combination of the foregoing.

In some embodiments, a nucleic acid sequence encoding a first gene product and a nucleic acid sequence encoding a second gene product are separated by a nucleic acid sequence encoding an internal ribosomal entry site (IRES). Examples of IRES sites are described, for example, by Mokrejs et al. (2006) Nucleic Acids Res. 34 (Database issue):D125-30. In some embodiments, a nucleic acid sequence encoding a first gene product and a nucleic acid sequence encoding a second gene product are separated by a nucleic acid sequence encoding a self-cleaving peptide. Examples of self-cleaving peptides include but are not limited to T2A, P2A, E2A, F2A, BmCPV 2A, and BmIFV 2A, and those described by Liu et al. (2017) Sci Rep. 7: 2193. In some embodiments, the self-cleaving peptide is a T2A peptide.

Pathologically, disorders such as PD and Gaucher disease are associated with accumulation of protein aggregates composed largely of α-Synuclein (α-Syn) protein. Accordingly, in some embodiments, isolated nucleic acids described herein comprise an inhibitory nucleic acid that reduces or prevents expression of α-Syn protein. A sequence encoding an inhibitory nucleic acid may be placed in an untranslated region (e.g., intron, 5′UTR, 3′UTR, etc.) of the expression vector.

In some embodiments, an inhibitory nucleic acid is positioned in an intron of an expression construct, for example in an intron upstream of the sequence encoding a first gene product. An inhibitory nucleic acid can be a double stranded RNA (dsRNA), siRNA, shRNA, micro RNA (miRNA), artificial miRNA (amiRNA), or an RNA aptamer. Generally, an inhibitory nucleic acid binds to (e.g., hybridizes with) between about 6 and about 30 (e.g., any integer between 6 and 30, inclusive) contiguous nucleotides of a target RNA (e.g., mRNA). In some embodiments, the inhibitory nucleic acid molecule is an miRNA or an amiRNA, for example an miRNA that targets SNCA (the gene encoding α-Syn protein) or TMEM106B (e.g., the gene encoding TMEM106B protein). In some embodiments, the miRNA does not comprise any mismatches with the region of SNCA mRNA to which it hybridizes (e.g., the miRNA is “perfected”). In some embodiments, the inhibitory nucleic acid is an shRNA (e.g., an shRNA targeting SNCA or TMEM106B). In some embodiments, an inhibitory nucleic acid is an artificial miRNA (amiRNA) that includes a miR-155 scaffold and a SNCA or TMEM106B targeting sequence.

The skilled artisan recognizes that when referring to nucleic acid sequences comprising or encoding inhibitory nucleic acids (e.g., dsRNA, siRNA, miRNA, amiRNA, etc.) any one or more thymidine (T) nucleotides or uridine (U) nucleotides in a sequence provided herein may be replaced with any other nucleotide suitable for base pairing (e.g., via a Watson-Crick base pair) with an adenosine nucleotide. For example, T may be replaced with U, and U may be replaced with T.

An isolated nucleic acid as described herein may exist on its own, or as part of a vector. Generally, a vector can be a plasmid, cosmid, phagemid, bacterial artificial chromosome (BAC), or a viral vector (e.g., adenoviral vector, adeno-associated virus (AAV) vector, retroviral vector, baculoviral vector, etc.). In some embodiments, the vector is a plasmid (e.g., a plasmid comprising an isolated nucleic acid as described herein). In some embodiments, an rAAV vector is single-stranded (e.g., single-stranded DNA). In some embodiments, the vector is a recombinant AAV (rAAV) vector. In some embodiments, a vector is a Baculovirus vector (e.g., an Autographa californica nuclear polyhedrosis (AcNPV) vector).

Typically an rAAV vector (e.g., rAAV genome) comprises a transgene (e.g., an expression construct comprising one or more of each of the following: promoter, intron, enhancer sequence, protein coding sequence, inhibitory RNA coding sequence, polyA tail sequence, etc.) flanked by two AAV inverted terminal repeat (ITR) sequences. In some embodiments the transgene of an rAAV vector comprises an isolated nucleic acid as described by the disclosure. In some embodiments, each of the two ITR sequences of an rAAV vector is a full-length ITR (e.g., approximately 145 bp in length, and containing functional Rep binding site (RBS) and terminal resolution site (trs)). In some embodiments, one of the ITRs of an rAAV vector is truncated (e.g., shortened or not full-length). In some embodiments, a truncated ITR lacks a functional terminal resolution site (trs) and is used for production of self-complementary AAV vectors (scAAV vectors). In some embodiments, a truncated ITR is a ΔITR, for example as described by McCarty et al. (2003) Gene Ther. 10(26):2112-8.

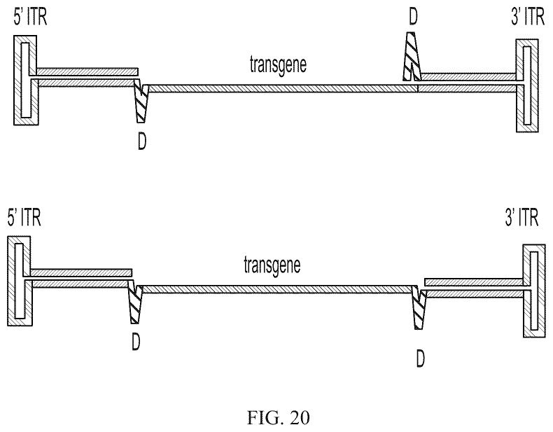

Aspects of the disclosure relate to isolated nucleic acids (e.g., rAAV vectors) comprising an ITR having one or more modifications (e.g., nucleic acid additions, deletions, substitutions, etc.) relative to a wild-type AAV ITR, for example relative to wild-type AAV2 ITR (e.g., SEQ ID NO: 29). The structure of wild-type AAV2 ITR is shown in FIG. 20. Generally, a wild-type ITR comprises a 125 nucleotide region that self-anneals to form a palindromic double-stranded T-shaped, hairpin structure consisting of two cross arms (formed by sequences referred to as B/B′ and C/C′, respectively), a longer stem region (formed by sequences A/A′), and a single-stranded terminal region referred to as the “D” region (FIG. 20). Generally, the “D” region of an ITR is positioned between the stem region formed by the A/A′ sequences and the insert containing the transgene of the rAAV vector (e.g., positioned on the “inside” of the ITR relative to the terminus of the ITR or proximal to the transgene insert or expression construct of the rAAV vector). In some embodiments, a “D” region comprises the sequence set forth in SEQ ID NO: 27. The “D” region has been observed to play an important role in encapsidation of rAAV vectors by capsid proteins, for example as disclosed by Ling et al. (2015) JMol GenetMed 9(3).

The disclosure is based, in part, on the surprising discovery that rAAV vectors comprising a “D” region located on the “outside” of the ITR (e.g., proximal to the terminus of the ITR relative to the transgene insert or expression construct) are efficiently encapsidated by AAV capsid proteins than rAAV vectors having ITRs with unmodified (e.g., wild-type) ITRs In some embodiments, rAAV vectors having a modified “D” sequence (e.g., a “D” sequence in the “outside” position) have reduced toxicity relative to rAAV vectors having wild-type ITR sequences.

In some embodiments, a modified “D” sequence comprises at least one nucleotide substitution relative to a wild-type “D” sequence (e.g., SEQ ID NO: 27). A modified “D” sequence may have at least 1, 2, 3, 4, 5, 6, 7, 8, 9, 10, or more than 10 nucleotide substitutions relative to a wild-type “D” sequence (e.g., SEQ ID NO: 27). In some embodiments, a modified “D” sequence comprises at least 10, 11, 12, 13, 14, 15, 16, 17, 18, or 19 nucleic acid substitutions relative to a wild-type “D” sequence (e.g., SEQ ID NO: 27). In some embodiments, a modified “D” sequence is between about 10% and about 99% (e.g., 10%, 15%, 20%, 25%, 30%, 40%, 45%, 50%, 55%, 60%, 65%, 70%, 75%, 80%, 85%, 90%, 95%, or 99%) identical to a wild-type “D” sequence (e.g., SEQ ID NO: 27). In some embodiments, a modified “D” sequence comprises the sequence set forth in SEQ ID NO: 26, also referred to as an “S” sequence as described in Wang et al. (1995) J Mol Biol 250(5):573-80.

An isolated nucleic acid or rAAV vector as described by the disclosure may further comprise a “TRY” sequence, for example as set forth in SEQ ID NO: 28 or as described by Francois et al., (2005) J. Virol. 79(17):11082-11094. In some embodiments, a TRY sequence is positioned between an ITR (e.g. a 5′ ITR) and an expression construct (e.g. a transgene-encoding insert) of an isolated nucleic acid or rAAV vector.

In some aspects, the disclosure relates to Baculovirus vectors comprising an isolated nucleic acid or rAAV vector as described by the disclosure. In some embodiments, the Baculovirus vector is an Autographa californica nuclear polyhedrosis (AcNPV) vector, for example as described by Urabe et al. (2002) Hum Gene Ther 13(16):1935-43 and Smith et al. (2009) Mol Ther 17(11):1888-1896.

In some aspects, the disclosure provides a host cell comprising an isolated nucleic acid or vector as described herein. A host cell can be a prokaryotic cell or a eukaryotic cell. For example, a host cell can be a mammalian cell, bacterial cell, yeast cell, insect cell, etc. In some embodiments, a host cell is a mammalian cell, for example a HEK293T cell. In some embodiments, a host cell is a bacterial cell, for example an E. coli cell.

rAAVs

In some aspects, the disclosure relates to recombinant AAVs (rAAVs) comprising a transgene that encodes a nucleic acid as described herein (e.g., an rAAV vector as described herein). The term “rAAVs” generally refers to viral particles comprising an rAAV vector encapsidated by one or more AAV capsid proteins. An rAAV described by the disclosure may comprise a capsid protein having a serotype selected from AAV1, AAV2, AAV3, AAV4, AAV5, AAV6, AAV7, AAV8, AAV9, and AAV10. In some embodiments, an rAAV comprises a capsid protein from a non-human host, for example a rhesus AAV capsid protein such as AAVrh.10, AAVrh.39, etc. In some embodiments, an rAAV described by the disclosure comprises a capsid protein that is a variant of a wild-type capsid protein, such as a capsid protein variant that includes at least 1, 2, 3, 4, 5, 6, 7, 8, 9, 10, or more than 10 (e.g., 15, 20 25, 50, 100, etc.) amino acid substitutions (e.g., mutations) relative to the wild-type AAV capsid protein from which it is derived. In some embodiments, an AAV capsid protein variant is an AAV1RX capsid protein, for example as described by Albright et al. Mol Ther. 2018 Feb. 7; 26(2):510-523. In some embodiments, a capsid protein variant is an AAV TM6 capsid protein, for example as described by Rosario et al. Mol Ther Methods Clin Dev. 2016; 3: 16026.

In some embodiments, rAAVs described by the disclosure readily spread through the CNS, particularly when introduced into the CSF space or directly into the brain parenchyma. Accordingly, in some embodiments, rAAVs described by the disclosure comprise a capsid protein that is capable of crossing the blood-brain barrier (BBB). For example, in some embodiments, an rAAV comprises a capsid protein having an AAV9 or AAVrh.10 serotype. Production of rAAVs is described, for example, by Samulski et al. (1989)J Virol. 63(9):3822-8 and Wright (2009) Hum Gene Ther. 20(7): 698-706. In some embodiments, an rAAV comprises a capsid protein that specifically or preferentially targets myeloid cells, for example microglial cells.

In some embodiments, the disclosure provides an rAAV referred to as “PR006A”. PR006A is a rAAV that delivers a functional human GRN gene, leading to increased expression of functional human PGRN. The PR006A vector insert comprises the chicken j-actin (CBA) promoter element, comprising 4 parts: the cytomegalovirus (CMV) enhancer, CBA promoter, exon 1, and intron (int) to constitutively express a codon-optimized coding sequence of human GRN (SEQ ID NO:68). The 3′ region also contains a woodchuck hepatitis virus post-transcriptional regulatory element (WPRE) followed by a bovine growth hormone polyadenylation signal tail. Three well described transcriptional regulatory activation

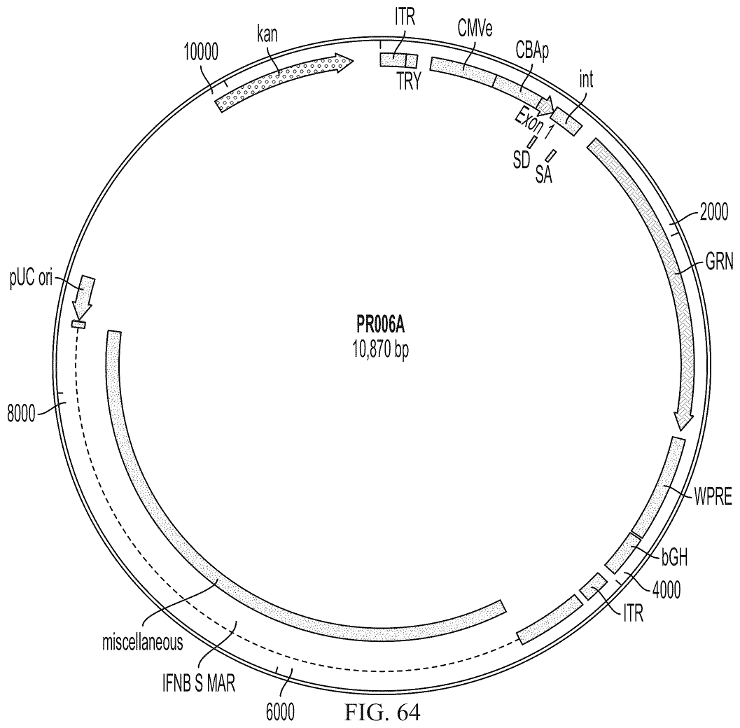

sites are included at the 5′ end of the promoter region: TATA, RBS, and YY1 (see, e.g., Francois et al., (2005) J. Virol. 79(17):11082-11094). The flanking inverted terminal repeats (ITRs) allow for the correct packaging of the intervening sequences. The backbone contains the gene to confer resistance to kanamycin as well as a stuffer sequence to prevent reverse packaging. A schematic depicting the rAAV vector is shown in FIG. 64. SEQ ID NO 90 provides the nucleotide sequence of the first strand (in 5′ to 3′ order) of the PR006A vector shown in FIG. 64. SEQ ID NO 91 provides the nucleotide sequence of the second strand (in 5′ to 3′ order) of the PR006A vector shown in FIG. 64. PR006A comprises AAV9 capsid proteins.

In some embodiments, an rAAV as described by the disclosure (e.g., comprising a recombinant rAAV genome encapsidated by AAV capsid proteins to form an rAAV capsid particle) is produced in a Baculovirus vector expression system (BEVS). Production of rAAVs using BEVS are described, for example by Urabe et al. (2002) Hum Gene Ther 13(16):1935-43, Smith et al. (2009) Mol Ther 17(11):1888-1896, U.S. Pat. Nos. 8,945,918, 9,879,282, and International PCT Publication WO 2017/184879. However, an rAAV can be produced using any suitable method (e.g., using recombinant rep and cap genes). In some embodiments, an rAAV as disclosed herein is produced in HEK293 (human embryonic kidney) cells.

### Pharmaceutical Compositions

In some aspects, the disclosure provides pharmaceutical compositions comprising an isolated nucleic acid or rAAV as described herein and a pharmaceutically acceptable carrier. As used herein, the term “pharmaceutically acceptable” refers to a material, such as a carrier or diluent, which does not abrogate the biological activity or properties of the compound, and is relatively non-toxic, e.g., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

Compositions (e.g., pharmaceutical compositions) provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

In some embodiments, the disclosure provides a PR006A finished drug product comprising the PR006A rAAV described above presented in aqueous solution. In some embodiments, the final formulation buffer comprises about 20 mM Tris [pH 8.0], about 1 mM MgCl₂, about 200 mM NaCl, and about 0.001% [w/v] poloxamer 188. In some embodiments, the finished drug product and the final formulation buffer are suitable for intra-cisterna magna (ICM) injection.

### Methods

Aspects of the disclosure relate to compositions for expression of one or more CNS disease-associated gene products in a subject to treat CNS-associated diseases. The one or more CNS disease-associated gene products may be encoded by one or more isolated nucleic acids or rAAV vectors. In some embodiments, a subject is administered a single vector (e.g., isolated nucleic acid, rAAV, etc.) encoding one or more (1, 2, 3, 4, 5, or more) gene products. In some embodiments, a subject is administered a plurality (e.g., 2, 3, 4, 5, or more) vectors (e.g., isolated nucleic acids, rAAVs, etc.), where each vector encodes a different CNS disease-associated gene product.

A CNS-associated disease may be a neurodegenerative disease, synucleinopathy, tauopathy, or a lysosomal storage disease. Examples of neurodegenerative diseases and their associated genes are listed in Table 12.

A “synucleinopathy” refers to a disease or disorder characterized by the accumulation of alpha-Synuclein (the gene product of SNCA) in a subject (e.g., relative to a healthy subject, for example a subject not having a synucleinopathy). Examples of synucleinopathies and their associated genes are listed in Table 13.

A “tauopathy” refers to a disease or disorder characterized by accumulation of abnormal Tau protein in a subject (e.g., relative to a healthy subject not having a tauopathy). Examples of tauopathies and their associated genes are listed in Table 14.

A “lysosomal storage disease” refers to a disease characterized by abnormal build-up of toxic cellular products in lysosomes of a subject. Examples of lysosomal storage diseases and their associated genes are listed in Table 15.

As used herein “treat” or “treating” refers to (a) preventing or delaying onset of a CNS disease; (b) reducing severity of a CNS disease; (c) reducing or preventing development of symptoms characteristic of a CNS disease; (d) and/or preventing worsening of symptoms characteristic of a CNS disease. Symptoms of CNS disease may include, for example, motor dysfunction (e.g., shaking, rigidity, slowness of movement, difficulty with walking, paralysis), cognitive dysfunction (e.g., dementia, depression, anxiety, psychosis), difficulty with memory, emotional and behavioral dysfunction.

The disclosure is based, in part, on compositions for expression of combinations of PD-associated gene products in a subject that act together (e.g., synergistically) to treat Parkinson's disease.

Accordingly, in some aspects, the disclosure provides a method for treating a subject having or suspected of having Parkinson's disease, the method comprising administering to the subject a composition (e.g., a composition comprising an isolated nucleic acid or a vector or a rAAV) as described by the disclosure.

The disclosure is based, in part, on compositions for expression of one or more CNS-disease associated gene products in a subject to treat Gaucher disease. In some embodiments, the Gaucher disease is a neuronopathic Gaucher disease, for example Type 2 Gaucher disease or Type 3 Gaucher disease. In some embodiments, a subject having Gaucher disease does not have PD or PD symptoms.

Accordingly, in some aspects, the disclosure provides a method for treating a subject having or suspected of having neuronopathic Gaucher disease, the method comprising administering to the subject a composition (e.g., a composition comprising an isolated nucleic acid or a vector or a rAAV) as described by the disclosure.

The disclosure is based, in part, on compositions for expression of one or more CNS-disease associated gene products in a subject to treat Alzheimer's disease or fronto-temporal dementia (FTD). In some embodiments, the subject does not have Alzheimer's disease. In some embodiments, the subject has FTD and does not have Alzheimer's disease. In some embodiments, the subject has FTD with GRN (progranulin) mutation. In some embodiments, the subject has FTD with GRN mutation, and the subject is heterozygous for a GRN mutation (e.g., a pathogenic GRN mutation). In some embodiments, a GRN mutation is a null mutation (e.g., a nonsense, a frameshift, or a splice site mutations, or a complete or partial (exonic) gene deletion). In some embodiments, a GRN mutation is a pathogenic mutation with proven functional deleterious effect. In some embodiments, a GRN mutation is a missense pathogenic mutation. In some embodiments, a GRN mutation is listed in the Molgen FTD database (molgen.ua.ac.be). In some embodiments, a GRN mutation produces a low plasma PGRN level (<70 ng/mL) in a subject.

In some embodiments, the subject has FTD, FTD with GRN mutation, FTD with tau mutation, FTD with C9orf72 mutation, neuronal ceroid lipofuscinosis, Parkinson's disease, Alzheimer's disease, corticobasal degeneration, motor neuron disease, or Gaucher disease.

In some embodiments, the subject has symptomatic FTD (e.g., behavioral-variant FTD (bvFTD), primary progressive aphasia (PPA)-FTD, FTD with corticobasal syndrome, or a combination of syndromes).

Accordingly, in some aspects, the disclosure provides a method for treating a subject having or suspected of having FTD with GRN mutation, the method comprising administering to the subject a composition (e.g., a composition comprising an isolated nucleic acid or a vector or a rAAV) as described by the disclosure.

In some embodiments, a subject having Alzheimer's disease or FTD (e.g. FTD with GRN mutation) is administered an rAAV encoding Progranulin (PGRN) or a portion thereof. In some embodiments, a subject having Alzheimer's disease or FTD (e.g. FTD with GRN mutation) is administered an rAAV encoding PGRN or a portion thereof, wherein the PGRN protein is encoded by a codon-optimized nucleic acid sequence or the nucleic acid sequence in SEQ ID NO:68. In some embodiments, the PGRN protein comprises the amino acid sequence in SEQ ID NO:67 or a portion thereof. In some embodiments, the rAAV encoding PGRN comprises a capsid protein having an AAV9 serotype.

In some embodiments, a composition comprising an rAAV encoding PGRN for treating FTD (e.g. FTD with GRN mutation) is administered to a subject at a dose ranging from about 1×10¹² vector genomes (vg) to about 1×10¹⁵ vg, or from about 1×10¹³ vg to about 7×10¹⁴ vg, or from about 1×10¹³ vg to about 5×10¹⁴ vg, or from about 2×10¹³ vg to about 2×10¹⁴ vg, or from about 3×10¹³ vg to about 2×10¹⁴ vg, or from about 3.5×10¹³ vg to about 1.4×10¹⁴ vg. In some embodiments, a composition comprising an rAAV encoding PGRN for treating FTD (e.g. FTD with GRN mutation) is administered to a subject at a dose of about 2×10¹³ vg, about 3×10¹³ vg, about 4×10¹³ vg, about 5×10¹³ vg, about 6×10¹³ vg, about 7×10¹³ vg, about 8×10¹³ vg, about 9×10¹³ vg, about 1×10¹⁴ vg, or about 2×10¹⁴ vg.

In some aspects, the disclosure provides a method for treating a subject having or suspected of having FTD (e.g. FTD with GRN mutation), the method comprising administering to the subject a composition comprising an rAAV encoding PGRN, wherein the composition is administered at a dose of about 3.5×10¹³ vector genomes (vg), about 7.0×10¹³ vg, or about 1.4×10¹⁴ vg.

In some aspects, the disclosure provides a method for treating a subject having or suspected of having FTD (e.g. FTD with GRN mutation), the method comprising administering to the subject a composition comprising an rAAV encoding PGRN, wherein the composition is administered at a dose of about 1×10¹⁴ vector genomes (vg), about 2.0×10¹⁴ vg, or about 4.0×10¹⁴ vg.

In some embodiments, a composition comprising an rAAV encoding PGRN for treating FTD (e.g. FTD with GRN mutation) to a subject as a single dose, and the composition is not administered to the subject subsequently.

In some embodiments, the composition comprising the rAAV is delivered via a single suboccipital injection into the cisterna magna. In some embodiments, the injection into the cisterna magna is performed under radiographic guidance.

In some embodiments, the disclosure provides a method for treating a symptom of a subject having or suspected of having FTD with GRN mutation, the method comprising administering to the subject a composition comprising an rAAV encoding the sequence for functional Progranulin (PGRN) protein, wherein the PGRN protein is encoded by a codon-optimized nucleic acid sequence or the nucleic acid sequence in SEQ ID NO:68. In some embodiments, a symptom of FTD with GRN mutation may be a personality change, impairment of executive function, disinhibition, apathy, slow speech production, misuse of grammar, multimodal agnosia, semantic aphasia, or impaired word comprehension. In some embodiments, the rAAV encoding PGRN comprises a capsid protein having an AAV9 serotype.

In some embodiments, the disclosure provides a method for reducing lipofuscin accumulation in the brain of a subject having or suspected of having FTD with GRN mutation, the method comprising administering to the subject a composition comprising an rAAV encoding Progranulin (PGRN), wherein the PGRN protein is encoded by a codon-optimized nucleic acid sequence or the nucleic acid sequence in SEQ ID NO:68. In some aspects, the disclosure provides a method for reducing ubiquitin accumulation in the brain of a subject having or suspected of having FTD with GRN mutation, the method comprising administering to the subject a composition comprising an rAAV encoding Progranulin (PGRN), wherein the PGRN protein is encoded by a codon-optimized nucleic acid sequence or the nucleic acid sequence in SEQ ID NO:68. In some aspects, the disclosure provides a method for reducing gene expression and/or protein expression of TNFα and/or CD68 in the brain of a subject having or suspected of having FTD with GRN mutation, the method comprising administering to the subject a composition comprising an rAAV encoding Progranulin (PGRN), wherein the PGRN protein is encoded by a codon-optimized nucleic acid sequence or the nucleic acid sequence in SEQ ID NO:68. In some aspects, the disclosure provides a method for increasing the maturation of cathepsin D in the brain of a subject having or suspected of having FTD with GRN mutation, the method comprising administering to the subject a composition comprising an rAAV encoding Progranulin (PGRN), wherein the PGRN protein is encoded by a codon-optimized nucleic acid sequence or the nucleic acid sequence in SEQ ID NO:68. In some aspects, the disclosure provides a method for increasing the level of nuclear TDP-43 (transactive response DNA binding protein 43 kDa) protein in the brain of a subject having or suspected of having FTD with GRN mutation, the method comprising administering to the subject a composition comprising an rAAV encoding Progranulin (PGRN), wherein the PGRN protein is encoded by a codon-optimized nucleic acid sequence or the nucleic acid sequence in SEQ ID NO:68. In some embodiments, the disclosure provides a method for reducing a level of neurofilament light chain (NFL) in blood or CSF of a subject having or suspected of having FTD with GRN mutation, the method comprising administering to the subject a composition comprising an rAAV encoding Progranulin (PGRN), wherein the PGRN protein is encoded by a codon-optimized nucleic acid sequence or the nucleic acid sequence in SEQ ID NO:68. In some embodiments, the rAAV encoding PGRN comprises a capsid protein having an AAV9 serotype.

A subject is typically a mammal, preferably a human. In some embodiments, a subject is between the ages of 1 month old and 10 years old (e.g., 1 month, 2 months, 3 months, 4, months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3, years, 4 years, 5 years, 6 years, 7 years, 8 years, 9 years, 10 years, or any age therebetween). In some embodiments, a subject is between 2 years old and 20 years old. In some embodiments, a subject is between 30 years old and 100 years old. In some embodiments, a subject is older than 55 years old.

In some embodiments, a composition is administered directly to the CNS of the subject, for example by direct injection into the brain and/or spinal cord of the subject. Examples of CNS-direct administration modalities include but are not limited to intracerebral injection, intraventricular injection, intracisternal injection, intraparenchymal injection, intrathecal injection, and any combination of the foregoing. In some embodiments, a composition is administered to a subject by intra-cisterna magna (ICM) injection. In some embodiments, direct injection into the CNS of a subject results in transgene expression (e.g., expression of the first gene product, second gene product, and if applicable, third gene product) in the midbrain, striatum and/or cerebral cortex of the subject. In some embodiments, direct injection into the CNS results in transgene expression (e.g., expression of the first gene product, second gene product, and if applicable, third gene product) in the spinal cord and/or CSF of the subject.

In some embodiments, direct injection to the CNS of a subject comprises convection enhanced delivery (CED). Convection enhanced delivery is a therapeutic strategy that involves surgical exposure of the brain and placement of a small-diameter catheter directly into a target area of the brain, followed by infusion of a therapeutic agent (e.g., a composition or rAAV as described herein) directly to the brain of the subject. CED is described, for example by Debinski et al. (2009) Expert Rev Neurother. 9(10):1519-27.

In some embodiments, a composition is administered peripherally to a subject, for example by peripheral injection. Examples of peripheral injection include subcutaneous injection, intravenous injection, intra-arterial injection, intraperitoneal injection, or any combination of the foregoing. In some embodiments, the peripheral injection is intra-arterial injection, for example injection into the carotid artery of a subject.

In some embodiments, a composition (e.g., a composition comprising an isolated nucleic acid or a vector or a rAAV) as described by the disclosure is administered both peripherally and directly to the CNS of a subject. For example, in some embodiments, a subject is administered a composition by intra-arterial injection (e.g., injection into the carotid artery) and by intraparenchymal injection (e.g., intraparenchymal injection by CED). In some embodiments, the direct injection to the CNS and the peripheral injection are simultaneous (e.g., happen at the same time). In some embodiments, the direct injection occurs prior (e.g., between 1 minute and 1 week, or more before) to the peripheral injection. In some embodiments, the direct injection occurs after (e.g., between 1 minute and 1 week, or more after) the peripheral injection.

In some embodiments, a subject is administered an immunosuppressant prior to (e.g., between 1 month and 1 minute prior to) or at the same time as a composition as described herein. In some embodiments, the immunosuppressant is a corticosteroid (e.g., prednisone, budesonide, etc.), an mTOR inhibitor (e.g., sirolimus, everolimus, etc.), an antibody (e.g., adalimumab, etanercept, natalizumab, etc.), or methotrexate.

The amount of composition (e.g., a composition comprising an isolated nucleic acid or a vector or a rAAV) as described by the disclosure administered to a subject will vary depending on the administration method. For example, in some embodiments, a rAAV as described herein is administered to a subject at a titer between about 10⁹ Genome copies (GC)/kg and about 10¹⁴ GC/kg (e.g., about 10⁹ GC/kg, about 10¹⁰ GC/kg, about 10¹¹ GC/kg, about 10¹² GC/kg, about 10¹² GC/kg, or about 10¹⁴ GC/kg). In some embodiments, a subject is administered a high titer (e.g., >10¹² Genome Copies GC/kg of an rAAV) by injection to the CSF space, or by intraparenchymal injection. In some embodiments, a rAAV as described herein is administered to a subject at a dose ranging from about 1×10¹⁰ vector genomes (vg) to about 1×10¹⁷ vg by intravenous injection. In some embodiments, a rAAV as described herein is administered to a subject at a dose ranging from about 1×10¹⁰ vg to about 1×10¹⁶ vg by injection into the cisterna magna.

A composition (e.g., a composition comprising an isolated nucleic acid or a vector or a rAAV) as described by the disclosure can be administered to a subject once or multiple times (e.g., 2, 3, 4, 5, 6, 7, 8, 9, 10, 20, or more) times. In some embodiments, a composition is administered to a subject continuously (e.g., chronically), for example via an infusion pump.

### EXAMPLES

### Example 1: rAAV Vectors

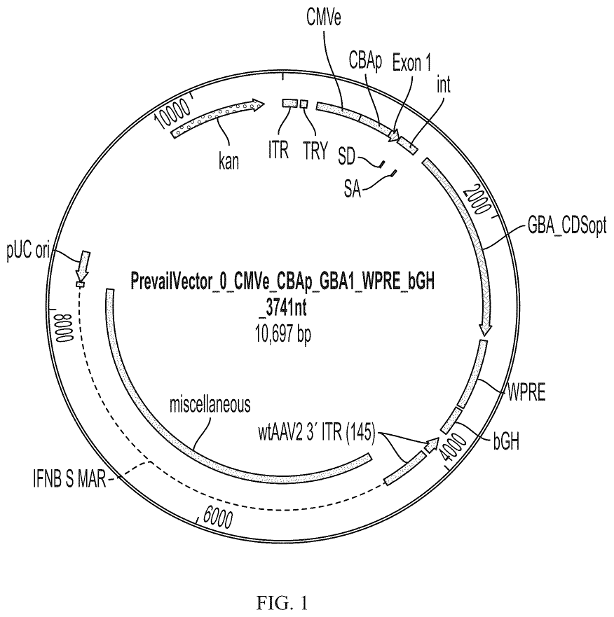

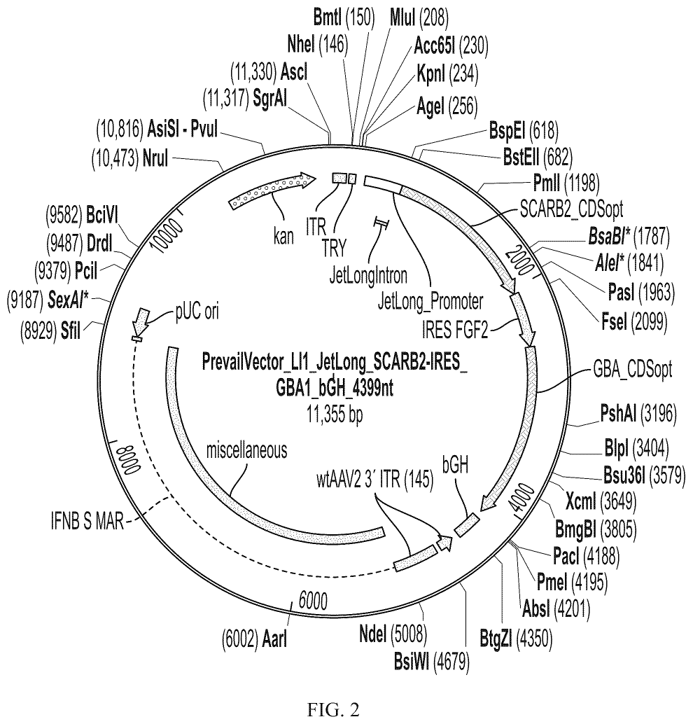

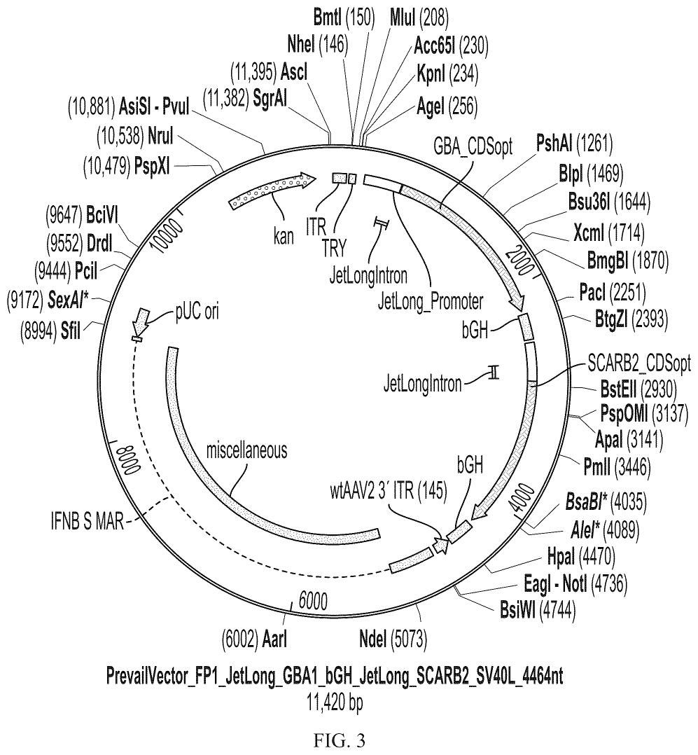

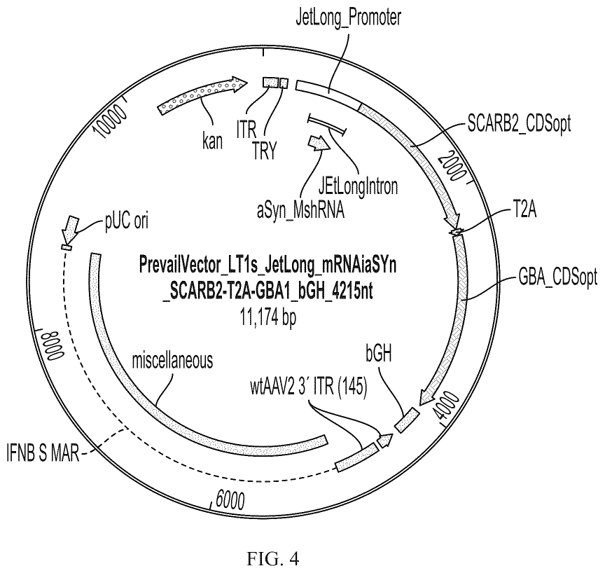

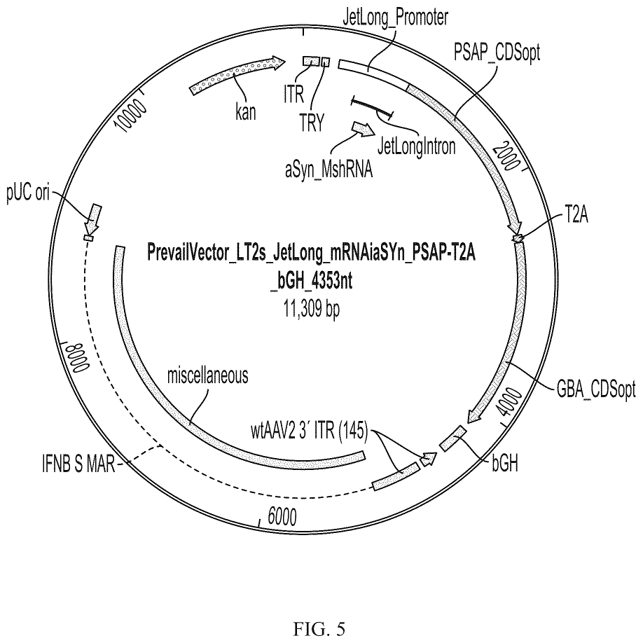

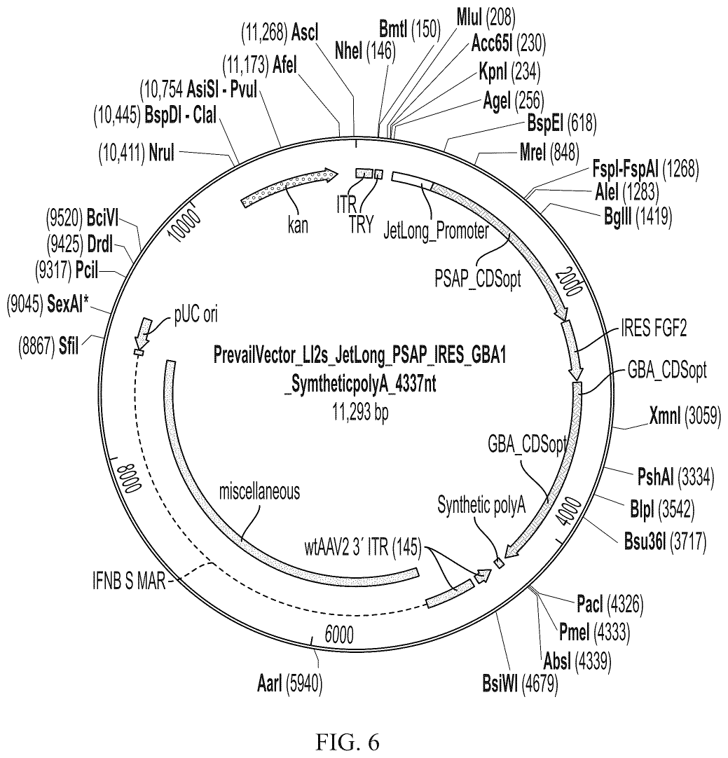

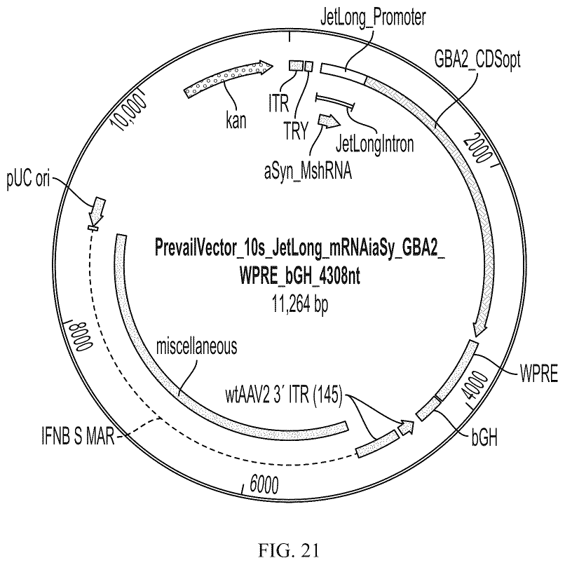

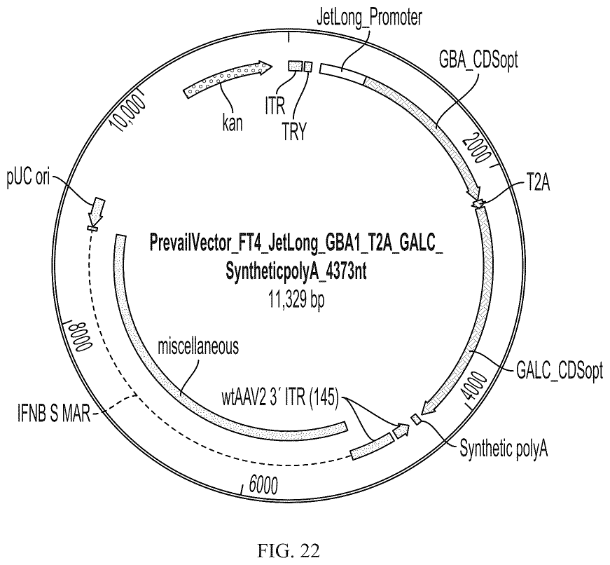

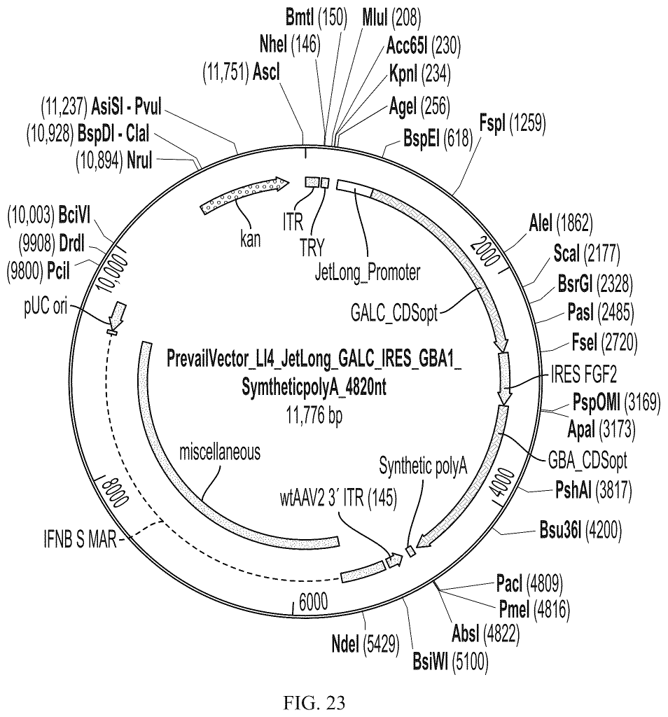

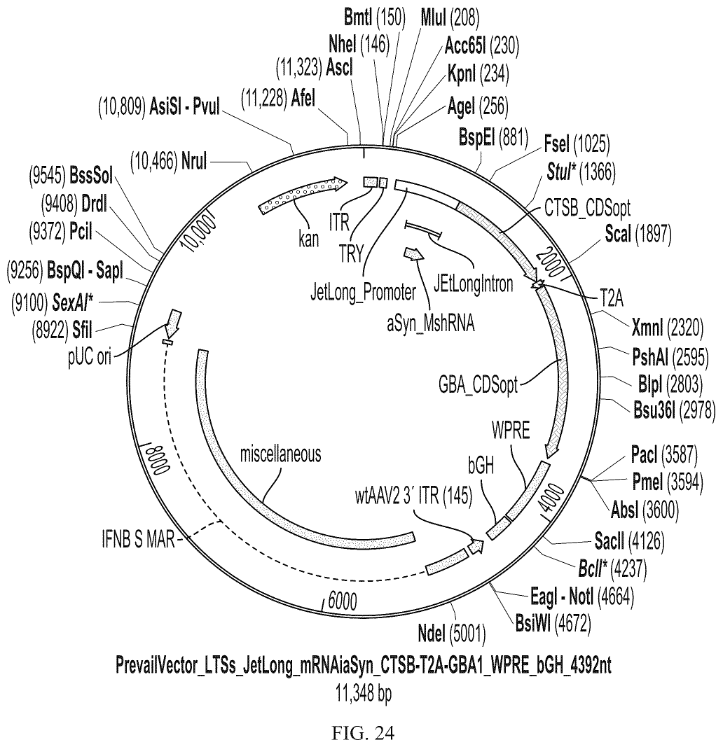

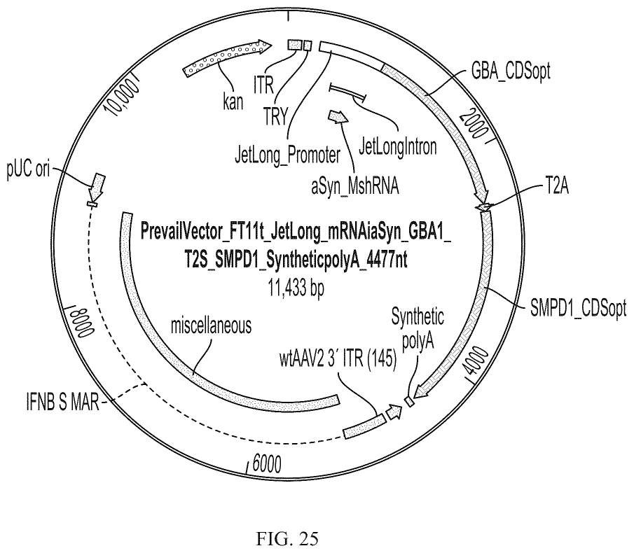

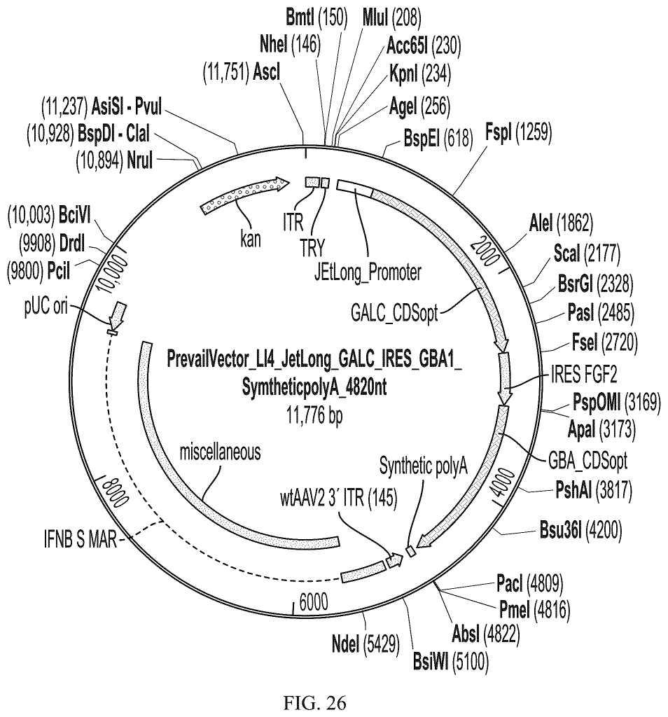

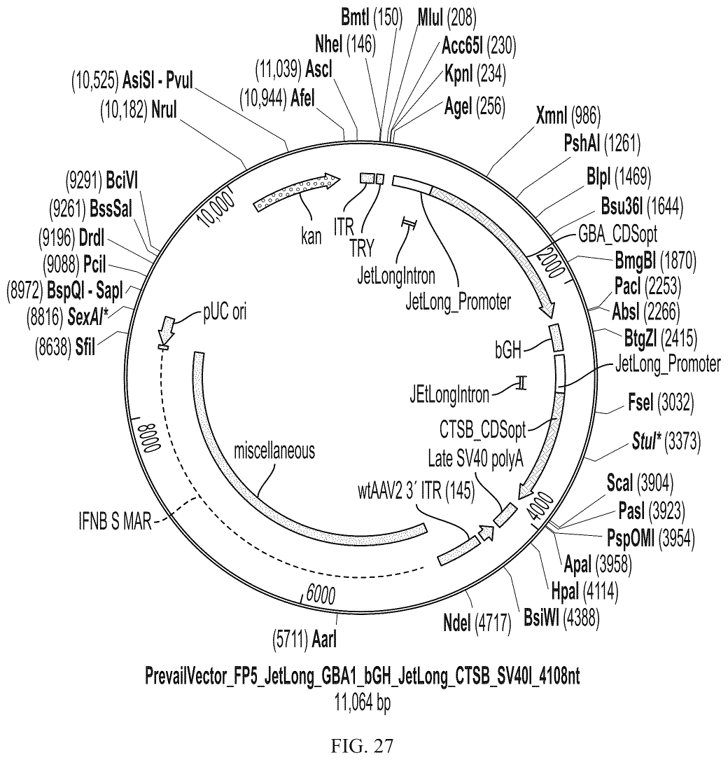

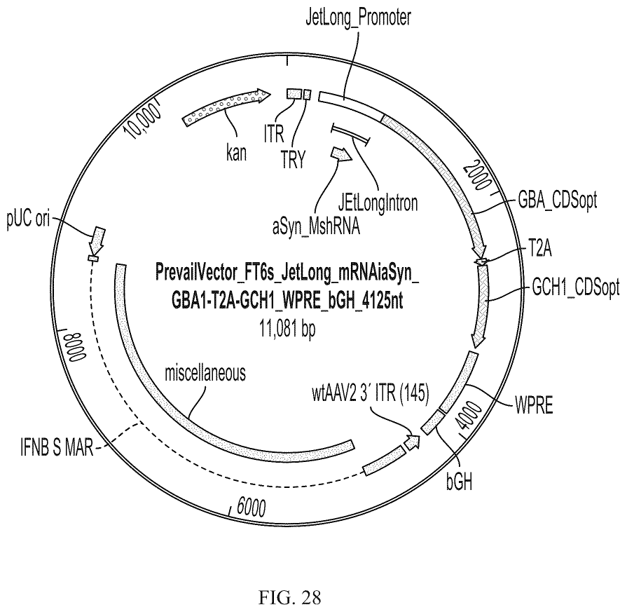

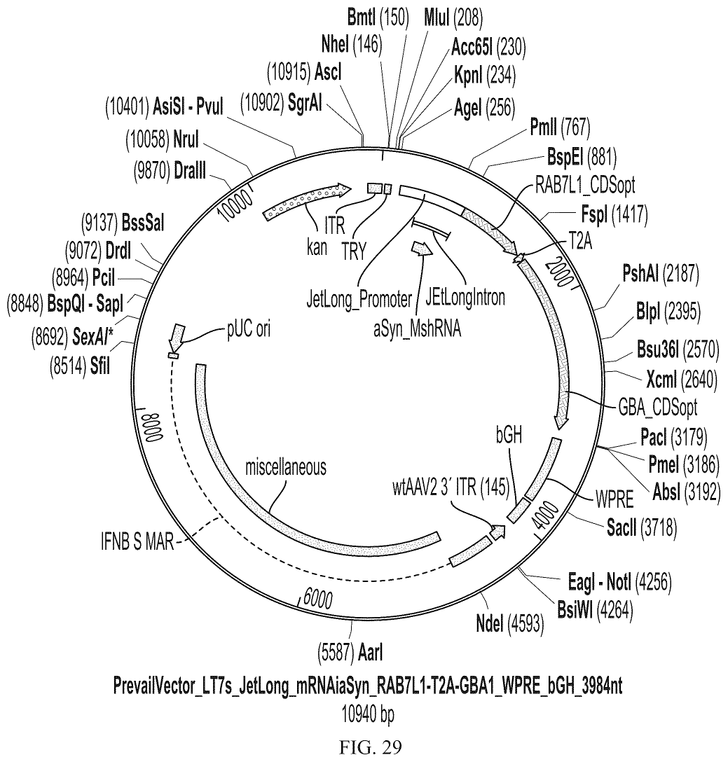

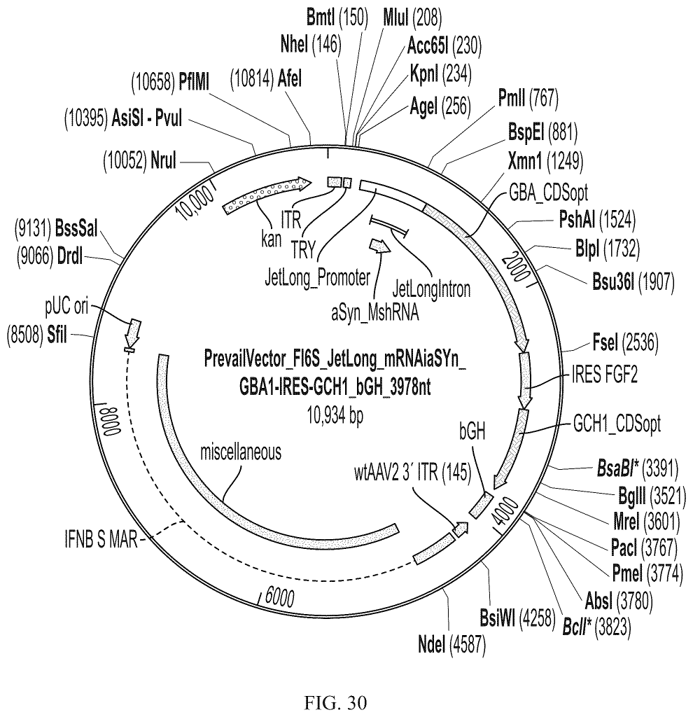

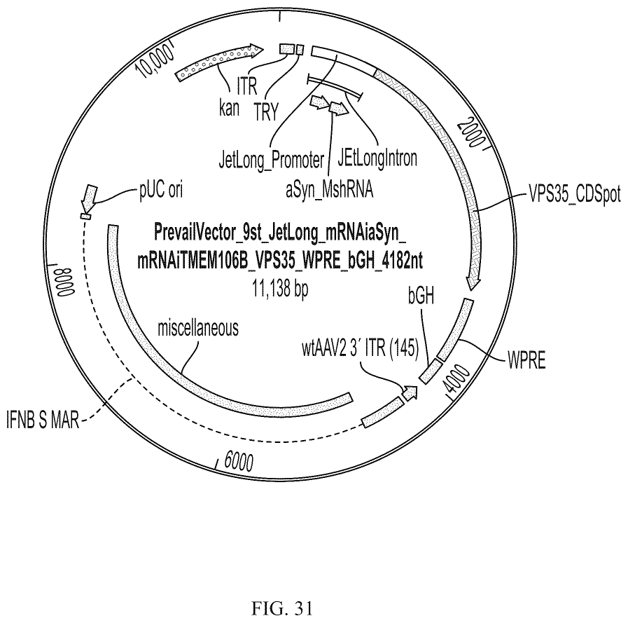

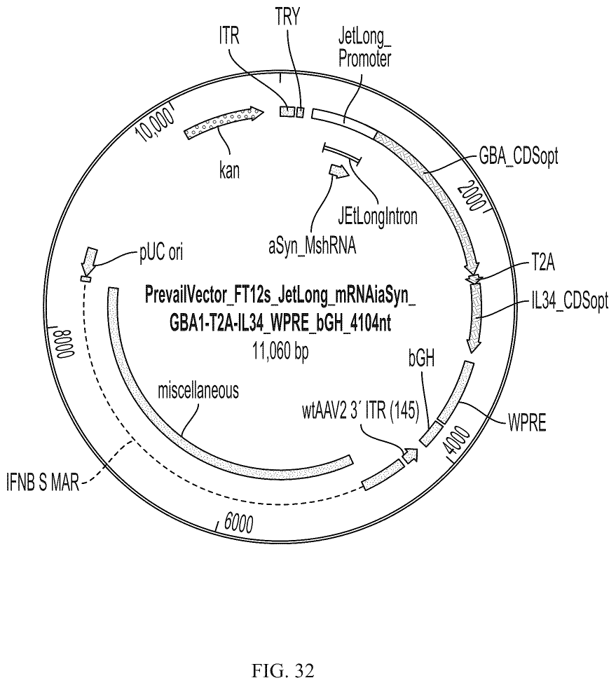

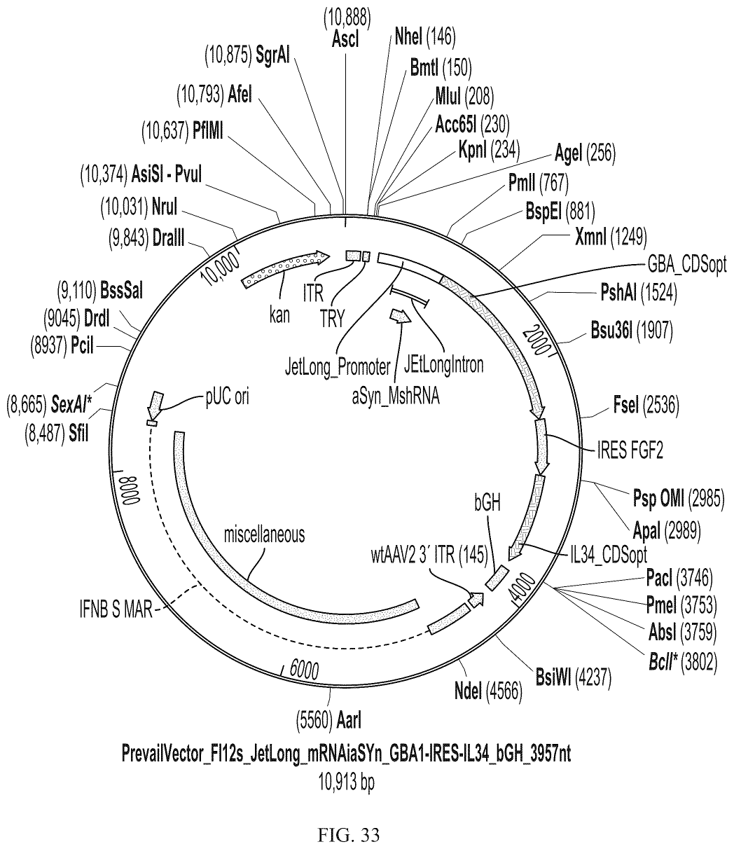

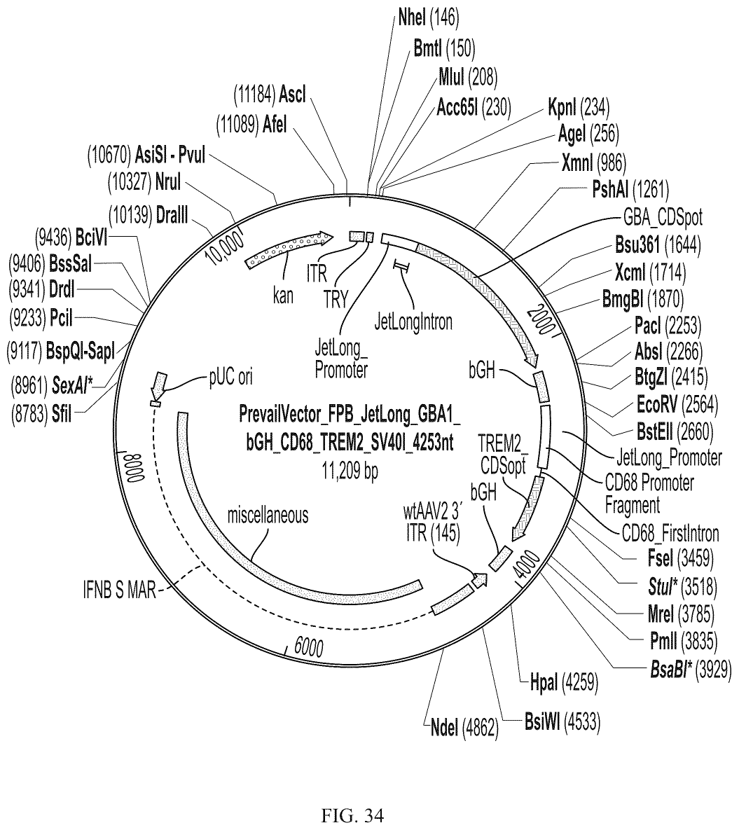

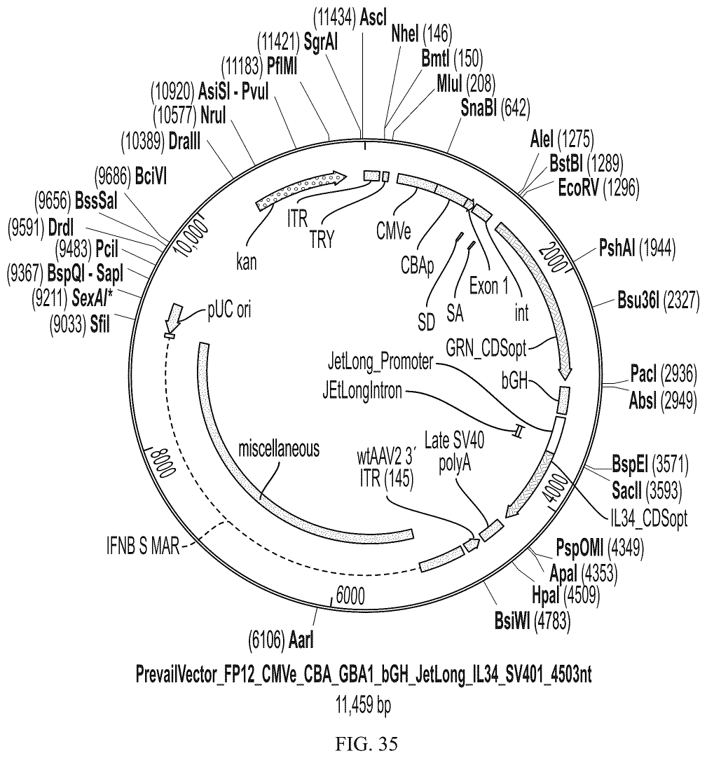

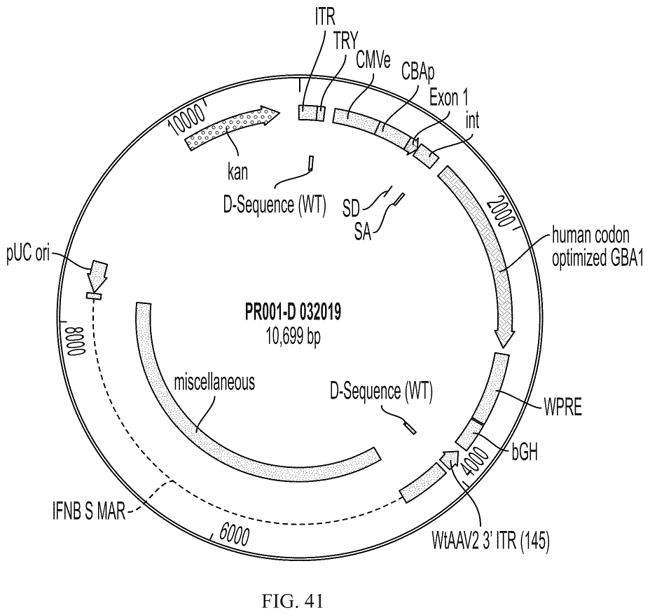

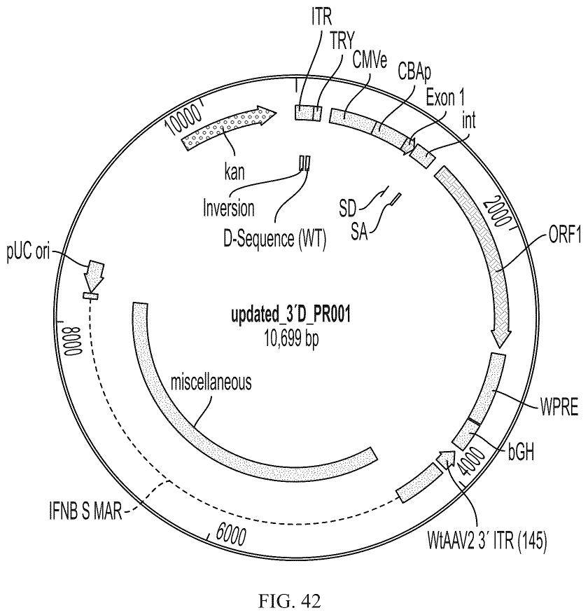

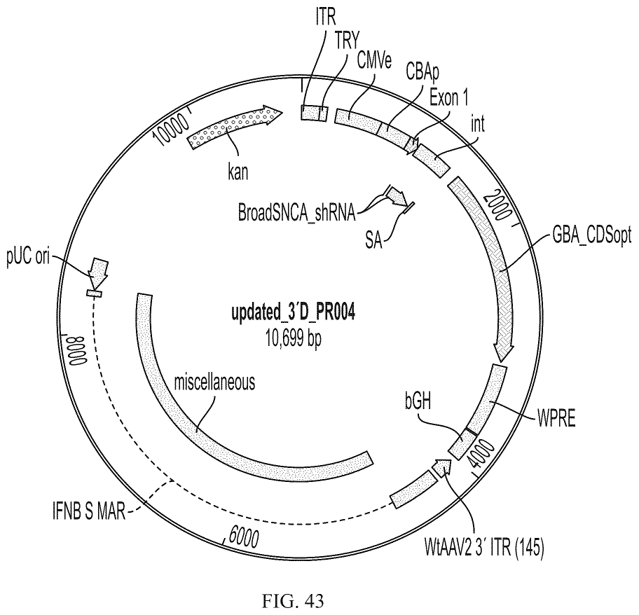

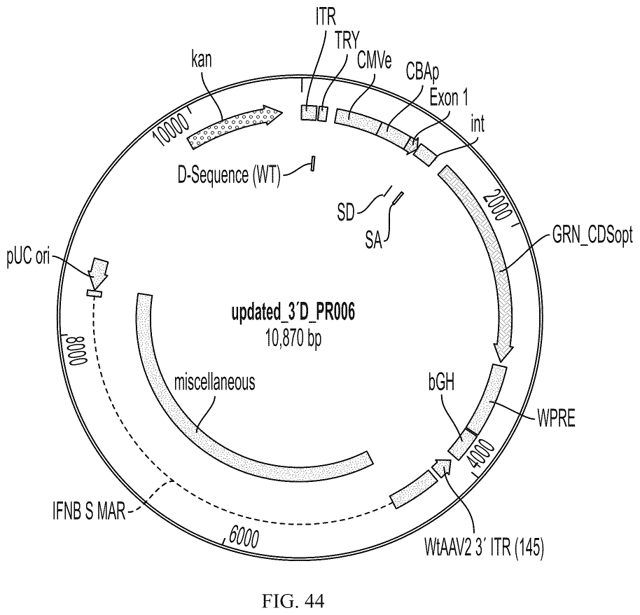

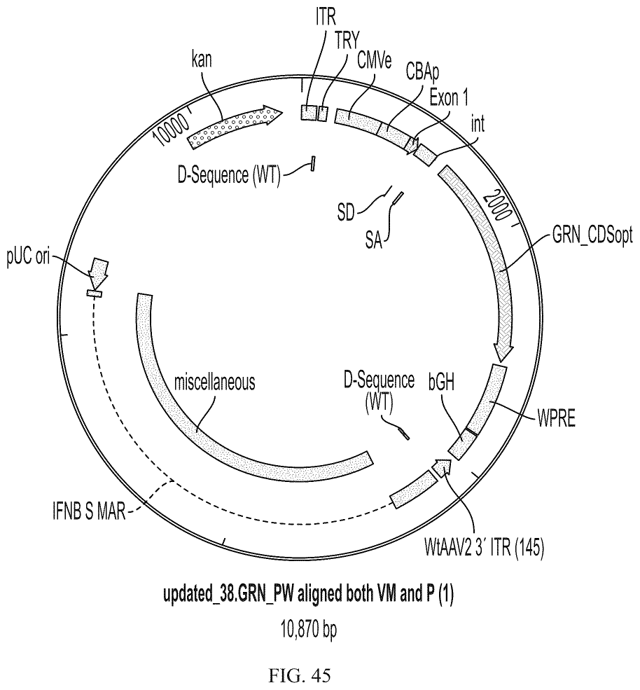

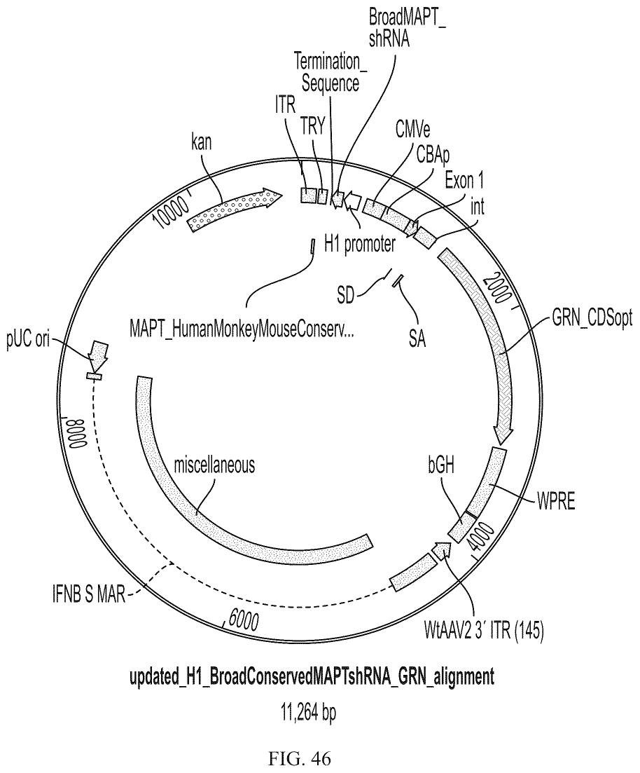

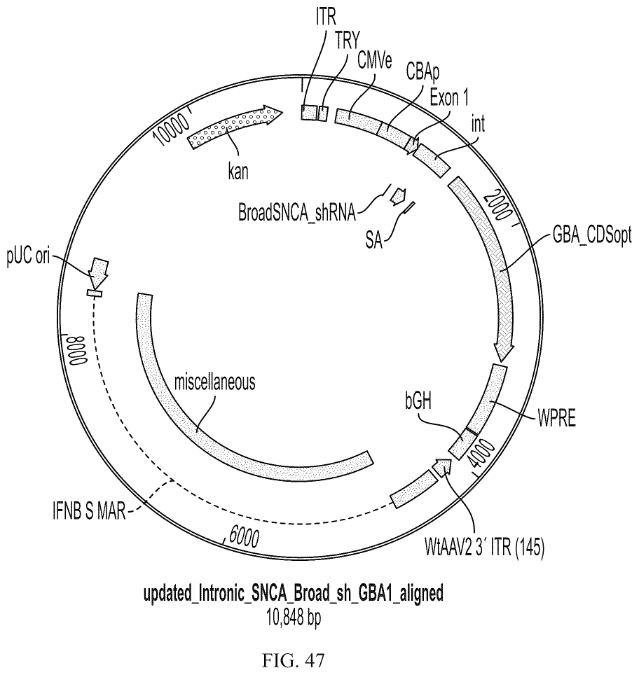

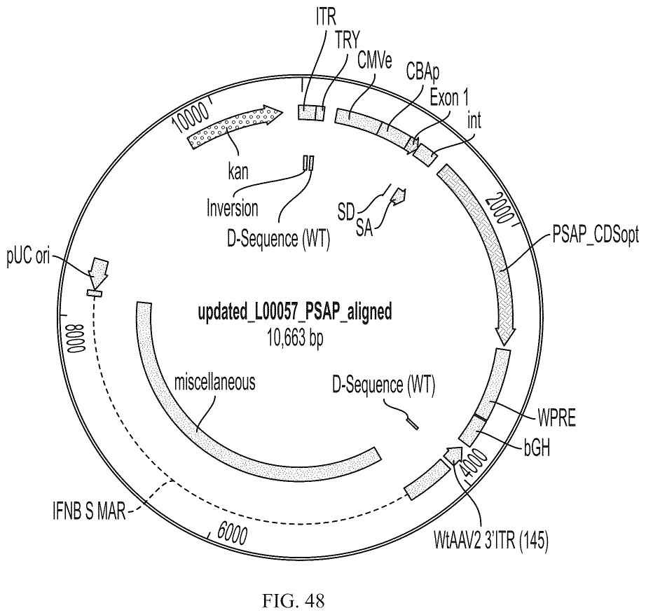

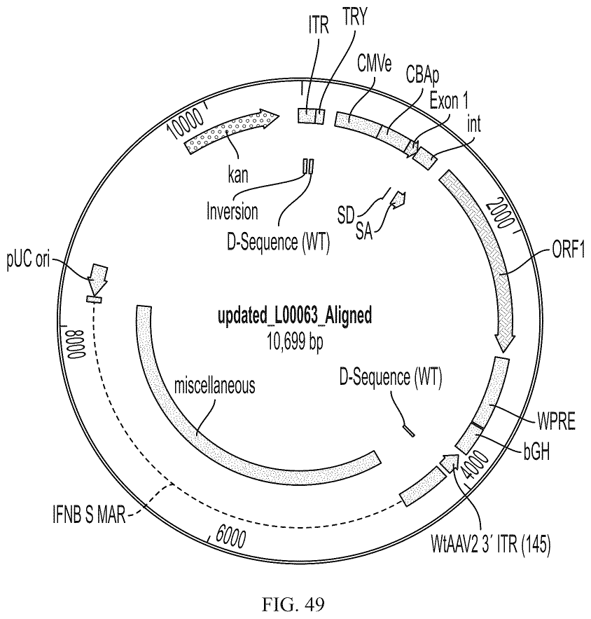

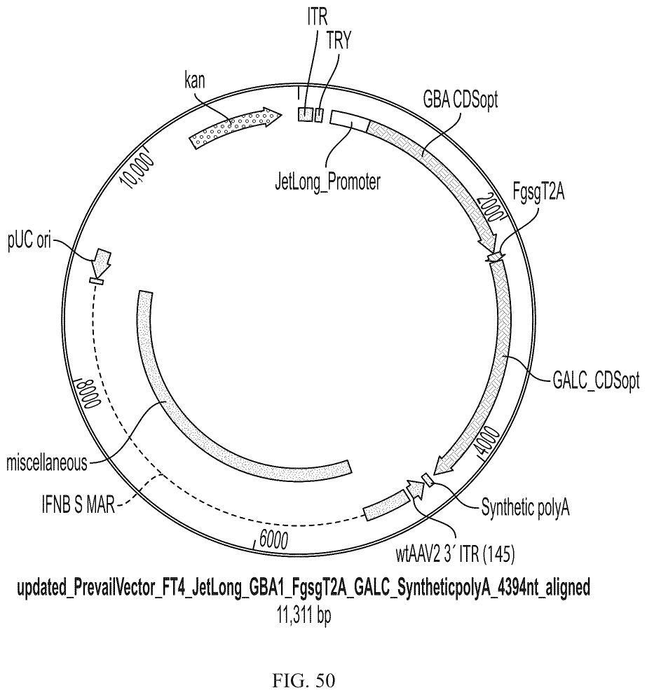

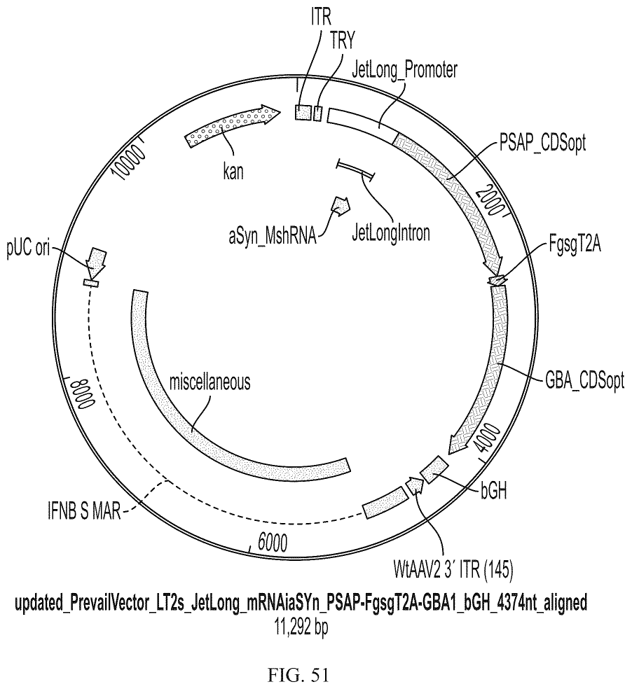

AAV vectors are generated using cells, such as HEK293 cells for triple-plasmid transfection. The ITR sequences flank an expression construct comprising a promoter/enhancer element for each transgene of interest, a 3′ polyA signal, and posttranslational signals such as the WPRE element. Multiple gene products can be expressed simultaneously such as GBA1 and LIMP2 and/or Prosaposin, by fusion of the protein sequences; or using a 2A peptide linker, such as T2A or P2A, which leads 2 peptide fragments with added amino acids due to prevention of the creation of a peptide bond; or using an IRES element; or by expression with 2 separate expression cassettes. The presence of a short intronic sequence that is efficiently spliced, upstream of the expressed gene, can improve expression levels. shRNAs and other regulatory RNAs can potentially be included within these sequences. Examples of expression constructs described by the disclosure are shown in FIGS. 1-8, 21-35, 39, 41-51 and 64 and in Table 2 below.

TABLE 2

| | Pro- | | | | |
|---|---|---|---|---|---|
| | moter | | | | |
| Name | 1 | shRNA | CDS1 | PolyA 1 | |
| CMVe_CBAp_GBA1_WPRE_bGH | CBA | | GBA1 | WPRE-bGH | |
| LT1s_JetLong_mRNAiaSYn_SCARB2-T2A-GBA1_bGH | JetLong | aSyn | SCARB2 | bGH | |
| LI1_JetLong_SCARB2-IRES-GBA1_bGH | JetLong | | SCARB2 | bGH | |
| FP1_JetLong_GBA1_bGH_JetLong_SCARB2_SV40L | JetLong | | GBA1 | bGH | |
| PrevailVector_LT2s_JetLong_mRNAiaSYn_PSAP-T2A-GBA1_bGH_4353nt | JetLong | aSyn | PSAP | bGH | |
| PrevailVector_LI2_JetLong_PSAP_TRES_GBA1_SymtheticpolyA_4337nt | JetLong | — | PSAP | Synthetic pA | |
| PrevailVector_10s_JetLong_mRNAiaSy_GBA2_WPRE_bGH_4308nt | JetLong | aSyn | GBA2 | WPRE_bGH | |
| PrevailVector_FT4_JetLong_GBA1_T2A_GALC_SyntheticpolyA_4373nt | JetLong | — | GBA1 | Synthetic pA | |
| PrevailVector_LT4_JetLong_GALC_T2A_GBA1_SyntheticpolyA_4373nt | JetLong | — | GALC | Synthetic pA | |
| PrevailVector_LT5s_JetLong_mRNAiaSyn_CTSB-T2A-GBA1_WPRE_bGH_4392nt | JetLong | aSyn | CTSB | WPRE_bGH | |
| Prevail_Vector_FT11t_JetLong_mRNAiaSyn_GBA1_T2S_SMPD1_SyntheticpolyA_4477nt | JetLong | aSyn | GBA1 | Synthetic pA | |
| PrevailVector_LI4_JetLong_GALC_TRES_GBA1_SymtheticpolyA_4820nt | JetLong | — | GALC | Synthetic pA | |
| PrevailVector_FP5_JetLong_GBA1_bGH_JetLong_CTSB_SV40l_4108nt | JetLong | — | GBA1 | bGH | |
| PrevailVector_FT6s_JetLong_mRNAiaSyn_GBA1-T2A-GCH1_WPRE_bGH_4125nt | JetLong | aSyn | GBA1 | WPRE_bGH | |
| PrevailVector_LT7s_JetLong_mRNAiaSyn_RAB7L1-T2A-GBA1_WPRE_bGH_3984nt | JetLong | aSyn | RAB7L1 | WPRE_bGH | |
| PrevailVector_FI6s_JetLong_mRNAiaSYn_GBA1-IRES-GCH1_bGH_3978nt | JetLong | aSyn | GBA1 | bGH | |
| PrevailVector_9st_JetLong_mRNAiaSyn_mRNAiTMEM106B_VPS35_WPRE_bGH_4182nt | JetLong | aSyn & | VPS35 | WPRE_bGH | |
| | | TMEM106B | | | |
| PrevailVector_FT12s_JetLong_mRNAiaSyn_GBA1-T2A-IL34_WPRE_bGH_4104nt | JetLong | aSyn | GBA1 | WPRE_bGH | |
| PrevailVector_FI12s_JetLong_mRNAiaSYn_GBA1-IRES-IL34_bGH_3957nt | JetLong | aSyn | GBA1 | bGH | |
| PrevailVector_FP8_JetLong_GBA1_bGH_CD68_TREM2_SV40l_4253nt | JetLong | — | GBA1 | bGH | |
| PrevailVector_FP12_CMVe_CBA_GBA1_bGH_JetLong_IL34_SV40l_4503nt | CBA | | GBA1 | bGH | |
| | Bicis- | Pro- | | | Length |
| | tronic | moter | | | between |
| Name | element | 2 | CDS2 | PolyA2 | ITRs |
| CMVe_CBAp_GBA1_WPRE_bGH | | | | | 3741 |
| LT1s_JetLong_mRNAiaSYn_SCARB2-T2A-GBA1_bGH | T2A | | GBA1 | | 4215 |
| LI1_JetLong_SCARB2-IRES-GBA1_bGH | IRES | | GBA1 | | 4399 |
| FP1_JetLong_GBA1_bGH_JetLong_SCARB2_SV40L | | JetLong | SCARB2 | SV40L | 4464 |
| PrevailVector_LT2s_JetLong_mRNAiaSYn_PSAP-T2A-GBA1_bGH_4353nt | T2A | — | GBA1 | — | 4353 |
| PrevailVector_LI2_JetLong_PSAP_TRES_GBA1_SymtheticpolyA_4337nt | IRES | — | GBA1 | — | 4337 |
| PrevailVector_10s_JetLong_mRNAiaSy_GBA2_WPRE_bGH_4308nt | — | — | — | — | 4308 |
| PrevailVector_FT4_JetLong_GBA1_T2A_GALC_SyntheticpolyA_4373nt | T2A | — | GALC | — | 4373 |
| PrevailVector_LT4_JetLong_GALC_T2A_GBA1_SyntheticpolyA_4373nt | T2A | — | GBA1 | — | 4373 |
| PrevailVector_LT5s_JetLong_mRNAiaSyn_CTSB-T2A-GBA1_WPRE_bGH_4392nt | T2A | — | GBA1 | — | 4392 |
| Prevail_Vector_FT11t_JetLong_mRNAiaSyn_GBA1_T2S_SMPD1_SyntheticpolyA_4477nt | T2A | — | SMPD1 | — | 4477 |
| PrevailVector_LI4_JetLong_GALC_TRES_GBA1_SymtheticpolyA_4820nt | IRES | — | GBA1 | — | 4820 |
| PrevailVector_FP5_JetLong_GBA1_bGH_JetLong_CTSB_SV40l_4108nt | — | JetLong | CTSB | SV40L | 4108 |
| PrevailVector_FT6s_JetLong_mRNAiaSyn_GBA1-T2A-GCH1_WPRE_bGH_4125nt | T2A | — | GCH1 | — | 4125 |
| PrevailVector_LT7s_JetLong_mRNAiaSyn_RAB7L1-T2A-GBA1_WPRE_bGH_3984nt | T2A | — | GBA1 | — | 3984 |
| PrevailVector_FI6s_JetLong_mRNAiaSYn_GBA1-IRES-GCH1_bGH_3978nt | IRES | — | GCH1 | — | 3978 |
| PrevailVector_9st_JetLong_mRNAiaSyn_mRNAiTMEM106B_VPS35_WPRE_bGH_4182nt | — | — | — | — | 4182 |
| PrevailVector_FT12s_JetLong_mRNAiaSyn_GBA1-T2A-IL34_WPRE_bGH_4104nt | T2A | — | IL34 | — | 4104 |
| PrevailVector_FI12s_JetLong_mRNAiaSYn_GBA1-IRES-IL34_bGH_3957nt | IRES | — | IL34 | — | 3957 |
| PrevailVector_FP8_JetLong_GBA1_bGH_CD68_TREM2_SV40l_4253nt | — | CD68 | TREM2 | SV40L | 4253 |
| PrevailVector_FP12_CMVe_CBA_GBA1_bGH_JetLong_IL34_SV40l_4503nt | | JetLong | IL34 | SV40L | 4503 |

### Example 2: Cell Based Assays of Viral Transduction into GBA-Deficient Cells

Cells deficient in GBA1 are obtained, for example as fibroblasts from GD patients, monocytes, or hES cells, or patient-derived induced pluripotent stem cells (iPSCs). These cells accumulate substrates such as glucosylceramide and glucosylsphingosine (GlcCer and GlcSph). Treatment of wild-type or mutant cultured cell lines with Gcase inhibitors, such as CBE, is also be used to obtain GBA deficient cells.

Using such cell models, lysosomal defects are quantified in terms of accumulation of protein aggregates, such as of α-Synuclein with an antibody for this protein or phospho-aSyn, followed by imaging using fluorescent microscopy. Imaging for lysosomal abnormalities by ICC for protein markers such as LAMP1, LAMP2, LIMP1, LIMP2, or using dyes such as Lysotracker, or by uptake through the endocytic compartment of fluorescent dextran or other markers is also performed. Imaging for autophagy marker accumulation due to defective fusion with the lysosome, such as for LC3, can also be performed. Western blotting and/or ELISA is used to quantify abnormal accumulation of these markers. Also, the accumulation of glycolipid substrates and products of GBA1 is measured using standard approaches.

Therapeutic endpoints (e.g., reduction of PD-associated pathology) are measured in the context of expression of transduction of the AAV vectors, to confirm and quantify activity and function. Gcase can is also quantified using protein ELISA measures, or by standard Gcase activity assays.

### Example 3: In Vivo Assays Using Mutant Mice

This example describes in vivo assays of AAV vectors using mutant mice. In vivo studies of AAV vectors as above in mutant mice are performed using assays described, for example, by Liou et al. (2006) J. Biol. Chem. 281(7): 4242-4253, Sun et al. (2005) J. Lipid Res. 46:2102-2113, and Farfel-Becker et al. (2011) Dis. ModelMech. 4(6):746-752.

The intrathecal or intraventricular delivery of vehicle control and AAV vectors (e.g., at a dose of 2×10¹¹ vg/mouse) are performed using concentrated AAV stocks, for example at an injection volume between 5-10 μL. Intraparenchymal delivery by convection enhanced delivery is performed.

Treatment is initiated either before onset of symptoms, or subsequent to onset. Endpoints measured are the accumulation of substrate in the CNS and CSF, accumulation of Gcase enzyme by ELISA and of enzyme activity, motor and cognitive endpoints, lysosomal dysfunction, and accumulation of α-Synuclein monomers, protofibrils or fibrils.

### Example 4: Chemical Models of Disease

This example describes in vivo assays of AAV vectors using a chemically-induced mouse model of Gaucher disease (e.g., the CBE mouse model). In vivo studies of these AAV vectors are performed in a chemically-induced mouse model of Gaucher disease, for example as described by Vardi et al. (2016) J Pathol. 239(4):496-509.

Intrathecal or intraventricular delivery of vehicle control and AAV vectors (e.g., at a dose of 2×10¹¹ vg/mouse) are performed using concentrated AAV stocks, for example with injection volume between 5-10 μL. Intraparenchymal delivery by convection enhanced delivery is performed. Peripheral delivery is achieved by tail vein injection.

Treatment is initiated either before onset of symptoms, or subsequent to onset. Endpoints measured are the accumulation of substrate in the CNS and CSF, accumulation of Gcase enzyme by ELISA and of enzyme activity, motor and cognitive endpoints, lysosomal dysfunction, and accumulation of α-Synuclein monomers, protofibrils or fibrils.

### Example 5: Clinical Trials in PD, LBD, Gaucher Disease Patients

In some embodiments, patients having certain forms of Gaucher disease (e.g., GD1) have an increased risk of developing Parkinson's disease (PD) or Lewy body dementia (LBD). This Example describes clinical trials to assess the safety and efficacy of rAAVs as described by the disclosure, in patients having Gaucher disease, PD and/or LBD.

Clinical trials of such vectors for treatment of Gaucher disease, PD and/or LBD are performed using a study design similar to that described in Grabowski et al. (1995) Ann. Intern. Med. 122(1):33-39.

### Example 6: Treatment of Peripheral Disease

In some embodiments, patients having certain forms of Gaucher disease exhibit symptoms of peripheral neuropathy, for example as described in Biegstraaten et al. (2010) Brain 133(10):2909-2919.

This example describes in vivo assays of AAV vectors as described herein for treatment of peripheral neuropathy associated with Gaucher disease (e.g., Type 1 Gaucher disease). Briefly, Type 1 Gaucher disease patients identified as having signs or symptoms of peripheral neuropathy are administered a rAAV as described by the disclosure. In some embodiments, the peripheral neuropathic signs and symptoms of the subject are monitored, for example using methods described in Biegstraaten et al., after administration of the rAAV.

Levels of transduced gene products as described by the disclosure present in patients (e.g., in serum of a patient, in peripheral tissue (e.g., liver tissue, spleen tissue, etc.)) of a patient are assayed, for example by Western blot analysis, enzymatic functional assays, or imaging studies.

### Example 7: Treatment of CNS Forms

This example describes in vivo assays of rAAVs as described herein for treatment of CNS forms of Gaucher disease. Briefly, Gaucher disease patients identified as having a CNS form of Gaucher disease (e.g., Type 2 or Type 3 Gaucher disease) are administered a rAAV as described by the disclosure. Levels of transduced gene products as described by the disclosure present in the CNS of patients (e.g., in serum of the CNS of a patient, in cerebrospinal fluid (CSF) of a patient, or in CNS tissue of a patient) are assayed, for example by Western blot analysis, enzymatic functional assays, or imaging studies.

### Example 8: Gene Therapy of Parkinson's Disease in Subjects Having Mutations in GBA1

This example describes administration of a recombinant adeno-associated virus (rAAV) encoding GBA1 to a subject having Parkinson's disease characterized by a mutation in GBA1 gene.

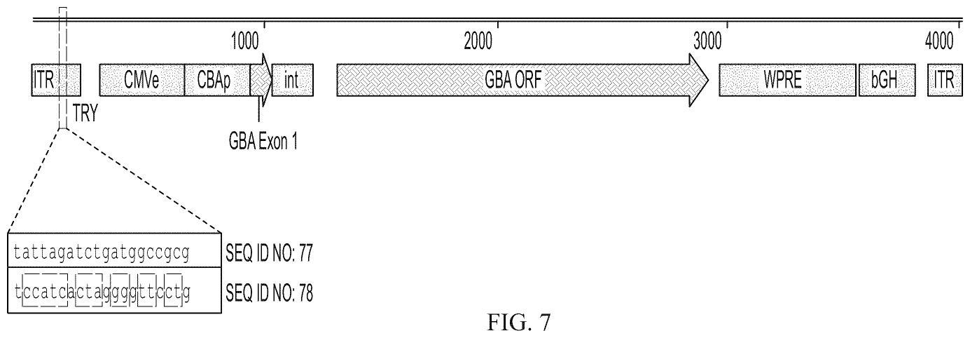

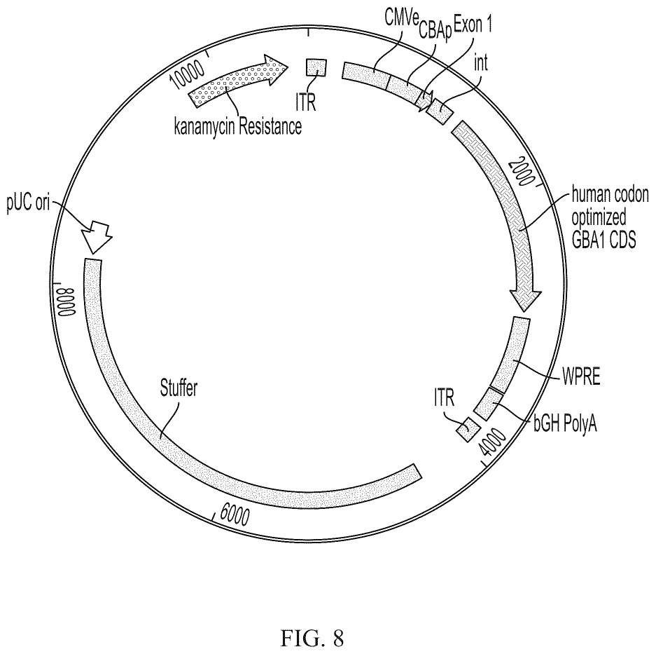

The rAAV-GBA1 vector insert contains the CBA promoter element (CBA), consisting of four parts: the CMV enhancer (CMVe), CBA promoter (CBAp), Exon 1, and intron (int) to constitutively express the codon optimized coding sequence (CDS) of human GBA1 (maroon). The 3′ region also contains a Woodchuck hepatitis virus Posttranscriptional Regulatory Element (WPRE) followed by a bovine Growth Hormone polyA signal (bGH polyA) tail. The flanking ITRs allow for the correct packaging of the intervening sequences. Two variants of the 5′ ITR sequence (FIG. 7, inset box, bottom sequence) were evaluated; these variants have several nucleotide differences within the 20-nucleotide “D” region of the ITR, which is believed to impact the efficiency of packaging and expression. The rAAV-GBA1 vector product contains the “D” domain nucleotide sequence shown in FIG. 7 (inset box, top sequence). A variant vector harboring a mutant “D” domain (termed an “S” domain herein, with the nucleotide changes shown by shading), performed similarly in preclinical studies. The backbone contains the gene to confer resistance to kanamycin as well as a stuffer sequence to prevent reverse packaging. A schematic depicting a rAAV-GBA1 vector is shown in FIG. 8. The rAAV-GBA1 vector is packaged into an rAAV using AAV9 serotype capsid proteins.

rAAV-GBA1 is administered to a subject as a single dose via a fluoroscopy guided sub-occipital injection into the cisterna magna (intracisternal magna; ICM). One embodiment of a rAAV-GBA1 dosing regimen study is as follows:

A single dose of rAAV-GBA1 is administered to patients (N=12) at one of two dose levels (3e13 vg (low dose); 1e14 vg (high dose), etc.) which are determined based on the results of nonclinical pharmacology and toxicology studies.

Initial studies were conducted in a chemical mouse model involving daily delivery of conduritol-b-epoxide (CBE), an inhibitor of GCase to assess the efficacy and safety of the rAAV-GBA1 vector and a rAAV-GBA1 S-variant construct (as described further below). Additionally, initial studies were performed in a genetic mouse model, which carries a homozygous GBA1 mutation and is partially deficient in saposins (4L/PS-NA). Additional dose-ranging studies in mice and nonhuman primates (NHPs) are conducted to further evaluate vector safety and efficacy.

Two slightly different versions of the 5′ inverted terminal repeat (ITR) in the AAV backbone were tested to assess manufacturability and transgene expression (FIG. 7). The 20 bp “D” domain within the 145 bp 5′ ITR is thought to be necessary for optimal viral vector production, but mutations within the “D” domain have also been reported to increase transgene expression in some cases. Thus, in addition to the viral vector rAAV-GBA1, which harbors an intact “D” domain, a second vector form with a mutant D domain (termed an “S” domain herein) was also evaluated. Both rAAV-GBA1 and the variant express the same transgene. While both vectors produced virus that was efficacious in vivo as detailed below, rAAV-GBA1, which contains a wild-type “D” domain, was selected for further development.

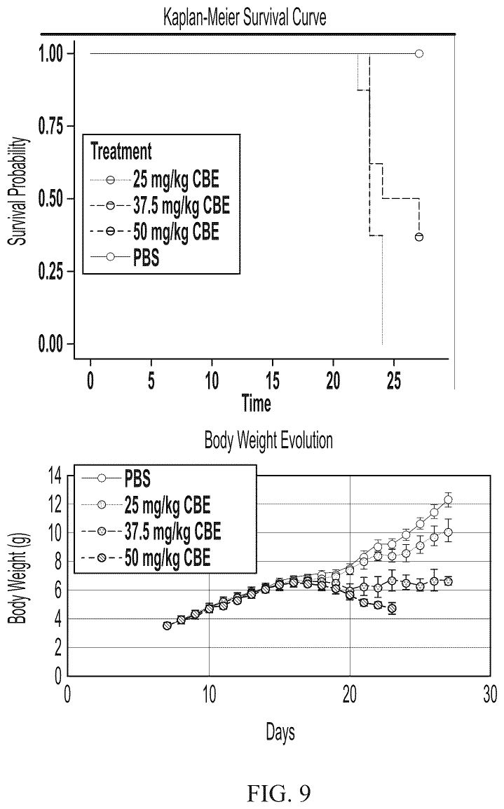

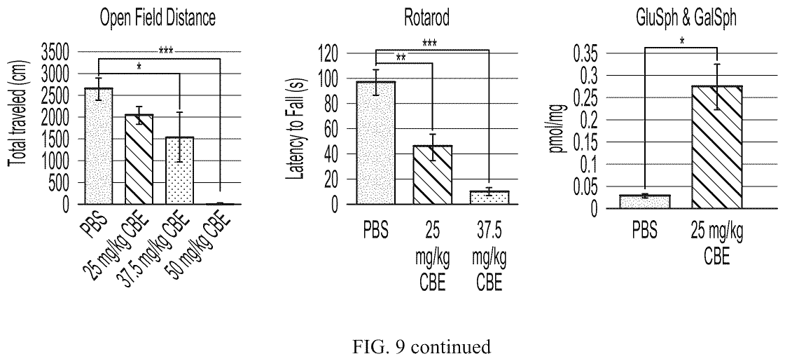

To establish the CBE model of GCase deficiency, juvenile mice were dosed with CBE, a specific inhibitor of GCase. Mice were given CBE by IP injection daily, starting at postnatal day 8 (P8). Three different CBE doses (25 mg/kg, 37.5 mg/kg, 50 mg/kg) and PBS were tested to establish a model that exhibits a behavioral phenotype (FIG. 9). Higher doses of CBE led to lethality in a dose-dependent manner. All mice treated with 50 mg/kg CBE died by P23, and 5 of the 8 mice treated with 37.5 mg/kg CBE died by P27. There was no lethality in mice treated with 25 mg/kg CBE. Whereas CBE-injected mice showed no general motor deficits in the open field assay (traveling the same distance and at the same velocity as mice given PBS), CBE-treated mice exhibited a motor coordination and balance deficit as measured by the rotarod assay.

Mice surviving to the end of the study were sacrificed on the day after their last CBE dose (P27, “Day 1”) or after three days of CBE withdrawal (P29, “Day 3”). Lipid analysis was performed on the cortex of mice given 25 mg/kg CBE to evaluate the accumulation of GCase substrates in both the Day 1 and Day 3 cohorts. GluSph and GalSph levels (measured in aggregate in this example) were significantly accumulated in the CBE-treated mice compared to PBS-treated controls, consistent with GCase insufficiency.

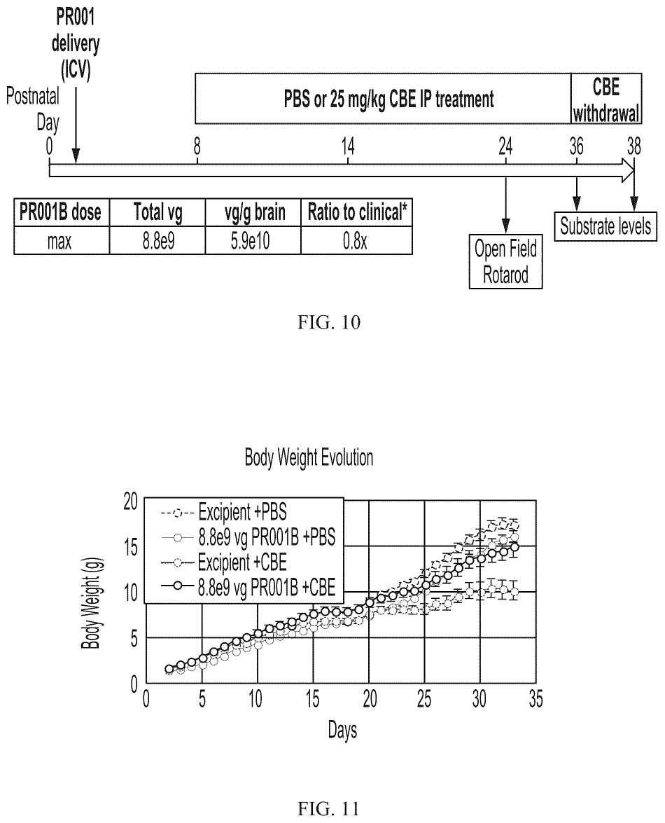

Based on the study described above, the 25 mg/kg CBE dose was selected since it produced behavioral deficits without impacting survival. To achieve widespread GBA1 distribution throughout the brain and transgene expression during CBE treatment, rAAV-GBA1 or excipient was delivered by intracerebroventricular (ICV) injection at postnatal day 3 (P3) followed by daily IP CBE or PBS treatment initiated at P8 (FIG. 10).

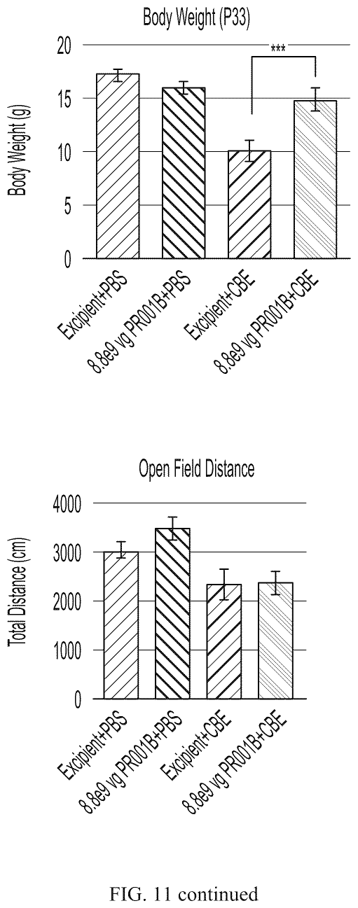

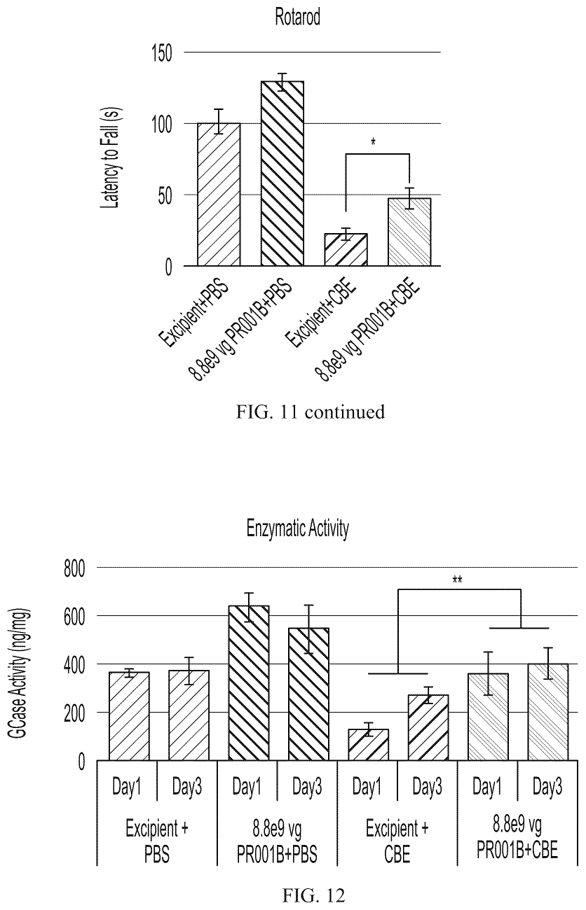

CBE-treated mice that received rAAV-GBA1 performed statistically significantly better on the rotarod than those that received excipient (FIG. 11). Mice in the variant treatment group did not differ from excipient treated mice in terms of other behavioral measures, such as the total distance traveled during testing (FIG. 11).

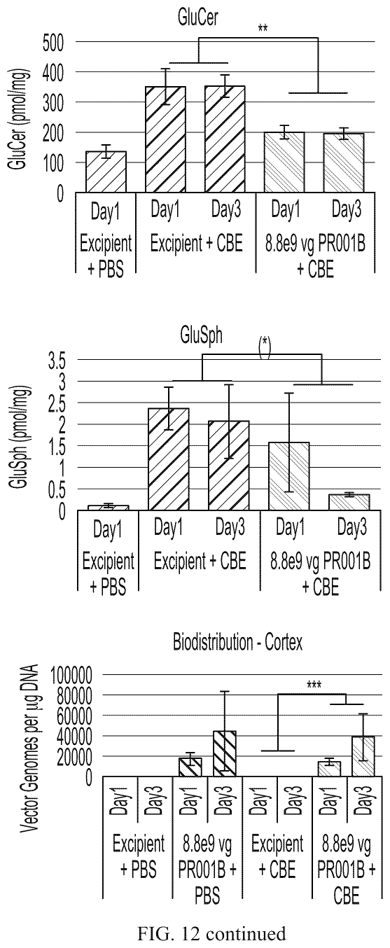

At the completion of the in-life study, half of the mice were sacrificed the day after the last CBE dose (P36, “Day 1”) or after three days of CBE withdrawal (P38, “Day 3”) for biochemical analysis (FIG. 12). Using a fluorometric enzyme assay performed in biological triplicate, GCase activity was assessed in the cortex. GCase activity was increased in mice that were treated with rAAV-GBA1, while CBE treatment reduced GCase activity. Additionally, mice that received both CBE and rAAV-GBA1 had GCase activity levels that were similar to the PBS-treated group, indicating that delivery of rAAV-GBA1 is able to overcome the inhibition of GCase activity induced by CBE treatment. Lipid analysis was performed on the motor cortex of the mice to examine levels of the substrates GluCer and GluSph. Both lipids accumulated in the brains of mice given CBE, and rAAV-GBA1 treatment significantly reduced substrate accumulation.

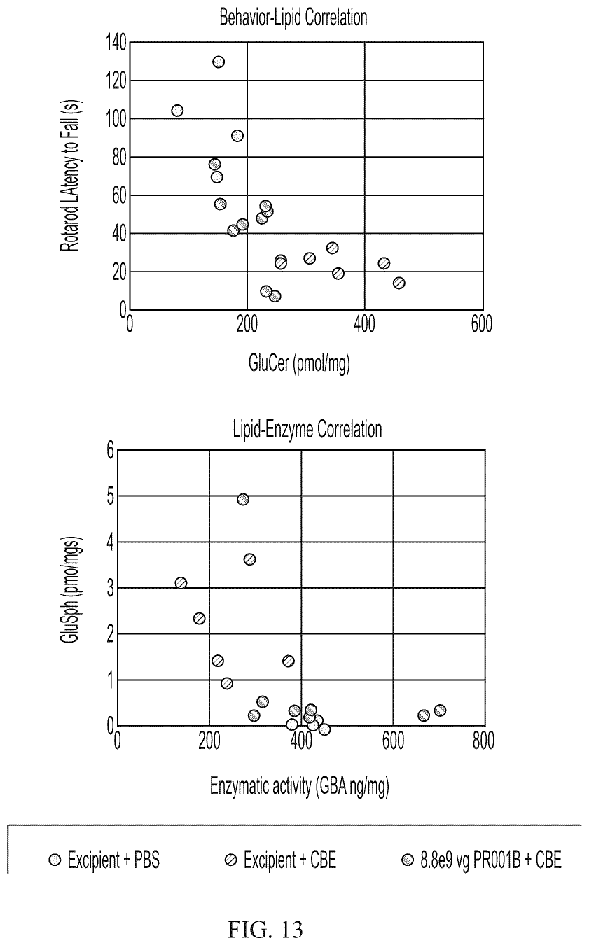

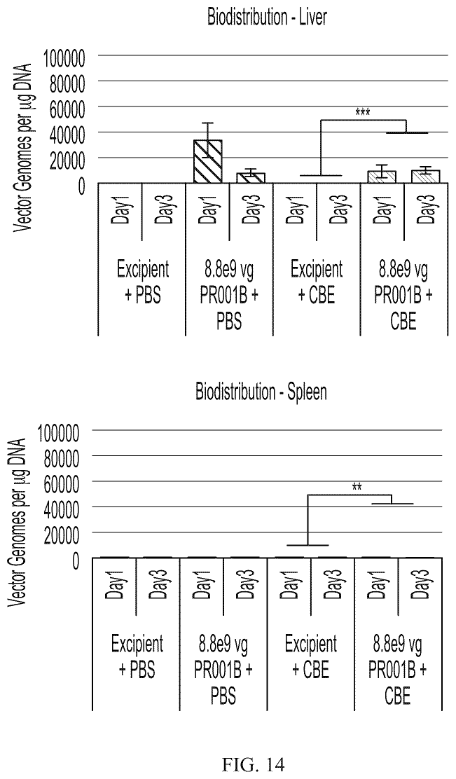

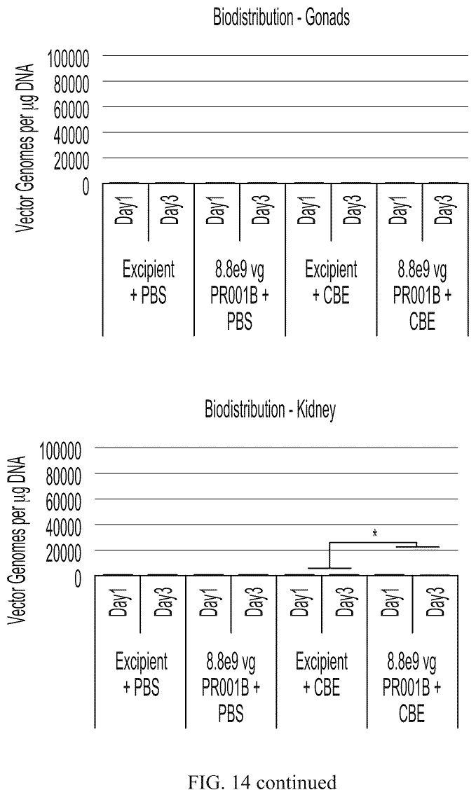

Lipid levels were negatively correlated with both GCase activity and performance on the Rotarod across treatment groups. The increased GCase activity after rAAV-GBA1 administration was associated with substrate reduction and enhanced motor function (FIG. 13). As shown in FIG. 14, preliminary biodistribution was assessed by vector genome presence, as measured by qPCR (with >100 vector genomes per 1 μg genomic DNA defined as positive). Mice that received rAAV-GBA1, both with and without CBE, were positive for rAAV-GBA1 vector genomes in the cortex, indicating that ICV delivery results in rAAV-GBA1 delivery to the cortex. Additionally, vector genomes were detected in the liver, few in spleen, and none in the heart, kidney or gonads. For all measures, there was no statistically significant difference between the Day 1 and Day 3 groups.

A larger study in the CBE model further explored efficacious doses of rAAV-GBA1 in the CBE model. Using the 25 mg/kg CBE dose model, excipient or rAAV-GBA1 was delivered via ICV at P3, and daily IP PBS or CBE treatment initiated at P8. Given the similarity between the groups with and without CBE withdrawal observed in the previous studies, all mice were sacrificed one day after the final CBE dose (P38-40). The effect of three different rAAV-GBA1 doses was assessed, resulting in the following five groups, with 10 mice (5M/5F) per group:
Excipient ICV+PBS IP
Excipient ICV+25 mg/kg CBE IP
3.2e9 vg (2.13e10 vg/g brain) rAAV-GBA1 ICV+25 mg/kg CBE IP
1.0e10 vg (6.67e10 vg/g brain) rAAV-GBA1 ICV+25 mg/kg CBE IP 3.2e10 vg (2.13e11 vg/g brain) rAAV-GBA1 ICV+25 mg/kg CBE IP.

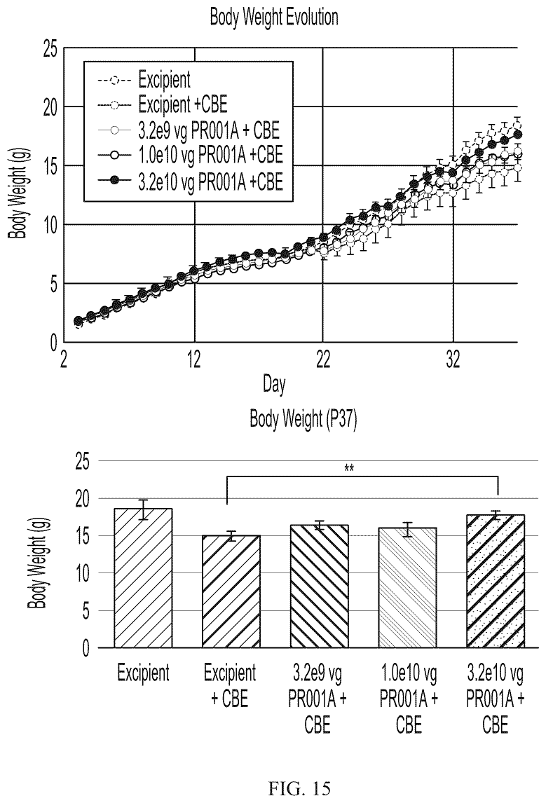

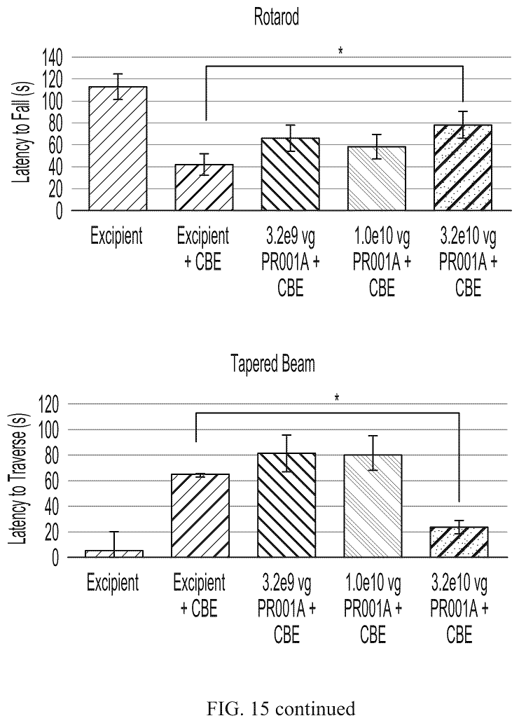

The highest dose of rAAV-GBA1 rescued the CBE treatment-related failure to gain weight at P37. Additionally, this dose resulted in a statistically significant increase in performance on the rotarod and tapered beam compared to the Excipient+CBE treated group (FIG. 15). Lethality was observed in several groups, including both excipient-treated and rAAV-GBA1-treated groups (Excipient+PBS: 0; Excipient+25 mg/kg CBE: 1; 3.2e9 vg rAAV-GBA1+25 mg/kg CBE: 4; 1.0e10 vg rAAV-GBA1+25 mg/kg CBE: 0; 3.2e10 vg rAAV-GBA1+25 mg/kg CBE: 3).

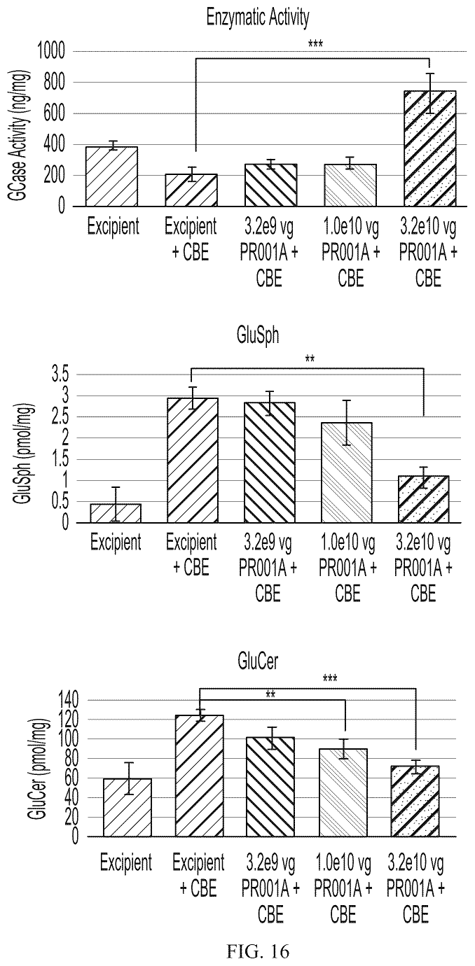

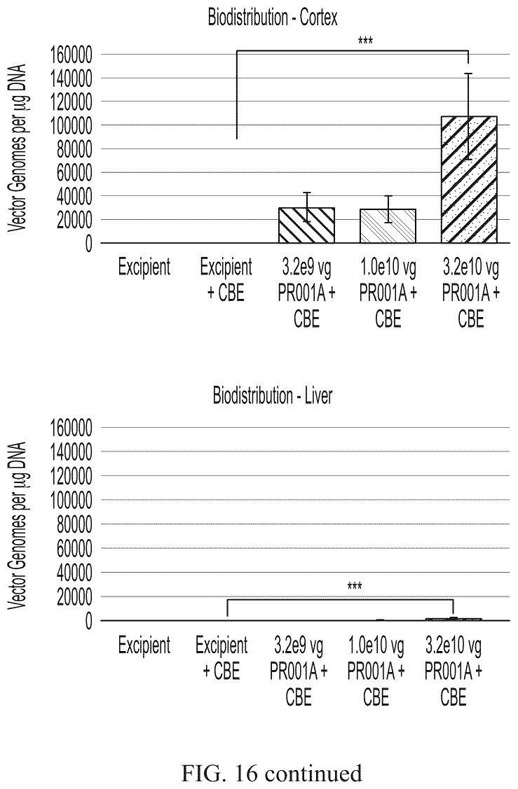

At the completion of the in-life study, mice were sacrificed for biochemical analysis (FIG. 16). GCase activity in the cortex was assessed in biological triplicates by a fluorometric assay. CBE-treated mice showed reduced GCase activity whereas mice that received a high rAAV-GBA1 dose showed a statistically significant increase in GCase activity compared to CBE treatment. CBE-treated mice also had accumulation of GluCer and GluSph, both of which were rescued by administering a high dose of rAAV-GBA1.

In addition to the established chemical CBE model, rAAV-GBA1 is also evaluated in the 4L/PS-NA genetic model, which is homozygous for the V394L GD mutation in Gba1 and is also partially deficient in saposins, which affect GCase localization and activity. These mice exhibit motor strength, coordination, and balance deficits, as evidenced by their performance in the beam walk, rotarod, and wire hang assays. Typically the lifespan of these mice is less than 22 weeks. In an initial study, 3 μl of maximal titer virus was delivered by ICV at P23, with a final dose of 2.4e10 vg (6.0e10 vg/g brain). With 6 mice per group, the treatment groups were:
WT+Excipient ICV
4L/PS-NA+Excipient ICV
4L/PS-NA+2.4e10 vg (6.0e10 vg/g brain) rAAV-GBA1 ICV

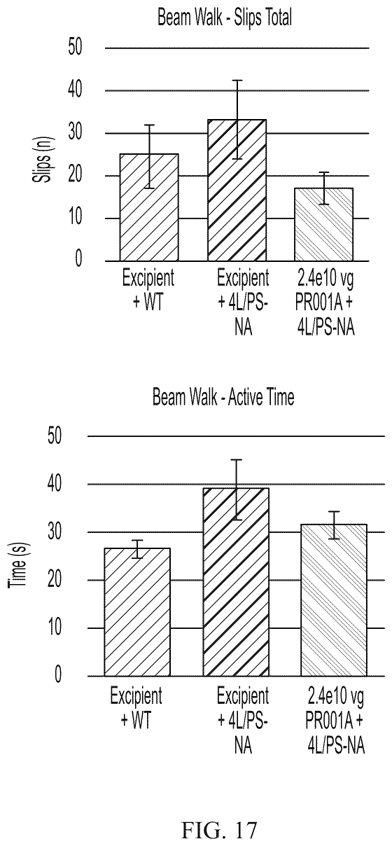

Motor performance by the beam walk test was assessed 4 weeks post-rAAV-GBA1 delivery. The group of mutant mice that received rAAV-GBA1 showed a trend towards fewer total slips and fewer slips per speed when compared to mutant mice treated with excipient, restoring motor function to near WT levels (FIG. 17). Since the motor phenotypes become more severe as these mice age, their performance on this and other behavioral tests is assessed at later time points. At the completion of the in-life study, lipid levels, GCase activity, and biodistribution are assessed in these mice.

Additional lower doses of rAAV-GBA1 are currently being tested using the CBE model, corresponding to 0.03×, 0.1×, and 1× the proposed phase 1 high clinical dose. Each group includes 10 mice (5M/5F) per group:

### Excipient ICV

Excipient ICV+25 mg/kg CBE IP
3.2e8 vg (2.13e9 vg/g brain) rAAV-GBA1 ICV+25 mg/kg CBE IP
1.0e9 vg (6.67e9 vg/g brain) rAAV-GBA1 ICV+25 mg/kg CBE IP
1.0e10 vg (6.67e10 vg/g brain) rAAV-GBA1 ICV+25 mg/kg CBE IP.

In addition to motor phenotypes, lipid levels and GCase activity are assessed in the cortex. Time course of treatments and analyses are also performed.

A larger dose ranging study was initiated to evaluate efficacy and safety data. 10 4L/PS-NA mice (5M/5F per group) were injected with 10 μl of rAAV-GBA1. Using an allometric brain weight calculation, the doses correlate to 0.15×, 1.5×, 4.4×, and 14.5× the proposed phase 1 high clinical dose. The injection groups consist of:
WT+Excipient ICV
4L/PS-NA+Excipient ICV
4L/PS-NA+4.3e9 vg (11.e10 vg/g brain) rAAV-GBA1 ICV
4L/PS-NA+4.3e10 vg (11.e11 vg/g/brain) rAAV-GBA1 ICV
4L/PS-NA+1.3e11 vg (3.2e11 vg/g brain) rAAV-GBA1 ICV
4L/PS-NA+4.3e11 vg (11.e12 vg/g brain) rAAV-GBA1 ICV.

### Example 9: In Vitro Analysis of rAAV Vectors

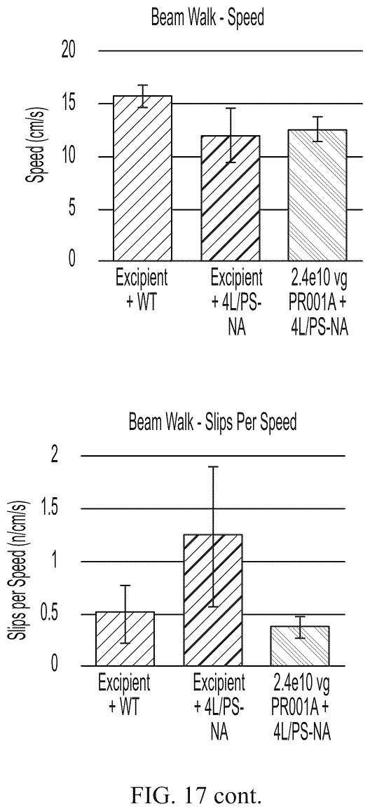

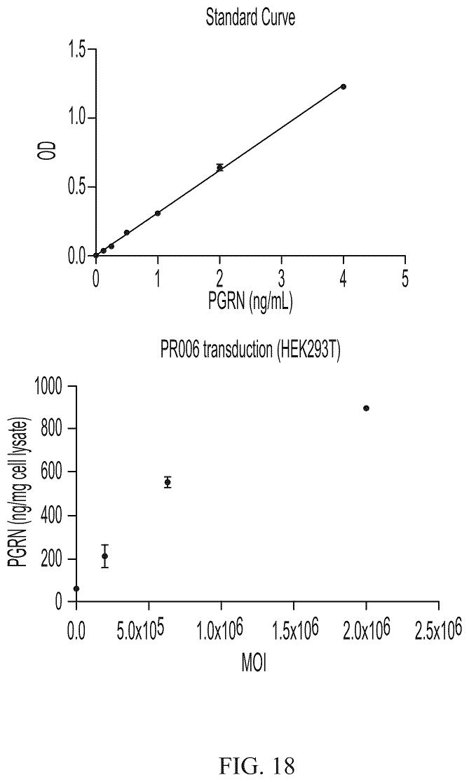

rAAV constructs were tested in vitro and in vivo. FIG. 18 shows representative data for in vitro expression of rAAV constructs encoding progranulin (PGRN) protein. The left panel shows a standard curve of progranulin (PGRN) ELISA assay. The bottom panel shows a dose-response of PGRN expression measured by ELISA assay in cell lysates of HEK293T cells transduced with rAAV. MOI=multiplicity of infection (vector genomes per cell).

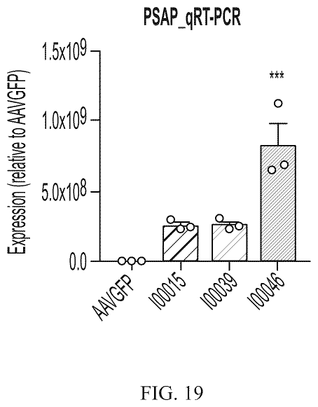

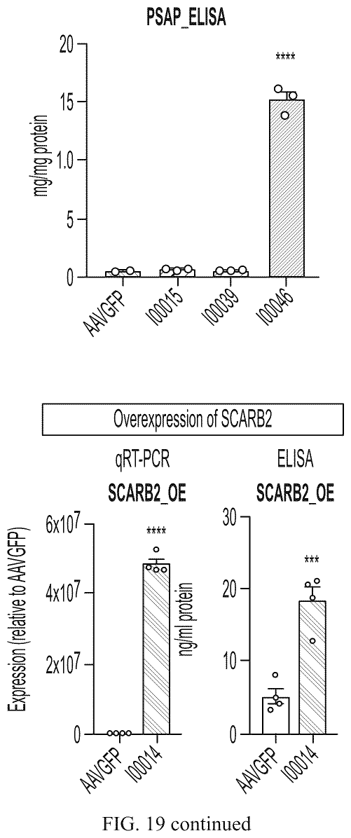

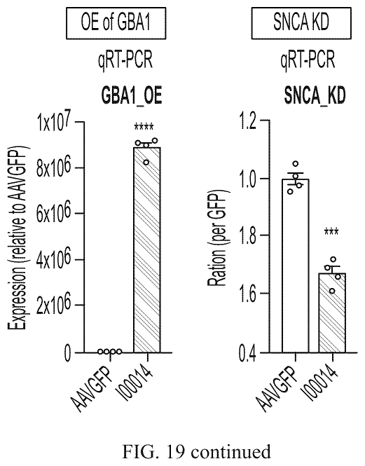

A pilot study was performed to assess in vitro activity of rAAV vectors encoding Prosaposin (PSAP) and SCARB2, alone or in combination with GBA1 and/or one or more inhibitory RNAs. One construct encoding PSAP and progranulin (PGRN) was also tested. Vectors tested include those shown in Table 3. “Opt” refers to a nucleic acid sequence codon optimized for expression in mammalian cells (e.g., human cells). FIG. 19 shows representative data indicating that transfection of HEK293 cells with each of the constructs resulted in overexpression of the corresponding gene product compared to mock transfected cells.

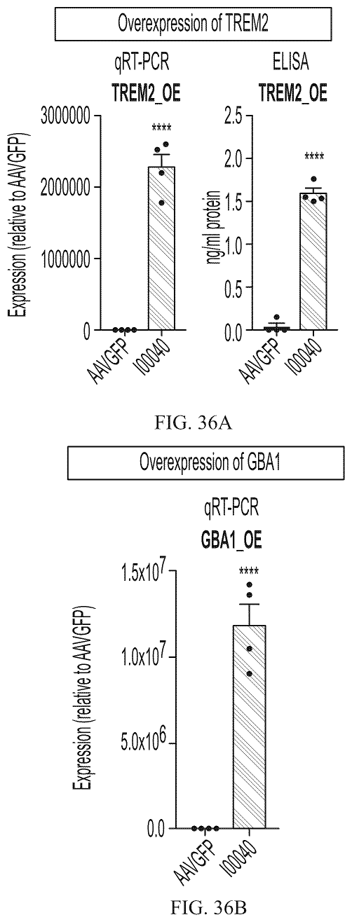

A pilot study was performed to assess in vitro activity of rAAV vectors encoding TREM2, alone or in combination with one or more inhibitory RNAs. Vectors tested include those shown in Table 3. “Opt” refers to a nucleic acid sequence codon optimized for expression in mammalian cells (e.g., human cells). FIGS. 36A-36B show representative data indicating that transfection of HEK293 cells with each of the constructs resulted in overexpression of the corresponding gene product compared to mock transfected cells.

TABLE 3

| ID | Promoter | Inhibitory RNA | Promoter | Transgene |
|---|---|---|---|---|
| I00015 | JL_intronic | SNCA | JetLong | Opt-PSAP_GBA1 |
| I00039 | — | — | JetLong | Opt-PSAP-GRN |
| I00046 | — | — | CBA | Opt-PSAP |
| I00014 | JetLong | SNCA | JetLong | Opt-SCARB2_GBA1 |
| I00040 | | | JL, CD68 | opt-GBA1, TREM2 |

### Example 10: Testing of SNCA and TMEM106B shRNA Constructs

### HEK293 Cells

Human embryonic kidney 293 cell line (HEK293) were used in this study (#85120602, Sigma-Aldrich). HEK293 cells were maintained in culture media (D-MEM [#11995065, Thermo Fisher Scientific] supplemented with 10% fetal bovine serum [FBS] [#10082147, Thermo Fisher Scientific]) containing 100 units/ml penicillin and 100 μg/ml streptomycin (#15140122, Thermo Fisher Scientific).

### Plasmid Transfection

Plasmid transfection was performed using Lipofectamine 2000 transfection reagent (#11668019, Thermo Fisher Scientific) according to the manufacture's instruction. Briefly, HEK293 cells (#12022001, Sigma-Aldrich) were plated at the density of 3×10⁵ cells/ml in culture media without antibiotics. On the following day, the plasmid and Lipofectamine 2000 reagent were combined in Opti-MEM solution (#31985062, Thermo Fisher Scientific). After 5 minutes, the mixtures were added into the HEK293 culture. After 72 hours, the cells were harvested for RNA or protein extraction, or subjected to the imaging analyses. For imaging analyses, the plates were pre-coated with 0.01% poly-L-Lysine solution (P8920, Sigma-Aldrich) before the plating of cells.

Gene Expression Analysis by Quantitative Real-Time PCR (qRT-PCR)

Relative gene expression levels were determined by quantitative real-time PCR (qRT-PCR) using Power SYBR Green Cells-to-CT Kit (#4402955, Thermo Fisher Scientific) according to the manufacturer's instruction. The candidate plasmids were transiently transfected into HEK293 cells plated on 48-well plates (7.5×10⁴ cells/well) using Lipofectamine 2000 transfection reagent (0.5 μg plasmid and 1.5 μl reagent in 50 μl Opti-MEM solution). After 72 hours, RNA was extracted from the cells and used for reverse transcription to synthesize cDNA according to the manufacturer's instruction. For quantitative PCR analysis, 2-5 μl of cDNA products were amplified in duplicates using gene specific primer pairs (250 nM final concentration) with Power SYBR Green PCR Master Mix (#4367659, Thermo Fisher Scientific). The primer sequences for SNCA, TMEM106B, and GAPDH genes were: 5′-AAG AGG GTG TTC TCT ATG TAG GC-3′ (SEQ ID NO: 71), 5′-GCT CCT CCA ACA TTT GTC ACT T-3′ (SEQ ID NO: 72) for SNCA, 5′-ACA CAG TAC CTA CCG TTA TAG CA-3′ (SEQ ID NO: 73), 5′-TGT TGT CAC AGT AAC TTG CAT CA-3′ (SEQ ID NO: 74) for TMEM106B, and 5′-CTG GGC TAC ACT GAG CAC C-3′ (SEQ ID NO: 75), 5′-AAG TGG TCG TTG AGG GCA ATG-3′ (SEQ ID NO: 76) for GAPDH. Quantitative PCR was performed in a QuantStudio 3 Real-Time PCR system (Thermo Fisher Scientific). Expression levels were normalized by the housekeeping gene GAPDH and calculated using the comparative CT method.

### Fluorescence Imaging Analysis

EGFP reporter plasmids, which contain 3′-UTR of human SNCA gene at downstream of EGFP coding region, were used for the validation of SNCA and TMEM106B knockdown plasmids. EGFP reporter plasmids and candidate knockdown plasmids were simultaneously transfected into HEK293 cells plated on poly-L-Lysine coated 96-well plates (3.0×10⁴ cells/well) using Lipofectamine 2000 transfection reagent (0.04 μg reporter plasmid, 0.06 μg knockdown plasmid and 0.3 μl reagent in 10 μl Opti-MEM solution). After 72 hours, the fluorescent intensities of EGFP signal were measured at excitation 488 nm/emission 512 nm using Varioskan LUX multimode reader (Thermo Fisher Scientific). Cells were fixed with 4% PFA at RT for 10 minutes, and incubated with D-PBS containing 40 μg/ml 7-aminoactinomycin D (7-AAD) for 30 min at RT. After washing with D-PBS, the fluorescent intensities of 7-AAD signal were measured at excitation 546 nm/emission 647 nm using Varioskan reader to quantify cell number. Normalized EGFP signal per 7-AAD signal levels were compared with the control knockdown samples.

### Enzyme-Linked Immunosorbent Assay (ELISA)

α-Synuclein reporter plasmids, which contain 3′-UTR of human SNCA gene or TMEM106B gene downstream of SNCA coding region, were used for the validation of knockdown plasmids at the protein level. Levels of α-synuclein protein were determined by ELISA (#KHB0061, Thermo Fisher Scientific) using the lysates extracted from HEK293 cells. The candidate plasmids were transiently transfected into HEK293 cells plated on 48-well plates (7.5×10⁴ cells/well) using Lipofectamine 2000 transfection reagent (0.1 μg reporter plasmid, 0.15 μg knockdown plasmid and 0.75 μl reagent in 25 μl Opti-MEM solution). After 72 hours, cells were lysed in radioimmunoprecipitation assay (RIPA) buffer (#89900, Thermo Fisher Scientific) supplemented with protease inhibitor cocktail (#P8340, Sigma-Aldrich), and sonicated for a few seconds. After incubation on ice for 30 min, the lysates were centrifuged at 20,000×g at 4° C. for 15 min, and the supernatant was collected. Protein levels were quantified. Plates were read in a Varioskan plate reader at 450 nm, and concentrations were calculated using SoftMax Pro 5 software. Measured protein concentrations were normalized to total protein concentration determined with a bicinchoninic acid assay (#23225, Thermo Fisher Scientific).

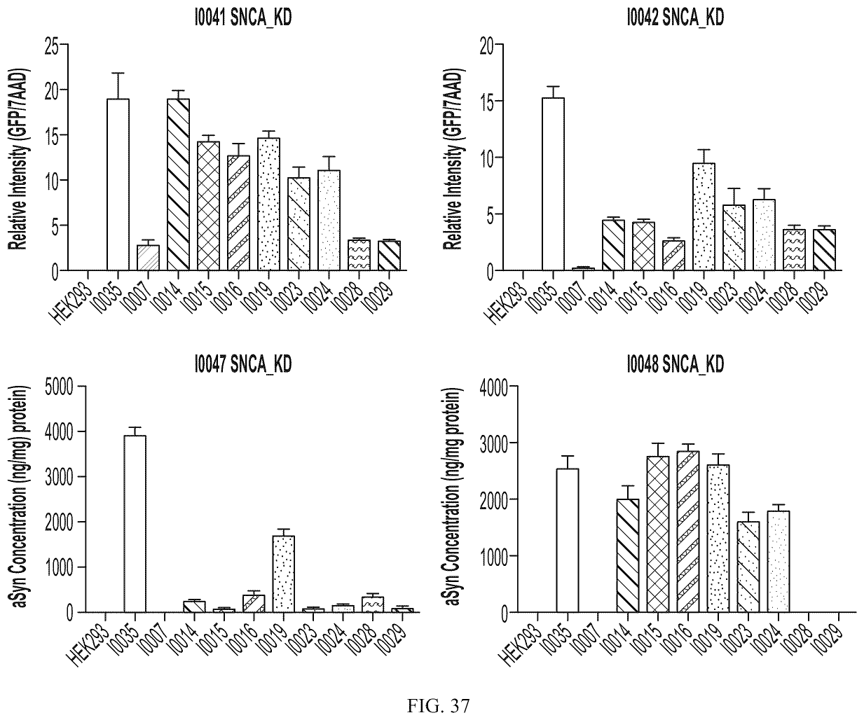

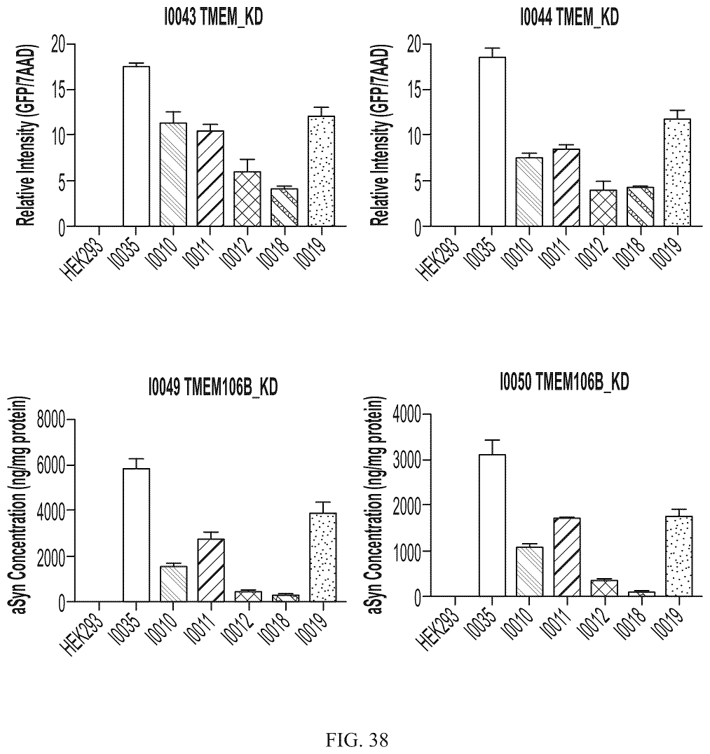

FIG. 37 and Table 4 show representative data indicating successful silencing of SNCA in vitro by GFP reporter assay (top) and α-Syn assay (bottom). FIG. 38 and Table 5 show representative data indicating successful silencing of TMEM106B in vitro by GFP reporter assay (top) and α-Syn assay (bottom).

TABLE 4

| ID | Promoter | Knockdown | Promoter | Overexpress |
|---|---|---|---|---|
| I00007 | CMV_intronic | SNCA_mi | CMV | opt-GBA1 |
| I00008 | H1 | SNCA_sh | CMV | opt-GBA1 |
| I00009 | H1 | SNCA_Pubsh4 | CMV | opt-GBA1 |
| I00014 | JL_intronic | SNCA_mi | JetLong | opt-SCARB2_GBA |
| I00015 | JL_intronic | SNCA_mi | JetLong | opt-PSAP_GBA |
| I00016 | JL_intronic | SNCA_mi | JetLong | opt-CTSB_GBA |
| I00019 | JL_intronic | SNCA_TMEM_mi | JetLong | opt-VPS35 |
| I00023 | JL_intronic | SNCA_mi | JetLong | opt-GBA1_IL34 |
| I00024 | JL_intronic | SNCA_mi | JetLong | opt-GBA2 |
| I00028 | intronic | SNCA_Broadsh | CMV | opt-GBA1 |
| I00029 | intronic | SNCA_Pubsh4 | CMV | opt-GBA1 |

TABLE 5

| ID | Promoter | Knockdown | Promoter | Overexpress |
|---|---|---|---|---|
| I00010 | H1 | TMEM_Pubsh | CMV | opt-GRN |
| I00011 | JL_intronic | TMEM_mi | JetLong | opt-GBA1_GRN |
| I00012 | H1 | TMEM_sh | CMV | opt-GRN |
| I00019 | JL_intronic | SNCA_TMEM_mi | JetLong | opt-VPS35 |

### Example 11: ITR “D” Sequence Placement and Cell Transduction

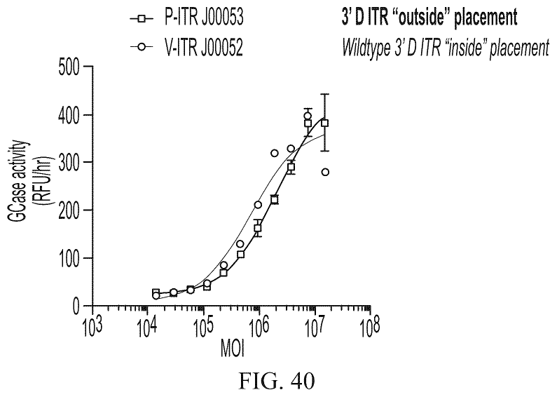

The effect of placement of ITR “D” sequence on cell transduction of rAAV vectors was investigated. HEK293 cells were transduced with Gcase-encoding rAAVs having 1) wild-type ITRs (e.g., “D” sequences proximal to the transgene insert and distal to the terminus of the ITR) or 2) ITRs with the “D” sequence located on the “outside” of the vector (e.g., “D” sequence located proximal to the terminus of the ITR and distal to the transgene insert), as shown in FIG. 20. Surprisingly, data indicate that rAAVs having the “D” sequence located in the “outside” position retain the ability to be packaged and transduce cells efficiently (FIG. 40).

### Example 12: In Vitro Testing of Progranulin rAAVs

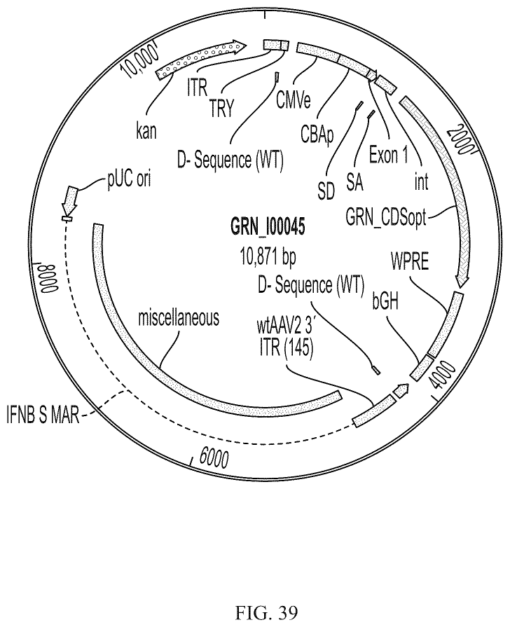

FIG. 39 is a schematic depicting one embodiment of a vector comprising an expression construct encoding PGRN. Progranulin is overexpressed in the CNS of rodents deficient in GRN, either heterozygous or homozygous for GRN deletion, by injection of an rAAV vector encoding PGRN (e.g., codon-optimized PGRN), either by intraparenchymal or intrathecal injection such as into the cisterna magna.

Mice are injected at 2 months or 6 months of age, and aged to 6 months or 12 months and analyzed for one or more of the following: expression level of GRN at the RNA and protein levels, behavioral assays (e.g., improved movement), survival assays (e.g., improved survival), microglia and inflammatory markers, gliosis, neuronal loss, Lipofuscinosis, and/or Lysosomal marker accumulation rescue, such as LAMP1. Assays on PGRN-deficient mice are described, for example by Arrant et al. (2017) Brain 140: 1477-1465; Arrant et al. (2018) J. Neuroscience 38(9):2341-2358; and Amado et al. (2018) doi:https://doi.org/10.1101/30869; the entire contents of which are incorporated herein by reference.

### Example 13: In Vitro and In Vivo Testing of Progranulin rAAV

In vitro and in vivo assays were performed to analyze the effects of an rAAV construct (PR006 (also referred to as PR006A); see FIG. 64) encoding progranulin (PGRN) protein. PR006 comprises a capsid having an AAV9 serotype.

### In Vitro Nonclinical Studies

Progranulin Expression Derived from PR006A in HEK293T Cells

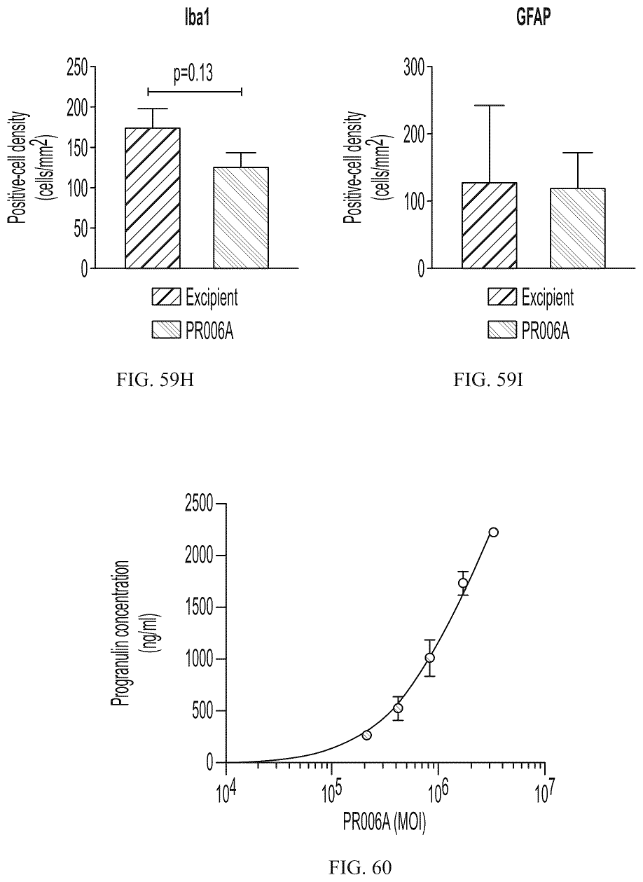

The ability of PR006A to induce progranulin protein production in a cellular context was investigated. HEK293T cells were transduced with PR006A over a range of multiplicities of infection (MOI) ranging from 2.1×10⁵ to 3.3×10⁶ vector genomes (vg)/cell. PR006A transduction resulted in a robust, dose-dependent increase in progranulin protein expression and secretion into the cell media (FIG. 60). Substantially lower progranulin protein levels, reflecting the expression derived from the endogenous human GRN gene, were detected in a negative control group treated with excipient (the intended clinical vehicle) alone.

Efficacy in FTD-GRN iPSC-Derived Neurons

An assay was performed to analyze the efficacy of the rAAV construct in vitro in human FTD-GRN (Frontotemporal dementia with GRN mutation) neuronal cultures. Cell lines were obtained from the National Institute of Neurological Disorders and Stroke (NINDS) Human Cell and Data Repository (NHCDR): Materials ND50015 (FTD-GRN, M1L), ND50060 (FTD-GRN, R493X) and ND38555 (control, wild-type) (see Table 6).

TABLE 6
Summary of iPSC cell line characteristics

| | NINDS | Clinical | | | | Source Cell/ |
|---|---|---|---|---|---|---|
| | Cell Line | Diagnosis | GRN | | | Reprogramming |
| Cell Line | ID # | of FTD? | mutation | Age | Gender | Method |
| FTD-GRN #1 | ND50015 | Yes | MIL | 54 | F | Fibroblast/Episomal |
| | | | | | | plasmids |
| FTD-GRN #2 | ND50060 | At risk | R493X | 60 | M | Fibroblast/Episomal |
| | | (sibling | | | | plasmids |
| | | affected | | | | |
| | | at 62 yrs) | | | | |
| Control | ND38555 | No | N/A | 48 | F | Fibroblast/Retroviral |
| | | | | | | plasmids |

To establish a cellular model that is pathologically relevant to FTD-GRN, iPSCs from each line were differentiated into neuronal cells using a two-step protocol. In the first step, iPSCs were differentiated into proliferating neuronal stem cell (NSC) lines, which lacked expression of pluripotency markers (i.e., Oct4 and SSEA1) and gained expression of neuronal stem cell markers (i.e., SOX2, Nestin, SOX1, and PAX6), as detected by immunofluorescence labeling.

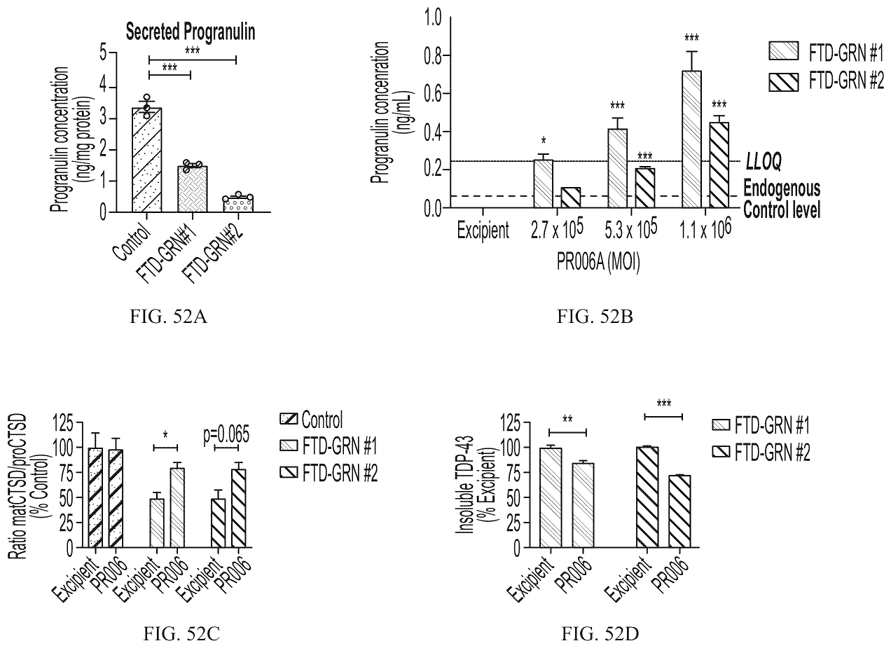

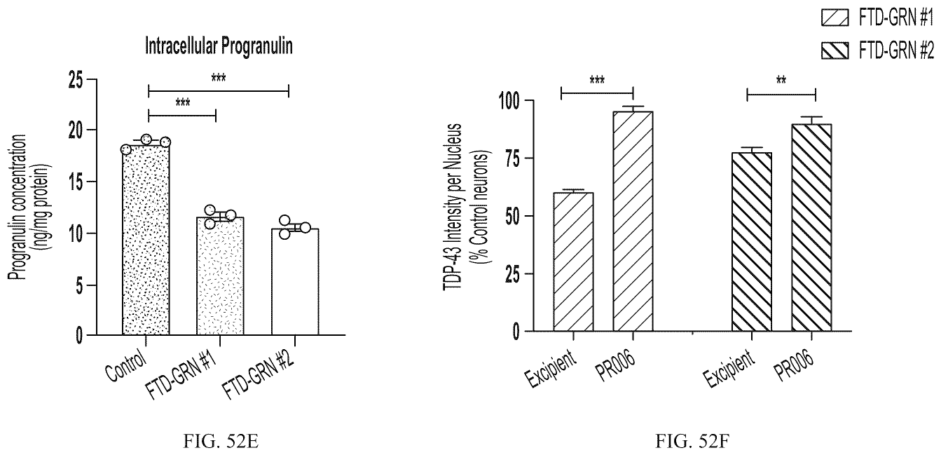

Control and FTD-GRN NSC lines were seeded at an equal density, and 48 hours later, progranulin expression was measured by an enzyme-linked immunosorbent assay (ELISA) in cell lysates (intracellular progranulin) (FIG. 52E) and cell media (secreted progranulin) (FIG. 52A). Progranulin expression was normalized to total protein concentration to account for differences in cell number (n=3; mean±SEM). The NSC lines with heterozygous GRN mutations had significantly lower intracellular and secreted progranulin levels compared to Control NSCs, with FTD-GRN NSCs expressing ˜25-50% of endogenous progranulin levels. This suggested that this FTD-GRN cell model recapitulates the clinical progranulin deficiency observed in FTD-GRN patients, who express one third to one half of normal progranulin levels in the plasma (Finch et al., Brain 132, 583-591 (2009); Ghidoni et al., Neurology 71, 1235-1239, (2008); Sleegers et al., Ann Neurol 65, 603-609 (2009)).

NSCs from all cell lines were differentiated into neuronal cultures. After establishing that the iPSC-derived NSCs exhibit reduced progranulin expression, the lines were differentiated into neurons to generate a clinically representative cell type for nonclinical efficacy studies of PR006A. NSCs were seeded into neuronal differentiation media, terminally differentiated into postmitotic neurons for a period of 7 days, and then assessed for expression of neuronal markers (i.e., MAP2, NeuN, Tau, Tuj 1, NF—H) by immunofluorescence (FIG. 52G). Both Control and FTD-GRN iPSC-derived NSC lines efficiently differentiated into neurons using this protocol.

FTD-GRN iPSC-derived neuronal cultures were used to evaluate the efficacy of PR006A in vitro. FTD-GRN neurons were treated with excipient or PR006A at MOIs of 2.7×10⁵, 5.3×10⁵, or 1.1×10⁶ vg/cell. PR006 transduction resulted in a robust, dose-dependent expression of secreted progranulin, as measured by ELISA, in all cell lines (FIG. 52B). Excipient-treated Control and FTD-GRN neurons were assessed for endogenous progranulin levels. Control neurons expressed endogenous secreted progranulin, while no secreted progranulin was detected in FTD-GRN neurons (FIG. 52B). Linear regression analysis confirmed a significant correlation between PR006A dose and progranulin levels across both FTD-GRN cell lines (p=3.5×10¹³). These results demonstrate that treatment with PR006A results in elevated secretion of progranulin in the FTD-GRN neuronal model.

Progranulin is known to stimulate maturation of the lysosomal protease cathepsin D (CTSD), whose loss of function has also been implicated in lysosomal storage disorders and neurodegeneration. CTSD is expressed as an inactive full-length pro-protein (proCTSD) that undergoes proteolytic processing into an enzymatically active mature protease (matCTSD). Progranulin has been reported to act as a molecular chaperone that binds to proCTSD to enhance its maturation into the matCTSD protease. In FTD-GRN neuronal cultures, PR006 transduction rescued the defective maturation of cathepsin D (FIG. 52C). Control, FTD-GRN #1, and FTD-GRN #2 neurons were transduced with PR006A or excipient. An MOI of 5.3×10⁵ PR006A was used for efficacy experiments since it restored progranulin levels to at least 2-fold those of Control cells (FIG. 52B). To evaluate efficacy, proCTSD and matCTSD expression levels were measured in cell lysates using the automated a Simple Western™ (Jess) platform (FIG. 52C). Excipient-treated FTD-GRN neurons had a lower ratio of matCTSD to proCTSD as compared to excipient-treated Control neurons; PR006A treatment significantly increased the ratio in both FTD-GRN neuronal lines (FIG. 52C). In Control neurons, the ratio of matCTSD to proCTSD was not significantly altered by PR006A treatment. These findings demonstrate that PR006A restores a lysosomal function-related phenotype in FTD-GRN neurons.

In normal neurons, TDP-43 (transactive response DNA binding protein 43 kDa) protein is localized in the nucleus. In post-mortem brains of FTD-GRN patients, aggregation of TDP-43 in the cytoplasm of neurons is observed, and nuclear accumulation of TDP-43 is reduced. FTD neurons have decreased nuclear TDP-43, leading to aggregation and downstream toxicity in neurons. Since Grn KO mice do not fully recapitulate this TDP-43 pathology, induced pluripotent stem cell (iPSC)-derived neurons are a valuable FTD-GRN model to study TDP-43 biology. Decreased accumulation of TDP-43 in the nucleus, and increased accumulation of insoluble TDP-43, have been reported in iPSC-derived neurons from patients with FTD-GRN, relative to control neurons that do not carry a GRN mutation, as described by Valdez et al. (Human Molecular Genetics 26, 4861-4872 (2017)). PR006A transduction of neuronal cultures from both FTD-GRN mutation carrier lines reversed TDP-43 abnormalities, resulting in decreased insoluble TDP-43 (measured using the Simple Western™ (Jess) platform (FIG. 52D)) and increased nuclear localization of TDP-43 (measured using immunofluorescence (FIG. 52F)).

To summarize, PR006 transduction restored defective maturation in the lysosomal enzyme, cathepsin D, and improved abnormal TDP-43 pathology in FTD-GRN neurons.

### In Vivo Nonclinical Studies

### Efficacy and Biodistribution in Aged Grn Knockout Mice

PR006A efficacy in vivo and the maximal dose PR006A were evaluated in the Grn knockout (KO) mouse model. In the Grn KO mouse model used in these studies (B6(Cg)-Grn^{tm1.1Aidi}/J (Jackson Laboratory, Bar Harbor, ME), exons 1-4 are deleted from the target progranulin (Grn) gene (Yin et al., J Exp Med 207, 117-128 (2010)). These animals have a complete loss of progranulin, display age-dependent phenotypes including lysosomal alterations, neuronal lipofuscin accumulation, ubiquitin accumulation, microgliosis, and neuroinflammation, and are therefore widely used to model FTD-GRN. All attempts were made to eliminate bias from the study; mice were assigned to treatment groups that were balanced for gender and body weight, and a blinded assessment of experimental endpoints was conducted by qualified personnel.

In the initial studies, PR006A was delivered to aged Grn KO mice at a dose of 9.7×10¹⁰ vg (2.4×10¹¹ vg/g brain), which was the highest achievable dose at the time of the study due to injection volume constraints and the physical titer of the virus lot used for the study. Aged mice were used since many of the FTD-GRN-related phenotypes, including CNS inflammation and microgliosis, develop in an age dependent manner, with the most pronounced manifestation of phenotypes occurring between 12-24 months of age.

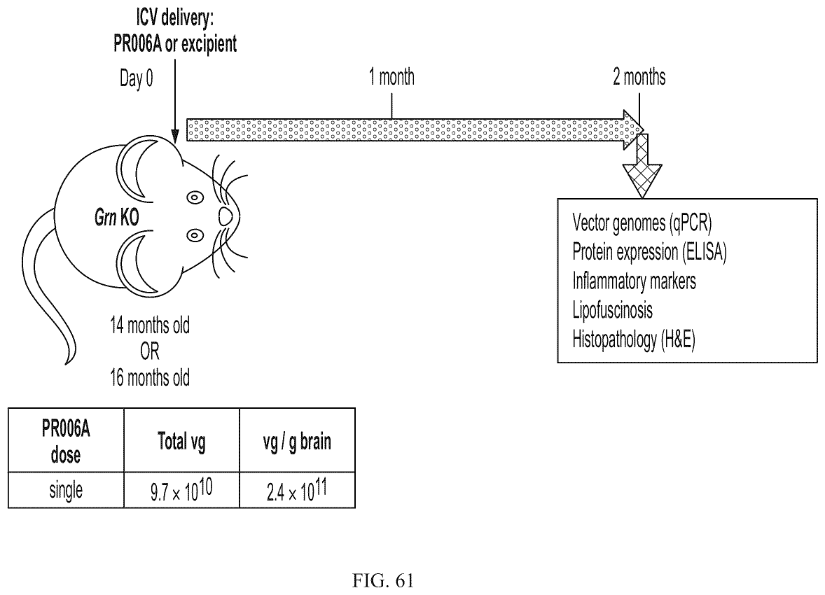

In the studies with aged Grn KO mice, PR006A was administered by single intracerebroventricular (ICV) injection. 10 μl excipient (the intended clinical vehicle; 20 mM Tris pH 8.0, 200 mM NaCl, and 1 mM MgCl₂+0.001% Pluronic F68) or 9.7×10¹⁰ vg PR006A (2.4×10¹¹ vg/g brain [based on an adult mouse brain weight of 400 mg]) was delivered by ICV injection into two cohorts of aged Grn KO mice: (1) 16-months-old at time of injection (n=4/group; PRV-2018-027; FIG. 61) and (2) 14-months-old at time of injection (planned n=3/group; PRV-2019-002; FIG. 61). The animals were sacrificed two months post-injection.

In study PRV-2018-027, a single dose of PR006A was delivered to 16-month-old mice with the following treatment groups:

| Model | ICV | ICV dose | N |
|---|---|---|---|
| Grn KO | Excipient | N/A | 4 (2M/2F) |
| Grn KO | PR006A | 9.7 × 10¹⁰ vg | 5 (3M/2F) |
| | | (2.4 × 10¹¹ vg/g brain) | |

Due to unforeseen study deviations (errors in genotyping and premature loss of animals), study PRV-2019-002 (14-month-old cohort) enrolled only 1 mouse in the excipient-treated group instead of the planned n=3. The low sample number made statistical analysis impossible, and therefore this study is excluded from further discussion here. However, the findings from the study were comparable to those from study PRV-2018-027.

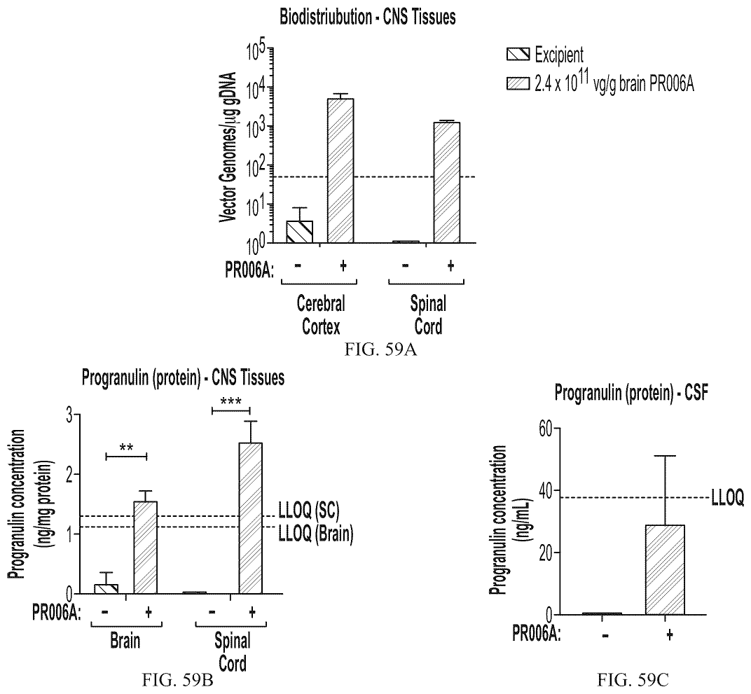

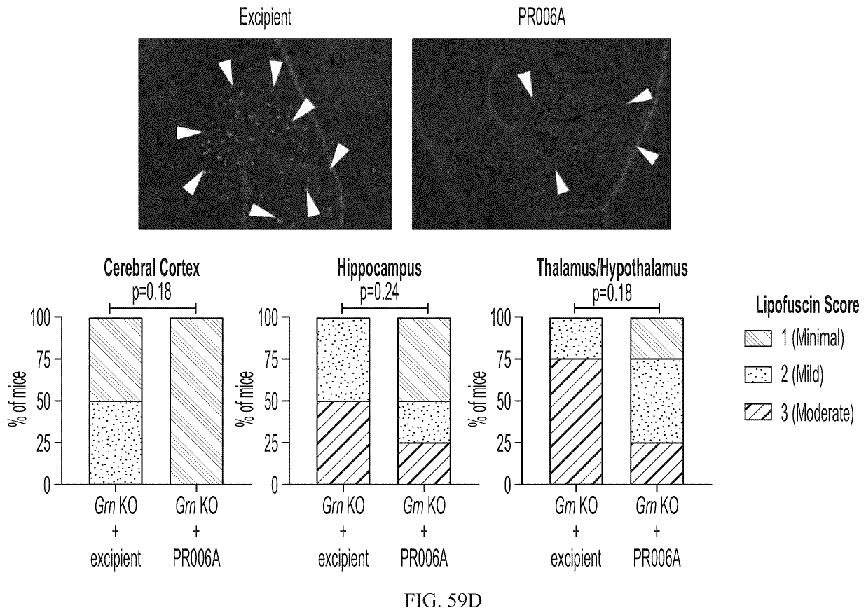

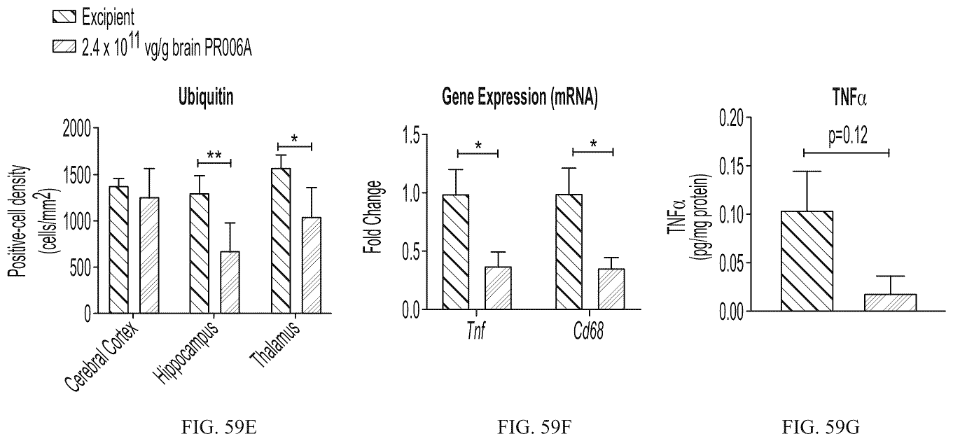

Biodistribution and Progranulin Expression: Biodistribution was determined by measuring vector genome presence using a qPCR assay that meets the current U.S. Food and Drug Administration Center for Biologics Evaluation and Research (CBER)/Office of Tissues and Advanced Therapies (OTAT) standards for PCR sensitivity (with >50 vector genomes per 1 μg genomic DNA defined as positive). All mice that received PR006A were positive for vector genomes in the cerebral cortex and spinal cord, indicating that ICV administration successfully results in PR006A transduction in the brain and CNS (FIG. 59A). ICV PR006A resulted in significant levels of human progranulin protein in the CNS (brain, spinal cord) of the Grn KO mice, whereas, as expected, human progranulin was not detectable in the mice that received excipient (FIG. 59B). Since progranulin is primarily a secreted protein, expression in the CSF can be considered a surrogate of protein production within the brain and represents a potential translational endpoint for FTD-GRN patients who have decreased CSF progranulin levels. We were able to detect human progranulin in the CSF of PR006A-treated mice, but because of the small sample volume and the technical limitations of obtaining sufficient volume of CSF in mice, the measurements of CSF progranulin level were below the lower limit of quantitation (LLOQ) of the assay (FIG. 59C).

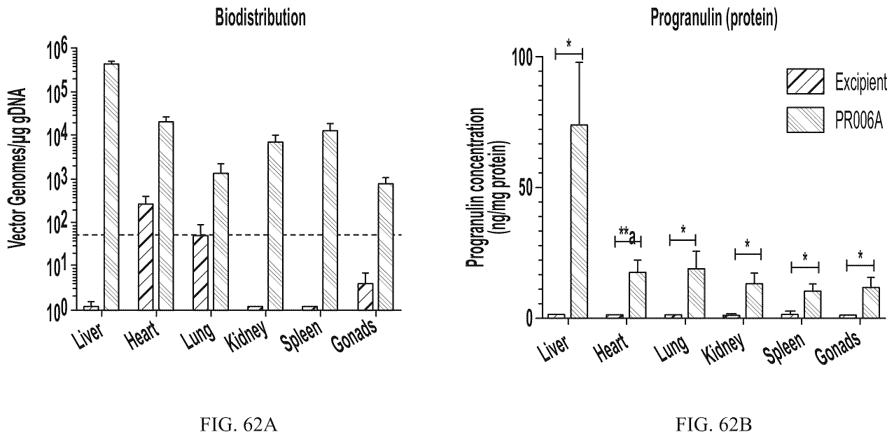

ICV administration also resulted in broad vector genome presence and progranulin protein levels in peripheral tissues, including liver, heart, lung, kidney, spleen, and gonads (FIG. 62A-FIG. 62B). In addition, significant levels of human progranulin were detectable in plasma of the PR006A-treated Grn KO mice. As expected, human progranulin was not detected in the excipient treated Grn KO mice.

Lipofuscin Accumulation: Accumulation of neuronal lipofuscin, an electron-dense, autofluorescent material that accumulates progressively over time in lysosomes of postmitotic cells and is an indicator of lysosomal dysfunction, is a hallmark age-dependent phenotype of Grn KO mice. Lipofuscin accumulation was assessed using two independent methods in adjacent brain sections: (1) in a more clinical approach, lipofuscin accumulation in the brain was scored by a blinded pathologist on a scale of 0 (no lipofuscin observed) to 4 (widespread lipofuscin accumulation) and (2) in a more quantitative approach, lipofuscin autofluorescence was detected by immunohistochemistry (IHC) and automatically quantified. Grn KO mice exhibited substantial lipofuscinosis throughout the brain, and ICV PR006A treatment reduced the lipofuscin score severity in the cerebral cortex, hippocampus, and thalamus (FIG. 59D). Quantitation of lipofuscin accumulation from IHC images also detected decreased lipofuscinosis with PR006A treatment in all three brain regions. Since ubiquitin-positive inclusions are a defining pathological feature of FTD-GRN patients that also accumulate in the Grn KO mouse model in an age-dependent manner, IHC was performed and quantified in the brain regions of interest (cerebral cortex, hippocampus, thalamus) to assess ubiquitin accumulation. PR006A treatment significantly reduced ubiquitin accumulation in Grn KO mice (FIG. 59E). These findings suggest that PR006A improves lysosomal dysfunction in the Grn KO mouse model of FTD-GRN.

Neuroinflammation: Chronic CNS inflammation is a pathological feature in the brain of patients with FTD-GRN that is recapitulated in Grn KO mice in an age-dependent manner. Progranulin has anti-inflammatory effects in mouse models of FTD-GRN, and loss of progranulin leads to upregulation of proinflammatory cytokines, including TNFα. In this study, treatment with PR006A suppressed inflammatory marker levels in aged Grn KO mice. ICV PR006A decreased gene expression of the proinflammatory cytokine Tnf (TNFα) and Cd68 (CD68), a marker of microglia, in the cerebral cortex (FIG. 59F). TNFα protein levels were also decreased in cerebral cortex samples from PR006A-treated Grn KO mice using the Mesoscale Discovery mouse pro-inflammatory cytokine assay (FIG. 59G). To further evaluate neuroinflammation, immunohistochemistry (IHC) was performed for Iba1, a marker of microgliosis, and GFAP, a marker of astrocytosis, and quantified in the brain regions of interest (cerebral cortex, hippocampus, thalamus). PR006A treatment resulted in a trend towards decreased microgliosis (Iba1) but did not affect astrocytosis (GFAP) in Grn KO mice (FIG. 59H; FIG. 59I). Taken together, these results indicate that PR006A treatment reduces neuroinflammation in the aged Grn KO mouse model of FTD-GRN.

Histopathology: thorough histopathological analysis by a blinded board-certified pathologist of hematoxylin and eosin (H&E) staining of the brain, thoracic spinal cord, liver, heart, spleen, lung, and kidney of all mice from these studies revealed no adverse events related to PR006A treatment. Administration of PR006A to Grn KO mice resulted in a decreased incidence and/or severity of findings that are characteristic of the model, including a reduction in frequency and/or severity scores of neuronal necrosis in the medulla and pons. Additionally, there was a reduction in both the incidence and severity of axonal degeneration in the thoracic spinal cord with PR006A treatment. These findings are discussed in detail in the Toxicology section below.

Conclusion: ICV PR006A at a dose of 9.7×10¹⁰ vg (2.4×10¹¹ vg/g brain) resulted in broad vector genome presence throughout the brain and peripheral tissues in aged Grn KO mice. PR006A treatment increased global progranulin expression. In addition, PR006A reduced accumulation of lipofuscin and ubiquitin in the brain, pathologies known to occur in both the Grn KO mouse model and patients with FTD-GRN. PR006A also reduced the expression of proinflammatory cytokines and immune cell activation in the cerebral cortex, phenotypes that are indicative of chronic CNS inflammation.

### Dose-Ranging Efficacy in Adult Grn Knockout Mice

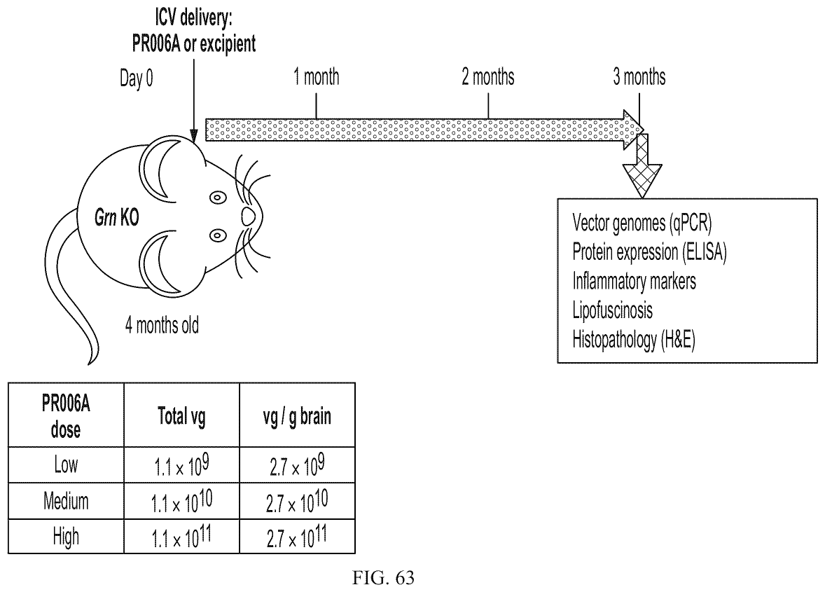

To further assess efficacious doses of PR006A, a larger, dose-ranging study in adult Grn KO mice was performed. In PRV-2019-004, 10 μl excipient (the intended clinical vehicle; 20 mM Tris pH 8.0, 200 mM NaCl, and 1 mM MgCl₂+0.001% Pluronic F68) or PR006A was delivered via ICV to 4-month-old animals. These adult mice were used instead of the aged Grn KO mice because the latter were not available in sufficient numbers for conducting a dose-ranging study. While the adult Grn KO mice have a milder phenotype than aged mice, they still exhibit lysosomal defects and neuroinflammatory changes and therefore are suitable for evaluating the efficacious dose range of PR006A. In order to assess PR006A efficacy over a broad range of viral doses, PR006A was administered at 1.1×10¹¹ vg (2.7×10¹¹ vg/g brain), the highest achievable dose at the time of the study due to injection volume constraints and the physical titer of the virus lot used for the study, a middle dose of 1.1×10¹⁰ vg (2.7×10¹⁰ vg/g brain), or a low dose of 1.1×10⁹ vg (2.7×10⁹ vg/g brain), with a full log difference spanning each dose. The details of the experimental design are given in FIG. 63.

Three doses of PR006A were assessed, with 10 mice (4M/6F) per group:

| Model | ICV | ICV dose | N |
|---|---|---|---|
| Grn KO | Excipient | N/A | 10 (4 M/6 F) |
| Grn KO | PR006A | 1.1 × 10⁹ vg | 10 (4 M/6 F) |
| | | (2.7 × 10⁹ vg/g brain) | |
| Grn KO | PR006A | 1.1 × 10¹⁰ vg | 10 (4 M/6 F) |
| | | (2.7 × 10¹⁰ vg/g brain) | |
| Grn KO | PR006A | 1.1 × 10¹¹ vg | 10 (4 M/6 F) |
| | | (2.7 × 10¹¹ vg/g brain) | |

Age-matched mice of the same background strain as the Grn KO mice with wildtype (WT) Grn alleles (7-month old C57BL/6J) served as controls for select efficacy endpoints in this study.

| Model | ICV | ICV dose | N |
|---|---|---|---|
| WT (C57BL/6J) | N/A | N/A | 10 (5 M/5 F) |

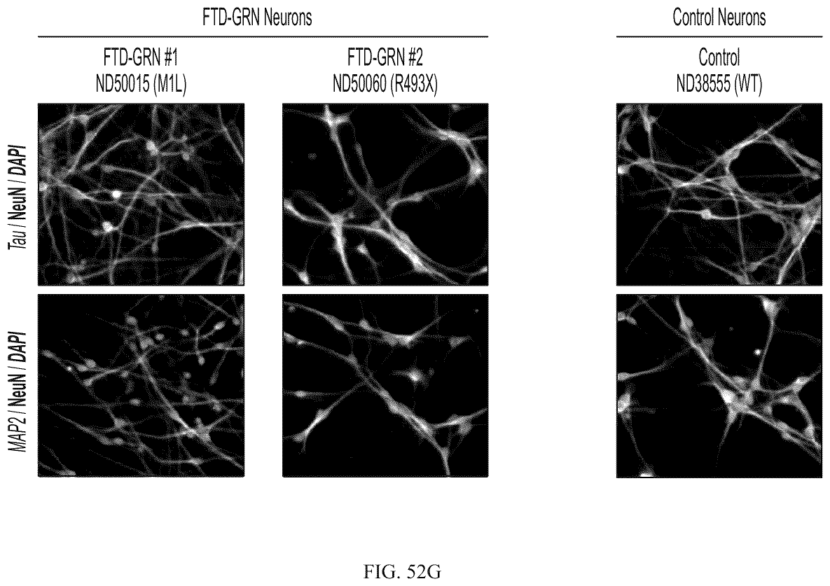

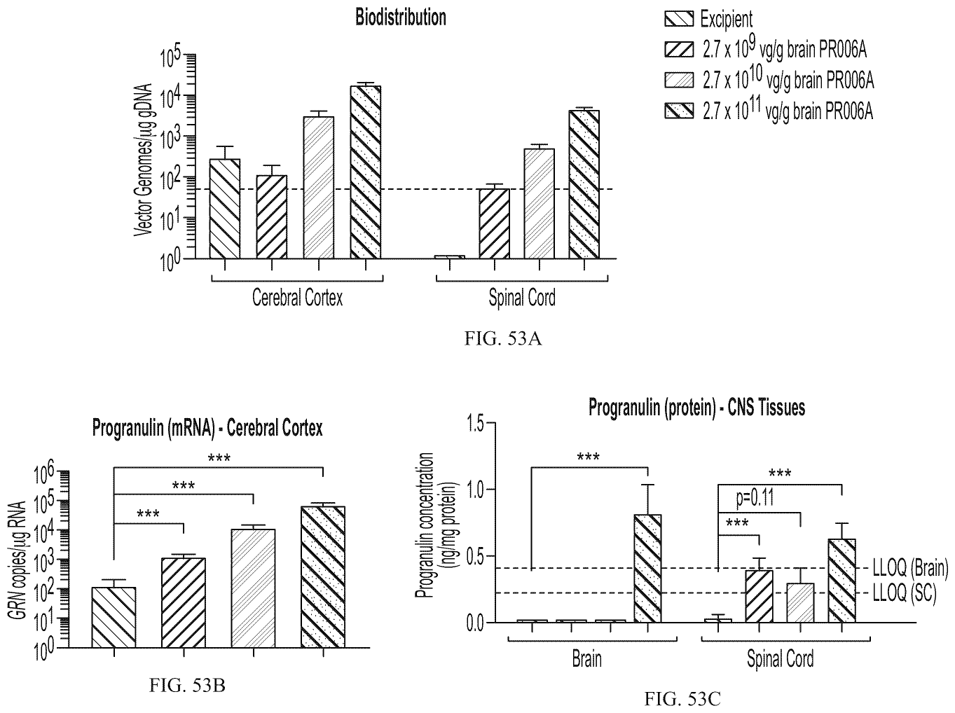

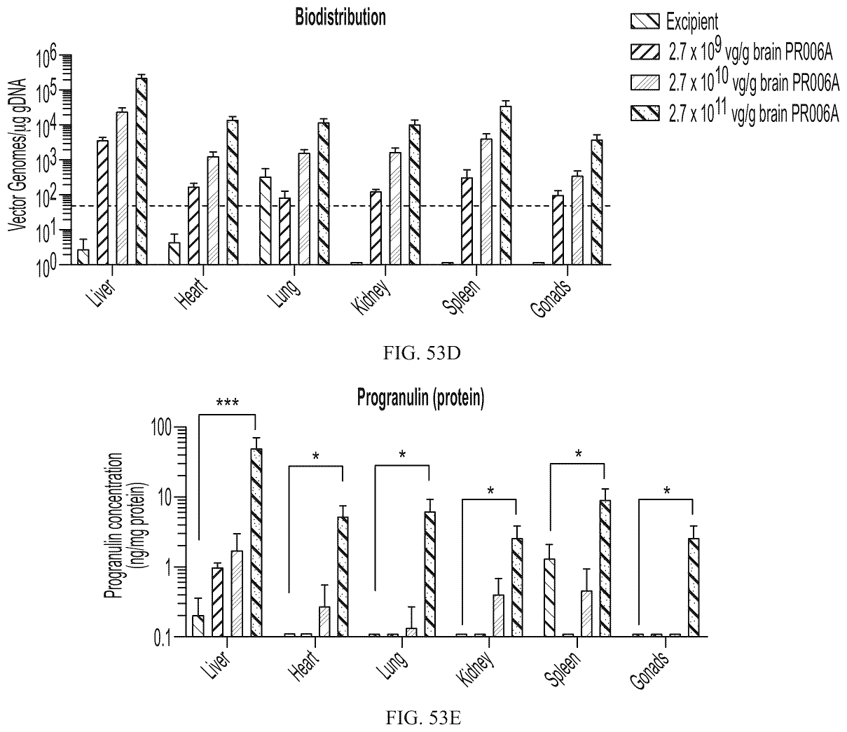

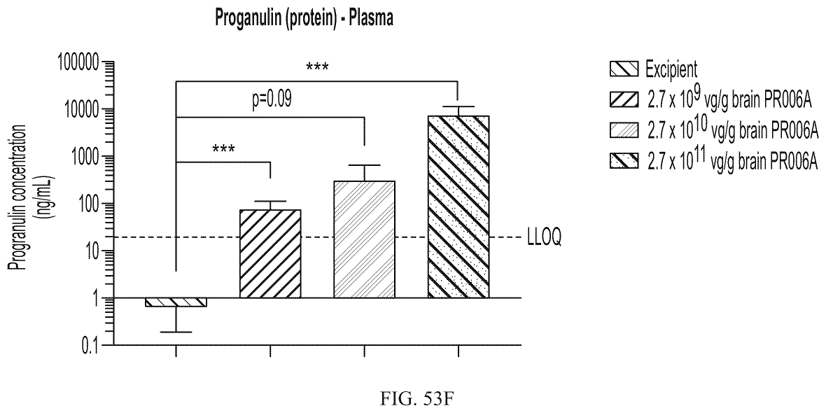

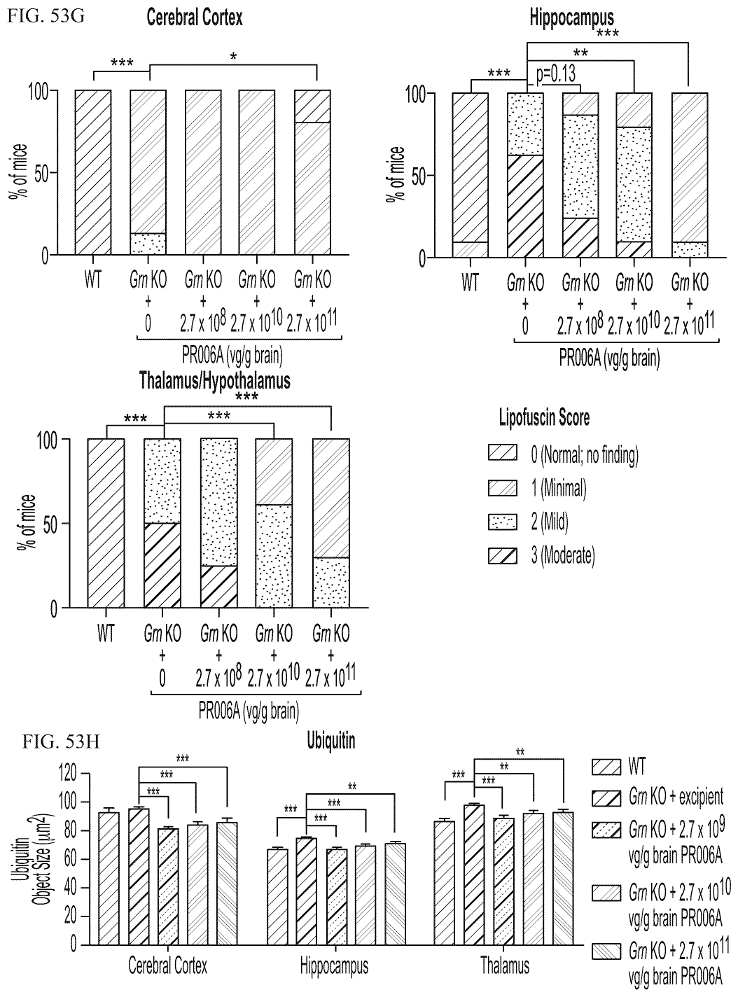

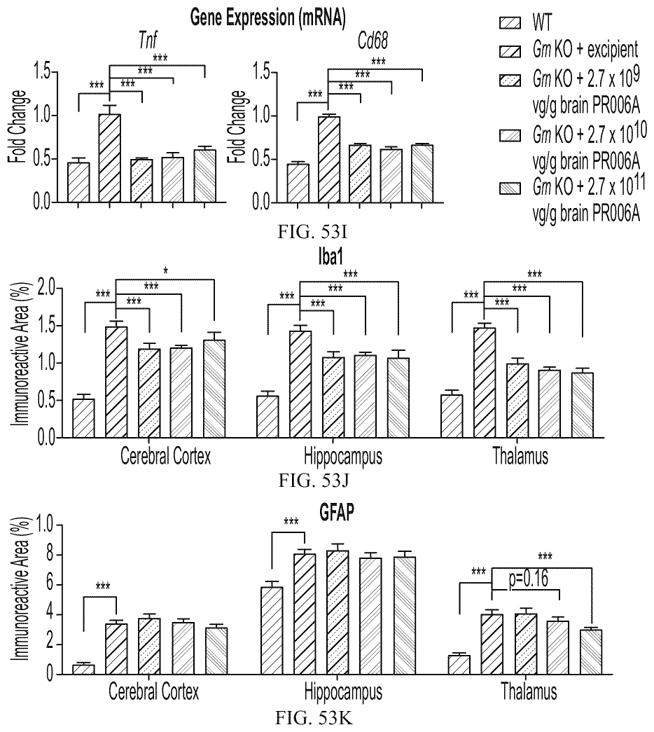

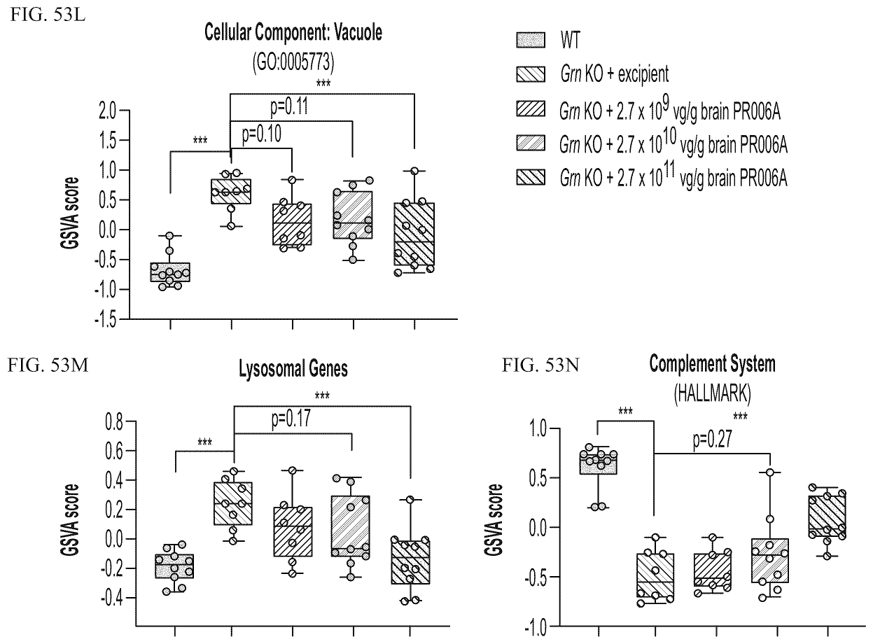

Biodistribution and Progranulin Expression: Biodistribution was determined by measuring vector genome presence using a qPCR assay that meets the current U.S. Food and Drug Administration CBER/OTAT standards for PCR sensitivity (with >50 vector genomes per g genomic DNA defined as positive). Mice that received PR006A were positive for vector genomes in the cerebral cortex and spinal cord in a dose-dependent manner, indicating that ICV administration successfully results in PR006A transduction in the CNS (FIG. 53A). qRT-PCR analysis of PR006A-encoded GRN revealed that ICV dosing of PR006A resulted in a dose-dependent induction of human GRN mRNA expression in the cerebral cortex (FIG. 53B). PR006A treatment increased levels of human progranulin protein in the brain and spinal cord (FIG. 53C). In brain tissue, human progranulin levels were detected and quantified at the highest PR006A dose; at lower doses, progranulin levels were below the assay limit of detection due to the high background in brain. However, based on the log-fold difference between doses, proportional estimation of expected progranulin levels at the lower doses would be well below the lower limit of quantitation (LLOQ) of the assay in brain tissue. The level of endogenous mouse progranulin was measured in age and strain-matched mice with wildtype (WT) Grn alleles; in both the cerebral cortex and spinal cord, the levels of human progranulin in PR006A-treated Grn KO mice did not exceed the level of endogenous progranulin in WT mice at any dose. Since different detection assays employing non-species-cross-reactive anti-progranulin antibodies were used to measure human and mouse progranulin, the absolute numbers cannot be compared with accuracy.

PR006A administration also resulted in broad vector genome presence and progranulin protein levels in peripheral tissues, including liver, heart, lung, kidney, spleen, and gonads (FIG. 53D; FIG. 53E).

In plasma, significant levels of human progranulin were detected in PR006A-treated Grn KO mice at all dose levels (FIG. 53F). In line with expectations, human progranulin was not detected in the excipient treated Grn KO mice. The levels of human progranulin in animals treated with the mid-dose of PR006A were in the same range as levels of mouse progranulin measured in mice with WT Grn alleles. Since different detection assays, employing non-species-cross-reactive anti-progranulin antibodies, were used to measure human and mouse progranulin, the absolute numbers cannot be compared with accuracy.

Lipofuscin Accumulation: Lipofuscin accumulation was assessed using two independent methods in adjacent brain sections: (1) in a more clinical approach, lipofuscin accumulation in the brain was scored by a blinded pathologist on a scale of 0 (no lipofuscin observed) to 4 (widespread lipofuscin accumulation) and (2) in a more quantitative approach, lipofuscin autofluorescence was detected by IHC and automatically quantified. Grn KO mice exhibited lipofuscinosis throughout the brain, whereas WT mice did not have detectable lipofuscin in the brain (FIG. 53G). ICV administration of PR006A led to a dose-dependent reduction in the severity scores of intracellular lipofuscin accumulation in the brains of Grn KO mice (FIG. 53G). PR006A efficacy with respect to a reduction in lipofuscinosis could be most readily quantified in brain regions that display the most robust lipofuscinosis phenotype in the Grn KO mouse model of FTD-GRN, including the hippocampus and thalamus. In addition to lipofuscin scoring by a pathologist, IHC performed in brain regions of interest (i.e., cerebral cortex, hippocampus, thalamus) to quantitatively assess lipofuscinosis detected a dose-dependent reduction in the amount of lipofuscin accumulation in the cerebral cortex and thalamic brain regions, with significant decreases occurring at the middle and high PR006A doses. IHC was also performed to assess ubiquitin accumulation in the brain, an additional FTD-GRN-related pathology that occurs in Grn KO mice. Compared to WT mice, Grn KO mice exhibited an increase in ubiquitin throughout the brain (FIG. 53H). PR006A significantly reduced ubiquitin immunoreactive object size to near WT levels at all three doses (FIG. 53H).

Neuroinflammation: Treatment with PR006A suppressed inflammatory marker levels in the brain of adult Grn KO mice. ICV PR006A decreased gene expression of the proinflammatory cytokine Tnf(TNFα) and Cd68 (CD68), a marker of microglia, in the cortex over a range of doses, from 2.7×10⁹ vg/g brain to 2.7×10¹¹ vg/g brain (FIG. 53I). In line with published data, we observed an increase in the gene expression of these neuroinflammatory markers in excipient-treated Grn KO mice compared to age-matched mice with wildtype Grn alleles (FIG. 53I). In contrast to the observations in 18-month-old aged Grn KO mice from PRV-2018-027 and reports of TNFα abnormalities in the literature, there was no robust increase in cerebral cortex TNFα protein levels in the 7-month-old adult excipient-treated Grn KO mice; additionally, no significant changes were observed with PR006A in Grn KO mice. These findings are consistent with previously published findings that robust neuroinflammatory phenotypes do not occur in the Grn KO mouse model until 12-24 months of age. Immunohistochemistry (IHC) was performed and quantified in the brain regions of interest (cerebral cortex, hippocampus, and thalamus) to further evaluate neuronal inflammation by staining for Iba1, a marker of microgliosis, and GFAP, a marker of astrocytosis. There was a significant increase in microgliosis (Iba1) and astrocytosis (GFAP) throughout the brain in Grn KO mice compared to WT mice (FIG. 53J-FIG. 53K). PR006A treatment significantly reduced microgliosis (Iba1) at all three doses (FIG. 53J). A trend toward decreased astrocytosis (GFAP) was observed at the middle PR006A dose and a significant decrease in astrocytosis (GFAP) was observed at the high PR006A dose in the thalamus brain region (FIG. 53K).

While many of the Grn KO mouse model phenotypes occur late in life, studies have reported that Grn KO mice exhibit widespread gene expression changes as early as 4 months of age, including changes in lysosomal- and immune-related pathways. Therefore, in addition to the targeted qRT-PCR analysis described above, a transcriptomics approach to evaluate changes in mRNA levels, which can be assessed globally with sensitive, high throughput technologies (RNA sequencing), and require minimal sample material, was employed. We performed RNA sequencing on cerebral cortices and used Gene Set Variation Analysis (GSVA) (Hanzelmann et al., BMC Bioinformatics 14, 7 (2013)) to determine which gene expression pathways are altered in the 7-month old excipient-treated Grn KO mice, as compared to age-matched WT mice of the same strain. We confirmed deficiencies in lysosomal- and immune-related pathways in mice lacking Grn, as reported in previously published studies. Significant changes were reported in a subset of the GO TERM (GO: 0005773) “Vacuole” genes (contains 4 genes reported to be dysregulated in Grn KO mice described by Lui et al (Cell 165, 921-935 (2016))), the “Lysosomal Genes” set (a subset of 25 μlysosomal-related genes shown to be dysregulated in Grn KO mice described by Evers et al (Cell Reports 20, 2565-2574 (2017))), and the “Complement” gene set from Gene Set Enrichment Analysis HALLMARK database (contains genes encoding components of the complement system, part of the innate immune system). We then measured and compared activity levels of these gene sets with PR006A treatment (FIG. 53L-FIG. 53N). Treatment with PR006A dose-dependently reversed the gene set deficiencies observed in the Grn KO mice.

Histopathology: A thorough histopathological analysis performed by a blinded board-certified pathologist on hematoxylin and eosin (H&E) staining of the brain, thoracic spinal cord, liver, heart, spleen, lung, kidney, and gonads of all mice from these studies found no evidence of toxicity related to PR006A treatment. The details of the toxicity analysis are provided in the section below.

Conclusion: ICV PR006A at doses ranging from 2.7×10⁹ vg/g brain to 2.7×10¹¹ vg/g brain resulted in broad vector genome presence throughout the brain and peripheral tissues in a dose-dependent manner. PR006A treatment also led to production of progranulin mRNA and protein in the CNS. A clear dose-response relationship between PR006A and decreased lipofuscinosis, a readout of lysosomal dysfunction, was observed throughout multiple brain regions. A robust and statistically significant reduction of lipofuscinosis was observed at the middle and highest dose level of PR006A. All PR006A doses reduced ubiquitin accumulation in the brain. Starting at the lowest dose of 2.7×10⁹ vg/g brain, PR006A reduced the expression of proinflammatory markers in the brain at the RNA and protein level.

### Summary: In Vivo Nonclinical Studies

PR006A effectively transduced Grn KO mice, resulting in a robust, dose-dependent biodistribution of the transgene and production of progranulin mRNA and protein in the CNS. PR006A dose-dependently reversed gene expression abnormalities in lysosomal and neuroinflammatory pathways. PR006A reduced many of the phenotypes that occur in the brain of this FTD-GRN mouse model, including lipofuscinosis, ubiquitin accumulation, and microgliosis. In the dose-ranging study, the lowest dose of 2.7×10⁹ vg/g brain PR006A significantly suppressed the expression of inflammatory markers in the cerebral cortex. The middle dose of 2.7×10¹⁰ vg/g brain PR006A improved both lysosomal defects (e.g., lipofuscinosis) and neuroinflammation, in a robust and statistically significant way. The high dose of 2.7×10¹¹ vg/g brain PR006A further increased progranulin expression with no evidence of toxicity.

TABLE 7
Summary of Biodistribution

| | | Cerebral | Spinal | | | | | | |
|---|---|---|---|---|---|---|---|---|---|
| Study | Dose | Cortex | Cord | Liver | Spleen | Heart | Kidney | Lung | Gonads |
| PRV-2018-027 | 9.7 × 10¹⁰ vg | + | + | + | + | + | + | + | + |
| | PR006A | | | | | | | | |
| PRV-2019-004 | 1.1 × 10⁹ vg | + | + | + | + | + | + | + | + |
| | PR006A | | | | | | | | |
| | 1.1 × 10¹⁰ vg | + | + | + | + | + | + | + | + |
| | PR006A | | | | | | | | |
| | 1.1 × 10¹¹ vg | + | + | + | + | + | + | + | + |
| | PR006A | | | | | | | | |

Positive biodistribution is defined as >50 vg/μg genomic DNA.

### Safety Pharmacology

Throughout these studies, there were no adverse events that can be attributed to the test article. Safety findings from in-life and histopathological analyses of the animals in PRV-2018-027, PRV-2019-002, and PRV-2019-004 are discussed in the section below.

### Single-Dose Toxicity

A series of nonclinical studies with PR006A were conducted investigating safety endpoints in mice and monkeys. Three of the studies were performed in a Grn KO mouse model, where endpoints included neuropathological evaluations and assessed both protective activity as well as potential toxicity resulting from PR006A administration via intracerebroventricular (ICV) injection; ICM administration is more technically difficult in mice. These mouse models are representative of FTD-GRN in which patients have a mutation in the GRN gene resulting in reduced progranulin levels. In cynomolgus monkeys, neuropathology was also performed as part of a pilot study in which PR006A was injected into the cisterna magna (ICM). A GLP study was conducted in cynomolgus monkeys in which PR006A was delivered to the ICM, and monkeys were sacrificed at Day 7, Day 30, or Day 183. The GLP study incorporated a comprehensive list of clinical endpoints in addition to anatomical pathology evaluations on a full list of tissues. To support single-dose administration in the clinic, the following single-dose studies were conducted.

### Maximal Dose PR006A in an Aged FTD-GRN Mouse Model (PRV-2018-027 and PRV-2019-002)

As part of these efficacy studies in Grn KO mice, neuropathological evaluations were conducted in mice treated ICV with either excipient or PR006A. Grn KO mice have a complete loss of progranulin and are widely used as models of FTD-GRN due to their age-dependent phenotypes, which include lysosomal alterations, neuronal lipofuscin accumulation, microgliosis, and neuroinflammation. Aspects of the pharmacology portions of the study are summarized in the sections above whereas toxicological-related endpoints assessed in this study are summarized below. Two studies of PR006A were conducted in the aged Grn KO mouse model. In the first study (PRV-2018-027), 9 mixed gender Grn KO mice 16 months of age received ICV administration of either PR006A or excipient. Animals were sacrificed 9 weeks post-administration. A single PR006A dose group was included in this study: 10 μl of undiluted virus, for a total dose of 9.7×10¹⁰ vg (2.4×10¹¹ vg/g brain), and the control group was treated with 10 μl of excipient.

TABLE 8
Study Design PRV-2018-027

| | | | PR006A | Total | | Post- |
|---|---|---|---|---|---|---|
| | | RoA | Dose | PR006A | Number | Treatment |
| Model | Treatment | (Dose Volume) | (vg/g brain) | Dose (vg) | of Mice | Necropsy |
| Grn KO | Excipient | ICV (10 μl) | 0 | 0 | 4 | 9 weeks |
| | | | | | (2 M/2 F) | |
| Grn KO | PR006A | ICV (10 μl) | 2.4 × 10¹¹ | 9.7 × 10¹⁰ | 5 | 9 weeks |
| | | | | | (3 M/2 F) | |

ROA: route of administration

Various post-mortem endpoints, such as biodistribution, lysosomal alterations, and inflammatory markers, were evaluated as part of this study protocol (see section above). Animals were also checked for survival twice per day, and body weight was measured once per day. After euthanasia at 2-months post-treatment, target tissues were harvested, drop fixed in chilled 4% paraformaldehyde, and stored at 4° C. The tissues from the 8 animals that completed the study were trimmed, processed, and embedded in paraffin blocks. They were then sectioned at ˜5 μm, stained with hematoxylin and eosin (H&E) and examined by a board-certified veterinary pathologist.

During this study, 1 mouse died prematurely from the treatment group; no abnormalities were recorded for the deceased animal during necropsy, and therefore there is no known cause of death. No other deaths or abnormalities were observed. All treatment groups tracked similarly in terms of body weights, with no significant differences present.

On histopathological examination, there were no PR006A-related adverse findings. There was widespread lipofuscin accumulation in the brain, consistent with expected findings in a Grn KO mouse. In PR006A-treated animals, there was a reduction in the score severity for lipofuscin accumulation in all regions of the brain. Morphologic changes also appeared to demonstrate a slight reduction in frequency and/or severity scores, particularly with respect to neuronal necrosis in the medulla and pons, with PR006A treatment. However, these trends in the morphologic changes were not as consistent as that of the lipofuscin scores.

In the thoracic spinal cord, there was axonal degeneration and, very rarely (1 out of 4 animals in each group), minimal neuronal necrosis observed. There was a minor reduction in both the incidence and severity of axonal degeneration in the animals treated with PR006A.

The following findings, which are presumably associated with the Grn homozygous knockout mouse, appeared to have a reduced incidence and/or severity in the animals treated with PR006A: dilated tubules in the medulla of the kidney, glomerulopathy in the kidney, and foreign material in the lung (characterized as linear, acellular, dark pink structures, usually within airways and frequently associated with foreign body giant cells and/or macrophages). A larger cohort of animals would be necessary for more definitive conclusions.

All other histopathologic findings observed were considered incidental and/or were of similar incidence and severity in excipient- and test article-treated animals and, therefore, were considered unrelated to administration of PR006A.

In the second study (PRV-2019-002), 5 mixed gender Grn KO mice 14 months of age received ICV administration of either PR006A or excipient. Animals were sacrificed 8 weeks post-administration. A single PR006A dose group was included in this study: 10 μl of undiluted virus, for a total dose of 9.7×10¹⁰ vg (2.4×10¹¹ vg/g brain), and the control group was treated with 10 μl of excipient.

TABLE 9
Study Design PRV-2019-002

| | | | PR006A | Total | | Post- |
|---|---|---|---|---|---|---|
| | | RoA | Dose | PR006A | Number | Treatment |
| Model | Treatment | (Dose Volume) | (vg/g brain) | Dose (vg) | of Mice | Necropsy |
| Grn KO | Excipient | ICV (10 μl) | 0 | 0 | 2 | 8 weeks |
| | | | | | (0 M/2 F)* | |
| Grn KO | PR006A | ICV (10 μl) | 2.4 × 10¹¹ | 9.7 × 10¹⁰ | 3 | 8 weeks |
| | | | | | (1 M/2 F) | |

*Genotype results at the end of the study confirmed that n = 1 animal from the excipient group to be Grn heterozygous KO instead of the expected Grn homozygous KO.

The animals were analyzed in an identical manner to study PRV-2018-027. Animals were checked for survival twice per day, and body weight was measured once per day. After euthanasia at 2-months post-treatment, target tissues were harvested, drop fixed in chilled 4% paraformaldehyde, and stored at 4° C., until evaluation.

In the CNS, findings consistent with those previously observed in the Grn KO mouse were observed in the brain (Yin et al., JExp Med 207(1):117-128 (2010)). Specifically, there was a widespread increase in lipofuscin accumulation throughout the brain. Rarely minimal neuronal necrosis was also observed (in the single untreated early death animal and in one Excipient animal).

Due to the low sample numbers it was not possible to demonstrate a consistent trend in the findings related to treatment. There was no consistent difference in response between the Test Article (PR006A) and Excipient.

For non-CNS tissues, findings that were considered to be consistent with the phenotype of the Grn KO mouse were observed in the kidney (tubular dilation and infiltrates of mononuclear inflammatory cells) and liver (vacuolation of Kupffer cells/sinusoidal lining cells, and Kupffer cell microgranulomas) (Yin et al., JExp Med 207(1):117-128 (2010)).

There was a finding of “glomerulopathy” observed in all animals that underwent surgery and were enrolled in the study. While published reports of this finding as a change associated with standard, unchallenged, Grn knockout mice were not found, one study has demonstrated progranulin-deficient mice treated with a diet that induces hyperhomocysteinemia, develop glomerular basement membrane thickening and podocyte foot process effacement (Fu et al., Hypertension 69(2):259-266 (2017)).

All other findings were consistent with those commonly observed in laboratory mice. Due to the low sample number, no conclusive difference related to treatment could be shown.

### Dose-Ranging PR006A in an Adult FTD-GRN Mouse Model (PRV-2019-004)

To further assess the safety of PR006A, a larger, dose-ranging study in adult Grn KO mice was performed. A total of 40 mixed-gender mice were divided into 4 groups and administered either excipient or one of three doses of PR006A by a single unilateral ICV injection into the left hemisphere; all animals, regardless of treatment group, received a total dose volume of 10 μl. Mice were treated at 4 months of age and euthanized 3 months post-treatment. An additional wildtype (WT) control group, which included untreated C57BL/6J mice (the same background strain) aged to approximately 7 months, were also euthanized and subjected to a similar necropsy.

The study was conducted according to the study design below:

TABLE 10
Study Design PRV-2019-004

| | | | RoA | Dose of | Total | Number | Post- |
|---|---|---|---|---|---|---|---|
| | | Treat- | (Dose | PR006A | PR006A | of | Treatment |
| Group | Model | ment | Volume) | (vg/g brain) | Dose (vg) | Mice | Necropsy |
| 1 | Grn KO | Excipient | ICV | 0 | 0 | 10 | Week 13 |
| | | | (10 μl) | | | (4 M/6 F) | |
| 2 | Grn KO | PR006A | ICV | 2.7 × 10¹¹ | 1.1 × 10¹¹ | 10 | Week 13 |
| | | | (10 μl) | | | (4 M/6 F) | |
| 3 | Grn KO | PR006A | ICV | 2.7 × 10¹⁰ | 1.1 × 10¹⁰ | 10 | |
| | | | (10 μl) | | | (4 M/6 F) | Week 13 |
| 4 | Grn KO | PR006A | ICV | 2.7 × 10⁹ | 1.1 × 10⁹ | 10 | Week 13 |
| | | | (10 μl) | | | (4 M/6 F) | |
| N/A | WT | None | N/A | 0 | 0 | 10 | N/A |
| | (C57BL/6J) | | | | | (5 M/5 F) | |

During the study, animals were checked for survival twice a day and weighed once a week. Mice were euthanized 3 months post-treatment, and various post-mortem evaluations were conducted to assess efficacy of PR006A (see section above). In addition, sections stained for H&E from brain, thoracic spinal cord, liver, heart, spleen, lung, kidney, and gonads were evaluated by a board-certified pathologist.

On histopathological examination, there were no adverse PR006A-related findings in any of the mice regardless of treatment group.

There were findings consistent with the Grn KO mouse model phenotype, such as accumulation of intracellular lipofuscin in various regions of the brain: cerebral cortex, cerebral nuclei, hippocampus, thalamus/hypothalamus, cerebellum and brainstem (particularly the pons and medulla). Clear evidence of morphologic changes on the H&E stained sections (vacuolation of neurons and gliosis) was not observed. Accumulation of lipofuscin pigment can precede easily detectable morphologic changes and, therefore, serves as an adequate biomarker of efficacy. While all Grn homozygous KO groups demonstrated lipofuscin accumulation, there were differences in the severity of this finding across treatment groups. The frequency of higher scores for lipofuscin accumulation was greatest for the group of animals treated with excipient (Group 1). Of those animals treated with PR006A, the frequency of higher scores were observed in Group 4 (low dose PR006A; 2.7×10⁹ vg/g brain), followed by Group 3 (middle dose PR006A; 2.7×10¹⁰ vg/g brain). The lowest severity scores were observed with in Group 2 (high dose PR006A; 2.7×10¹¹ vg/g brain). These findings demonstrate a dose-dependent reduction in the severity scores of intracellular lipofuscin accumulation in the brains of Grn homozygous knock-out mice. All other histopathologic findings were considered incidental and/or were of similar incidence and severity in excipient and test article-treated animals and, therefore, were considered unrelated to administration of PR006A.

### GLP Single-Dose Study in Monkeys (PRV-2018-028)

### Study Design

The purpose of this GLP study was to evaluate the toxicity and biodistribution of the test article, PR006A, when administered once via ICM injection in cynomolgus monkeys with a 6-day, 29-day, or 182-day post-administration observation period; animals were sacrificed at study Day 7, Day 30, or Day 183. The study was designed to evaluate 2 dose levels: the highest dose is the maximum feasible dose achievable with 1.2 mL volume (the highest volume there was experience in administering) of undiluted PR006A, and a lower dose that is equivalent to one log unit lower than the high dose. The doses equate to a low dose of 4.8×10¹¹ vg and a high dose of 4.8×10¹² vg; with a brain weight estimate of 74 g in a cynomolgus monkey, the NHP species used in this study, this translates to approximately 6.5×10⁹ vg/g brain and 6.5×10¹⁰ vg/g brain. The study also includes a control arm in which animals receive 1.2 ml of excipient only (20 mM Tris pH 8.0, 200 mM NaCl, and 1 mM MgCl₂+0.001% [w/v] Pluronic F68). This study utilized both male and female cynomolgus macaques. The Day 7 group included 1 female at the highest dose and was designed as a sentinel for early toxicity; the remaining two timepoints (Day 30 and Day 183) included 2 males and 1 female at each dose. In addition to samples from multiple brain regions, peripheral tissue samples were collected for qPCR analysis. All samples that were positive with qPCR were analyzed for transgene expression. A tabulated summary of this study's design is provided in Table 11.

TABLE 11
Overview of the GLP NHP Study PRV-2018-028

| Purpose | Assess the tolerance and biodistribution of PR006A in | | | | |
|---|---|---|---|---|---|
| | NHPs | | | | |
| Regulatory Compliance | GLP | | | | |
| Test Article | PR006A | | | | |
| Total No. of Animals | 19 cynomolgus monkeys | | | | |
| Weight (age) | 2-5 kg (25-50 months) | | | | |
| Study Design | Group Assignments: | | | | |
| | | Dose | Number of Animals | | |
| | | (vg/g | Necropsy | Necropsy | Necropsy |
| | Group | brain) | (Day 7) | (Day 30) | (Day 183) |
| | 1 | 0 | 0 | 2M/1F | 2M/1F |
| | 2 | 6.5 × 10⁹ | 0 | 2M/1F | 2M/1F |
| | 3 | 6.5 × 10¹⁰ | 1F | 2M/1F | 2M/1F |

| Dosing Route and Frequency | Intra-cisterna magna using a polypropylene 1-3 cc syringe | |
|---|---|---|
| | and spinal needle (Pencan 25 G × 2.5 cm BBraun); single | |
| | slow bolus delivered at a maximum rate of 0.5 cc/min | |
| Formulations | Dosing solution provided at concentration of 4.01 × 10¹² | |
| | vg/mL | |
| Clinical Signs | Daily (including food consumption); Detailed Observations weekly | |
| Body weights | Weekly | |
| Neurological, Ophthalmic, | Once pre-dose and during Weeks 2 and 26 | |
| ECG Examinations | | |
| Clinical Pathology | All groups hematology, clinical chemistry, coagulation parameters | |
| Hematology | red blood cell count | mean corpuscular volume |
| | hemoglobin | platelet count |
| | hematocrit | white blood cell count |
| | mean corpuscular | absolute neutrophil count |
| | hemoglobin | absolute lymphocyte count |
| | mean corpuscular | absolute monocyte count |
| | hemoglobin concentration | absolute reticulocyte count |
| | absolute eosinophil count | differential blood cell count |
| | absolute basophil count | blood smear |
| Clinical Chemistry | glucose | alanine aminotransferase |
| | urea nitrogen | alkaline phosphatase |
| | creatinine | gamma glutamyltransferase |
| | total protein | aspartate aminotransferase |
| | albumin | calcium |
| | globulin | inorganic phosphorus |
| | albumin/globulin ratio | sodium |
| | cholesterol | potassium |
| | total bilirubin | chloride |
| | creatine kinase | triglycerides |

| Coagulation | prothrombin time | | |
|---|---|---|---|
| | fibrinogen | | |
| | activated partial thromboplastin time | | |
| Vector Shedding (urine/feces) | At sacrifice | | |
| Necropsy | Day 7, Day 30, Day 183 | | |
| Tissue Preservation for | The following tissues from each animal will be collected in | | |
| Histopathology | 10% neutral-buffered formalin (unless otherwise indicated) | | |
| | or recorded as missing, if applicable. | | |
| Tissue Preservation, | Adrenalª | Injection site | Prostate^{a} |
| continued | Aorta | (overlying skin) | Rectum |
| | Bone, femur with | Jejunum | Salivary gland |
| | bone marrow | Kidneyª | Sciatic nerve |
| | Bone, sternum | Lesions | Seminal vesicle^{a} |
| | with bone | Liverª | Skin/subcutis |
| | marrow | Lung with large | Spinal cord |
| | Brain^{a} | bronchi | (cervical, |
| | Cecum | Lymph node | thoracic, lumbar) |
| | Cervix | (mandibular) | Spleen^{a} |
| | Colon | Lymph node | Stomach |
| | Duodenum | (mesenteric) | Testis^{a} |
| | Epididymis^{a} | Mammary gland | Thymus^{a} |
| | Esophagus | Muscle, biceps | Thyroid with |
| | Eye^{b} | femoris | parathyroidª |
| | Gall bladder | Optic nerve | Tongue |
| | GALT (Peyer’s | Ovaryª | Trachea |
| | Patch) | Oviducts | Urinary bladder |
| | Heartª | Pancreas | Uterus^{a} |
| | Ileum | Pituitary glandª | Vagina |

| Histopathology | All groups-all tissues | |
|---|---|---|
| Biodistribution | The following tissues/biofluids will be analyzed for | |
| | biodistribution by qPCR: | |
| | Frontal cortex | Liver |
| | Hippocampus | DRG (cervical) |
| | Ventral mesencephalon | DRG (thoracic) |
| | Periventricular gray | DRG (lumbar) |
| | Putamen | Spinal cord (thoracic) |
| | Testis | Spinal cord (lumbar) |
| | Ovary | Spinal cord (cervical) |
| | Kidney | Spleen |
| | Stomach (pyloric) | Heart (apex) |
| | Blood | CSF |
| | Lung | |

| | |
|---|---|
| Transgene Expression | All samples that are positive for qPCR will be evaluated for |
| | progranulin expression |

Abbreviations:
F, female;
ICM; intra-cisterna magna;
M, male;
MgCl2; magnesium chloride;
NaCl, sodium chloride;
vg, vector genome(s);
DRG, dorsal root ganglia;
GALT, gut-associated lymphoid tissue.

TABLE 11A
Group Assignments

| | Dose | Number of Animals | | |
|---|---|---|---|---|
| | (vg/g | Necropsy | Necropsy | Necropsy |
| Group | brain) | (Day 7) | (Day 30) | (Day 183) |
| 1 | 0 | 0 | 2M/1F | 2M/1F |
| 2 | 6.5 × 109 | 0 | 2M/1F | 2M/1F |
| 3 | 6.5× 1010 | 1F | 2M/1F | 2M/1F |

Cynomolgus NHPs were assessed by multiple in-life observations and measurements, including mortality/morbidity (daily), clinical observations (daily), body weight (baseline and weekly thereafter), visual inspection of food consumption (daily), neurological observations (baseline and during Week 2 and 26), indirect ophthalmoscopy (baseline and during Weeks 2 and 26), and electrocardiographic (ECG) measurement (baseline and during Weeks 2 and 26).

Analysis of neutralizing antibodies (nAb) to the AAV9 capsid was performed at baseline and at sacrifice on Days 7, 30, or 183. Clinical pathology consisting of hematology, coagulation, clinical chemistry, and urinalysis was performed twice at baseline (blood tests; once for urinalysis) and once during Weeks 1 and 13 of the dosing phase.

Animals were euthanized and tissues harvested on Day 7, Day 30, or Day 183. The tissues outlined in Table 11, if present, were collected from all animals, weighed (if applicable), and divided into replicates. One replicate was preserved in 10% neutral-buffered formalin (except when special fixatives are required for optimum fixation) for histopathological evaluation (all animals). Additional replicates were collected for qPCR and transgene expression analysis.

### Safety and Toxicology

There were no unscheduled deaths, and all animals survived until the scheduled necropsy. There were no adverse PR006A-related clinical observations, body weight changes, ophthalmic observations, or physical or neurological examination findings; gross macroscopic examination at necropsy showed no drug-related abnormalities in any of the cohorts. In addition, there were no PR006A-related changes in PR interval, QRS duration, QT interval, corrected QT (QTc) interval, or heart rate observed in males or combined sexes administered 6.5×10⁹ or 6.5×10¹⁰ vg/g brain. No abnormal ECG waveforms or arrhythmias were observed during the qualitative assessment of the ECGs.

### Biodistribution

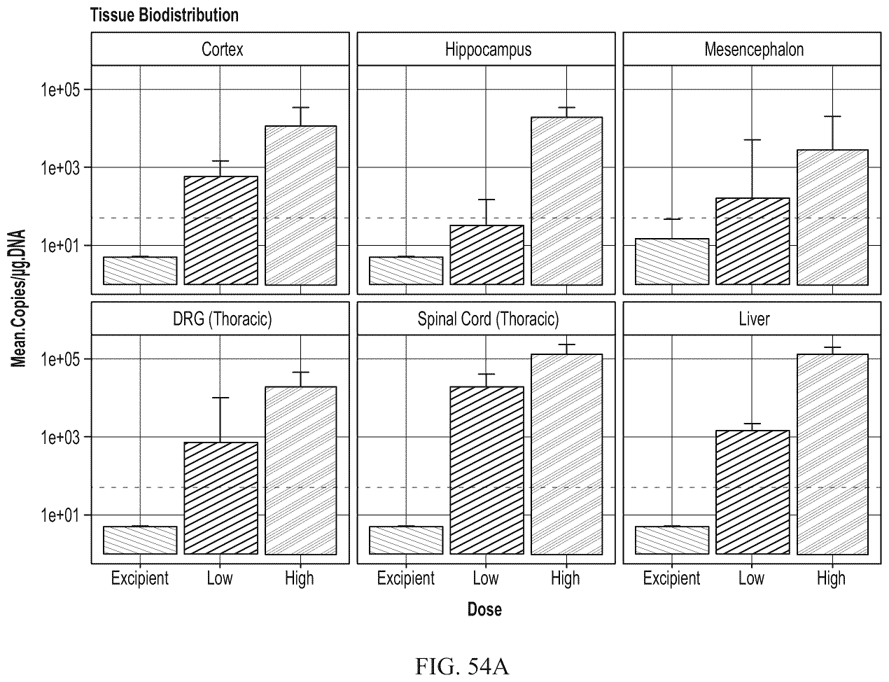

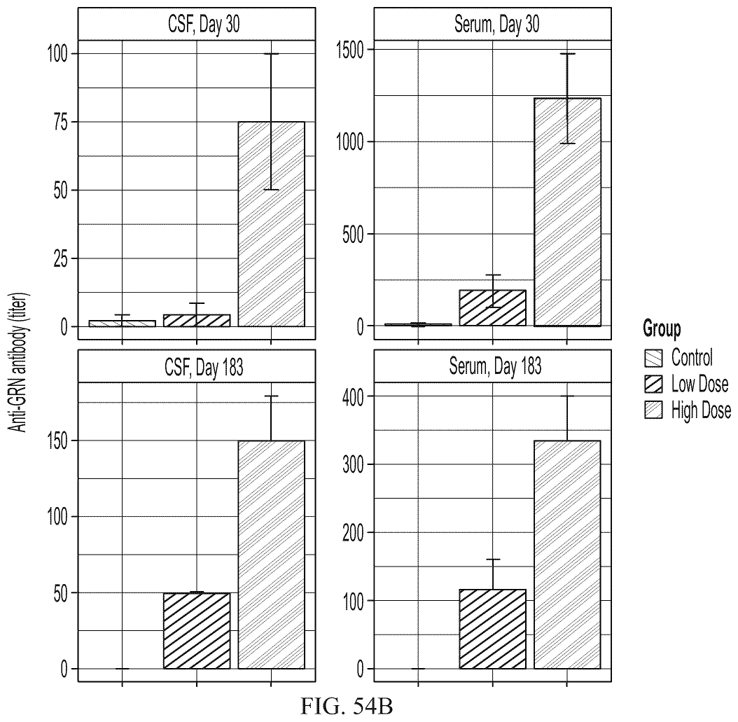

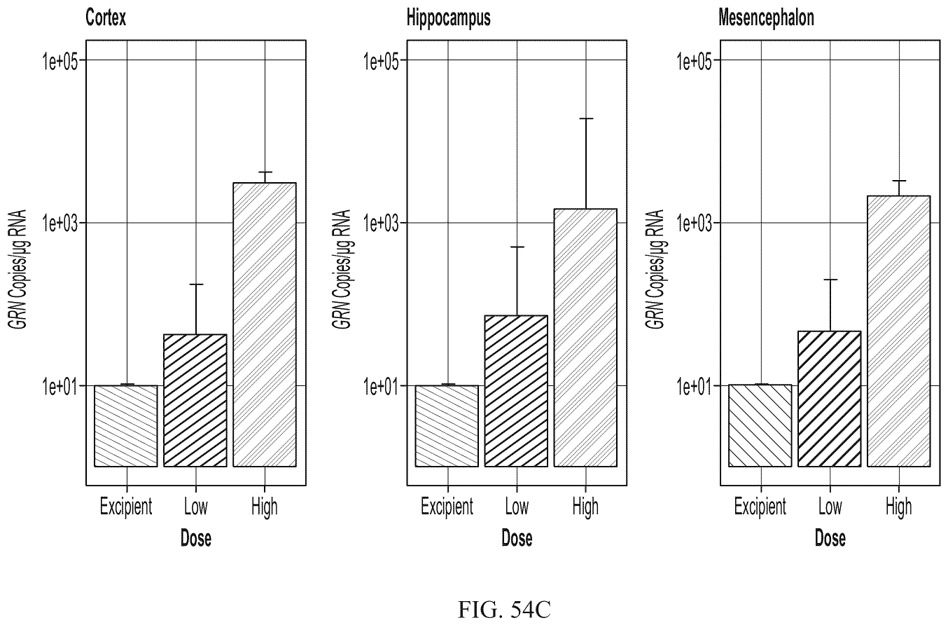

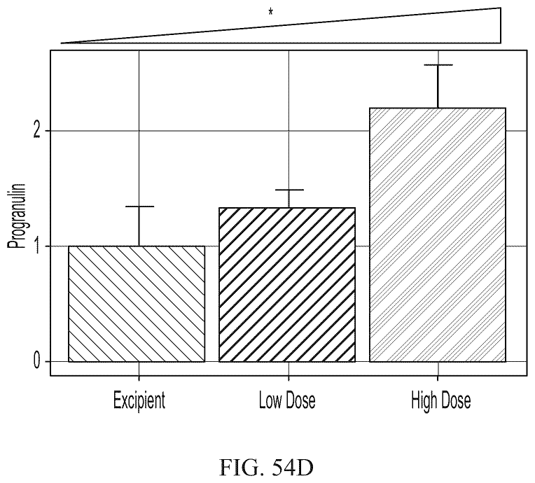

Biodistribution analysis of the PR006A transgene was performed using a qPCR-based assay. At Day 183 in the high dose group (6.5×10¹⁰ vg/g brain), there was widespread transduction throughout the CNS and periphery, with all tissues examined positive for vector presence with a cutoff of 50 vg/μg DNA, the lower limit of quantitation for the qPCR assay. Data from select representative regions from Day 183 are shown in FIG. 54A; Day 30 data is not shown. At Day 30 in the high dose group (6.5×10¹⁰ vg/g brain), all CNS tissues examined were positive for transduction, with the exception of the putamen. Tissues from animals treated with the low dose (6.5×10⁹ vg/g brain) were positive in the CNS at Day 183, but only the spleen and liver were positive from the peripheral tissues. In addition, the one female NHP treated with the high dose of PR006A was positive in the ovaries at Day 7, and males treated with the high dose were positive in the testes at Day 30 and Day 183. PR006A transduction was most robust in liver and tissues of the nervous system, and consistently lower in the other peripheral organs examined. In the brain, vector transduction stabilized at Day 183 when compared to Day 30, demonstrating a robust and durable transduction of the transgene.

In the NHPs receiving ICM administration of PR006A, there was a significant allogeneic immune response to the transgene product, progranulin, with anti-progranulin antibodies detected in serum and CSF samples collected at Day 30 and Day 183 post-treatment; the immune response indicates that the human progranulin protein was expressed in the NHPs. The antidrug antibody (ADA) levels were determined using established immune assay technologies. The data are illustrated in FIG. 54B.

Expression of PR006A (GRN) was measured at the mRNA level using a RT-qPCR-based assay, and at the protein level using a Simple Western™ (Jess) analysis. Concomitant with levels of PR006A transduction, expression of the transgene was observed by mRNA measurements using RT-qPCR in select brain regions (FIG. 54C), liver, gonads, spinal cord and DRG collected on Day 183.

Expression of the transgene was measurable in brain and liver at both doses of PR006A, and the expression levels were both dose-dependent and durable. In gonads, expression was measurable in the males at the high dose only; at both doses in the females, expression was measurable at Day 7 and Day 30, but not at Day 183.

To confirm that human progranulin was produced in the treated NHPs, protein levels in CSF were evaluated on a Simple Western™ (Jess) platform. Details of the method are provided in Example 14. The method was qualified by measuring progranulin levels in CSF samples from FTD-GRN patients and establishing that they were approximately half of the levels measured in CSF samples from healthy human controls and from FTD patients without a GRN mutation. Results from the CSF indicate that levels of progranulin are elevated in a dose-dependent manner in animals treated with both the low and high doses of PR006A (FIG. 54D). These results indicate that the effective and broad transduction of PR006A in NHPs following ICM administration leads to increased levels of progranulin.

Progranulin protein measurements focused on CSF because the Simple Western™ (Jess) assay is not suitable to measure progranulin levels in brain tissue due to the high level of nonspecific background bands. The assays currently available do not reliably measure levels of transgene-derived human progranulin in NHP tissues due to the high levels of nonspecific background. CSF levels are generally believed to reflect relevant brain concentrations, and they are of particular value as translational biomarkers to clinical studies.

### SUMMARY

There have been no adverse safety findings or toxicity concerns in any of the nonclinical studies, including a small pilot non-GLP study in NHPs and a GLP study in NHPs through Day 183, that preclude the initiation of a clinical study. The pathology findings in the GLP study were consistently minimal in severity with a low number of affected cells across both dose groups. There were no other in-life or post-mortem PR006A-related adverse findings.

Phase ½ Trial in Human Subjects with FTD-GRN

Human subjects (n=15) will be enrolled in an open-label trial of the PR006 recombinant AAV. The subject inclusion criteria comprise: 30-80 years old (inclusive), has a pathogenic GR mutation, is at a symptomatic disease stage, and has stable use of background medications prior to investigational product dosing. Each subject will receive the investigational product as a single ICM (intra-cisterna magna) injection. The trial will include a 3-month biomarker readout, a 12-month clinical readout and a 5-year safety and clinical follow-up. The trial will analyze: (1) safety and tolerability: (2) key biomarkers, including: progranulin, NfL (neurofilament light chain), and volumetric MRI (magnetic resonance imaging); and (3) Efficacy: CDR plus NACC FTLD (Clinical Dementia Rating plus National Alzheimer's Coordinating Center Frontal Temporal Lobar Dementia); measures of behavior, cognition, language, function, and QoL (quality of life).

TABLE 12
Examples of neurodegenerative diseases

| Disease | Associated genes |
|---|---|
| Alzheimer’s disease | APP, PSEN1, PSEN2, APOE |
| Parkinson’s disease | LRRK2, PARK7, PINK1, PRKN, |
| | SNCA, GBA, UCHL1, ATP13A2, |
| | VPS35 |
| Huntington’s disease | HTT |
| Amyotrophic lateral sclerosis | ALS2, ANG, ATXN2, C9orf72, |
| | CHCHD10, CHMP2B, DCTN1, |
| | ERBB4, FIG4, FUS, HNRNPA1, |
| | MATR3, NEFH, OPTN, PFN1, PRPH, |
| | SETX, SIGMAR1, SMN1, SOD1, |
| | SPG11, SQSTM1, TARDBP, TBK1, |
| | TRPM7, TUBA4A, UBQLN2, VAPB, |
| | VCP |
| Batten disease (Neuronal | PPT1, TPP1, CLN3, CLN5, CLN6, |
| ceroid lipofunscinosis) | MFSD8, CLN8, CTSD, DNAJC5, |
| | CTSF, ATP13A2, GRN, KCTD7 |
| Friedreich’s ataxia | FXN |
| Lewy body disease | APOE, GBA, SNCA, SNCB |
| Spinal muscular atrophy | SMN1, SMN2 |
| Multiple sclerosis | CYP2781, HLA-DRB1, IL2RA, IL7R, |
| | TNFRSF1A |
| Prion disease (Creutzfeldt-Jakob | PRNP |
| disease, Fatal familial insomnia, | |
| Gertsmann-Straussler-Scheinker | |
| syndrome, Variably protease- | |
| sensitive prionopathy) | |

TABLE 13
Examples of synucleinopathies

| Disease | Associated genes |
|---|---|
| Parkinson’s disease | LRRK2, PARK7, PINK1, PRKN, |
| | SNCA, GBA, UCHL1, |
| | ATP13A2, VPS35 |
| Dementia with Lewy bodies | APOE, GBA, SNCA, SNCB |
| Multiple system atrophy | COQ2, SNCA |

TABLE 14
Examples of tauopathies

| Disease | Associated genes |
|---|---|
| Alzheimer’s disease | APP, PSEN1, PSEN2, APOE |
| Primary age-related tauopathy | MAPT |
| Progressive supranuclear palsy | MAPT |
| Corticobasal degeneration | MAPT, GRN, C9orf72, VCP, |
| | CHMP2B, TARDBP, FUS |
| Frontotemporal dementia with | MAPT |
| parkinsonism-17 | |
| Subacute sclerosing panencephalitis | SCN1A |
| Lytico-Bodig disease | |
| Gangioglioma, gangliocytoma | |
| Meningioangiomatosis | |
| Postencephalitic parkinsonism | |
| Chronic traumatic encephalopathy | |

TABLE 15
Examples of lysosomal storage diseases

| Disease | Associated genes |
|---|---|
| Niemann-Pick disease | NPC1, NPC2, SMPD1 |
| Fabry disease | GLA |
| Krabbe disease | GALC |
| Gaucher disease | GBA |
| Tach-Sachs disease | HEXA |
| Metachromatic leukodystrophy | ARSA, PSAP |
| Farber disease | ASAH1 |
| Galactosialidosis | CTSA |
| Schindler disease | NAGA |
| GM1 gangliosidosis | GLB1 |
| GM2 gangliosidosis | GM2A |
| Sandhoff disease | HEXB |
| Lysosomal acid lipase deficiency | LIPA |
| Multiple sulfatase deficiency | SUMF1 |
| Mucopolysaccharidosis Type I | IDUA |
| Mucopolysaccharidosis Type II | IDS |
| Mucopolysaccharidosis Type III | GNS, HGSNAT, NAGLU, SGSH |
| Mucopolysaccharidosis Type IV | GALNS, GLB1 |
| Mucopolysaccharidosis Type VI | ARSB |
| Mucopolysaccharidosis Type VII | GUSB |
| Mucopolysaccharidosis Type IX | HYAL1 |
| Mucolipidosis Type II | GNPTAB |
| Mucolipidosis Type III alpha/beta | GNPTAB |
| Mucolipidosis Type III gamma | GNPTG |
| Mucolipidosis Type IV | MCOLN1 |
| Neuronal ceroid lipofuscinosis | PPT1, TPP1, CLN3, CLN5, CLN6, |
| | MFSD8, CLN8, CTSD, DNAJC5, |
| | CTSF, ATP13A2, GRN, KCTD7 |
| Alpha-mannosidosis | MAN2B1 |
| Beta-mannosidosis | MANBA |
| Aspartylglucosaminuria | AGA |
| Fucosidosis | FUCA1 |

### Example 14: Automated Western Assay for Detection of Progranulin in Cerebrospinal Fluid

The purpose of this experiment was to quantify the protein levels of progranulin (PGRN) in cerebrospinal fluid (CSF) using the ProteinSimple (San Jose, CA) Automated Western platform Jess. This test method may be used to analyze non-human primate (NHP) CSF samples. To determine the expression levels of human progranulin protein, the transgene product of PR006A, CSF samples from non-human primate subjects were analyzed on a Simple Western™ (Jess) platform using an antibody that specifically detects human progranulin protein. The Simple Western™ platform is a capillary-based automated Western blot immunoassay platform, where all steps, including protein separation, immunoprobing, washing, and detection by chemiluminescence occur in a capillary cartridge. Samples (at 4-fold dilution) and primary antibody to human progranulin (Adipogen PG-359-7, at 10-fold dilution), in addition to secondary antibodies and all buffers manufactured by ProteinSimple, were loaded onto a customized cartridge which was run on the Jess platform. Semi-quantitative data analysis occurred automatically after each run was completed, where parameters such as signal intensity, peak area, and signal-to-noise ratio were calculated using the Jess instrument. For each individual sample, the level of progranulin was measured as the peak area of immunoreactivity to the antibody. All analyses were performed with blinded samples.

The assay described here was performed on CSF samples from a non-human primate animal study. CSF samples were tested for presence and levels of progranulin protein to study efficacy of gene therapy using an rAAV construct (PR006; see FIG. 64) encoding progranulin (PGRN) protein. In this study, either the excipient or PR006 were delivered at low dose of PR006 (1.8×10¹⁰ vg/g brain weight) or high dose of PR006 (1.8×10¹¹ vg/g brain weight) by intra-cisterna magna (ICM) injection into NHP animals. Each group consisted of 3 animals. Nine NHP animals were sacrificed at day 180 post-infection (Table 16), and CSF samples were analyzed using the Jess-based assay.

TABLE 16
NHP animal summary with grouping and dosing

| | Dose of PR006 | Number of animals |
|---|---|---|
| Group | (vg/g brain weight) | Necropsy (Day 180) |
| 1 | 0 | 2 M/1 F |
| 2 | 1.8 × 10¹⁰ | 2 M/1 F |
| 3 | 1.8 × 10¹¹ | 2 M/1 F |

TABLE 17
Materials for automated Western assay

| Material Description | Manufacturer | Item Number |
|---|---|---|
| 12-230 kDa Jess Separation | ProteinSimple | SM-W004 |
| Module, 25 capillary cartridges | | |
| EZ Standard Pack 1, 12-230 kDa | ProteinSimple | PS-ST01EZ-8 |
| Anti-mouse detection module | ProteinSimple | DM-002 |
| for Jess | | |
| Progranulin monoclonal antibody | Adipogen | AG-20A- |
| (human), clone PG359-7 (primary | | 0052-C100 |
| antibody) | | |

Note:
all reagents should be allowed to warm to room temperature prior to opening vials.

The following procedures were followed in performing this method:

### Preparation of Stock Solutions:

1. Prepare 400 mM DTT solution by adding 40 μL of water to clear tube in the separation module EZ Standard Pack. Mix gently.
2. To prepare master mix, add 20 μL of 10× sample buffer and 20 μL of 400 mM DTT into the EZ pink master mix tube. Mix gently.
3. To prepare the biotinylated ladder, Pipette 20 μL of water into the EZ clear biotinylated ladder tube with pink pellet. Mix gently.
4. Prepare luminol and peroxide mix by adding equal amounts of each. For one run, add 200 μL of luminol to 200 μL of peroxide.
5. Prepare primary antibody dilution (10 fold-dilution) by mixing 25 μL of primary antibody and 225 μL of antibody diluent 2.

### Preparation of Samples:

1. Samples are diluted in 0.1× sample buffer. Prepare 0.1× sample buffer by adding 10 μL of 10× sample buffer into 990 μL of water.
2. Dilute samples as necessary. For example, NHP CSF samples were diluted 4-fold prior to addition of master mix. Add 5 μL of NHP CSF to 15L 0.1× sample buffer.
3. Prepare samples by adding 1× of master mix to 4× of sample. To run technical duplicates, prepare a total of 15 μL of sample plus master mix per sample. For example, add 3 μL of master mix to 12 μL of diluted sample. Mix gently.
4. Boil samples at 95° C. for 5 minutes.
5. Spin down samples briefly using desktop mini-centrifuge. Vortex before loading the sample.
   Load Reagents and Samples into Cartridge:
1. Pipette all samples according to cartridge map.
   a. Pipette 15 μL of luminol+peroxide mix to each well in lane E.
   b. Pipette 10 μL of streptavidin to first well in lane D.
   c. Pipette 10 μL of secondary antibody to remaining 24 wells in lane D.
   d. Pipette 10 μL of antibody dilution to first well in lane C.
   e. Pipette 10 μL of primary antibody dilution to remaining 24 wells in lane C.
   f. Pipette 10 μL of antibody diluent to all wells in lane B.
   g. Pipette 10 μL of prepared EZ ladder to first well in lane A.
   h. Pipette 5 μL of sample and master mix solution to duplicate lanes in lane A.
2. Spin cartridge at room temperature at 2500RPM for 5 minutes.
   Load Capillaries and Cartridge into Instrument:
1. Load capillaries into slot. Make sure light turns blue.
2. Load spun cartridge into instrument.
3. Press start button after blue light stops blinking at the instrument.

The assay system suitability was considered acceptable if CV (coefficient of variance) percentage for duplicates was ≤30%.

Before the assay was used to detect progranulin in NHP CSF samples, the assay was tested as follows. Qualification of Jess assays included assessment of dilution linearity, selectivity and specificity. Normal CSF samples from BioIVT were used to determine dilution linearity of Jess assay. CSF samples from fronto-temporal dementia (FTD) patients with PGRN mutation (obtained from National Centralized Repository for Alzheimer's Disease and Related Dementias (NCRAD; Indianapolis, Indiana)) were used to determine selectivity and specificity of Jess assay.

TABLE 18
Results summary

| | Elements | Acceptance Criteria | Results |
|---|---|---|---|
| Dilution | Investigate endogenous | The MRD is defined | Pass |
| Linearity | PGRN levels in naïve | as the lowest dilution | All tested matrices |
| | CSF samples (BioIVT). | required where a linear | passed by having a |
| | Conduct an analysis of | raw signal or | linear dilution range |
| | blank sample in the | concentration is | with ±30% of the |
| | matrix. | observed. Within the | MRD (see Results |
| | Minimal required | linear range, the | and Discussion |
| | dilution (MRD) is | corrected observed | section, Dilution |
| | determined by diluting a | concentrations should | Linearity. |
| | neat matrix in 2-fold serial | be ±30% of the MRD. | |
| | dilution. | | |
| | If endogenous levels of | | |
| | PGRN are too low in | | |
| | matrix, dilutions will be | | |
| | performed using spiked | | |
| | matrix. | | |
| Selectivity and | Investigate PGRN levels | The MRD is defined | Pass |
| Specificity | in FTD patient CSF | through Dilution | All tested matrices |
| | samples. | Linearity test. | passed by having a |
| | | | % CV of technical |
| | | | replicate with 20% |
| | | | (see Results and |
| | | | Discussion section, |
| | | | Selectivity and |
| | | | Specificity. |

### Results and Discussion

### Dilution Linearity

Dilution linearity of PGRN protein detected by Jess was tested in CSF samples from commercially available (BioIVT) normal individuals. Endogenous levels of PGRN in CSF samples were measured to determine dilution linearity. Two individuals were tested in 2-fold serial dilution that ranges from 2 to 64 fold dilution.

Table 19 reported the peak area of PGRN protein at 58 kDa detected by Jess and the 00 differences of each dilution from 16-fold dilution. Results within the linearity range are in bold font (within 100±30% difference). Dilution linearity was established to be within 4 to 16 fold dilution.

TABLE 19
Dilution linearity in CSF samples

| | CSF #1 | | CSF #2 | |
|---|---|---|---|---|
| | 58 kDa | | 58 kDa | |
| | Peak Area | | Peak Area | |
| Dilution | (Dilution | | (Dilution | |
| factor | Adjusted) | % Difference | Adjusted) | % Difference |
| 1:2 | 3915099 | −41.2 | 6392991 | −38.8 |
| 1:4 | 6040885 | −9.2 | 8020821 | −23.2 |
| 1:8 | 5773987 | −13.3 | 12615004 | 20.8 |
| 1:16 | 6656474 | 0.0 | 10446186 | 0.0 |
| 1:32 | 8911479 | 33.9 | 11782404 | 12.8 |
| 1:64 | 12056943 | 81.1 | 6795118 | −35.0 |

In summary, all of the tested matrices had an acceptable linear range that passed the acceptance criteria of a % difference that is 0±30%, though the size of the range and amount of dilution varied between matrices. Sample linearity MRD was established to be 4-fold dilution. Dilution linearity was established to be within 4- to 16-fold dilution. A summary of the MIRD and linear dilution range that passes acceptance criteria for CSF is depicted in Table 20.

TABLE 20
MRD and linear dilution range of the CSF

| | Linearity | Linear Dilution |
|---|---|---|
| Tissue | MRD | Range |
| CSF | 1:4 | 1:4-1:16 |

### Selectivity and Specificity

Selectivity and specificity of PGRN protein detected by Jess were tested in CSF samples from the PR006 FTD patient samples from NCRAD. Three groups (group A, B, and C) of CSF samples were collected form heterozygous FTD patients (group A), familial non-carrier (group B or C), and normal individuals (group B or C). Six samples were analyzed for each group. The groups of samples are listed in Table 16 FTD Patient CSF sample information.

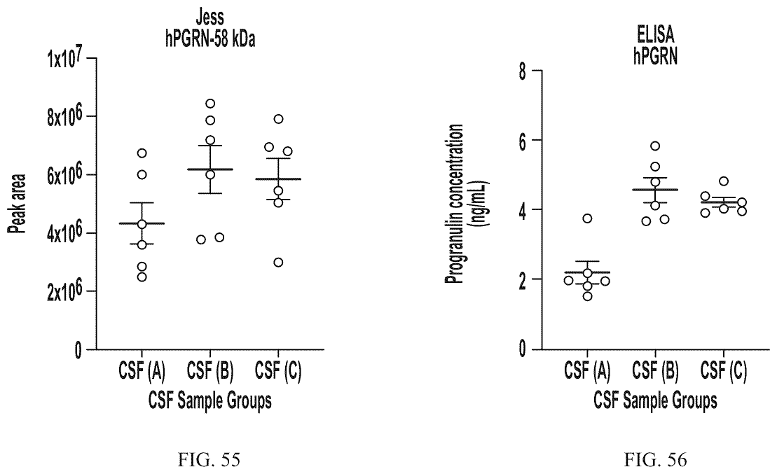

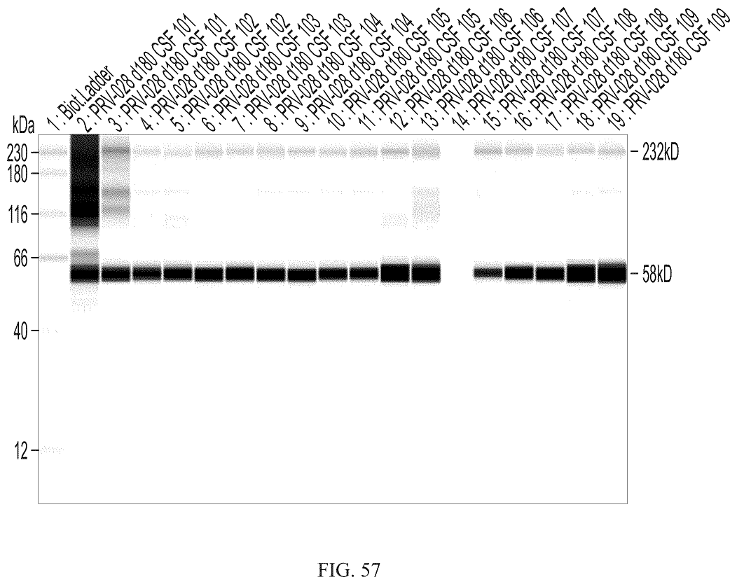

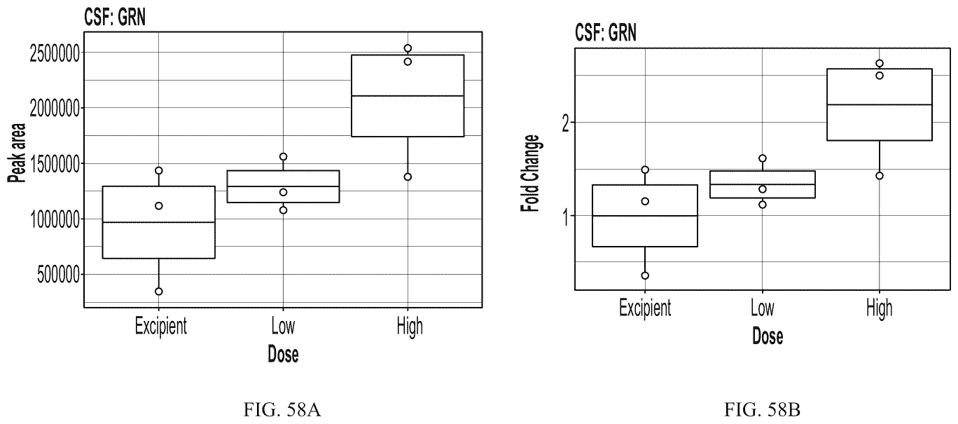

CSF samples were 4-fold diluted in 0.1× sample buffer provided by ProteinSimple and tested in technical duplicates. Samples duplicates with result % CV more than 20% were re-analyzed. Results with % CV less than 20% were reported in Table 22. Table 22 reported the peak area of PGRN protein at 58 kDa detected by Jess and the % CV between duplicates. Results showed about two fold higher of PGRN levels in group B and C as compared to group A, which indicates the selectivity and specificity of Jess assay in determine PGRN levels for CSF samples (FIG. 55).

TABLE 21
FTD patient CSF sample information

| | Alternate | | Kit | Specimen | Box | | |
|---|---|---|---|---|---|---|---|
| Barcode | MRN | Visit | Number | Type | Name | Position | Group |
| 0003355598 | ST-20000108 | Cycle 2-CSF | 257282 | CSF | 27488 | 1 | C |
| | | | | | CSF | | |
| 0004777338 | ST-20000118 | Cycle 2-CSF | 267633 | CSF | 27488 | 2 | C |
| | | | | | CSF | | |
| 0004777329 | ST-20000306 | Cycle 1-CSF | 260551 | CSF | 27488 | 3 | A |
| | | | | | CSF | | |
| 0004777326 | ST-20000328 | Cycle 2-CSF | 260544 | CSF | 27488 | 4 | C |
| | | | | | CSF | | |
| 0004777335 | ST-20000386 | Cycle 1-CSF | 267110 | CSF | 27488 | 5 | A |
| | | | | | CSF | | |
| 0004777345 | ST-20000590 | Cycle 2-CSF | 267859 | CSF | 27488 | 6 | B |
| | | | | | CSF | | |
| 0004777332 | ST-20000621 | Cycle 1-CSF | 266413 | CSF | 27488 | 7 | B |
| | | | | | CSF | | |
| 0004628923 | ST-20000757 | Cycle 1-CSF | 269817 | CSF | 27488 | 8 | B |
| | | | | | CSF | | |
| 0004695103 | ST-20001142 | Cycle 1-CSF | 308149 | CSF | 27488 | 9 | A |
| | | | | | CSF | | |
| 0004074629 | ST-20000107 | Cycle 2-CSF | 258212 | CSF | 27488 | 10 | C |
| | | | | | CSF | | |
| 0003358475 | ST-20000110 | Cycle 2-CSF | 258210 | CSF | 27488 | 11 | C |
| | | | | | CSF | | |
| 0003358463 | ST-20000274 | Cycle 2-CSF | 257292 | CSF | 27488 | 12 | A |
| | | | | | CSF | | |
| 0004788828 | ST-20000309 | Cycle 2-CSF | 303093 | CSF | 27488 | 13 | C |
| | | | | | CSF | | |
| 0003358781 | ST-20000615 | Cycle 1-CSF | 257278 | CSF | 27488 | 14 | B |
| | | | | | CSF | | |
| 0003358793 | ST-20000616 | Cycle 1-CSF | 257305 | CSF | 27488 | 15 | A |
| | | | | | CSF | | |
| 0004777321 | ST-20000637 | Cycle 1-CSF | 257307 | CSF | 27488 | 16 | B |
| | | | | | CSF | | |
| 0004777341 | ST-20000768 | Cycle 1-CSF | 267857 | CSF | 27488 | 17 | B |
| | | | | | CSF | | |
| 0004695106 | ST-20001165 | Cycle 1-CSF | 317396 | CSF | 27488 | 18 | A |
| | | | | | CSF | | |

TABLE 22
Selectivity and specificity results

| | Sample | 58 kD Peak Area | |
|---|---|---|---|
| Groups | Barcode | (Dilution Adjusted) | % CV |
| Group (A) | 0004777329 | 2838645 | 5.08 |
| Heterozygous | 0004777335 | 4293344 | 1.20 |
| FTD patients | 0004695103 | 6738165 | 1.08 |
| | 0003358463 | 3594249 | 11.10 |
| | 0003358793 | 5992434 | 2.49 |
| | 0004695106 | 2472462 | 10.40 |
| Group (B) | 0004777345 | 3836185 | 11.18 |
| Normal or | 0004777332 | 6006224 | 3.05 |
| familial non- | 0004628923 | 3758940 | 1.44 |
| carrier | 0003358781 | 7860294 | 17.08 |
| | 0004777321 | 7187172 | 0.69 |
| | 0004777341 | 8450410 | 0.50 |
| Group (C) | 0003355598 | 2981005 | 1.70 |
| Normal or | 0004777338 | 6803428 | 0.18 |
| familial non- | 0004777326 | 5030695 | 3.56 |
| carrier | 0004074629 | 5448863 | 3.47 |
| | 0003358475 | 7892529 | 1.17 |
| | 0004788828 | 6944800 | 1.85 |

CSF samples from FTD patient study (Table 21) were also analyzed with a human PGRN ELISA kit (Adipogen, AG-45A-0018YEK-KI01). Results from ELISA (FIG. 56) showed similar trends of PGRN levels between groups as Jess and demonstrated the Jess assay is suitable to use for the assessment of PGRN levels in CSF samples.

In conclusion, this ProteinSimple Automated Western Jess assay was determined to be suitable to use for the assessment of PGRN levels in NUP CSF samples.

Jess data for NHP CSF samples is shown in Table 23. Each sample represents the average across two technical replicates. The peak area for 58 kD band in the sample lane is reported. Data is presented as mean peak area of technical replicate and dilution folds adjusted.

TABLE 23
Jess data for NHP CSF samples

| | | Peak area |
|---|---|---|
| Sample ID | Dose Group | (58 kD) |
| PRV-028 d180 CSF 101 | Low dose | 4944754 |
| PRV-028 d180 CSF 102 | Control | 4449066 |
| PRV-028 d180 CSF 103 | Low dose | 6222881 |
| PRV-028 d180 CSF 104 | High dose | 5499901 |
| PRV-028 d180 CSF 105 | Low dose | 4293853 |
| PRV-028 d180 CSF 106 | High dose | 10149400 |
| PRV-028 d180 CSF 107 | Control | 1360173 |
| PRV-028 d180 CSF 108 | Control | 5742081 |
| PRV-028 d180 CSF 109 | High dose | 9658597 |

The goal of this assay was to confirm the level of progranulin (PGRN) protein expression levels following the transduction of PR006 in tissue regions of interest for the NHP study. This was done using an automated Western platform, in which progranulin protein was detected using a monoclonal antibody. Progranulin expression was measurable in CSF in both control and PR006-treated NHP; the assay does not differentiate between endogenous progranulin protein and PR006A-induced progranulin protein.

This Application incorporates by reference the contents of the following documents in their entirety: International PCT Application Publication No. WO 2019/070893; International PCT Application Publication No. WO 2019/070891; U.S. Provisional Application Ser. No. 62/567,296, filed Oct. 3, 2017, entitled “GENE THERAPIES FOR LYSOSOMAL DISORDERS”; 62/567,311, filed Oct. 3, 2017, entitled “GENE THERAPIES FOR LYSOSOMAL DISORDERS”; 62/567,319, filed Oct. 3, 2017, entitled “GENE THERAPIES FOR LYSOSOMAL DISORDERS”; 62/567,301, filed Oct. 3, 2017, entitled “GENE THERAPIES FOR LYSOSOMAL DISORDERS”; 62/567,310, filed Oct. 3, 2017, entitled “GENE THERAPIES FOR LYSOSOMAL DISORDERS”; 62/567,303, filed Oct. 3, 2017, entitled “GENE THERAPIES FOR LYSOSOMAL DISORDERS”; and 62/567,305, filed Oct. 3, 2017, entitled “GENE THERAPIES FOR LYSOSOMAL DISORDERS”.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

It should also be understood that, unless clearly indicated to the contrary, in any methods claimed herein that include more than one step or act, the order of the steps or acts of the method is not necessarily limited to the order in which the steps or acts of the method are recited.

Each of the U.S. patents, U.S. patent application publications, U.S. patent applications, foreign patents, foreign patent applications and non-patent publications referred to in this application is incorporated herein by reference, in its entirety.

### SEQUENCES

In some embodiments, an expression cassette encoding one or more gene products (e.g., a first, second and/or third gene product) comprises or consists of (or encodes a peptide having) a sequence set forth in any one of SEQ ID NOs: 1-91. In some embodiments, a gene product is encoded by a portion (e.g., fragment) of any one of SEQ ID NOs: 1-91.

### NUMBERED EMBODIMENTS

Notwithstanding the appended claims, the disclosure sets forth the following numbered embodiments:

1. An isolated nucleic acid comprising an expression construct encoding a Gcase protein flanked by two adeno-associated virus (AAV) inverted terminal repeats (ITRs), wherein
(i) at least one of the ITRs comprises a modified “D” region relative to a wild-type AAV2 ITR (SEQ ID NO: 29); and/or
(ii) the Gcase protein is encoded by a codon-optimized nucleic acid sequence.

2. The isolated nucleic acid of embodiment 1, wherein the Gcase protein comprises the amino acid sequence set forth in SEQ ID NO: 14 or a portion thereof.

3. The isolated nucleic acid of embodiment 1 or 2, wherein the Gcase protein is encoded by a codon-optimized nucleic acid sequence, optionally the nucleic acid sequence set forth in SEQ ID NO: 15.

4. The isolated nucleic acid of any one of embodiments 1 to 3, wherein the modified “D” region is a “D” sequence located on the outside of the ITR relative to the expression construct.

5. The isolated nucleic acid of any one of embodiments 1 to 4, wherein the ITR comprising the modified “D” sequence is a 3′ ITR.

6. The isolated nucleic acid of any one of embodiments 1 to 5, further comprising a TRY sequence, optionally wherein the TRY sequence is set forth in SEQ ID NO: 28.

7. An isolated nucleic acid comprising an expression construct encoding a prosaposin protein flanked by two adeno-associated virus (AAV) inverted terminal repeats (ITRs), wherein
(i) at least one of the ITRs comprises a modified “D” region relative to a wild-type AAV2 ITR (SEQ ID NO: 29); and/or
(ii) the prosaposin protein is encoded by a codon-optimized nucleic acid sequence.

8. The isolated nucleic acid of embodiment 7, wherein the prosaposin protein comprises the amino acid sequence set forth in SEQ ID NO: 16 or a portion thereof.

9. The isolated nucleic acid of embodiment 7 or 8, wherein the prosaposin protein is encoded by a codon-optimized nucleic acid sequence, optionally the nucleic acid sequence set forth in SEQ ID NO: 17.

10. The isolated nucleic acid of any one of embodiments 7 to 9, wherein the modified “D” region is a “D” sequence located on the outside of the ITR relative to the expression construct.

11. The isolated nucleic acid of any one of embodiments 7 to 10, wherein the ITR comprising the modified “D” sequence is a 3′ ITR.

12. The isolated nucleic acid of any one of embodiments 7 to 11, further comprising a TRY sequence, optionally wherein the TRY sequence is set forth in SEQ ID NO: 28.

13. An isolated nucleic acid comprising an expression construct encoding a SCARB2 protein flanked by two adeno-associated virus (AAV) inverted terminal repeats (ITRs), wherein
(i) at least one of the ITRs comprises a modified “D” region relative to a wild-type AAV2 ITR (SEQ ID NO: 29); and/or
(ii) the SCARB2 protein is encoded by a codon-optimized nucleic acid sequence.

14. The isolated nucleic acid of embodiment 13, wherein the SCARB2 protein comprises the amino acid sequence set forth in SEQ ID NO: 18 or a portion thereof.

15. The isolated nucleic acid of embodiment 13 or 14, wherein the SCARB2 protein is encoded by a codon-optimized nucleic acid sequence or the nucleic acid sequence set forth in SEQ ID NO: 19.

16. The isolated nucleic acid of any one of embodiments 13 to 15, wherein the modified “D” region is a “D” sequence located on the outside of the ITR relative to the expression construct.

17. The isolated nucleic acid of any one of embodiments 13 to 16, wherein the ITR comprising the modified “D” sequence is a 3′ ITR.

18. The isolated nucleic acid of any one of embodiments 13 to 17, further comprising a TRY sequence, optionally wherein the TRY sequence is set forth in SEQ ID NO: 28.

19. An isolated nucleic acid comprising an expression construct encoding a GBA2 protein flanked by two adeno-associated virus (AAV) inverted terminal repeats (ITRs), wherein
(i) at least one of the ITRs comprises a modified “D” region relative to a wild-type AAV2 ITR (SEQ ID NO: 29); and/or
(ii) the GBA2 protein is encoded by a codon-optimized nucleic acid sequence.

20. The isolated nucleic acid of embodiment 19, wherein the GBA2 protein comprises the amino acid sequence set forth in SEQ ID NO: 30 or a portion thereof.

21. The isolated nucleic acid of embodiment 19 or 20, wherein the GBA2 protein is encoded by a codon-optimized nucleic acid sequence or the nucleic acid sequence set forth in SEQ ID NO: 31.

22. The isolated nucleic acid of any one of embodiments 19 to 21, wherein the modified “D” region is a “D” sequence located on the outside of the ITR relative to the expression construct.

23. The isolated nucleic acid of any one of embodiments 19 to 22, wherein the ITR comprising the modified “D” sequence is a 3′ ITR.

24. The isolated nucleic acid of any one of embodiments 19 to 23, further comprising a TRY sequence, optionally wherein the TRY sequence is set forth in SEQ ID NO: 28.

25. An isolated nucleic acid comprising an expression construct encoding a GALC protein flanked by two adeno-associated virus (AAV) inverted terminal repeats (ITRs), wherein
(i) at least one of the ITRs comprises a modified “D” region relative to a wild-type AAV2 ITR (SEQ ID NO: 29); and/or
(ii) the GALC protein is encoded by a codon-optimized nucleic acid sequence.

26. The isolated nucleic acid of embodiment 25, wherein the GALC protein comprises the amino acid sequence set forth in SEQ ID NO: 33 or a portion thereof.

27. The isolated nucleic acid of embodiment 25 or 26, wherein the GALC protein is encoded by a codon-optimized nucleic acid sequence or the nucleic acid sequence set forth in SEQ ID NO: 34.

28. The isolated nucleic acid of any one of embodiments 25 to 27, wherein the modified “D” region is a “D” sequence located on the outside of the ITR relative to the expression construct.

29. The isolated nucleic acid of any one of embodiments 25 to 28, wherein the ITR comprising the modified “D” sequence is a 3′ ITR.

30. The isolated nucleic acid of any one of embodiments 25 to 29, further comprising a TRY sequence, optionally wherein the TRY sequence is set forth in SEQ ID NO: 28.

31. An isolated nucleic acid comprising an expression construct encoding a CTSB protein flanked by two adeno-associated virus (AAV) inverted terminal repeats (ITRs), wherein
(i) at least one of the ITRs comprises a modified “D” region relative to a wild-type AAV2 ITR (SEQ ID NO: 29); and/or
(ii) the CTSB protein is encoded by a codon-optimized nucleic acid sequence.

32. The isolated nucleic acid of embodiment 31, wherein the CTSB protein comprises the amino acid sequence set forth in SEQ ID NO: 30 or a portion thereof.

33. The isolated nucleic acid of embodiment 31 or 32, wherein the CTSB protein is encoded by a codon-optimized nucleic acid sequence or the nucleic acid sequence set forth in SEQ ID NO: 36.

34. The isolated nucleic acid of any one of embodiments 31 to 33, wherein the modified “D” region is a “D” sequence located on the outside of the ITR relative to the expression construct.

35. The isolated nucleic acid of any one of embodiments 31 to 34, wherein the ITR comprising the modified “D” sequence is a 3′ ITR.

36. The isolated nucleic acid of any one of embodiments 31 to 35, further comprising a TRY sequence, optionally wherein the TRY sequence is set forth in SEQ ID NO: 28.

37. An isolated nucleic acid comprising an expression construct encoding a SMPD1 protein flanked by two adeno-associated virus (AAV) inverted terminal repeats (ITRs), wherein
(i) at least one of the ITRs comprises a modified “D” region relative to a wild-type AAV2 ITR (SEQ ID NO: 29); and/or
(ii) the SMPD1 protein is encoded by a codon-optimized nucleic acid sequence.

38. The isolated nucleic acid of embodiment 37, wherein the SMPD1 protein comprises the amino acid sequence set forth in SEQ ID NO: 37 or a portion thereof.

39. The isolated nucleic acid of embodiment 37 or 38, wherein the SMPD1 protein is encoded by a codon-optimized nucleic acid sequence or the nucleic acid sequence set forth in SEQ ID NO: 38.

40. The isolated nucleic acid of any one of embodiments 37 to 39, wherein the modified “D” region is a “D” sequence located on the outside of the ITR relative to the expression construct.

41. The isolated nucleic acid of any one of embodiments 37 to 40, wherein the ITR comprising the modified “D” sequence is a 3′ ITR.

42. The isolated nucleic acid of any one of embodiments 37 to 41, further comprising a TRY sequence, optionally wherein the TRY sequence is set forth in SEQ ID NO: 28.

43. An isolated nucleic acid comprising an expression construct encoding a GCH1 protein flanked by two adeno-associated virus (AAV) inverted terminal repeats (ITRs), wherein
(i) at least one of the ITRs comprises a modified “D” region relative to a wild-type AAV2 ITR (SEQ ID NO: 29); and/or
(ii) the GCH1 protein is encoded by a codon-optimized nucleic acid sequence.

44. The isolated nucleic acid of embodiment 43, wherein the GCH1 protein comprises the amino acid sequence set forth in SEQ ID NO: 45 or a portion thereof.

45. The isolated nucleic acid of embodiment 43 or 44, wherein the GCH1 protein is encoded by a codon-optimized nucleic acid sequence or the nucleic acid sequence set forth in SEQ ID NO: 46.

46. The isolated nucleic acid of any one of embodiments 43 to 45, wherein the modified “D” region is a “D” sequence located on the outside of the ITR relative to the expression construct.

47. The isolated nucleic acid of any one of embodiments 43 to 46, wherein the ITR comprising the modified “D” sequence is a 3′ ITR.

48. The isolated nucleic acid of any one of embodiments 43 to 47, further comprising a TRY sequence, optionally wherein the TRY sequence is set forth in SEQ ID NO: 28.

49. An isolated nucleic acid comprising an expression construct encoding a RAB7L protein flanked by two adeno-associated virus (AAV) inverted terminal repeats (ITRs), wherein
(i) at least one of the ITRs comprises a modified “D” region relative to a wild-type AAV2 ITR (SEQ ID NO: 29); and/or
(ii) the RAB7L protein is encoded by a codon-optimized nucleic acid sequence.

50. The isolated nucleic acid of embodiment 49, wherein the RAB7L protein comprises the amino acid sequence set forth in SEQ ID NO: 47 or a portion thereof.

51. The isolated nucleic acid of embodiment 49 or 50, wherein the RAB7L protein is encoded by a codon-optimized nucleic acid sequence or the nucleic acid sequence set forth in SEQ ID NO: 48.

52. The isolated nucleic acid of any one of embodiments 49 to 51, wherein the modified “D” region is a “D” sequence located on the outside of the ITR relative to the expression construct.

53. The isolated nucleic acid of any one of embodiments 49 to 52, wherein the ITR comprising the modified “D” sequence is a 3′ ITR.

54. The isolated nucleic acid of any one of embodiments 49 to 53, further comprising a TRY sequence, optionally wherein the TRY sequence is set forth in SEQ ID NO: 28.

55. An isolated nucleic acid comprising an expression construct encoding a VPS35 protein flanked by two adeno-associated virus (AAV) inverted terminal repeats (ITRs), wherein
(i) at least one of the ITRs comprises a modified “D” region relative to a wild-type AAV2 ITR (SEQ ID NO: 29); and/or
(ii) the VPS35 protein is encoded by a codon-optimized nucleic acid sequence.

56. The isolated nucleic acid of embodiment 55, wherein the VPS35 protein comprises the amino acid sequence set forth in SEQ ID NO: 49 or a portion thereof.

57. The isolated nucleic acid of embodiment 55 or 56, wherein the VPS35 protein is encoded by a codon-optimized nucleic acid sequence or the nucleic acid sequence set forth in SEQ ID NO: 50.

58. The isolated nucleic acid of any one of embodiments 55 to 57, wherein the modified “D” region is a “D” sequence located on the outside of the ITR relative to the expression construct.

59. The isolated nucleic acid of any one of embodiments 55 to 58, wherein the ITR comprising the modified “D” sequence is a 3′ ITR.

60. The isolated nucleic acid of any one of embodiments 55 to 59, further comprising a TRY sequence, optionally wherein the TRY sequence is set forth in SEQ ID NO: 28.

61. An isolated nucleic acid comprising an expression construct encoding a IL-34 protein flanked by two adeno-associated virus (AAV) inverted terminal repeats (ITRs), wherein
(i) at least one of the ITRs comprises a modified “D” region relative to a wild-type AAV2 ITR (SEQ ID NO: 29); and/or
(ii) the IL-34 protein is encoded by a codon-optimized nucleic acid sequence.

62. The isolated nucleic acid of embodiment 61, wherein the IL-34 protein comprises the amino acid sequence set forth in SEQ ID NO: 55 or a portion thereof.

63. The isolated nucleic acid of embodiment 61 or 62, wherein the IL-34 protein is encoded by a codon-optimized nucleic acid sequence or the nucleic acid sequence set forth in SEQ ID NO: 56.

64. The isolated nucleic acid of any one of embodiments 61 to 63, wherein the modified “D” region is a “D” sequence located on the outside of the ITR relative to the expression construct.

65. The isolated nucleic acid of any one of embodiments 61 to 64, wherein the ITR comprising the modified “D” sequence is a 3′ ITR.

66. The isolated nucleic acid of any one of embodiments 61 to 65, further comprising a TRY sequence, optionally wherein the TRY sequence is set forth in SEQ ID NO: 28.

67. An isolated nucleic acid comprising an expression construct encoding a TREM2 protein flanked by two adeno-associated virus (AAV) inverted terminal repeats (ITRs), wherein
(i) at least one of the ITRs comprises a modified “D” region relative to a wild-type AAV2 ITR (SEQ ID NO: 29); and/or
(ii) the TREM2 protein is encoded by a codon-optimized nucleic acid sequence.

68. The isolated nucleic acid of embodiment 67, wherein the TREM2 protein comprises the amino acid sequence set forth in SEQ ID NO: 57 or a portion thereof.

69. The isolated nucleic acid of embodiment 67 or 68, wherein the TREM2 protein is encoded by a codon-optimized nucleic acid sequence or the nucleic acid sequence set forth in SEQ ID NO: 58.

70. The isolated nucleic acid of any one of embodiments 67 to 69, wherein the modified “D” region is a “D” sequence located on the outside of the ITR relative to the expression construct.

71. The isolated nucleic acid of any one of embodiments 67 to 70, wherein the ITR comprising the modified “D” sequence is a 3′ ITR.

72. The isolated nucleic acid of any one of embodiments 67 to 71, further comprising a TRY sequence, optionally wherein the TRY sequence is set forth in SEQ ID NO: 28.

73. An isolated nucleic acid comprising an expression construct encoding a TMEM106B protein flanked by two adeno-associated virus (AAV) inverted terminal repeats (ITRs), wherein
(i) at least one of the ITRs comprises a modified “D” region relative to a wild-type AAV2 ITR (SEQ ID NO: 29); and/or
(ii) the TMEM106B protein is encoded by a codon-optimized nucleic acid sequence.

74. The isolated nucleic acid of embodiment 73, wherein the TMEM106B protein comprises the amino acid sequence set forth in SEQ ID NO: 63 or a portion thereof.

75. The isolated nucleic acid of embodiment 73 or 74, wherein the TMEM106B protein is encoded by a codon-optimized nucleic acid sequence or the nucleic acid sequence set forth in SEQ ID NO: 64.

76. The isolated nucleic acid of any one of embodiments 73 to 75, wherein the modified “D” region is a “D” sequence located on the outside of the ITR relative to the expression construct.

77. The isolated nucleic acid of any one of embodiments 73 to 76, wherein the ITR comprising the modified “D” sequence is a 3′ ITR.

78. The isolated nucleic acid of any one of embodiments 73 to 77, further comprising a TRY sequence, optionally wherein the TRY sequence is set forth in SEQ ID NO: 28.

79. An isolated nucleic acid comprising an expression construct encoding a Progranulin (PGRN) protein flanked by two adeno-associated virus (AAV) inverted terminal repeats (ITRs), wherein
(i) at least one of the ITRs comprises a modified “D” region relative to a wild-type AAV2 ITR (SEQ ID NO: 29); and/or
(ii) the PGRN protein is encoded by a codon-optimized nucleic acid sequence.

80. The isolated nucleic acid of embodiment 79, wherein the PGRN protein comprises the amino acid sequence set forth in SEQ ID NO: 67 or a portion thereof.

81. The isolated nucleic acid of embodiment 79 or 80, wherein the PGRN protein is encoded by a codon-optimized nucleic acid sequence or the nucleic acid sequence set forth in SEQ ID NO: 68.

82. The isolated nucleic acid of any one of embodiments 79 to 81, wherein the modified “D” region is a “D” sequence located on the outside of the ITR relative to the expression construct.

83. The isolated nucleic acid of any one of embodiments 79 to 82, wherein the ITR comprising the modified “D” sequence is a 3′ ITR.

84. The isolated nucleic acid of any one of embodiments 79 to 83, further comprising a TRY sequence, optionally wherein the TRY sequence is set forth in SEQ ID NO: 28.

85. An isolated nucleic acid comprising an expression construct encoding a first gene product and a second gene product, wherein each gene product independently is selected from the gene products, or portions thereof, set forth in Table 1.

86. The isolated nucleic acid of embodiment 85, wherein the first gene product is a Gcase protein, or a portion thereof.

87. The isolated nucleic acid of embodiment 85 or 86, wherein the second gene product is LIMP2 or a portion thereof, or Prosaposin or a portion thereof.

88. The isolated nucleic acid of any one of embodiments 85 to 87, further encoding an interfering nucleic acid (e.g., shRNA, miRNA, dsRNA, etc.), optionally wherein the interfering nucleic acid inhibits expression of α-Syn or TMEM106B.

89. The isolated nucleic acid of any one of embodiments 85 to 88, further comprising one or more promoters, optionally wherein each of the one or more promoters is independently a chicken-beta actin (CBA) promoter, a CAG promoter, a CD68 promoter, or a JeT promoter.

90. The isolated nucleic acid of any one of embodiments 85 to 89, further comprising an internal ribosomal entry site (IRES), optionally wherein the IRES is located between the first gene product and the second gene product.

91. The isolated nucleic acid of any one of embodiments 85 to 90, further comprising a self-cleaving peptide coding sequence, optionally wherein the self-cleaving peptide is T2A.

92. The isolated nucleic acid of any one of embodiments 85 to 91, wherein the expression construct comprises two adeno-associated virus (AAV) inverted terminal repeat (ITR) sequences flanking the first gene product and the second gene product, optionally wherein one of the ITR sequences lacks a functional terminal resolution site.

93. The isolated nucleic acid of embodiment 92, wherein at least one of the ITRs comprises a modified “D” region relative to a wild-type AAV2 ITR (SEQ ID NO: 29).

94. The isolated nucleic acid of embodiment 93, wherein the modified “D” region is a “D” sequence located on the outside of the ITR relative to the expression construct.

95. The isolated nucleic acid of embodiment 93 or 94, wherein the ITR comprising the modified “D” sequence is a 3′ ITR.

96. The isolated nucleic acid of any one of embodiments 85 to 95, further comprising a TRY sequence, optionally wherein the TRY sequence is set forth in SEQ ID NO: 28.

97. An isolated nucleic acid having the sequence set forth in any one of SEQ ID NOs: 1 to 91.

98. A vector comprising the isolated nucleic acid of any one of embodiments 1 to 97.

99. The vector of embodiment 98, wherein the vector is a plasmid.

100. The vector of embodiment 98, wherein the vector is a viral vector, optionally wherein the viral vector is a recombinant AAV (rAAV) vector or a Baculovirus vector.

101. A composition comprising the isolated nucleic acid of any one of embodiments 1 to 97 or the vector of any one of embodiments 98 to 100.

102. A host cell comprising the isolated nucleic acid of any one of embodiments 1 to 97 or the vector of any one of embodiments 98 to 100.

103. A recombinant adeno-associated virus (rAAV) comprising:
(i) a capsid protein; and
(ii) the isolated nucleic acid of any one of embodiments 1 to 97, or the vector of any one of embodiments 98 to 100.

104. The rAAV of embodiment 103, wherein the capsid protein is capable of crossing the blood-brain barrier, optionally wherein the capsid protein is an AAV9 capsid protein or an AAVrh.10 capsid protein.

105. The rAAV of embodiment 103 or 104, wherein the rAAV transduces neuronal cells and non-neuronal cells of the central nervous system (CNS).

106. A method for treating a subject having or suspected of having Parkinson's disease, the method comprising administering to the subject an isolated nucleic acid of any one of embodiments 1 to 97, the vector of any one of embodiments 98 to 100, the composition of embodiment 101, or the rAAV of any one of embodiments 103 to 105.

107. The method of embodiment 106, wherein the administration comprises direct injection to the CNS of the subject, optionally wherein the direct injection is intracerebral injection, intraparenchymal injection, intrathecal injection, intra-cisterna magna injection or any combination thereof.

108. The method of embodiment 107, wherein the direct injection to the CNS of the subject comprises convection enhanced delivery (CED).

109. The method of any one of embodiments 106 to 108, wherein the administration comprises peripheral injection, optionally wherein the peripheral injection is intravenous injection.

110. A method for treating a subject having or suspected of having fronto-temporal dementia with a GRN mutation, the method comprising administering to the subject a recombinant adeno-associated virus (rAAV) comprising:
(i) a rAAV vector comprising a nucleic acid comprising an expression construct comprising a promoter operably linked to a transgene insert encoding a PGRN protein, wherein the transgene insert comprises the nucleotide sequence of SEQ ID NO: 68; and
(ii) an AAV9 capsid protein.

111. The method of embodiment 110, wherein the rAAV is administered to the subject at a dose ranging from about 1×10¹³ vector genomes (vg) to about 7×10¹⁴ vg.

112. The method of embodiment 110 or 111, wherein the rAAV is administered via an injection into the cisterna magna.

113. The method of any one of embodiments 110-112, wherein the promoter is a chicken beta actin (CBA) promoter.

114. The method of any one of embodiments 110-113, wherein the rAAV vector further comprises a cytomegalovirus (CMV) enhancer.

115. The method of any one of embodiments 110-114, wherein the rAAV vector further comprises a Woodchuck Hepatitis Virus Posttranscriptional Regulatory Element (WPRE).

116. The method of any one of embodiments 110-115, wherein the rAAV vector further comprises a Bovine Growth Hormone polyA signal tail.

117. The method of any one of embodiments 110-116, wherein the nucleic acid comprises two adeno-associated virus inverted terminal repeats (ITR) sequences flanking the expression construct.

118. The method of embodiment 117, wherein each ITR sequence is a wild-type AAV2 ITR sequence.

119. The method of any one of embodiments 110-118, wherein the rAAV vector further comprises a TRY region between the 5′ ITR and the expression construct, wherein the TRY region comprises SEQ ID NO: 28.

120. A method for treating a subject having or suspected of having fronto-temporal dementia with a GRN mutation, the method comprising administering to the subject a rAAV comprising:
(i) a rAAV vector comprising a nucleic acid comprising, in 5′ to 3′ order:
   (a) an AAV2 ITR;
   (b) a CMV enhancer;
   (c) a CBA promoter;
   (d) a transgene insert encoding a PGRN protein, wherein the transgene insert comprises the nucleotide sequence of SEQ ID NO: 68;
   (e) a WPRE;
   (f) a Bovine Growth Hormone polyA signal tail; and
   (g) an AAV2 ITR; and
(ii) an AAV9 capsid protein.

121. The method of embodiment 120, wherein the rAAV is administered to the subject at a dose ranging from about 1×10¹³ vg to about 7×10¹⁴ vg.

122. The method of embodiment 120 or 121, wherein the rAAV is administered via an injection into the cisterna magna.

123. The method of any one of embodiments 110-122, wherein the rAAV is administered in a formulation comprising about 20 mM Tris, pH 8.0, about 1 mM MgCl₂, about 200 mM NaCl, and about 0.001% w/v poloxamer 188.

124. A pharmaceutical composition comprising
(i) a rAAV comprising:
   (a) a rAAV vector comprising a nucleic acid comprising an expression construct comprising a promoter operably linked to a transgene insert encoding a PGRN protein, wherein the transgene insert comprises the nucleotide sequence of SEQ ID NO: 68; and
   (b) an AAV9 capsid protein; and
(ii) about 20 mM Tris, pH 8.0,
(iii) about 1 mM MgCl₂,
(iv) about 200 mM NaCl, and
(v) about 0.001% w/v poloxamer 188.

125. A rAAV comprising:
(a) a rAAV vector comprising a nucleic acid comprising an expression construct comprising a promoter operably linked to a transgene insert encoding a PGRN protein, wherein the transgene insert comprises the nucleotide sequence of SEQ ID NO: 68; and
(b) an AAV9 capsid protein, for use in a method of treating fronto-temporal dementia with a GRN mutation in a subject.

126. A method of quantifying a PGRN protein level in a cerebrospinal fluid (CSF) sample, the method comprising:
(1) diluting the CSF sample in a master mix containing dithiothreitol (DTT) and sample buffer;
(2) loading the diluted CSF sample, an anti-progranulin antibody, a secondary antibody that detects the anti-progranulin antibody, luminol and peroxide into wells of a capillary cartridge;
(3) loading the capillary cartridge into an automated Western blot immunoassay instrument;
(4) using the automated Western blot immunoassay instrument to calculate signal intensity, peak area, and signal-to-noise ratio; and
(5) quantifying a progranulin protein level in the CSF sample as the peak area of immunoreactivity to the anti-progranulin antibody.

What is claimed is:

1. A pharmaceutical composition comprising:
(i) a recombinant adeno-associated virus (rAAV) comprising:
(a) a rAAV vector comprising a nucleic acid comprising an expression construct comprising a promoter operably linked to a transgene insert encoding a progranulin (PGRN) protein, wherein the transgene insert comprises the nucleotide sequence of SEQ ID NO: 68; and
(b) an adeno-associated virus (AAV) 9 capsid protein;
(ii) about 20 mM Tris, pH 8.0,
(iii) about 1 mM MgCl₂,
(iv) about 200 mM NaCl, and
(v) about 0.001% w/v poloxamer 188.

2. The pharmaceutical composition of claim 1, wherein the promoter is a chicken beta actin (CBA) promoter.

3. The pharmaceutical composition of claim 1, wherein the rAAV vector further comprises a cytomegalovirus (CMV) enhancer.

4. The pharmaceutical composition of claim 1, wherein the rAAV vector further comprises a Woodchuck Hepatitis Virus Posttranscriptional Regulatory Element (WPRE).

5. The pharmaceutical composition of claim 1, wherein the rAAV vector further comprises a Bovine Growth Hormone polyA signal tail.

6. The pharmaceutical composition of claim 1, wherein the nucleic acid comprises two adeno-associated virus inverted terminal repeat (ITR) sequences flanking the expression construct, wherein the first ITR sequence is a 5′ ITR, and the second ITR sequence is a 3′ ITR.

7. The pharmaceutical composition of claim 6, wherein each of the two ITR sequences is a wild-type AAV2 ITR sequence.

8. The pharmaceutical composition of claim 6, wherein the rAAV vector further comprises a TRY region between the 5′ ITR and the expression construct, wherein the TRY region comprises SEQ ID NO: 28.

9. A pharmaceutical composition comprising:
(i) a recombinant adeno-associated virus (rAAV) comprising:
(a) a rAAV vector comprising a nucleic acid comprising, in 5′ to 3′ order:
an AAV2 inverted terminal repeat (ITR);
a cytomegalovirus (CMV) enhancer;
a chicken beta actin (CBA) promoter;
a transgene insert encoding a progranulin (PGRN) protein, wherein the transgene insert comprises the nucleotide sequence of SEQ ID NO: 68;
a Woodchuck Hepatitis Virus Posttranscriptional Regulatory Element (WPRE);
a Bovine Growth Hormone polyA signal tail; and
an AAV2 ITR; and
(b) an adeno-associated virus (AAV) 9 capsid protein;
(ii) about 20 mM Tris, pH 8.0,
(iii) about 1 mM MgCl₂,
(iv) about 200 mM NaCl, and
(v) about 0.001% w/v poloxamer 188.
